SEQUENCE LISTING (35 sequences)

SEQ ID NO: 1 - protein, 450 aa, synthetic construct
  REGION 1..450: note = Heavy chain of anti-HER2 antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
  REGION 1..214: note = Light chain of anti-HER2 antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 447 aa, synthetic construct
  REGION 1..447: note = Heavy chain of anti-HER3 antibody
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 220 aa, synthetic construct
  REGION 1..220: note = Light chain of anti-HER3 antibody
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 470 aa, synthetic construct
  REGION 1..470: note = Heavy chain of anti-TROP2 antibody
MKHLWFFLLLVAAPRWVLSQVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 234 aa, synthetic construct
  REGION 1..234: note = Light chain of anti-TROP2 antibody
MVLQTQVFISLLLWISGAYGDIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 471 aa, synthetic construct
  REGION 1..471: note = Heavy chain of anti-B7-H3 antibody
MKHLWFFLLLVAAPRWVLSQVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 233 aa, synthetic construct
  REGION 1..233: note = Light chain of anti-B7-H3 antibody
MVLQTQVFISLLLWISGAYGEIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 472 aa, synthetic construct
  REGION 1..472: note = Heavy chain of anti-GPR20 antibody
MKHLWFFLLLVAAPRWVLSEVQLVQSGAEVKKPGASVKVSCKASGYTFTSYYISWIRQAPGQGLKYMGFINPGSGHTNYNEKFKGRVTITADKSSSTATMELSSLRSEDTAVYYCARGAGGFLRIITKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 234 aa, synthetic construct
  REGION 1..234: note = Light chain of anti-GPR20 antibody
MVLQTQVFISLLLWISGAYGDTQLTQSPSSLSASVGDRVTITCRASKSVSTYIHWYQQKPGKQPKLLIYSAGNLESGVPSRFSGSGSGTDFTLTISSLQPEDFANYYCQQINELPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 471 aa, synthetic construct
  REGION 1..471: note = Heavy chain of anti-CDH6 antibody
MKHLWFFLLLVAAPRWVLSEVQLVQSGAEVKKPGASVKVSCKASGYTFTRNFMHWVRQAPGQGLEWMGWIYPGDGETEYAQKFQGRVTITADTSTSTAYMELSSLRSEDTAVYYCARGVYGGFAGGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 233 aa, synthetic construct
  REGION 1..233: note = Light chain of anti-CDH6 antibody
MVLQTQVFISLLLWISGAYGDIQMTQSPSSLSASVGDRVTITCKASQNIYKNLAWYQQKPGKAPKLLIYDANTLQTGVPSRFSGSGSGSDFTLTISSLQPEDFATYFCQQYYSGWAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 447 aa, synthetic construct
  REGION 1..447: note = heavy chain
EVQLVESGGGLVQPGGSMRLSCVASGFPFSNYWMNWVRQAPGKGLEWVGEIRLKSNQYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 14 - protein, 447 aa, synthetic construct
  REGION 1..447: note = heavy chain
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 15 - protein, 219 aa, synthetic construct
  REGION 1..219: note = light chain
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 468 aa, synthetic construct
  REGION 1..468: note = Heavy chain of an anti-CD37 antibody (hmAb-H541)
MKHLWFFLLLVAAPRWVLSEVQLVQSGAEVKKPGASVKVSCKASGYSFTDYNMYWVRQAPGQSLEWMGYIDPYNGDTTYNQKFQGRVTMTRDTSISTAYMELSRLRSDDTAVYYCARSPYGHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 468 aa, synthetic construct
  REGION 1..468: note = Heavy chain of an anti-CD37 antibody (hmAb-H551)
MKHLWFFLLLVAAPRWVLSEVQLVQSGAEVKKPGASVKVSCKASGYSFTDYNMYWVRQAPGQSLEWMGYIDPYNGDTTYNQKFQGRVTMTRDTSSSTAYMELSRLRSDDTAVYYCARSPYGHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 468 aa, synthetic construct
  REGION 1..468: note = Heavy chain of an anti-CD37 antibody (hmAb-H11a)
MKHLWFFLLLVAAPRWVLSEVQLVQSGAEVKKPGASVKVSCKASGYSFTDYNMYWVRQAPGQSLEWMGYIDPYNGDTTYNQKFQGRVTITADKSKSTAYMELSSLRSEDTAVYYCARSPYGHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 234 aa, synthetic construct
  REGION 1..234: note = Light chain of the anti-CD37 antibodies (hmAb-L11)
MVLQTQVFISLLLWISGAYGDIQMTQSPSSLSASVGDRVTITCKASQDVSTAVDWYQQKPGKAPKLLINWASTRHTGVPSRFSGSGSGTDFTLTISSLQPEDFATYYCRQHYSTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 8 aa, synthetic construct
  REGION 1..8: note = CDRH1 of heavy chain of anti-HER2 antibody
GFNIKDTY

SEQ ID NO: 21 - protein, 8 aa, synthetic construct
  REGION 1..8: note = CDRH2 of heavy chain of anti-HER2 antibody
IYPTNGYT

SEQ ID NO: 22 - protein, 13 aa, synthetic construct
  REGION 1..13: note = CDRH3 of heavy chain of anti-HER2 antibody
SRWGGDGFYAMDY

SEQ ID NO: 23 - protein, 6 aa, synthetic construct
  REGION 1..6: note = CDRL1 of light chain of anti-HER2 antibody
QDVNTA

SEQ ID NO: 24 - skipped

SEQ ID NO: 25 - protein, 9 aa, synthetic construct
  REGION 1..9: note = CDRL3 of light chain of anti-HER2 antibody
QQHYTTPPT

SEQ ID NO: 26 - protein, 120 aa, synthetic construct [sequence omitted]

SEQ ID NO: 27 - protein, 107 aa, synthetic construct [sequence omitted]

SEQ ID NO: 28 - protein, 5 aa, synthetic construct
  REGION 1..5: note = CDRH1 of heavy chain of anti-TROP2 antibody
TAGMQ

SEQ ID NO: 29 - protein, 17 aa, synthetic construct
  REGION 1..17: note = CDRH2 of heavy chain of anti-TROP2 antibody
WINTHSGVPKYAEDFKG

SEQ ID NO: 30 - protein, 12 aa, synthetic construct
  REGION 1..12: note = CDRH3 of heavy chain of anti-TROP2 antibody
SGFGSSYWYFDV

SEQ ID NO: 31 - protein, 11 aa, synthetic construct
  REGION 1..11: note = CDRL1 of light chain of anti-TROP2 antibody
KASQDVSTAVA

SEQ ID NO: 32 - protein, 7 aa, synthetic construct
  REGION 1..7: note = CDRL2 of light chain of anti-TROP2 antibody
SASYRYT

SEQ ID NO: 33 - protein, 9 aa, synthetic construct
  REGION 1..9: note = CDRL3 of light chain of anti-TROP2 antibody
QQHYITPLT

SEQ ID NO: 34 - protein, 121 aa, synthetic construct
  REGION 1..121: note = Heavy chain variable region of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 35 - protein, 109 aa, synthetic construct
  REGION 1..109: note = Light chain variable region of anti-TROP2 antibody
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRT

(19) United States
(12) Patent Application Publication
(10) Pub. No.: US 2025/0276079 A1
(43) Pub. Date: Sep. 4, 2025

(54) COMBINATION OF ANTIBODY-DRUG CONJUGATE WITH EZH1 and/or EZH2 INHIBITOR

(71) Applicants: DAIICHI SANKYO COMPANY, LIMITED, Tokyo (JP); AstraZeneca UK Limited, Cambridge (GB)

(72) Inventors: Daisuke HONMA, Tokyo (JP); Yasuki KAMAI, Tokyo (JP)

(73) Assignees: DAIICHI SANKYO COMPANY, LIMITED, Tokyo (JP); AstraZeneca UK Limited, Cambridge (GB)

(21) Appl. No.: 18/859,141

(22) PCT Filed: Apr. 26, 2023
(86) PCT No.: PCT/IB2023/054298

§ 371 Date: Oct. 22, 2024

Related U.S. Application Data
(60) Provisional application No. 63/335,558, filed on Apr. 27, 2022.

(51) Int. Cl. A61K 47/68; A61K 31/443; A61P 35/00

(52) U.S. Cl. CPC A61K 47/68037; A61K 31/443; A61K 47/6851; A61K 47/6889; A61P 35/00

(57) ABSTRACT

A pharmaceutical product, wherein an antibody-drug conjugate in which a drug-linker represented by the following formula (I) (wherein A represents a connecting position to an antibody) is conjugated to the antibody via a thioether bond, and an inhibitor selected from the group consisting of an EZH1 inhibitor, an EZH2 inhibitor and an EZH1/2 dual inhibitor are administered in combination, and/or a method of treatment, wherein the antibody-drug conjugate and the inhibitor are administrated in combination to a subject.

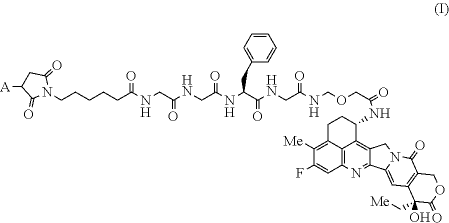

### TECHNICAL FIELD

The present invention relates to a pharmaceutical product, wherein a specific antibody-drug conjugate and an inhibitor selected from the group consisting of an EZH1 inhibitor, an EZH2 inhibitor and an EZH1/2 dual inhibitor are administered in combination, and/or a method of treatment, wherein a specific antibody-drug conjugate and an inhibitor selected from the group consisting of an EZH1 inhibitor, an EZH2 inhibitor and an EZH1/2 dual inhibitor are administered in combination to a subject.

### BACKGROUND ART

The polycomb family negatively regulates gene expression through chromatin control mediated by histone modification. Enhancer of zeste homologue 1/2 (EZH1/2) is an active center of polycomb repressive complex 2 (PRC2), which tri-methylates histone H3K27. EZH1 and EZH2 mutually compensate each other's functions and maintain an epigenome within a cell. Inhibition of EZH2 reduces the methylation level at H3K27 of a whole cell; however, the effect is limited by the compensation effect of EZH1. If EZH1 and EZH2 are simultaneously inhibited, methylation is more effectively eliminated (Non-Patent Reference 1). Abnormalities of components of PRC2 cause cancer and functional abnormalities of stem cells. Particularly, abnormalities of the EZH2 gene and elevated expression thereof induce accumulation of methylated H3K27me3, which is identified in many cancers. Studies have been aggressively conducted focusing on EZH2 as a new molecular target for cancer (Non-Patent References 2, 3).

As an example of the compounds having EZH1 and/or EZH2 inhibition activity, EZH1/2 dual inhibitor, (2R)-7-chloro-2-[trans-4-(dimethylamino)cyclohexyl]-N-[(4,6-dimethyl-2-oxo-1,2-dihydropyridin-3-yl)methyl]-2,4-dimethyl-1,3-benzodioxole-5-carboxamide and a pharmaceutically acceptable salt thereof is known (Patent Reference 1).

Schlafen 11 (SLFN11), a putative DNA/RNA helicase that is recruited to the stressed replication fork and irreversibly triggers replication block and cell death, has emerged as a promising predictor of sensitivity to cytotoxic chemotherapies, specifically DNA-damaging agents (DDA), such as topoisomerase (TOP) I and TOP II (irinotecan and etoposide, respectively), DNA synthesis inhibitors (e.g. gemcitabine) and DNA cross-linkers and alkylating agents (e.g. cisplatin) (Non-Patent Reference 4).

One study found that EZH1/2 inhibitors could reverse an epigenetic mechanism of acquired chemoresistance caused by epigenetic silencing of SLFN11. In this context, inhibition of EZH1/2 could restore SLFN11 expression and synergize with a variety of DNA damaging agents in vitro and in vivo (Non-Patent Reference 5).

In terms of cancer therapy, it is known that therapies using a plurality of anticancer agents in combination are effective, and various studies (on combination therapy) have been actively conducted. For example, a patent application discloses that a combination drug containing an EZH1/2 dual inhibitor and another medical agent in combination exerts an excellent anticancer effect (Patent Reference 12). Moreover, a phase I and II trial evaluating the safety and tolerability of valemetostat (DS-3201b), a potent dual EZH1/2 inhibitor, in combination with irinotecan in patients with recurrent SCLC (NCT03879798) is ongoing.

An antibody-drug conjugate (ADC) having a drug with cytotoxicity conjugated to an antibody capable of binding to an antigen expressed on the surface of cancer cells and cellular internalization, can deliver the drug selectively to cancer cells and can thus be expected to cause accumulation of the drug within cancer cells and to kill the cancer cells (Non-Patent References 6 to 10).

As one such antibody-drug conjugate, an antibody-drug conjugate comprising an antibody and a derivative of exatecan, which is a topoisomerase I inhibitor, as its components is known (Patent References 2 to 10, Non-Patent References 11 to 15). Moreover, a patent application discloses that the expression amounts of the hTROP2 gene and the SLFN11 gene at mRNA level in combination makes it possible to identify a subject to be given a medicament containing an anti-hTROP2 antibody more accurately (Patent Reference 11).

However, none of the references describes any test result showing a combined effect of the foregoing antibody-drug conjugate and an EZH1 and/or EZH2 inhibitor, or any scientific basis for suggesting such a test result.

### CITATION LIST

### Patent Literature

Patent Reference 1: International Publication No. WO2015/141616
Patent Reference 2: International Publication No. WO 2014/057687
Patent Reference 3: International Publication No. WO 2014/061277
Patent Reference 4: International Publication No. WO 2015/098099
Patent Reference 5: International Publication No. WO 2015/115091
Patent Reference 6: International Publication No. WO 2015/146132
Patent Reference 7: International Publication No. WO 2015/155976
Patent Reference 8: International Publication No. WO 2015/155998
Patent Reference 9: International Publication No. WO 2018/135501
Patent Reference 10: International Publication No. WO 2018/212136
Patent Reference 11: International Publication No. WO2020/040245
Patent Reference 12: International Publication No. WO2020/111234

### Non-Patent Literature

Non-Patent Reference 1: Shen, X et al., Mol Cell 2008; 32(4): 491-502.
Non-Patent Reference 2: Sparmann A, van Lohuizen M., Nat Rev Cancer 2006; 6: 846.
Non-Patent Reference 3: Lund, Adams, Copland., Leukemia 2014; 28(1): 44-9.
Non-Patent Reference 4: Coleman, N., et al., British Journal of Cancer. (2021) 124: 857-859
Non-Patent Reference 5: Poirier, J. T., et. Al., Journal of Thoracic Oncology (2020) 15, 4: 520-540
Non-Patent Reference 6: Ducry, L., et al., Bioconjugate Chem. (2010) 21, 5-13.
Non-Patent Reference 7: Alley, S. C., et al., Current Opinion in Chemical Biology (2010) 14, 529-537.
Non-Patent Reference 8: Damle N. K. Expert Opin. Biol. Ther. (2004) 4, 1445-1452.
Non-Patent Reference 9: Senter P. D., et al., Nature Biotechnology (2012) 30, 631-637.
Non-Patent Reference 10: Burris H A et al., J. Clin. Oncol. (2011) 29(4): 398-405.
Non-Patent Reference 11: Ogitani Y. et al., Clinical Cancer Research (2016) 22 (20), 5097-5108.
Non-Patent Reference 12: Ogitani Y. et al., Cancer Science (2016) 107, 1039-1046.
Non-Patent Reference 13: Doi T, et al., Lancet Oncol. (2017) 18, 1512-22.
Non-Patent Reference 14: Takegawa N, et al., Int. J. Cancer (2017) 141, 1682-1689.
Non-Patent Reference 15: Yonesaka K, et al., Oncogene (2019) 38: 1398-1409.

### SUMMARY OF INVENTION

### Technical Problem

The antibody-drug conjugates used in the present invention (antibody-drug conjugates containing an exatecan derivative as a component) have been confirmed to exert a superior antitumor effect even as a single agent. However, there has been a demand for obtaining a method of treatment which can suppress growth of cancer cells in multiple manners and exert a further superior antitumor effect by using the antibody-drug conjugate in combination with another anticancer agent having a different mechanism of action.

An object of the present invention is to provide a pharmaceutical product for administration of a specific antibody-drug conjugate in combination with an inhibitor selected from the group consisting of an EZH1 inhibitor, an EZH2 inhibitor and an EZH1/2 dual inhibitor, and/or a therapeutic use and method, wherein the specific antibody-drug conjugate and an inhibitor selected from the group consisting of an EZH1 inhibitor, an EZH2 inhibitor and an EZH1/2 dual inhibitor are administered in combination to a subject.

### Solution to Problem

As a result of diligent studies in order to solve the above problems, the present inventors have found that combined administration of a specific antibody-drug conjugate and an inhibitor selected from the group consisting of an EZH1 inhibitor, an EZH2 inhibitor and an EZH1/2 dual inhibitor exhibits a superior combined effect, and thereby completed the present invention.

Thus, the present invention provides the following [1] to [261].

[1] A pharmaceutical product comprising an antibody-drug conjugate and an inhibitor selected from the group consisting of an EZH1 inhibitor, an EZH2 inhibitor and an EZH1/2 dual inhibitor for administration in combination, wherein
the antibody-drug conjugate is an antibody-drug conjugate in which a drug-linker represented by the following formula:

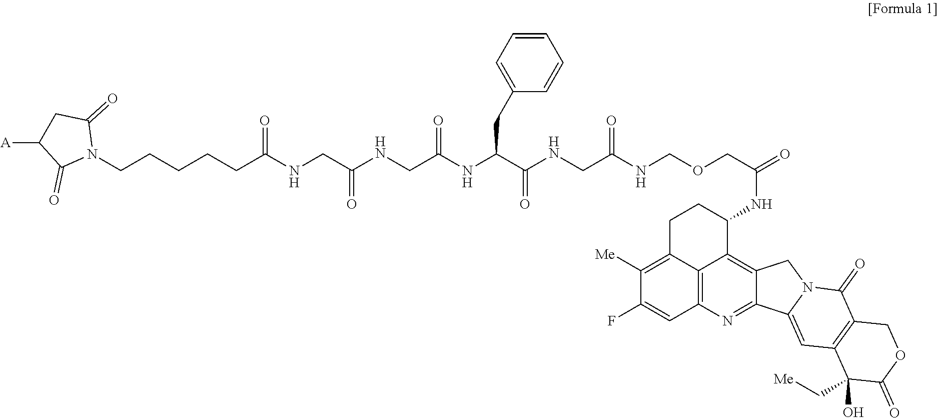

wherein A represents a connecting position to an antibody,
is conjugated to the antibody via a thioether bond.

[2] The pharmaceutical product according to [1], wherein the antibody in the antibody-drug conjugate is an anti-HER2 antibody, an anti-HER3 antibody, an anti-TROP2 antibody, an anti-B7-H3 antibody, an anti-GPR20 antibody, an anti-CDH6 antibody, or an anti-MUC1 antibody.

[3] The pharmaceutical product according to [2], wherein the antibody in the antibody-drug conjugate is an anti-HER2 antibody.

[4] The pharmaceutical product according to [3], wherein the anti-HER2 antibody is an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1, and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2, and CDRL3 consisting of an amino acid sequence consisting of amino acid residues 89 to 97 of SEQ ID NO: 2.

[5] The pharmaceutical product according to [3], wherein the anti-HER2 antibody is an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2.

[6] The pharmaceutical product according to [3], wherein the anti-HER2 antibody is an antibody comprising a heavy chain consisting of an amino acid sequence represented by SEQ ID NO: 1 and a light chain consisting of an amino acid sequence represented by SEQ ID NO: 2.

[7] The pharmaceutical product according to [3], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

[8] The pharmaceutical product according to any one of [3] to [7], wherein the antibody-drug conjugate is represented by the following formula:

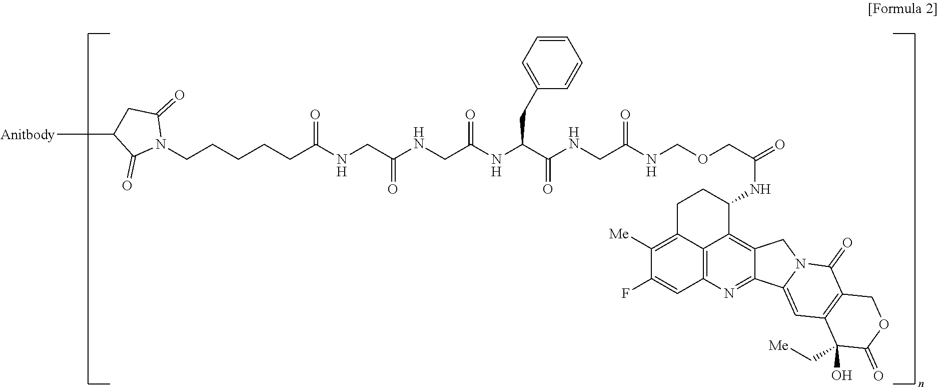

wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8.

[9] The pharmaceutical product according to [2], wherein the antibody in the antibody-drug conjugate is an anti-HER3 antibody.

[10] The pharmaceutical product according to [9], wherein the anti-HER3 antibody is an antibody comprising a heavy chain consisting of an amino acid sequence represented by SEQ ID NO: 3 and a light chain consisting of an amino acid sequence represented by SEQ ID NO: 4.

[11] The pharmaceutical product according to [10], wherein the anti-HER3 antibody lacks a lysine residue at the carboxyl terminus of the heavy chain.

[12] The pharmaceutical product according to any one of [9] to [11], wherein the antibody-drug conjugate is represented by the following formula:

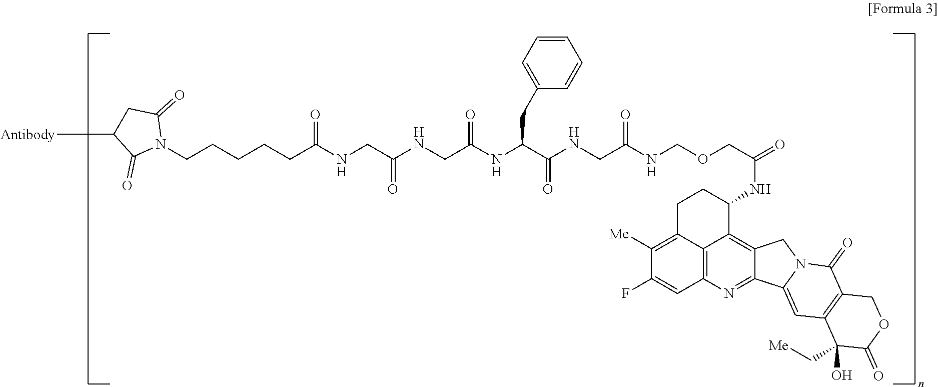

wherein ‘Antibody’ indicates the anti-HER3 antibody conjugated to the drug-linker via a thioether bond, and n indicates an average number of units of the drug-linker conjugated per antibody molecule in the antibody-drug conjugate, wherein n is in the range of from 7 to 8.

[13] The pharmaceutical product according to [2], wherein the antibody in the antibody-drug conjugate is an anti-TROP2 antibody.

[14] The pharmaceutical product according to [13], wherein the anti-TROP2 antibody is an antibody comprising a heavy chain comprising CDRH1 consisting of an amino acid sequence consisting of amino acid residues 50 to 54 of SEQ ID NO: 5, CDRH2 consisting of an amino acid sequence consisting of amino acid residues 69 to 85 of SEQ ID NO: 5, and CDRH3 consisting of an amino acid sequence consisting of amino acid residues 118 to 129 of SEQ ID NO: 5, and a light chain comprising CDRL1 consisting of an amino acid sequence consisting of amino acid residues 44 to 54 of SEQ ID NO: 6, CDRL2 consisting of an amino acid sequence consisting of amino acid residues 70 to 76 of SEQ ID NO: 6, and CDRL3 consisting of an amino acid sequence consisting of amino acid residues 109 to 117 of SEQ ID NO: 6.

[15] The pharmaceutical product according to [13], wherein the anti-TROP2 antibody is an antibody comprising a heavy chain comprising a heavy chain variable region consisting of an amino acid sequence consisting of amino acid residues 20 to 140 of SEQ ID NO: 5, and a light chain comprising a light chain variable region consisting of an amino acid sequence consisting of amino acid residues 21 to 129 of SEQ ID NO: 6.

[16] The pharmaceutical product according to [13], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

[17] The pharmaceutical product according to [16], wherein the anti-TROP2 antibody lacks a lysine residue at the carboxyl terminus of the heavy chain.

[18] The pharmaceutical product according to any one of [13] to [17], wherein the antibody-drug conjugate is represented by the following formula:

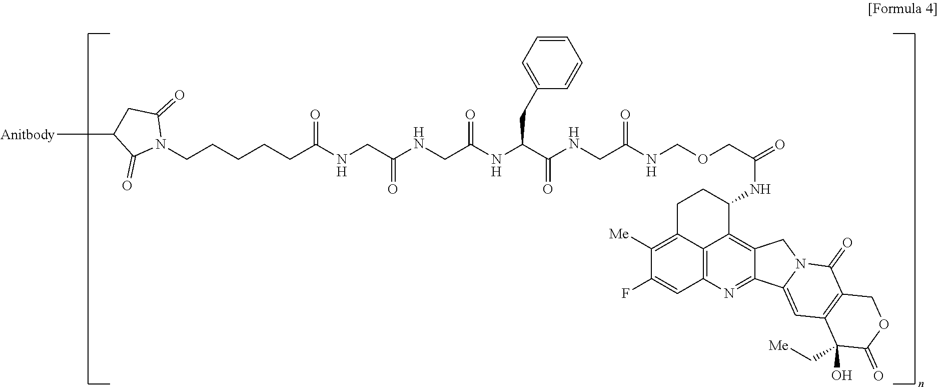

wherein ‘Antibody’ indicates the anti-TROP2 antibody conjugated to the drug-linker via a thioether bond, and n indicates an average number of units of the drug-linker conjugated per antibody molecule in the antibody-drug conjugate, wherein n is in the range of from 3.5 to 4.5.

[19] The pharmaceutical product according to [2], wherein the antibody in the antibody-drug conjugate is an anti-B7-H3 antibody.

[20] The pharmaceutical product according to [19], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

[21] The pharmaceutical product according to [20], wherein the anti-B7-H3 antibody lacks a lysine residue at the carboxyl terminus of the heavy chain.

[22] The pharmaceutical product according to any one of [19] to [21], wherein the antibody-drug conjugate is represented by the following formula:

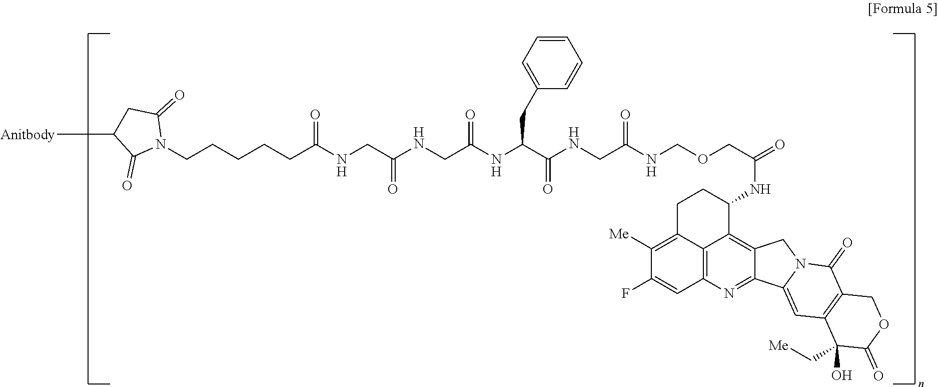

wherein ‘Antibody’ indicates the anti-B7-H3 antibody conjugated to the drug-linker via a thioether bond, and n indicates an average number of units of the drug-linker conjugated per antibody molecule in the antibody-drug conjugate, wherein n is in the range of from 3.5 to 4.5.

[23] The pharmaceutical product according to [2], wherein the antibody in the antibody-drug conjugate is an anti-GPR20 antibody.

[24] The pharmaceutical product according to [23], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

[25] The pharmaceutical product according to [24], wherein the anti-GPR20 antibody lacks a lysine residue at the carboxyl terminus of the heavy chain.

[26] The pharmaceutical product according to any one of [23] to [25], wherein the antibody-drug conjugate is represented by the following formula:

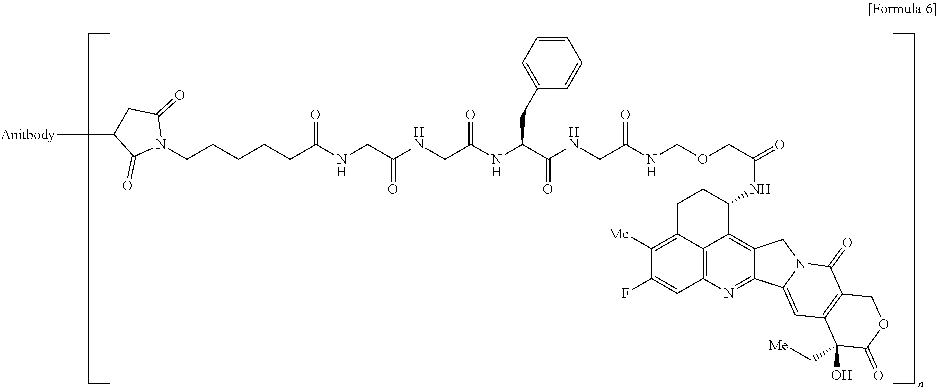

wherein ‘Antibody’ indicates the anti-GPR20 antibody conjugated to the drug-linker via a thioether bond, and n indicates an average number of units of the drug-linker conjugated per antibody molecule in the antibody-drug conjugate, wherein n is in the range of from 7 to 8.

[27] The pharmaceutical product according to [2], wherein the antibody in the antibody-drug conjugate is an anti-CDH6 antibody.

[28] The pharmaceutical product according to [27], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

[29] The pharmaceutical product according to [28], wherein the anti-CDH6 antibody lacks a lysine residue at the carboxyl terminus of the heavy chain.

[30] The pharmaceutical product according to any one of [27] to [29], wherein the antibody-drug conjugate is represented by the following formula:

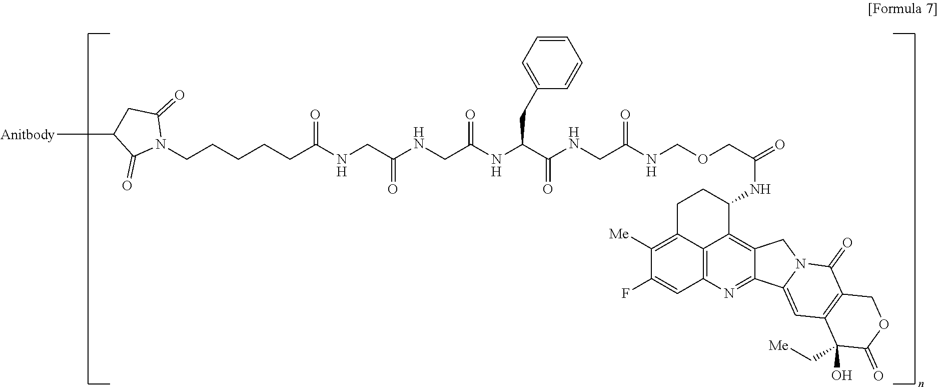

wherein ‘Antibody’ indicates the anti-CDH6 antibody conjugated to the drug-linker via a thioether bond, and n indicates an average number of units of the drug-linker conjugated per antibody molecule in the antibody-drug conjugate, wherein n is in the range of from 7 to 8.

[31] The pharmaceutical product according to [2], wherein the antibody in the antibody-drug conjugate is an anti-MUC1 antibody.

[32] The pharmaceutical product according to [31], wherein the anti-MUC1 antibody is an antibody comprising a heavy chain consisting of an amino acid sequence represented by SEQ ID NO: 13 and a light chain consisting of an amino acid sequence represented by SEQ ID NO: 15.

[33] The pharmaceutical product according to [32], wherein the anti-MUC1 antibody lacks a lysine residue at the carboxyl terminus of the heavy chain.

[34] The pharmaceutical product according to [31], wherein the anti-MUC1 antibody is an antibody comprising a heavy chain consisting of an amino acid sequence represented by SEQ ID NO: 14 and a light chain consisting of an amino acid sequence represented by SEQ ID NO: 15.

[35] The pharmaceutical product according to [34], wherein the anti-MUC1 antibody lacks a lysine residue at the carboxyl terminus of the heavy chain.

[36] The pharmaceutical product according to any one of [31] to [35], wherein the antibody-drug conjugate is represented by the following formula:

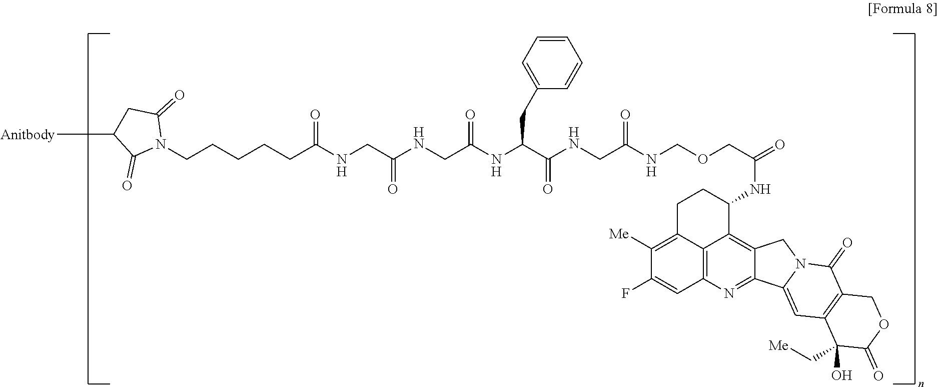

wherein ‘Antibody’ indicates the anti-MUC1 antibody conjugated to the drug-linker via a thioether bond, and n indicates an average number of units of the drug-linker conjugated per antibody molecule in the antibody-drug conjugate, wherein n is in the range of from 7 to 8.

[37] The pharmaceutical product according to any one of [1] to [8], wherein the antibody-drug conjugate is trastuzumab deruxtecan (DS-8201a).

[38] The pharmaceutical product according to any one of [1] to [2] and [13] to [18], wherein the antibody-drug conjugate is datopotamab deruxtecan (DS-1062).

[39] The pharmaceutical product according to any one of [1] to [38], wherein the inhibitor is an EZH2 inhibitor.

[40] The pharmaceutical product according to [39], wherein the inhibitor is tazemetostat or a pharmaceutically acceptable salt thereof.

[41] The pharmaceutical product according to any one of [1] to [38], wherein the inhibitor is an EZH1/2 dual inhibitor.

[42] The pharmaceutical product according to [41], wherein the inhibitor is valemetostat or a pharmaceutically acceptable salt thereof.

[43] The pharmaceutical product according to [41] or [42], wherein the inhibitor is valemetostat tosylate.

[44] The pharmaceutical product according to any one of [1] to [43], wherein the antibody-drug conjugate and the inhibitor are separately contained as active components in different formulations, and are administered simultaneously or at different times.

[45] The pharmaceutical product according to any one of [1] to [44] and [257], wherein the pharmaceutical product is for use in treating at least one selected from the group consisting of breast cancer, gastric cancer, colorectal cancer, lung cancer, esophageal cancer, head-and-neck cancer, gastroesophageal junction adenocarcinoma, biliary tract cancer, Paget's disease, pancreatic cancer, ovarian cancer, bladder cancer, prostate cancer, uterine carcinosarcoma, gastrointestinal stromal tumor, kidney cancer, and sarcoma.

[46] The pharmaceutical product according to [45], wherein the pharmaceutical product is for use in treating breast cancer.

[47] The pharmaceutical product according to [46], wherein the pharmaceutical product is for use in treating triple-negative breast cancer.

[48] The pharmaceutical product according to [45], wherein the pharmaceutical product is for use in treating gastric cancer.

[49] The pharmaceutical product according to [45], wherein the pharmaceutical product is for use in treating ovarian cancer.

[50] The pharmaceutical product according to [45], wherein the pharmaceutical product is for use in treating lung cancer.

[51] The pharmaceutical product according to [45], wherein the pharmaceutical product is for use in treating pancreatic cancer.

[52] A method of treatment, comprising administering an antibody-drug conjugate and an inhibitor selected from the group consisting of an EZH1 inhibitor, an EZH2 inhibitor and an EZH1/2 dual inhibitor in combination to a subject in need of the treatment, wherein the antibody-drug conjugate is an antibody-drug conjugate in which a drug-linker represented by the following formula:

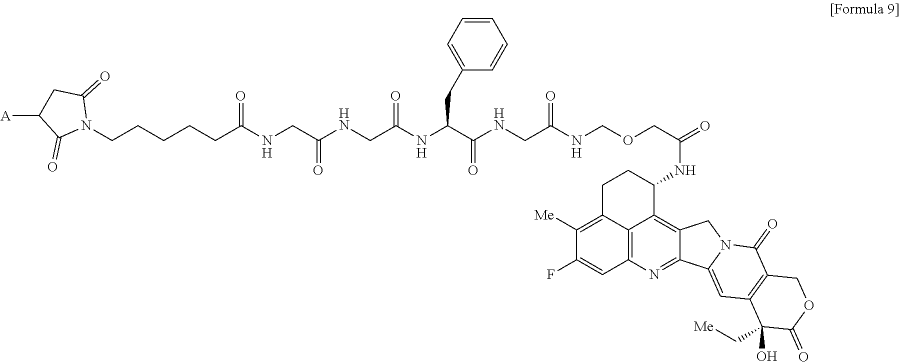

wherein A represents a connecting position to an antibody,
is conjugated to the antibody via a thioether bond.

[53] The method of treatment according to [52], wherein the antibody in the antibody-drug conjugate is an anti-HER2 antibody, an anti-HER3 antibody, an anti-TROP2 antibody, an anti-B7-H3 antibody, an anti-GPR20 antibody, an anti-CDH6 antibody, or an anti-MUC1 antibody.

[54] The method of treatment according to [53], wherein the antibody in the antibody-drug conjugate is an anti-HER2 antibody.

[55] The method of treatment according to [54], wherein the anti-HER2 antibody is an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1, and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2, and CDRL3 consisting of an amino acid sequence consisting of amino acid residues 89 to 97 of SEQ ID NO: 2.

[56] The method of treatment according to [54], wherein the anti-HER2 antibody is an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2.

[57] The method of treatment according to [54], wherein the anti-HER2 antibody is an antibody comprising a heavy chain consisting of an amino acid sequence represented by SEQ ID NO: 1 and a light chain consisting of an amino acid sequence represented by SEQ ID NO: 2.

[58] The method of treatment according to [54], wherein the anti-HER2 antibody is an antibody comprising a heavy chain consisting of an amino acid sequence consisting of amino acid residues 1 to 449 of SEQ ID NO: 1 and a light chain consisting of an amino acid sequence represented by amino acid residues 1 to 214 of SEQ ID NO: 2.

[59] The method of treatment according to any one of [54] to [58], wherein the antibody-drug conjugate is represented by the following formula:

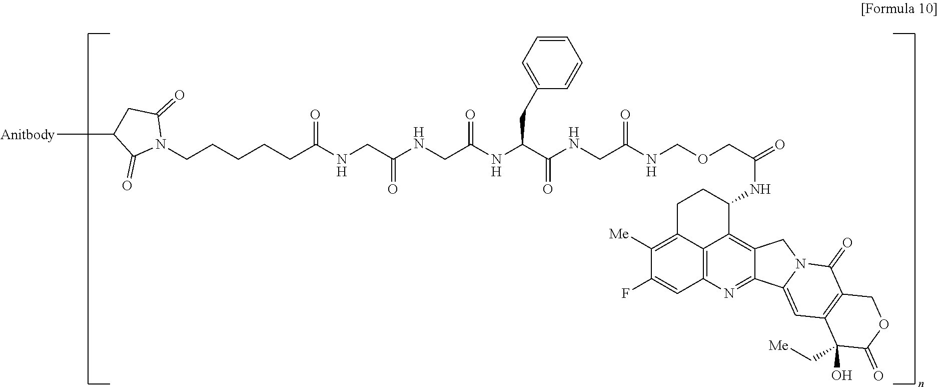

wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8.

[60] The method of treatment according to [53], wherein the antibody in the antibody-drug conjugate is an anti-HER3 antibody.

[61] The method of treatment according to [60], wherein the anti-HER3 antibody is an antibody comprising a heavy chain consisting of an amino acid sequence represented by SEQ ID NO: 3 and a light chain consisting of an amino acid sequence represented by SEQ ID NO: 4.

[62] The method of treatment according to [61], wherein the anti-HER3 antibody lacks a lysine residue at the carboxyl terminus of the heavy chain.

[63] The method of treatment according to any one of [60] to [62], wherein the antibody-drug conjugate is represented by the following formula:

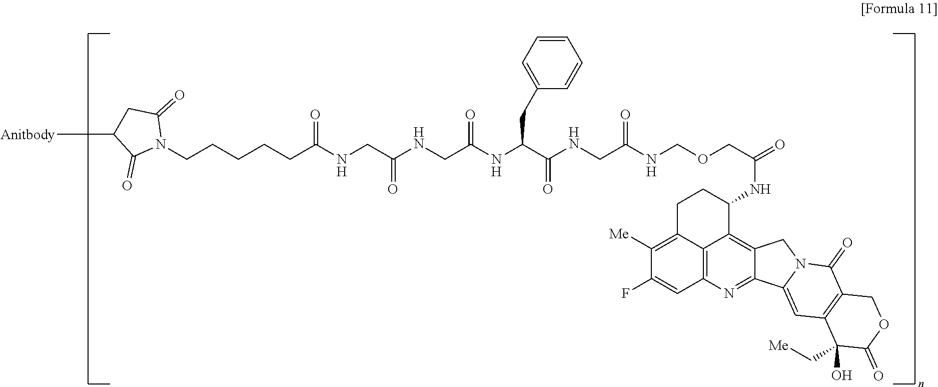

wherein ‘Antibody’ indicates the anti-HER3 antibody conjugated to the drug-linker via a thioether bond, and n indicates an average number of units of the drug-linker conjugated per antibody molecule in the antibody-drug conjugate, wherein n is in the range of from 7 to 8.

[64] The method of treatment according to [53], wherein the antibody in the antibody-drug conjugate is an anti-TROP2 antibody.

[65] The method of treatment according to [64], wherein the anti-TROP2 antibody is an antibody comprising a heavy chain comprising CDRH1 consisting of an amino acid sequence consisting of amino acid residues 50 to 54 of SEQ ID NO: 5, CDRH2 consisting of an amino acid sequence consisting of amino acid residues 69 to 85 of SEQ ID NO: 5, and CDRH3 consisting of an amino acid sequence consisting of amino acid residues 118 to 129 of SEQ ID NO: 5, and a light chain comprising CDRL1 consisting of an amino acid sequence consisting of amino acid residues 44 to 54 of SEQ ID NO: 6, CDRL2 consisting of an amino acid sequence consisting of amino acid residues 70 to 76 of SEQ ID NO: 6, and CDRL3 consisting of an amino acid sequence consisting of amino acid residues 109 to 117 of SEQ ID NO: 6.

[66] The method of treatment according to [64], wherein the anti-TROP2 antibody is an antibody comprising a heavy chain comprising a heavy chain variable region consisting of an amino acid sequence consisting of amino acid residues 20 to 140 of SEQ ID NO: 5, and a light chain comprising a light chain variable region consisting of an amino acid sequence consisting of amino acid residues 21 to 129 of SEQ ID NO: 6.

[67] The method of treatment according to [64], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

[68] The method of treatment according to [67], wherein the anti-TROP2 antibody lacks a lysine residue at the carboxyl terminus of the heavy chain.

[69] The method of treatment according to any one of [64] to [68], wherein the antibody-drug conjugate is represented by the following formula:

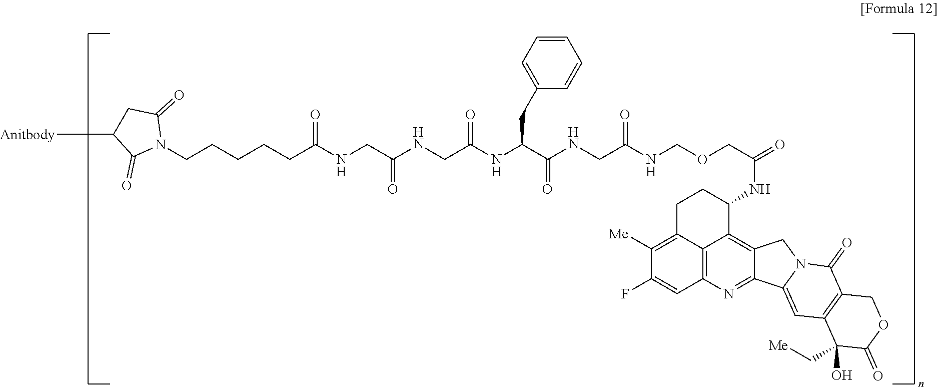

wherein ‘Antibody’ indicates the anti-TROP2 antibody conjugated to the drug-linker via a thioether bond, and n indicates an average number of units of the drug-linker conjugated per antibody molecule in the antibody-drug conjugate, wherein n is in the range of from 3.5 to 4.5.

[70] The method of treatment according to [53], wherein the antibody in the antibody-drug conjugate is an anti-B7-H3 antibody.

[71] The method of treatment according to [70], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

[72] The method of treatment according to [71], wherein the anti-B7-H3 antibody lacks a lysine residue at the carboxyl terminus of the heavy chain.

[73] The method of treatment according to any one of [70] to [72], wherein the antibody-drug conjugate is represented by the following formula:

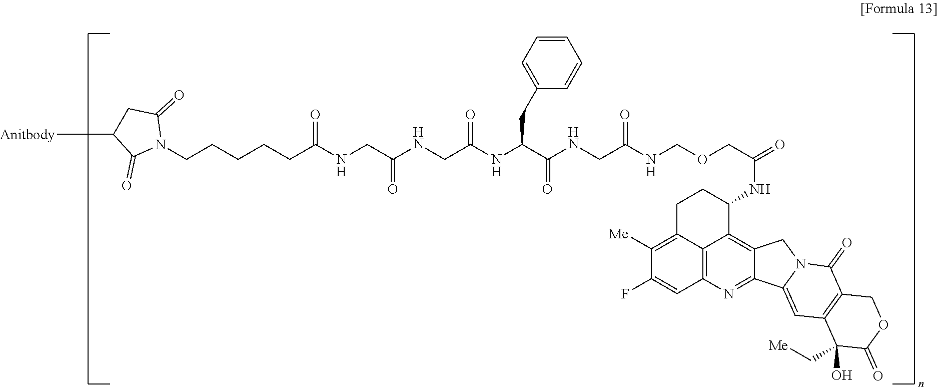

wherein ‘Antibody’ indicates the anti-B7-H3 antibody conjugated to the drug-linker via a thioether bond, and n indicates an average number of units of the drug-linker conjugated per antibody molecule in the antibody-drug conjugate, wherein n is in the range of from 3.5 to 4.5.

[74] The method of treatment according to [53], wherein the antibody in the antibody-drug conjugate is an anti-GPR20 antibody.

[75] The method of treatment according to [74], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

[76] The method of treatment according to [75], wherein the anti-GPR20 antibody lacks a lysine residue at the carboxyl terminus of the heavy chain.

[77] The method of treatment according to any one of [74] to [76], wherein the antibody-drug conjugate is represented by the following formula:

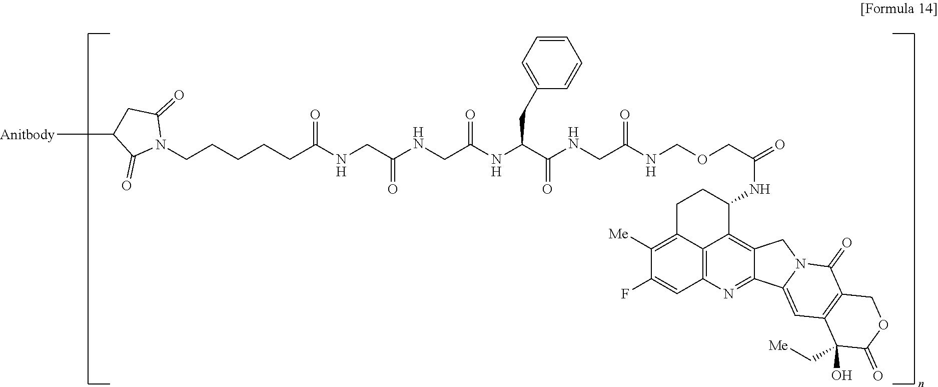

wherein ‘Antibody’ indicates the anti-GPR20 antibody conjugated to the drug-linker via a thioether bond, and n indicates an average number of units of the drug-linker conjugated per antibody molecule in the antibody-drug conjugate, wherein n is in the range of from 7 to 8.

[78] The method of treatment according to [53], wherein the antibody in the antibody-drug conjugate is an anti-CDH6 antibody.

[79] The method of treatment according to [78], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

[80] The method of treatment according to [79], wherein the anti-CDH6 antibody lacks a lysine residue at the carboxyl terminus of the heavy chain.

[81] The method of treatment according to any one of [78] to [80], wherein the antibody-drug conjugate is represented by the following formula:

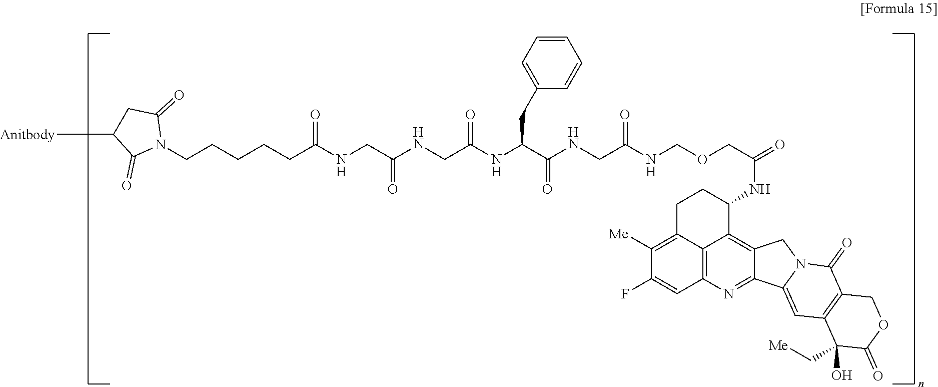

wherein ‘Antibody’ indicates the anti-CDH6 antibody conjugated to the drug-linker via a thioether bond, and n indicates an average number of units of the drug-linker conjugated per antibody molecule in the antibody-drug conjugate, wherein n is in the range of from 7 to 8.

[82] The method according to [53], wherein the antibody in the antibody-drug conjugate is an anti-MUC1 antibody.

[83] The method according to [82], wherein the anti-MUC1 antibody is an antibody comprising a heavy chain consisting of an amino acid sequence represented by SEQ ID NO: 13 and a light chain consisting of an amino acid sequence represented by SEQ ID NO: 15.

[84] The method according to [83], wherein the anti-MUC1 antibody lacks a lysine residue at the carboxyl terminus of the heavy chain.

[85] The method according to [82], wherein the anti-MUC1 antibody is an antibody comprising a heavy chain consisting of an amino acid sequence represented by SEQ ID NO: 14 and a light chain consisting of an amino acid sequence represented by SEQ ID NO: 15.

[86] The method according to [85], wherein the anti-MUC1 antibody lacks a lysine residue at the carboxyl terminus of the heavy chain.

[87] The method according to any one of [82] to [86], wherein the antibody-drug conjugate is represented by the following formula:

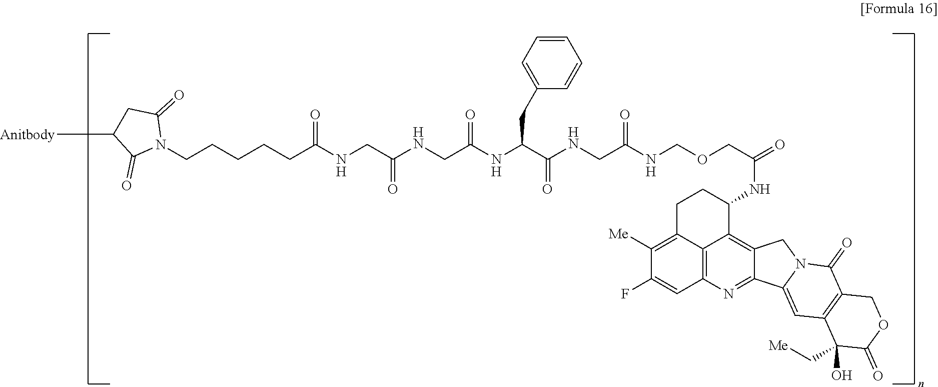

wherein ‘Antibody’ indicates the anti-MUC1 antibody conjugated to the drug-linker via a thioether bond, and n indicates an average number of units of the drug-linker conjugated per antibody molecule in the antibody-drug conjugate, wherein n is in the range of from 7 to 8.

[88] The method of treatment according to any one of [52] to [59], wherein the antibody-drug conjugate is trastuzumab deruxtecan (DS-8201a).

[89] The method of treatment according to any one of [52] to [53] and [64] to [69], wherein the antibody-drug conjugate is datopotamab deruxtecan (DS-1062).

[90] The method of treatment according to any one of [52] to [89], wherein the inhibitor is an EZH2 inhibitor.

[91] The method of treatment according to [90], wherein the inhibitor is tazemetostat or a pharmaceutically acceptable salt thereof.

[92] The method of treatment according to any one of [52] to [89], wherein the inhibitor is an EZH1/2 dual inhibitor.

[93] The method of treatment according to [92], wherein the inhibitor is valemetostat or a pharmaceutically acceptable salt thereof.

[94] The method of treatment according to [92] or [93], wherein the inhibitor is valemetostat tosylate.

[95] The method of treatment according to any one of [52] to [94], wherein the antibody-drug conjugate and the inhibitor are separately contained as active components in different formulations, and administered simultaneously or at different times.

[96] The method of treatment according to any one of [52] to [95] and [258], wherein the method of treatment is for treating at least one selected from the group consisting of breast cancer, gastric cancer, colorectal cancer, lung cancer, esophageal cancer, head-and-neck cancer, gastroesophageal junction adenocarcinoma, biliary tract cancer, Paget's disease, pancreatic cancer, ovarian cancer, bladder cancer, prostate cancer, uterine carcinosarcoma, gastrointestinal stromal tumor, kidney cancer, and sarcoma.

[97] The method of treatment according to [96], wherein the method of treatment is for treating breast cancer.

[98] The method of treatment according to [97], wherein the method of treatment is for treating triple-negative breast cancer.

[99] The method of treatment according to [96], wherein the method of treatment is for treating gastric cancer.

[100] The method of treatment according to [96], wherein the method of treatment is for treating ovarian cancer.

[101] The method of treatment according to [96], wherein the method of treatment is for treating lung cancer.

[102] The method of treatment according to [96], wherein the method of treatment is for treating pancreatic cancer.

[103] An antibody-drug conjugate for use in a method of treating a disease, wherein the method comprises administering the antibody-drug conjugate in combination with an inhibitor selected from the group consisting of an EZH1 inhibitor, an EZH2 inhibitor and an EZH1/2 dual inhibitor, wherein the antibody-drug conjugate is an antibody-drug conjugate in which a drug-linker represented by the following formula:

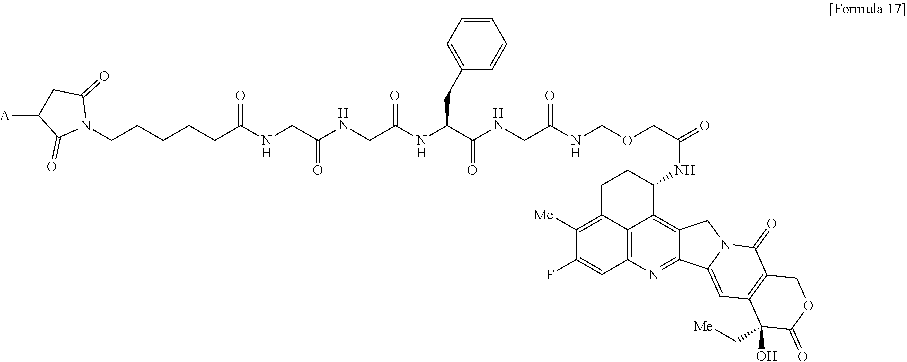

wherein A represents a connecting position to an antibody,
is conjugated to the antibody via a thioether bond in the antibody-drug conjugate.

[104] The antibody-drug conjugate for use according to [103], wherein the antibody in the antibody-drug conjugate is an anti-HER2 antibody, an anti-HER3 antibody, an anti-TROP2 antibody, an anti-B7-H3 antibody, an anti-GPR20 antibody, an anti-CDH6 antibody, or an anti-MUC1 antibody.

[105] The antibody-drug conjugate for use according to [104], wherein the antibody in the antibody-drug conjugate is an anti-HER2 antibody.

[106] The antibody-drug conjugate for use according to [105], wherein the anti-HER2 antibody is an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1, and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2, and CDRL3 consisting of an amino acid sequence consisting of amino acid residues 89 to 97 of SEQ ID NO: 2.

[107] The antibody-drug conjugate for use according to [105], wherein the anti-HER2 antibody is an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2.

[108] The antibody-drug conjugate for use according to [105], wherein the anti-HER2 antibody is an antibody comprising a heavy chain consisting of an amino acid sequence represented by SEQ ID NO: 1 and a light chain consisting of an amino acid sequence represented by SEQ ID NO: 2.

[109] The antibody-drug conjugate for use according to [105], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

[110] The antibody-drug conjugate for use according to any one of [105] to [109], wherein the antibody-drug conjugate is represented by the following formula:

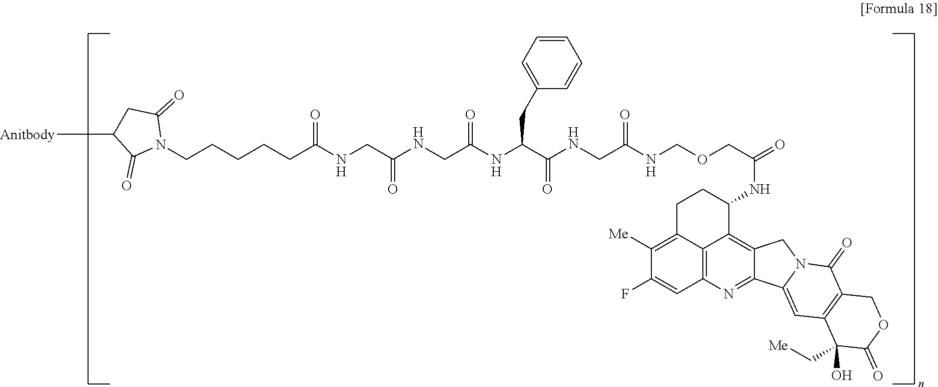

wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8.

[111] The antibody-drug conjugate for use according to [104], wherein the antibody in the antibody-drug conjugate is an anti-HER3 antibody.

[112] The antibody-drug conjugate for use according to [111], wherein the anti-HER3 antibody is an antibody comprising a heavy chain consisting of an amino acid sequence represented by SEQ ID NO: 3 and a light chain consisting of an amino acid sequence represented by SEQ ID NO: 4.

[113] The antibody-drug conjugate for use according to [112], wherein the anti-HER3 antibody lacks a lysine residue at the carboxyl terminus of the heavy chain.

[114] The antibody-drug conjugate for use according to any one of [111] to [113], wherein the antibody-drug conjugate is represented by the following formula:

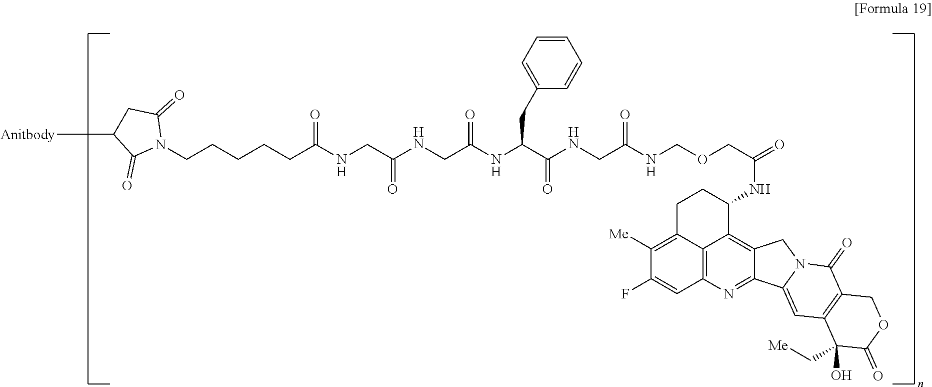

wherein ‘Antibody’ indicates the anti-HER3 antibody conjugated to the drug-linker via a thioether bond, and n indicates an average number of units of the drug-linker conjugated per antibody molecule in the antibody-drug conjugate, wherein n is in the range of from 7 to 8.

[115] The antibody-drug conjugate for use according to [104], wherein the antibody in the antibody-drug conjugate is an anti-TROP2 antibody.

[116] The antibody-drug conjugate for use according to [115], wherein the anti-TROP2 antibody is an antibody comprising a heavy chain comprising CDRH1 consisting of an amino acid sequence consisting of amino acid residues 50 to 54 of SEQ ID NO: 5, CDRH2 consisting of an amino acid sequence consisting of amino acid residues 69 to 85 of SEQ ID NO: 5, and CDRH3 consisting of an amino acid sequence consisting of amino acid residues 118 to 129 of SEQ ID NO: 5, and a light chain comprising CDRL1 consisting of an amino acid sequence consisting of amino acid residues 44 to 54 of SEQ ID NO: 6, CDRL2 consisting of an amino acid sequence consisting of amino acid residues 70 to 76 of SEQ ID NO: 6, and CDRL3 consisting of an amino acid sequence consisting of amino acid residues 109 to 117 of SEQ ID NO: 6.

[117] The antibody-drug conjugate for use according to [115], wherein the anti-TROP2 antibody is an antibody comprising a heavy chain comprising a heavy chain variable region consisting of an amino acid sequence consisting of amino acid residues 20 to 140 of SEQ ID NO: 5, and a light chain comprising a light chain variable region consisting of an amino acid sequence consisting of amino acid residues 21 to 129 of SEQ ID NO: 6.

[118] The antibody-drug conjugate for use according to [115], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

[119] The antibody-drug conjugate for use according to [118], wherein the anti-TROP2 antibody lacks a lysine residue at the carboxyl terminus of the heavy chain.

[120] The antibody-drug conjugate for use according to any one of [115] to [119], wherein the antibody-drug conjugate is represented by the following formula:

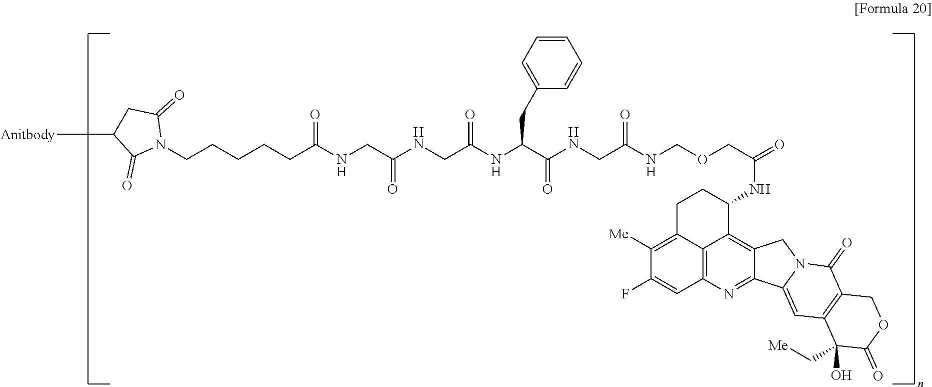

wherein ‘Antibody’ indicates the anti-TROP2 antibody conjugated to the drug-linker via a thioether bond, and n indicates an average number of units of the drug-linker conjugated per antibody molecule in the antibody-drug conjugate, wherein n is in the range of from 3.5 to 4.5.

[121] The antibody-drug conjugate for use according to [104], wherein the antibody in the antibody-drug conjugate is an anti-B7-H3 antibody.

[122] The antibody-drug conjugate for use according to [121], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

[123] The antibody-drug conjugate for use according to [122], wherein the anti-B7-H3 antibody lacks a lysine residue at the carboxyl terminus of the heavy chain.

[124] The antibody-drug conjugate for use according to any one of [121] to [123], wherein the antibody-drug conjugate is represented by the following formula:

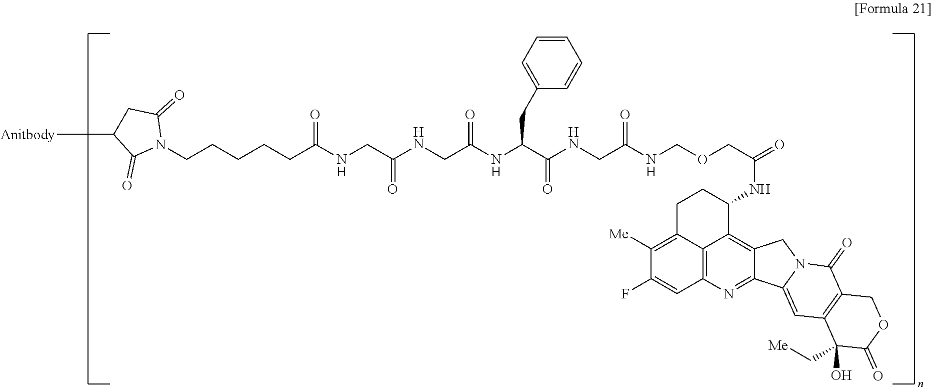

wherein ‘Antibody’ indicates the anti-B7-H3 antibody conjugated to the drug-linker via a thioether bond, and n indicates an average number of units of the drug-linker conjugated per antibody molecule in the antibody-drug conjugate, wherein n is in the range of from 3.5 to 4.5.

[125] The antibody-drug conjugate for use according to [104], wherein the antibody in the antibody-drug conjugate is an anti-GPR20 antibody.

[126] The antibody-drug conjugate for use according to [125], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

[127] The antibody-drug conjugate for use according to [126], wherein the anti-GPR20 antibody lacks a lysine residue at the carboxyl terminus of the heavy chain.

[128] The antibody-drug conjugate for use according to any one of [125] to [127], wherein the antibody-drug conjugate is represented by the following formula:

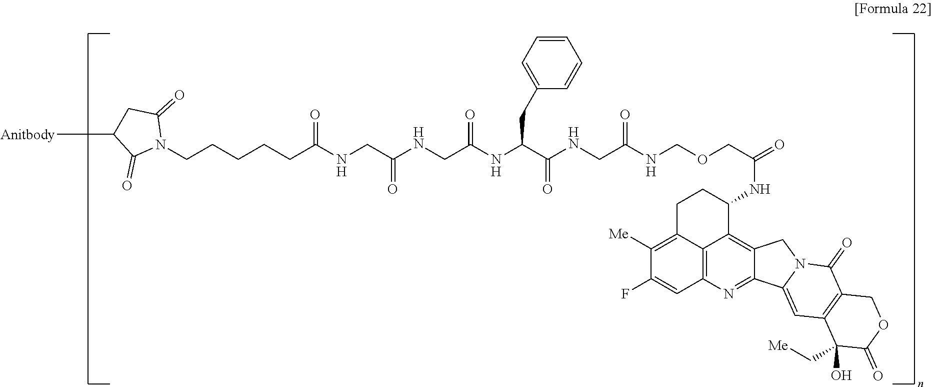

wherein ‘Antibody’ indicates the anti-GPR20 antibody conjugated to the drug-linker via a thioether bond, and n indicates an average number of units of the drug-linker conjugated per antibody molecule in the antibody-drug conjugate, wherein n is in the range of from 7 to 8.

[129] The antibody-drug conjugate for use according to [104], wherein the antibody in the antibody-drug conjugate is an anti-CDH6 antibody.

[130] The antibody-drug conjugate for use according to [129], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

[131] The antibody-drug conjugate for use according to [130], wherein the anti-CDH6 antibody lacks a lysine residue at the carboxyl terminus of the heavy chain.

[132] The antibody-drug conjugate for use according to any one of [129] to [131], wherein the antibody-drug conjugate is represented by the following formula:

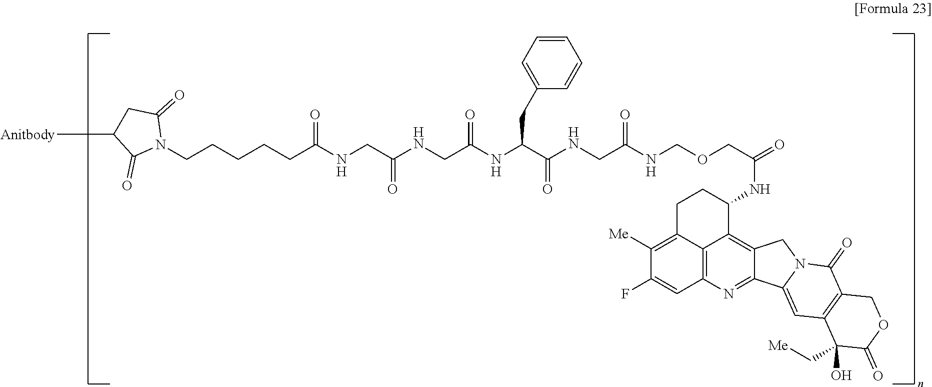

wherein ‘Antibody’ indicates the anti-CDH6 antibody conjugated to the drug-linker via a thioether bond, and n indicates an average number of units of the drug-linker conjugated per antibody molecule in the antibody-drug conjugate, wherein n is in the range of from 7 to 8.

[133] The antibody-drug conjugate for use according to [104], wherein the antibody in the antibody-drug conjugate is an anti-MUC1 antibody.

[134] The antibody-drug conjugate for use according to [133], wherein the anti-MUC1 antibody is an antibody comprising a heavy chain consisting of an amino acid sequence represented by SEQ ID NO: 13 and a light chain consisting of an amino acid sequence represented by SEQ ID NO: 15.

[135] The antibody-drug conjugate for use according to [134], wherein the anti-MUC1 antibody lacks a lysine residue at the carboxyl terminus of the heavy chain.

[136] The antibody-drug conjugate for use according to [133], wherein the anti-MUC1 antibody is an antibody comprising a heavy chain consisting of an amino acid sequence represented by SEQ ID NO: 14 and a light chain consisting of an amino acid sequence represented by SEQ ID NO: 15.

[137] The antibody-drug conjugate for use according to [136], wherein the anti-MUC1 antibody lacks a lysine residue at the carboxyl terminus of the heavy chain.

[138] The antibody-drug conjugate for use according to any one of [133] to [137], wherein the antibody-drug conjugate is represented by the following formula:

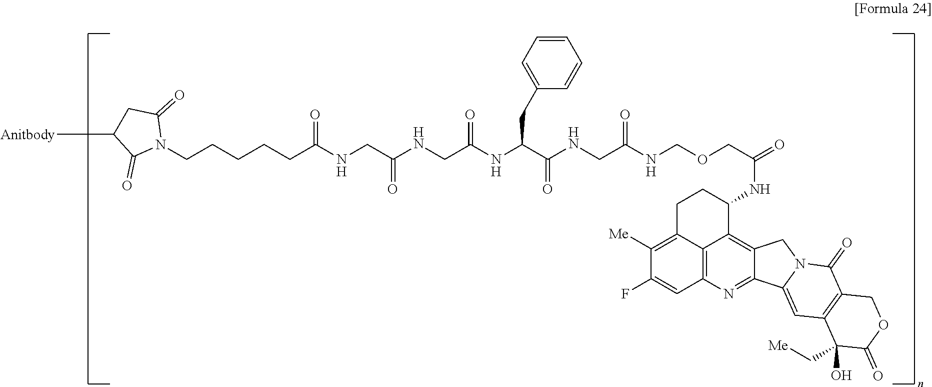

wherein ‘Antibody’ indicates the anti-MUC1 antibody conjugated to the drug-linker via a thioether bond, and n indicates an average number of units of the drug-linker conjugated per antibody molecule in the antibody-drug conjugate, wherein n is in the range of from 7 to 8.

[139] The antibody-drug conjugate for use according to any one of [103] to [110], wherein the antibody-drug conjugate is trastuzumab deruxtecan (DS-8201a).

[140] The antibody-drug conjugate for use according to any one of [103] to [104] and [115] to [120], wherein the antibody-drug conjugate is datopotamab deruxtecan (DS-1062).

[141] The antibody-drug conjugate for use according to any one of [103] to [140], wherein the inhibitor is an EZH2 inhibitor.

[142] The antibody-drug conjugate for use according to [141], wherein the inhibitor is tazemetostat or a pharmaceutically acceptable salt thereof.

[143] The antibody-drug conjugate for use according to any one of [103] to [140], wherein the inhibitor is an EZH1/2 dual inhibitor.

[144] The antibody-drug conjugate for use according to [143], wherein the inhibitor is valemetostat or a pharmaceutically acceptable salt thereof.

[145] The antibody-drug conjugate for use according to [143] or [144], wherein the inhibitor is valemetostat tosylate.

[146] The antibody-drug conjugate for use according to any one of [103] to [145], wherein the antibody-drug conjugate and the inhibitor are separately contained as active components in different formulations, and are administered simultaneously or at different times.

[147] The antibody-drug conjugate for use according to any one of [103] to [146] and [259], wherein the disease is at least one selected from the group consisting of breast cancer, gastric cancer, colorectal cancer, lung cancer, esophageal cancer, head-and-neck cancer, gastroesophageal junction adenocarcinoma, biliary tract cancer, Paget's disease, pancreatic cancer, ovarian cancer, bladder cancer, prostate cancer, uterine carcinosarcoma, gastrointestinal stromal tumor, kidney cancer, and sarcoma.

[148] The antibody-drug conjugate for use according to [147], wherein the disease is breast cancer.

[149] The antibody-drug conjugate for use according to [148], wherein the disease is triple-negative breast cancer.

[150] The antibody-drug conjugate for use according to [147], wherein the disease is gastric cancer.

[151] The antibody-drug conjugate for use according to [147], wherein the disease is ovarian cancer.

[152] The antibody-drug conjugate for use according to [147], wherein the disease is lung cancer.

[153] The antibody-drug conjugate for use according to [147], wherein the disease is pancreatic cancer.

[154] Use of an antibody-drug conjugate or an inhibitor selected from the group consisting of an EZH1 inhibitor, an EZH2 inhibitor and an EZH1/2 dual inhibitor for the manufacture of a medicament for treating a disease by administration of the antibody-drug conjugate and the inhibitor in combination, wherein the antibody-drug conjugate is an antibody-drug conjugate in which a drug-linker represented by the following formula:

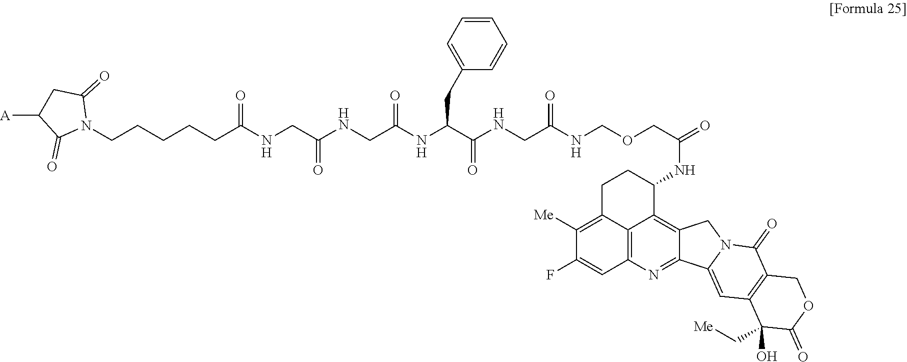

wherein A represents a connecting position to an antibody,
is conjugated to the antibody via a thioether bond in the antibody-drug conjugate.

[155] The use according to [154], wherein the antibody in the antibody-drug conjugate is an anti-HER2 antibody, an anti-HER3 antibody, an anti-TROP2 antibody, an anti-B7-H3 antibody, an anti-GPR20 antibody, an anti-CDH6 antibody, or an anti-MUC1 antibody.

[156] The use according to [155], wherein the antibody in the antibody-drug conjugate is an anti-HER2 antibody.

[157] The use according to [156], wherein the anti-HER2 antibody is an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1, and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2, and CDRL3 consisting of an amino acid sequence consisting of amino acid residues 89 to 97 of SEQ ID NO: 2.

[158] The use according to [156], wherein the anti-HER2 antibody is an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2.

[159] The use according to [156], wherein the anti-HER2 antibody is an antibody comprising a heavy chain consisting of an amino acid sequence represented by SEQ ID NO: 1 and a light chain consisting of an amino acid sequence represented by SEQ ID NO: 2.

[160] The use according to [156], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

[161] The use according to any one of [156] to [160], wherein the antibody-drug conjugate is represented by the following formula:

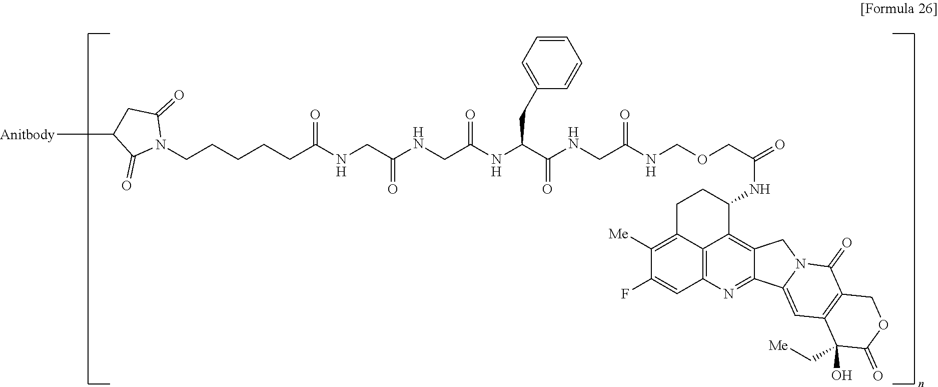

wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8.

[162] The use according to [155], wherein the antibody in the antibody-drug conjugate is an anti-HER3 antibody.

[163] The use according to [162], wherein the anti-HER3 antibody is an antibody comprising a heavy chain consisting of an amino acid sequence represented by SEQ ID NO: 3 and a light chain consisting of an amino acid sequence represented by SEQ ID NO: 4.

[164] The use according to [163], wherein the anti-HER3 antibody lacks a lysine residue at the carboxyl terminus of the heavy chain.

[165] The use according to any one of [162] to [164], wherein the antibody-drug conjugate is represented by the following formula:

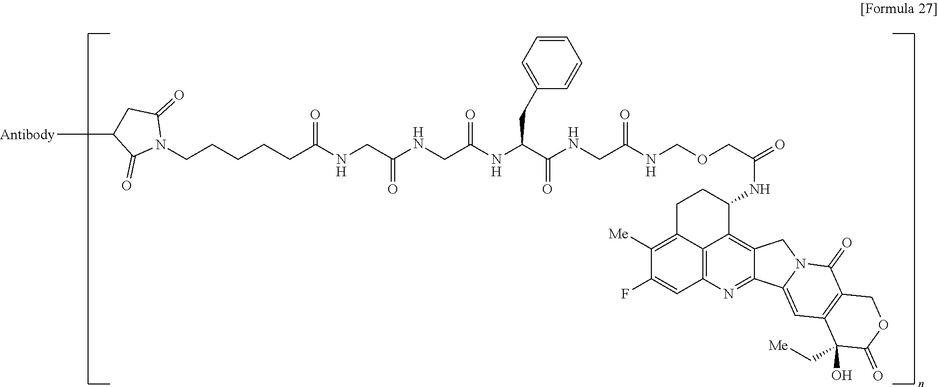

wherein ‘Antibody’ indicates the anti-HER3 antibody conjugated to the drug-linker via a thioether bond, and n indicates an average number of units of the drug-linker conjugated per antibody molecule in the antibody-drug conjugate, wherein n is in the range of from 7 to 8.

[166] The use according to [155], wherein the antibody in the antibody-drug conjugate is an anti-TROP2 antibody.

[167] The use according to [166], wherein the anti-TROP2 antibody is an antibody comprising a heavy chain comprising CDRH1 consisting of an amino acid sequence consisting of amino acid residues 50 to 54 of SEQ ID NO: 5, CDRH2 consisting of an amino acid sequence consisting of amino acid residues 69 to 85 of SEQ ID NO: 5, and CDRH3 consisting of an amino acid sequence consisting of amino acid residues 118 to 129 of SEQ ID NO: 5, and a light chain comprising CDRL1 consisting of an amino acid sequence consisting of amino acid residues 44 to 54 of SEQ ID NO: 6, CDRL2 consisting of an amino acid sequence consisting of amino acid residues 70 to 76 of SEQ ID NO: 6, and CDRL3 consisting of an amino acid sequence consisting of amino acid residues 109 to 117 of SEQ ID NO: 6.

[168] The use according to [166], wherein the anti-TROP2 antibody is an antibody comprising a heavy chain comprising a heavy chain variable region consisting of an amino acid sequence consisting of amino acid residues 20 to 140 of SEQ ID NO: 5, and a light chain comprising a light chain variable region consisting of an amino acid sequence consisting of amino acid residues 21 to 129 of SEQ ID NO: 6.

[169] The use according to [166], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

[170] The use according to [169], wherein the anti-TROP2 antibody lacks a lysine residue at the carboxyl terminus of the heavy chain.

[171] The use according to any one of [166] to [170], wherein the antibody-drug conjugate is represented by the following formula:

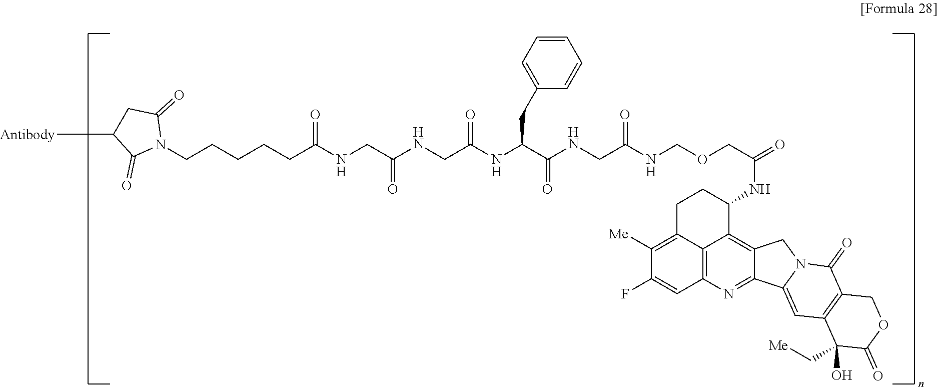

wherein ‘Antibody’ indicates the anti-TROP2 antibody conjugated to the drug-linker via a thioether bond, and n indicates an average number of units of the drug-linker conjugated per antibody molecule in the antibody-drug conjugate, wherein n is in the range of from 3.5 to 4.5.

[172] The use according to [155], wherein the antibody in the antibody-drug conjugate is an anti-B7-H3 antibody.

[173] The use according to [172], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

[174] The use according to [173], wherein the anti-B7-H3 antibody lacks a lysine residue at the carboxyl terminus of the heavy chain.

[175] The use according to any one of [172] to [174], wherein the antibody-drug conjugate is represented by the following formula:

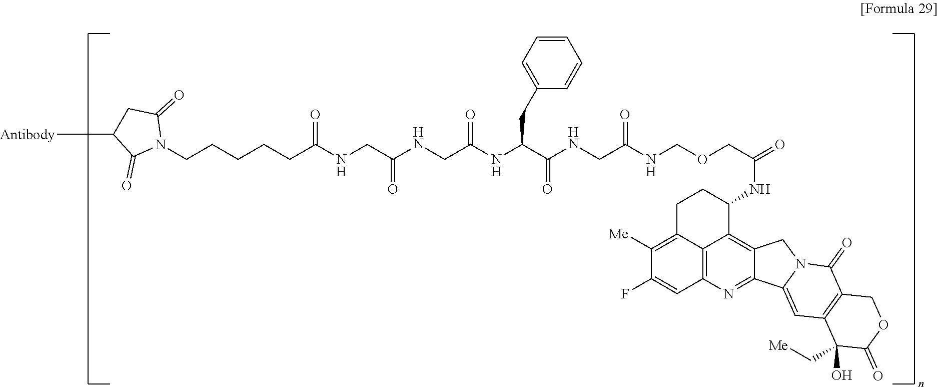

wherein ‘Antibody’ indicates the anti-B7-H3 antibody conjugated to the drug-linker via a thioether bond, and n indicates an average number of units of the drug-linker conjugated per antibody molecule in the antibody-drug conjugate, wherein n is in the range of from 3.5 to 4.5.

[176] The use according to [155], wherein the antibody in the antibody-drug conjugate is an anti-GPR20 antibody.

[177] The use according to [176], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

[178] The use according to [177], wherein the anti-GPR20 antibody lacks a lysine residue at the carboxyl terminus of the heavy chain.

[179] The use according to any one of [176] to [178], wherein the antibody-drug conjugate is represented by the following formula:

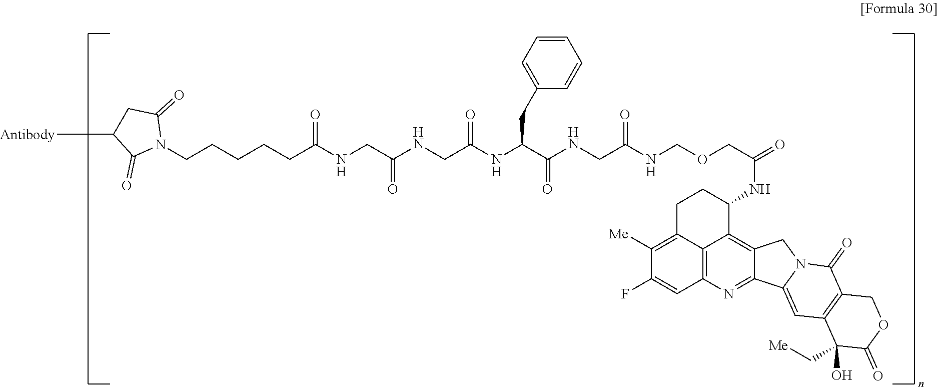

wherein ‘Antibody’ indicates the anti-GPR20 antibody conjugated to the drug-linker via a thioether bond, and n indicates an average number of units of the drug-linker conjugated per antibody molecule in the antibody-drug conjugate, wherein n is in the range of from 7 to 8.

[180] The use according to [155], wherein the antibody in the antibody-drug conjugate is an anti-CDH6 antibody.

[181] The use according to [180], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

[182] The use according to [181], wherein the anti-CDH6 antibody lacks a lysine residue at the carboxyl terminus of the heavy chain.

[183] The use according to any one of [180] to [182], wherein the antibody-drug conjugate is represented by the following formula:

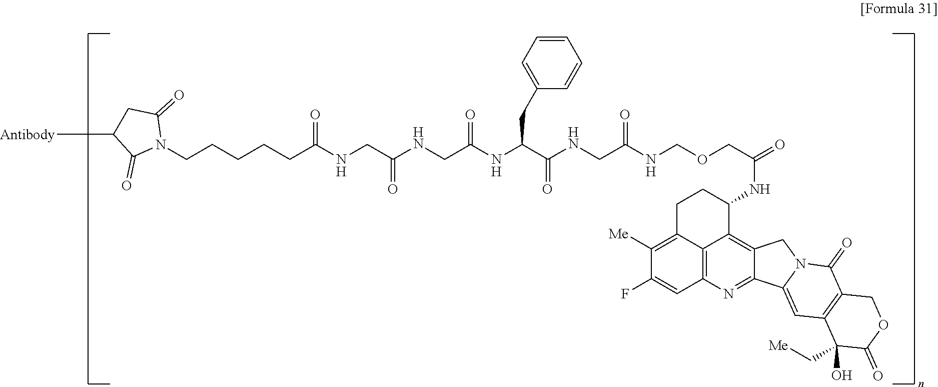

wherein ‘Antibody’ indicates the anti-CDH6 antibody conjugated to the drug-linker via a thioether bond, and n indicates an average number of units of the drug-linker conjugated per antibody molecule in the antibody-drug conjugate, wherein n is in the range of from 7 to 8.

[184] The use according to [155], wherein the antibody in the antibody-drug conjugate is an anti-MUC1 antibody.

[185] The use according to [184], wherein the anti-MUC1 antibody is an antibody comprising a heavy chain consisting of an amino acid sequence represented by SEQ ID NO: 13 and a light chain consisting of an amino acid sequence represented by SEQ ID NO: 15.

[186] The use according to [185], wherein the anti-MUC1 antibody lacks a lysine residue at the carboxyl terminus of the heavy chain.

[187] The use according to [184], wherein the anti-MUC1 antibody is an antibody comprising a heavy chain consisting of an amino acid sequence represented by SEQ ID NO: 14 and a light chain consisting of an amino acid sequence represented by SEQ ID NO: 15.

[188] The use according to [187], wherein the anti-MUC1 antibody lacks a lysine residue at the carboxyl terminus of the heavy chain.

[189] The use according to any one of [184] to [188], wherein the antibody-drug conjugate is represented by the following formula:

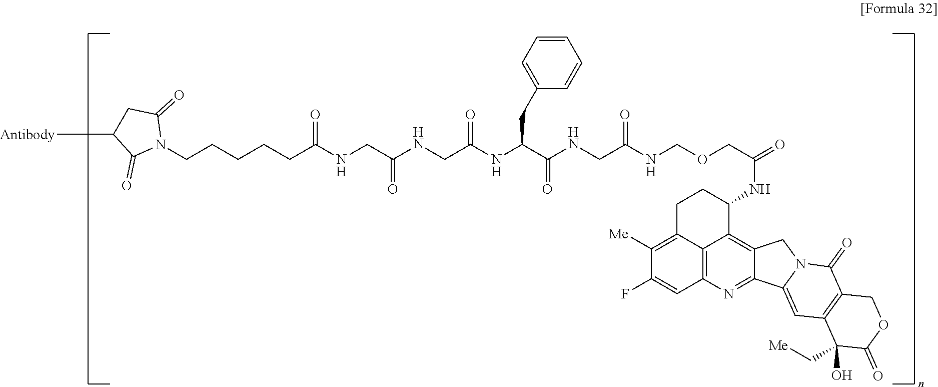

wherein ‘Antibody’ indicates the anti-MUC1 antibody conjugated to the drug-linker via a thioether bond, and n indicates an average number of units of the drug-linker conjugated per antibody molecule in the antibody-drug conjugate, wherein n is in the range of from 7 to 8.

[190] The use according to any one of [154] to [161], wherein the antibody-drug conjugate is trastuzumab deruxtecan (DS-8201a).

[191] The use according to any one of [154] to [155] and [166] to [171], wherein the antibody-drug conjugate is datopotamab deruxtecan (DS-1062).

[192] The use according to any one of [154] to [191], wherein the inhibitor is an EZH2 inhibitor.

[193] The use according to [192], wherein the inhibitor is tazemetostat or a pharmaceutically acceptable salt thereof.

[194] The use according to any one of [154] to [191], wherein the inhibitor is an EZH1/2 dual inhibitor.

[195] The use according to [194], wherein the inhibitor is valemetostat or a pharmaceutically acceptable salt thereof.

[196] The use according to [194] or [195], wherein the inhibitor is valemetostat tosylate.

[197] The use according to any one of [154] to [196], wherein the antibody-drug conjugate and the inhibitor are separately contained as active components in different formulations, and are administered simultaneously or at different times.

[198] The use according to any one of [154] to [197] and [260], wherein the disease is at least one selected from the group consisting of breast cancer, gastric cancer, colorectal cancer, lung cancer, esophageal cancer, head-and-neck cancer, gastroesophageal junction adenocarcinoma, biliary tract cancer, Paget's disease, pancreatic cancer, ovarian cancer, bladder cancer, prostate cancer, uterine carcinosarcoma, gastrointestinal stromal tumor, kidney cancer, and sarcoma.

[199] The use according to [198], wherein the disease is breast cancer.

[200] The use according to [199], wherein the disease is triple-negative breast cancer.

[201] The use according to [198], wherein disease is gastric cancer.

[202] The use according to [198], wherein the disease is ovarian cancer.

[203] The use according to [198], wherein the disease is lung cancer.

[204] The use according to [198], wherein the disease is pancreatic cancer.

[205] An inhibitor selected from the group consisting of an EZH1 inhibitor, an EZH2 inhibitor and an EZH1/2 dual inhibitor for use in a method of treating a disease, wherein the method comprises administering the inhibitor in combination with an antibody-drug conjugate, wherein the antibody-drug conjugate is an antibody-drug conjugate in which a drug-linker represented by the following formula:

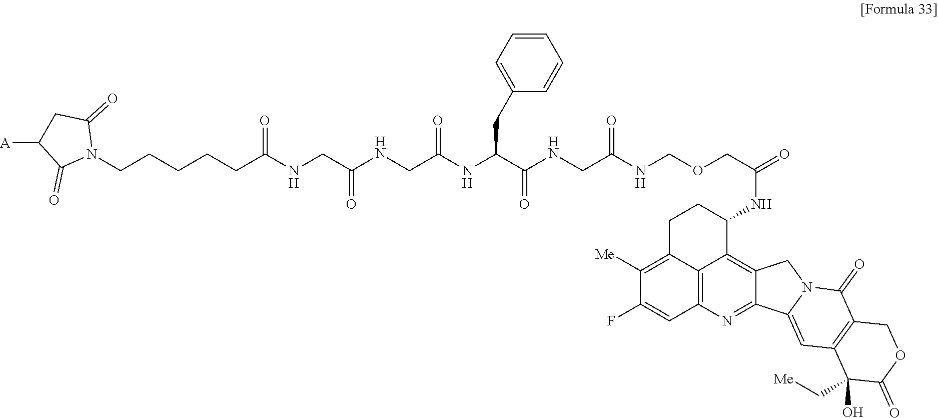

wherein A represents a connecting position to an antibody,
is conjugated to the antibody via a thioether bond in the antibody-drug conjugate.

[206] The inhibitor for use according to [205], wherein the antibody in the antibody-drug conjugate is an anti-HER2 antibody, an anti-HER3 antibody, an anti-TROP2 antibody, an anti-B7-H3 antibody, an anti-GPR20 antibody, an anti-CDH6 antibody, or an anti-MUC1 antibody.

[207] The inhibitor for use according to [206], wherein the antibody in the antibody-drug conjugate is an anti-HER2 antibody.

[208] The inhibitor for use according to [207], wherein the anti-HER2 antibody is an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1, and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2, and CDRL3 consisting of an amino acid sequence consisting of amino acid residues 89 to 97 of SEQ ID NO: 2.

[209] The inhibitor for use according to [207], wherein the anti-HER2 antibody is an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2.

[210] The inhibitor for use according to [207], wherein the anti-HER2 antibody is an antibody comprising a heavy chain consisting of an amino acid sequence represented by SEQ ID NO: 1 and a light chain consisting of an amino acid sequence represented by SEQ ID NO: 2.

[211] The inhibitor for use according to [207], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

[212] The inhibitor for use according to any one of [207] to [211], wherein the antibody-drug conjugate is represented by the following formula:

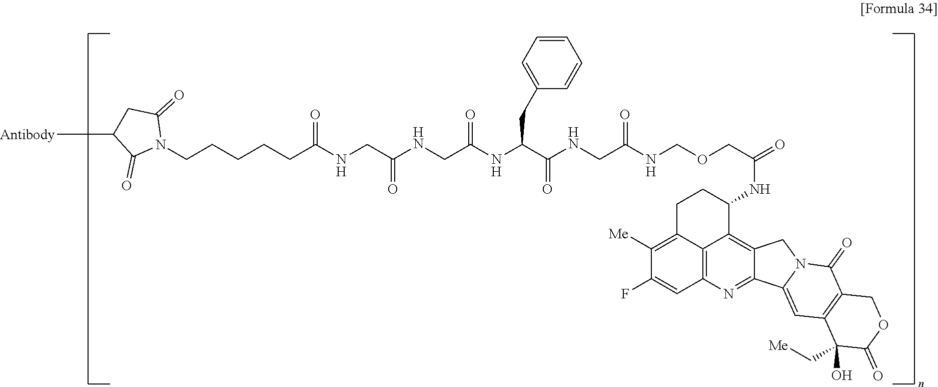

wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8.

[213] The inhibitor for use according to [206], wherein the antibody in the antibody-drug conjugate is an anti-HER3 antibody.

[214] The inhibitor for use according to [213], wherein the anti-HER3 antibody is an antibody comprising a heavy chain consisting of an amino acid sequence represented by SEQ ID NO: 3 and a light chain consisting of an amino acid sequence represented by SEQ ID NO: 4.

[215] The inhibitor for use according to [214], wherein the anti-HER3 antibody lacks a lysine residue at the carboxyl terminus of the heavy chain.

[216] The inhibitor for use according to any one of [213] to [215], wherein the antibody-drug conjugate is represented by the following formula:

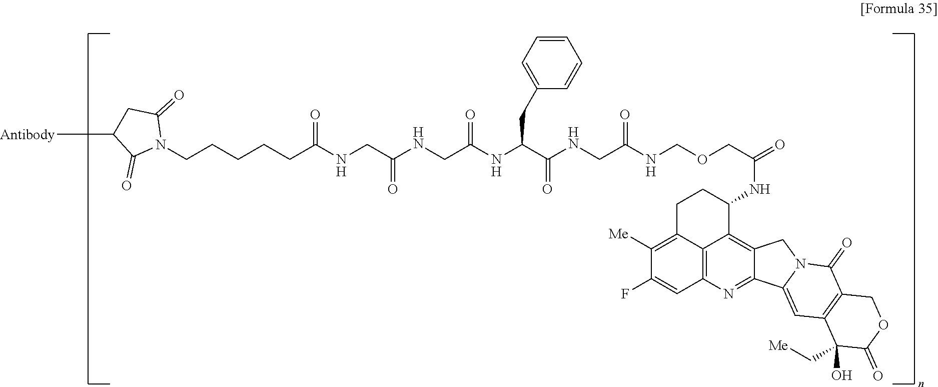

wherein ‘Antibody’ indicates the anti-HER3 antibody conjugated to the drug-linker via a thioether bond, and n indicates an average number of units of the drug-linker conjugated per antibody molecule in the antibody-drug conjugate, wherein n is in the range of from 7 to 8.

[217] The inhibitor for use according to [206], wherein the antibody in the antibody-drug conjugate is an anti-TROP2 antibody.

[218] The inhibitor for use according to [217], wherein the anti-TROP2 antibody is an antibody comprising a heavy chain comprising CDRH1 consisting of an amino acid sequence consisting of amino acid residues 50 to 54 of SEQ ID NO: 5, CDRH2 consisting of an amino acid sequence consisting of amino acid residues 69 to 85 of SEQ ID NO: 5, and CDRH3 consisting of an amino acid sequence consisting of amino acid residues 118 to 129 of SEQ ID NO: 5, and a light chain comprising CDRL1 consisting of an amino acid sequence consisting of amino acid residues 44 to 54 of SEQ ID NO: 6, CDRL2 consisting of an amino acid sequence consisting of amino acid residues 70 to 76 of SEQ ID NO: 6, and CDRL3 consisting of an amino acid sequence consisting of amino acid residues 109 to 117 of SEQ ID NO: 6.

[219] The inhibitor for use according to [217], wherein the anti-TROP2 antibody is an antibody comprising a heavy chain comprising a heavy chain variable region consisting of an amino acid sequence consisting of amino acid residues 20 to 140 of SEQ ID NO: 5, and a light chain comprising a light chain variable region consisting of an amino acid sequence consisting of amino acid residues 21 to 129 of SEQ ID NO: 6.

[220] The inhibitor for use according to [217], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

[221] The inhibitor for use according to [220], wherein the anti-TROP2 antibody lacks a lysine residue at the carboxyl terminus of the heavy chain.

[222] The inhibitor for use according to any one of [217] to [221], wherein the antibody-drug conjugate is represented by the following formula:

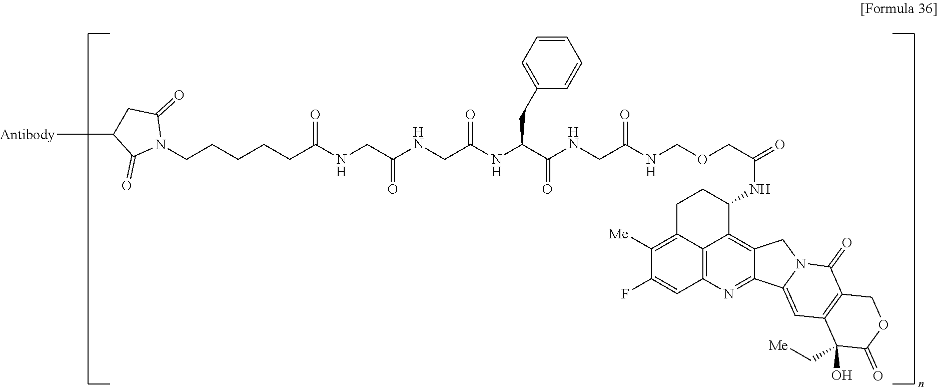

wherein ‘Antibody’ indicates the anti-TROP2 antibody conjugated to the drug-linker via a thioether bond, and n indicates an average number of units of the drug-linker conjugated per antibody molecule in the antibody-drug conjugate, wherein n is in the range of from 3.5 to 4.5.

[223] The inhibitor for use according to [206], wherein the antibody in the antibody-drug conjugate is an anti-B7-H3 antibody.

[224] The inhibitor for use according to [223], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

[225] The inhibitor for use according to [224], wherein the anti-B7-H3 antibody lacks a lysine residue at the carboxyl terminus of the heavy chain.

[226] The inhibitor for use according to any one of [223] to [225], wherein the antibody-drug conjugate is represented by the following formula:

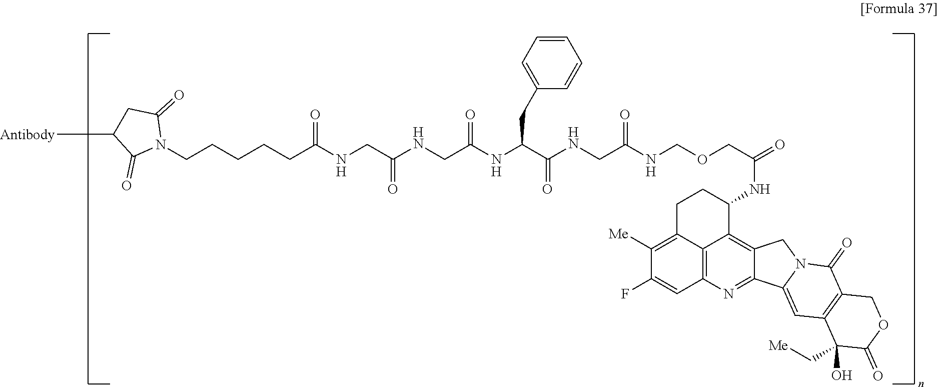

wherein ‘Antibody’ indicates the anti-B7-H3 antibody conjugated to the drug-linker via a thioether bond, and n indicates an average number of units of the drug-linker conjugated per antibody molecule in the antibody-drug conjugate, wherein n is in the range of from 3.5 to 4.5.

[227] The inhibitor for use according to [206], wherein the antibody in the antibody-drug conjugate is an anti-GPR20 antibody.

[228] The inhibitor for use according to [227], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

[229] The inhibitor for use according to [228], wherein the anti-GPR20 antibody lacks a lysine residue at the carboxyl terminus of the heavy chain.

[230] The inhibitor for use according to any one of [227] to [229], wherein the antibody-drug conjugate is represented by the following formula:

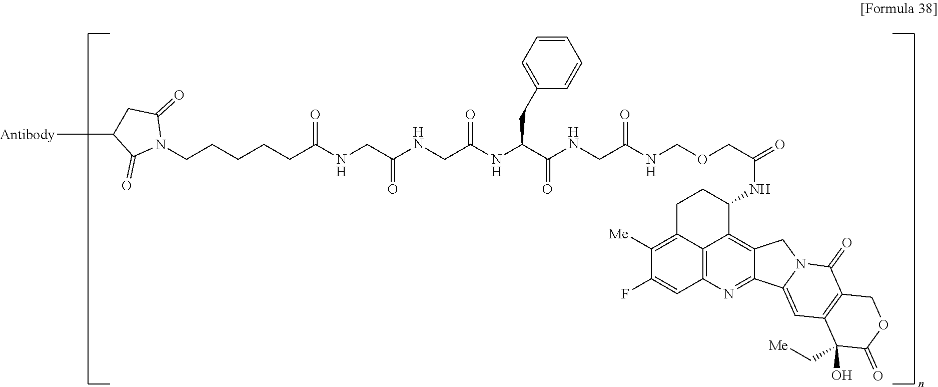

wherein ‘Antibody’ indicates the anti-GPR20 antibody conjugated to the drug-linker via a thioether bond, and n indicates an average number of units of the drug-linker conjugated per antibody molecule in the antibody-drug conjugate, wherein n is in the range of from 7 to 8.

[231] The inhibitor for use according to [206], wherein the antibody in the antibody-drug conjugate is an anti-CDH6 antibody.

[232] The inhibitor for use according to [231], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

[233] The inhibitor for use according to [232], wherein the anti-CDH6 antibody lacks a lysine residue at the carboxyl terminus of the heavy chain.

[234] The inhibitor for use according to any one of [231] to [233], wherein the anti-CDH6 antibody-drug conjugate is represented by the following formula:

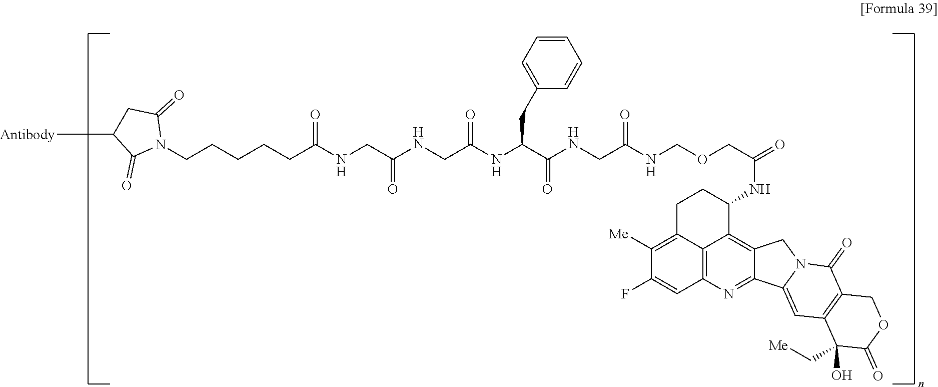

wherein ‘Antibody’ indicates the anti-CDH6 antibody conjugated to the drug-linker via a thioether bond, and n indicates an average number of units of the drug-linker conjugated per antibody molecule in the antibody-drug conjugate, wherein n is in the range of from 7 to 8.

[235] The inhibitor for use according to [206], wherein the antibody in the antibody-drug conjugate is an anti-MUC1 antibody.

[236] The inhibitor for use according to [235], wherein the anti-MUC1 antibody is an antibody comprising a heavy chain consisting of an amino acid sequence represented by SEQ ID NO: 13 and a light chain consisting of an amino acid sequence represented by SEQ ID NO: 15.

[237] The inhibitor for use according to [236], wherein the anti-MUC antibody lacks a lysine residue at the carboxyl terminus of the heavy chain.

[238] The inhibitor for use according to [235], wherein the anti-MUC1 antibody is an antibody comprising a heavy chain consisting of an amino acid sequence represented by SEQ ID NO: 14 and a light chain consisting of an amino acid sequence represented by SEQ ID NO: 15.

[239] The inhibitor for use according to [238], wherein the anti-MUC1 antibody lacks a lysine residue at the carboxyl terminus of the heavy chain.

[240] The inhibitor for use according to any one of [235] to [239], wherein the antibody-drug conjugate is represented by the following formula:

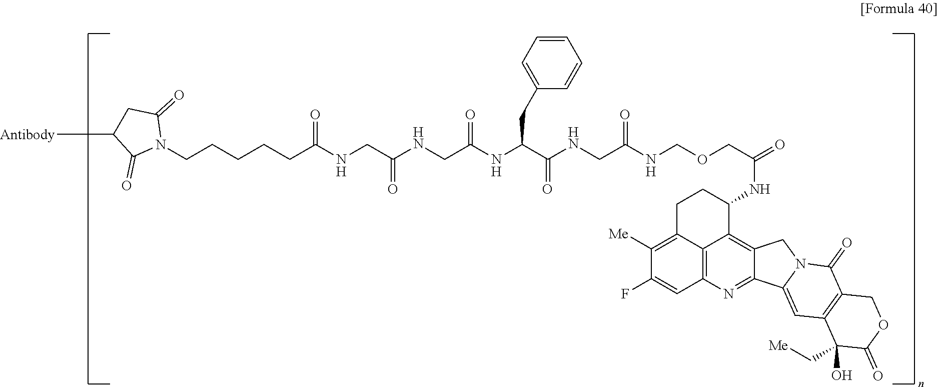

wherein ‘Antibody’ indicates the anti-MUC1 antibody conjugated to the drug-linker via a thioether bond, and n indicates an average number of units of the drug-linker conjugated per antibody molecule in the antibody-drug conjugate, wherein n is in the range of from 7 to 8.

[241] The inhibitor for use according to any one of [205] to [212], wherein the antibody-drug conjugate is trastuzumab deruxtecan (DS-8201a).

[242] The inhibitor for use according to any one of [205] to [206] and [217] to [222], wherein the antibody-drug conjugate is datopotamab deruxtecan (DS-1062).

[243] The inhibitor for use according to any one of [205] to [242], wherein the inhibitor is an EZH2 inhibitor.

[244] The inhibitor for use according to [243], wherein the inhibitor is tazemetostat or a pharmaceutically acceptable salt thereof.

[245] The inhibitor for use according to any one of [205] to [242], wherein the inhibitor is an EZH1/2 dual inhibitor.

[246] The inhibitor for use according to [245], wherein the inhibitor is valemetostat or a pharmaceutically acceptable salt thereof.

[247] The inhibitor for use according to [245] or [246], wherein the inhibitor is valemetostat tosylate.

[248] The inhibitor for use according to any one of [205] to [247], wherein the antibody-drug conjugate and the inhibitor are separately contained as active components in different formulations, and are administered simultaneously or at different times.

[249] The inhibitor for use according to any one of [205] to [248] and [261], wherein the disease is at least one selected from the group consisting of breast cancer, gastric cancer, colorectal cancer, lung cancer, esophageal cancer, head-and-neck cancer, gastroesophageal junction adenocarcinoma, biliary tract cancer, Paget's disease, pancreatic cancer, ovarian cancer, bladder cancer, prostate cancer, uterine carcinosarcoma, gastrointestinal stromal tumor, kidney cancer, and sarcoma.

[250] The inhibitor for use according to [249], wherein the disease is breast cancer.

[251] The inhibitor for use according to [250], wherein the disease is triple-negative breast cancer.

[252] The inhibitor for use according to [249], wherein the disease is gastric cancer.

[253] The inhibitor for use according to [249], wherein the disease is ovarian cancer.

[254] The inhibitor for use according to [249], wherein the disease is lung cancer.

[255] The inhibitor for use according to [249], wherein the disease is pancreatic cancer.

[256] The pharmaceutical product according to any one of [1] to [51] and [257], wherein the pharmaceutical product is a pharmaceutical composition.

[257] The pharmaceutical product according to any one of [1] to [44], wherein the pharmaceutical product is for use in treating cancer.

[258] The method of treatment according to any one of [52] to [95], wherein the method of treatment is for treating cancer.

[259] The antibody-drug conjugate for use according to any one of [103] to [146], wherein the disease is cancer.

[260] The use according to any one of [154] to [197], wherein the disease is cancer.

[261] The inhibitor for use according to any one of [205] to [248], wherein the disease is cancer.

Other embodiments of the present invention are described as follows.

[1-1]A pharmaceutical product comprising an antibody-drug conjugate and an inhibitor selected from the group consisting of an EZH1 inhibitor, an EZH2 inhibitor and an EZH1/2 dual inhibitor for administration in combination, wherein
the antibody-drug conjugate is an antibody-drug conjugate in which a drug-linker represented by the following formula:

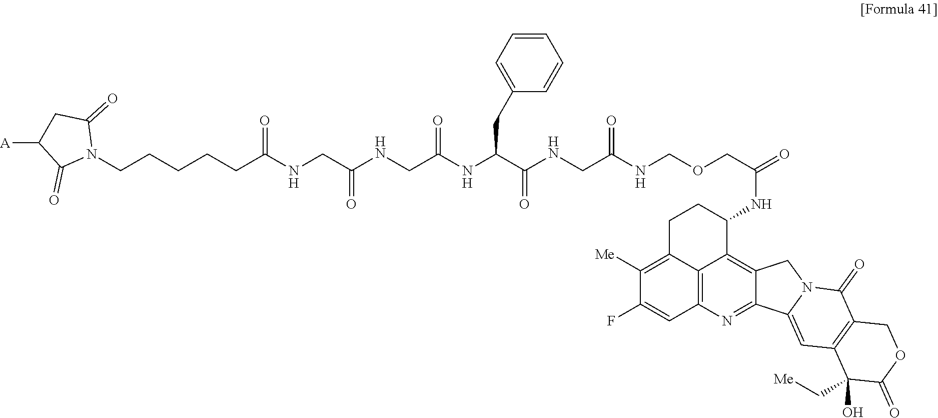

wherein A represents a connecting position to an antibody,
is conjugated to the antibody via a thioether bond.

[2-1] The pharmaceutical product according to [1-1], wherein the antibody in the antibody-drug conjugate is an anti-HER2 antibody, an anti-HER3 antibody, an anti-TROP2 antibody, an anti-B7-H3 antibody, an anti-GPR20 antibody, an anti-CDH6 antibody, an anti-MUC1 antibody, or an anti-CD37 antibody.

[3-1] The pharmaceutical product according to [2-1], wherein the antibody in the antibody-drug conjugate is an anti-HER2 antibody.

[4-1] The pharmaceutical product according to [3-1], wherein the anti-HER2 antibody is an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1, and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2, and CDRL3 consisting of an amino acid sequence consisting of amino acid residues 89 to 97 of SEQ ID NO: 2.

[5-1] The pharmaceutical product according to [3-1], wherein the anti-HER2 antibody is an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2.

[6-1] The pharmaceutical product according to [3-1], wherein the anti-HER2 antibody is an antibody comprising a heavy chain consisting of an amino acid sequence represented by SEQ ID NO: 1 and a light chain consisting of an amino acid sequence represented by SEQ ID NO: 2.

[7-1] The pharmaceutical product according to [3-1], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

[8-1] The pharmaceutical product according to any one of [3-1] to [7-1], wherein the antibody-drug conjugate is represented by the following formula:

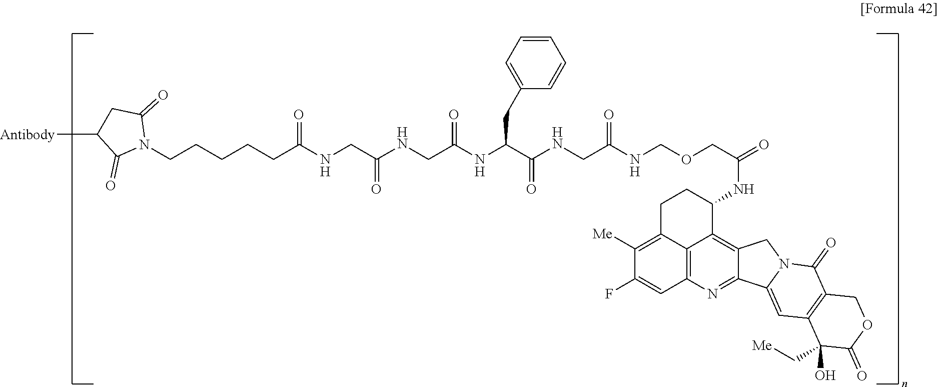

wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8.

[9-1] The pharmaceutical product according to [2-1], wherein the antibody in the antibody-drug conjugate is an anti-HER3 antibody.

[A1] The pharmaceutical product according to [9-1], wherein the anti-HER3 antibody is an antibody comprising a heavy chain comprising CDRH1 consisting of an amino acid sequence consisting of amino acid residues 26 to 35 of SEQ ID NO: 3, CDRH2 consisting of an amino acid sequence consisting of amino acid residues 50 to 65 of SEQ ID NO: 3, and CDRH3 consisting of an amino acid sequence consisting of amino acid residues 98 to 106 of SEQ ID NO: 3, and a light chain comprising CDRL1 consisting of an amino acid sequence consisting of amino acid residues 24 to 39 of SEQ ID NO: 4, CDRL2 consisting of an amino acid sequence consisting of amino acid residues 56 to 62 of SEQ ID NO: 4, and CDRL3 consisting of an amino acid sequence consisting of amino acid residues 95 to 103 of SEQ ID NO: 4.

[A2] The pharmaceutical product according to [9-1], wherein the anti-HER3 antibody is an antibody comprising a heavy chain comprising a heavy chain variable region consisting of an amino acid sequence consisting of amino acid residues 1 to 117 of SEQ ID NO: 3, and a light chain comprising a light chain variable region consisting of an amino acid sequence consisting of amino acid residues 1 to 113 of SEQ ID NO: 4.

[10-1] The pharmaceutical product according to [9-1], wherein the anti-HER3 antibody is an antibody comprising a heavy chain consisting of an amino acid sequence represented by SEQ ID NO: 3 and a light chain consisting of an amino acid sequence represented by SEQ ID NO: 4.

[11-1] The pharmaceutical product according to [10-1], wherein the anti-HER3 antibody lacks a lysine residue at the carboxyl terminus of the heavy chain.

[12-1] The pharmaceutical product according to any one of [9-1] to [11-1] and [A1] to [A2], wherein the antibody-drug conjugate is represented by the following formula:

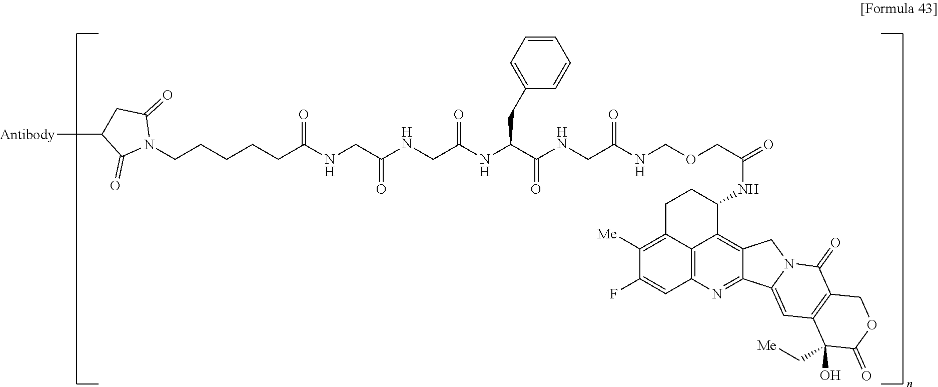

wherein ‘Antibody’ indicates the anti-HER3 antibody conjugated to the drug-linker via a thioether bond, and n indicates an average number of units of the drug-linker conjugated per antibody molecule in the antibody-drug conjugate, wherein n is in the range of from 7 to 8.

[13-1] The pharmaceutical product according to [2-1], wherein the antibody in the antibody-drug conjugate is an anti-TROP2 antibody.

[14-1] The pharmaceutical product according to [13-1], wherein the anti-TROP2 antibody is an antibody comprising a heavy chain comprising CDRH1 consisting of an amino acid sequence consisting of amino acid residues 50 to 54 of SEQ ID NO: 5, CDRH2 consisting of an amino acid sequence consisting of amino acid residues 69 to 85 of SEQ ID NO: 5, and CDRH3 consisting of an amino acid sequence consisting of amino acid residues 118 to 129 of SEQ ID NO: 5, and a light chain comprising CDRL1 consisting of an amino acid sequence consisting of amino acid residues 44 to 54 of SEQ ID NO: 6, CDRL2 consisting of an amino acid sequence consisting of amino acid residues 70 to 76 of SEQ ID NO: 6, and CDRL3 consisting of an amino acid sequence consisting of amino acid residues 109 to 117 of SEQ ID NO: 6.

[15-1] The pharmaceutical product according to [13-1], wherein the anti-TROP2 antibody is an antibody comprising a heavy chain comprising a heavy chain variable region consisting of an amino acid sequence consisting of amino acid residues 20 to 140 of SEQ ID NO: 5, and a light chain comprising a light chain variable region consisting of an amino acid sequence consisting of amino acid residues 21 to 129 of SEQ ID NO: 6.

[16-1] The pharmaceutical product according to [13-1], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

[17-1] The pharmaceutical product according to [16-1], wherein the anti-TROP2 antibody lacks a lysine residue at the carboxyl terminus of the heavy chain.

[18-1] The pharmaceutical product according to any one of [13-1] to [17-1], wherein the antibody-drug conjugate is represented by the following formula:

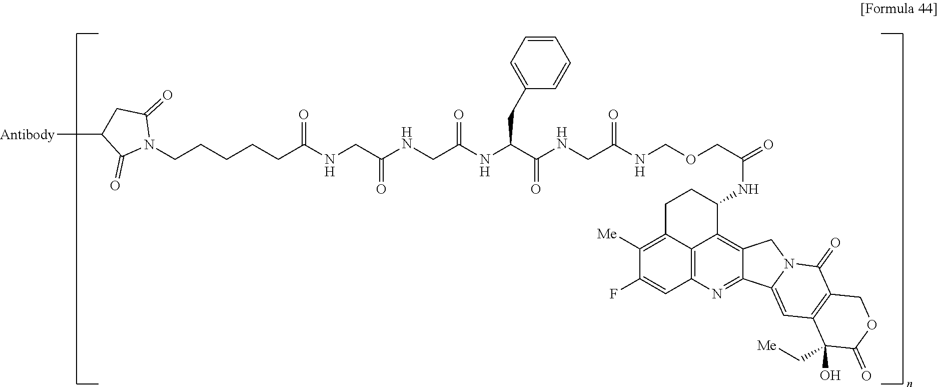

wherein ‘Antibody’ indicates the anti-TROP2 antibody conjugated to the drug-linker via a thioether bond, and n indicates an average number of units of the drug-linker conjugated per antibody molecule in the antibody-drug conjugate, wherein n is in the range of from 3.5 to 4.5.

[19-1] The pharmaceutical product according to [2-1], wherein the antibody in the antibody-drug conjugate is an anti-B7-H3 antibody.

[20-1] The pharmaceutical product according to [19-1], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

[21-1] The pharmaceutical product according to [20-1], wherein the anti-B7-H3 antibody lacks a lysine residue at the carboxyl terminus of the heavy chain.

[22-1] The pharmaceutical product according to any one of [19-1] to [21-1], wherein the antibody-drug conjugate is represented by the following formula:

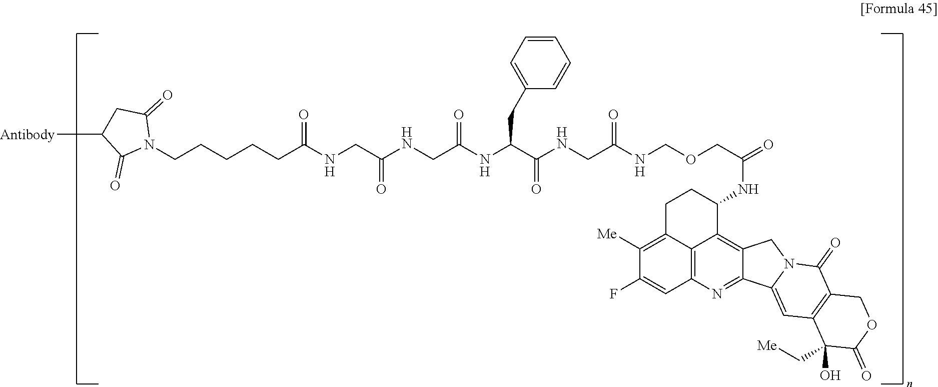

wherein ‘Antibody’ indicates the anti-B7-H3 antibody conjugated to the drug-linker via a thioether bond, and n indicates an average number of units of the drug-linker conjugated per antibody molecule in the antibody-drug conjugate, wherein n is in the range of from 3.5 to 4.5.

[23-1] The pharmaceutical product according to [2-1], wherein the antibody in the antibody-drug conjugate is an anti-GPR20 antibody.

[24-1] The pharmaceutical product according to [23-1], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

[25-1] The pharmaceutical product according to [24-1], wherein the anti-GPR20 antibody lacks a lysine residue at the carboxyl terminus of the heavy chain.

[26-1] The pharmaceutical product according to any one of [23-1] to [25-1], wherein the antibody-drug conjugate is represented by the following formula:

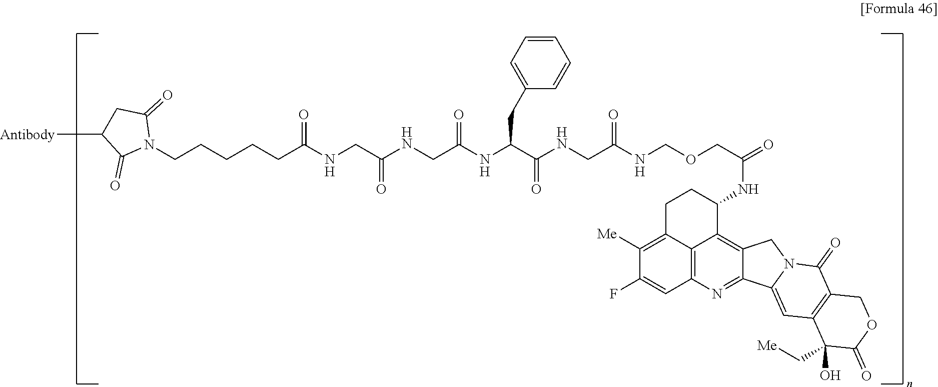

wherein ‘Antibody’ indicates the anti-GPR20 antibody conjugated to the drug-linker via a thioether bond, and n indicates an average number of units of the drug-linker conjugated per antibody molecule in the antibody-drug conjugate, wherein n is in the range of from 7 to 8.

[27-1] The pharmaceutical product according to [2-1], wherein the antibody in the antibody-drug conjugate is an anti-CDH6 antibody.

[28-1] The pharmaceutical product according to [27-1], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

[29-1] The pharmaceutical product according to [28-1], wherein the anti-CDH6 antibody lacks a lysine residue at the carboxyl terminus of the heavy chain.

[30-1] The pharmaceutical product according to any one of [27-1] to [29-1], wherein the antibody-drug conjugate is represented by the following formula:

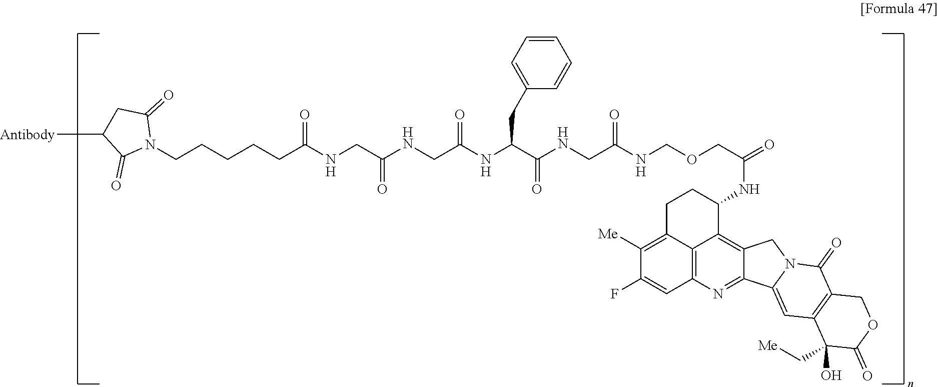

wherein ‘Antibody’ indicates the anti-CDH6 antibody conjugated to the drug-linker via a thioether bond, and n indicates an average number of units of the drug-linker conjugated per antibody molecule in the antibody-drug conjugate, wherein n is in the range of from 7 to 8.

[31-1] The pharmaceutical product according to [2-1], wherein the antibody in the antibody-drug conjugate is an anti-MUC1 antibody.

[32-1] The pharmaceutical product according to [31-1], wherein the anti-MUC1 antibody is an antibody comprising a heavy chain consisting of an amino acid sequence represented by SEQ ID NO: 13 and a light chain consisting of an amino acid sequence represented by SEQ ID NO: 15.

[33-1] The pharmaceutical product according to [32-1], wherein the anti-MUC1 antibody lacks a lysine residue at the carboxyl terminus of the heavy chain.

[34-1] The pharmaceutical product according to [31-1], wherein the anti-MUC1 antibody is an antibody comprising a heavy chain consisting of an amino acid sequence represented by SEQ ID NO: 14 and a light chain consisting of an amino acid sequence represented by SEQ ID NO: 15.

[35-1] The pharmaceutical product according to [34-1], wherein the anti-MUC1 antibody lacks a lysine residue at the carboxyl terminus of the heavy chain.

[36-1] The pharmaceutical product according to any one of [31-1] to [35-1], wherein the antibody-drug conjugate is represented by the following formula:

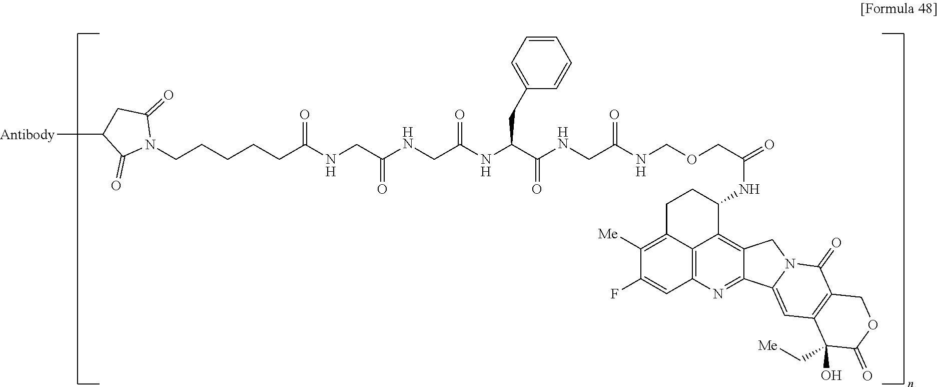

wherein ‘Antibody’ indicates the anti-MUC1 antibody conjugated to the drug-linker via a thioether bond, and n indicates an average number of units of the drug-linker conjugated per antibody molecule in the antibody-drug conjugate, wherein n is in the range of from 7 to 8.

[B1] The pharmaceutical product according to [2-1], wherein the antibody in the antibody-drug conjugate is an anti-CD37 antibody.

[B2] The pharmaceutical product according to [B1], wherein the anti-CD37 antibody is an antibody selected from the group consisting of
(a) an antibody comprising a heavy chain comprising a heavy chain variable region consisting of an amino acid sequence consisting of amino acid residues 20 to 138 of SEQ ID NO: 16 and a light chain comprising a light chain variable region consisting of an amino acid sequence consisting of amino acid residues 21 to 128 of SEQ ID NO: 19,
(b) an antibody comprising a heavy chain comprising a heavy chain variable region consisting of an amino acid sequence consisting of amino acid residues 20 to 138 of SEQ ID NO: 17 and a light chain comprising a light chain variable region consisting of an amino acid sequence consisting of amino acid residues 21 to 128 of SEQ ID NO: 19, and
(c) an antibody comprising a heavy chain comprising a heavy chain variable region consisting of an amino acid sequence consisting of amino acid residues 20 to 138 of SEQ ID NO: 18 and a light chain comprising a light chain variable region consisting of an amino acid sequence consisting of amino acid residues 21 to 128 of SEQ ID NO: 19.

[B3] The pharmaceutical product according to [B1], wherein the anti-CD37 antibody is an antibody selected from the group consisting of
(d) an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19,
(e) an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19, and
(f) an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

[B4] The pharmaceutical product according to [B1], wherein the anti-CD37 antibody is an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19.

[B5] The pharmaceutical product according to [B4], wherein the anti-CD37 antibody lacks one or two amino acid residues at the carboxyl terminus of the heavy chain.

[B6] The pharmaceutical product according to [B4] or [B5], wherein a proline residue at the carboxyl terminus of the heavy chain is amidated.

[B7] The pharmaceutical product according to [B1], wherein the anti-CD37 antibody is an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19.

[B8] The pharmaceutical product according to [B7], wherein the anti-CD37 antibody lacks one or two amino acid residues at the carboxyl terminus of the heavy chain.

[B9] The pharmaceutical product according to [B7] or [B8], wherein a proline residue at the carboxyl terminus of the heavy chain is amidated.

[B10] The pharmaceutical product according to [B1], wherein the anti-CD37 antibody is an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

[B11] The pharmaceutical product according to [B10], wherein the anti-CD37 antibody lacks one or two amino acid residues at the carboxyl terminus of the heavy chain.

[B12] The pharmaceutical product according to [B10] or [B11], wherein a proline residue at the carboxyl terminus of the heavy chain is amidated.

[B13] The pharmaceutical product according to any one of [B1] to [B12], wherein the antibody-drug conjugate is represented by the following formula:

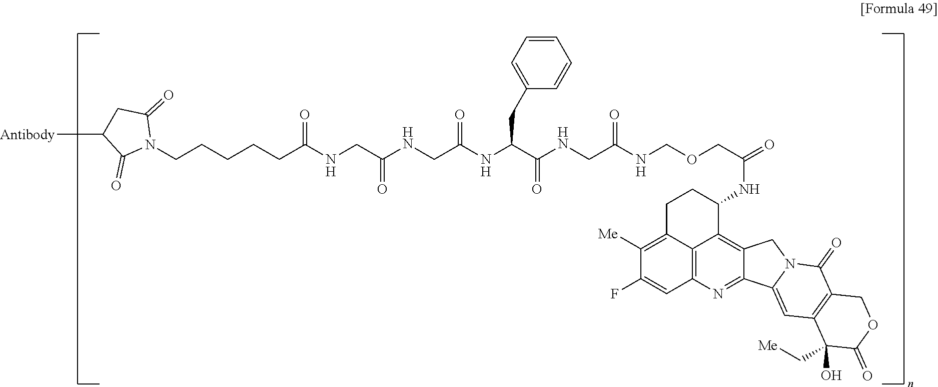

wherein ‘Antibody’ indicates the anti-CD37 antibody conjugated to the drug-linker via a thioether bond, and n indicates an average number of units of the drug-linker conjugated per antibody molecule in the antibody-drug conjugate, wherein n is in the range of from 7 to 8.

[37-1] The pharmaceutical product according to any one of [1-1] to [8-1], wherein the antibody-drug conjugate is trastuzumab deruxtecan (DS-8201a).

[38-1] The pharmaceutical product according to any one of [1-1] to [2-1] and [13-1] to [18-1], wherein the antibody-drug conjugate is datopotamab deruxtecan (DS-1062).

[39-1] The pharmaceutical product according to any one of [1-1] to [38-1], [A1] to [A2] and [B1] to [B13], wherein the inhibitor is an EZH2 inhibitor.

[40-1] The pharmaceutical product according to [39-1], wherein the inhibitor is tazemetostat or a pharmaceutically acceptable salt thereof.

[41-1] The pharmaceutical product according to any one of [1-1] to [38-1], [A1] to [A2] and [B1] to [B13], wherein the inhibitor is an EZH1/2 dual inhibitor.

[42-1] The pharmaceutical product according to [41-1], wherein the inhibitor is valemetostat or a pharmaceutically acceptable salt thereof.

[43-1] The pharmaceutical product according to [41-1] or [42-1], wherein the inhibitor is valemetostat tosylate.

[44-1] The pharmaceutical product according to any one of [1-1] to [43-1], [A1] to [A2] or [B1] to [B13], wherein the antibody-drug conjugate and the inhibitor are separately contained as active components in different formulations, and are administered simultaneously or at different times.

[45-1] The pharmaceutical product according to any one of [1-1] to [44-1], [A1] to [A2], [B1] to [B13], or [257-1], wherein the pharmaceutical product is for use in treating at least one selected from the group consisting of breast cancer, gastric cancer, colorectal cancer, lung cancer, esophageal cancer, head-and-neck cancer, gastroesophageal junction adenocarcinoma, biliary tract cancer, Paget's disease, pancreatic cancer, ovarian cancer, bladder cancer, prostate cancer, uterine carcinosarcoma, gastrointestinal stromal tumor, kidney cancer, and sarcoma.

[46-1] The pharmaceutical product according to [45-1], wherein the pharmaceutical product is for use in treating breast cancer.

[47-1] The pharmaceutical product according to [46-1], wherein the pharmaceutical product is for use in treating triple-negative breast cancer.

[48-1] The pharmaceutical product according to [45-1], wherein the pharmaceutical product is for use in treating gastric cancer.

[49-1] The pharmaceutical product according to [45-1], wherein the pharmaceutical product is for use in treating ovarian cancer.

[50-1] The pharmaceutical product according to [45-1] or, wherein the pharmaceutical product is for use in treating lung cancer.

[51-1] The pharmaceutical product according to [45-1], wherein the pharmaceutical product is for use in treating pancreatic cancer.

[52-1]A method of treatment, comprising administering an antibody-drug conjugate and an inhibitor selected from the group consisting of an EZH1 inhibitor, an EZH2 inhibitor and an EZH1/2 dual inhibitor in combination to a subject in need of the treatment, wherein the antibody-drug conjugate is an antibody-drug conjugate in which a drug-linker represented by the following formula:

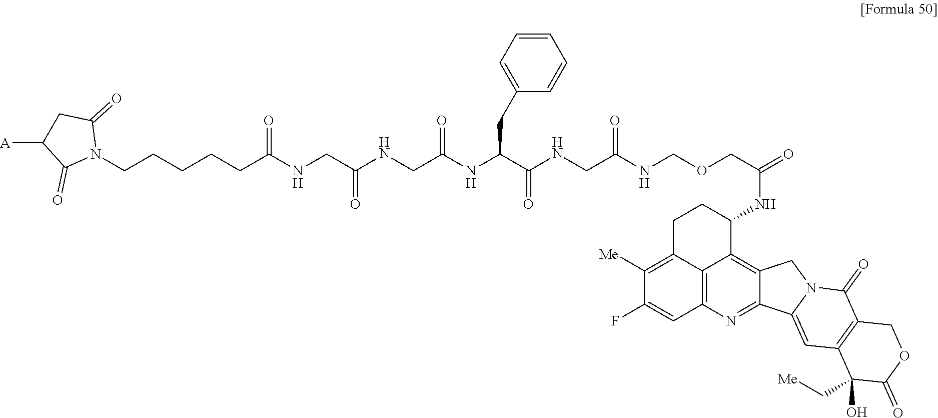

wherein A represents a connecting position to an antibody,
is conjugated to the antibody via a thioether bond.

[53-1] The method of treatment according to [52-1], wherein the antibody in the antibody-drug conjugate is an anti-HER2 antibody, an anti-HER3 antibody, an anti-TROP2 antibody, an anti-B7-H3 antibody, an anti-GPR20 antibody, an anti-CDH6 antibody, an anti-MUC1 antibody, or an anti-CD37 antibody.

[54-1] The method of treatment according to [53-1], wherein the antibody in the antibody-drug conjugate is an anti-HER2 antibody.

[55-1] The method of treatment according to [54-1], wherein the anti-HER2 antibody is an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1, and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2, and CDRL3 consisting of an amino acid sequence consisting of amino acid residues 89 to 97 of SEQ ID NO: 2.

[56-1] The method of treatment according to [54-1], wherein the anti-HER2 antibody is an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2.

[57-1] The method of treatment according to [54-1], wherein the anti-HER2 antibody is an antibody comprising a heavy chain consisting of an amino acid sequence represented by SEQ ID NO: 1 and a light chain consisting of an amino acid sequence represented by SEQ ID NO: 2.

[58-1] The method of treatment according to [54-1], wherein the anti-HER2 antibody is an antibody comprising a heavy chain consisting of an amino acid sequence consisting of amino acid residues 1 to 449 of SEQ ID NO: 1 and a light chain consisting of an amino acid sequence represented by amino acid residues 1 to 214 of SEQ ID NO: 2.

[59-1] The method of treatment according to any one of [54-1] to [58-1], wherein the antibody-drug conjugate is represented by the following formula:

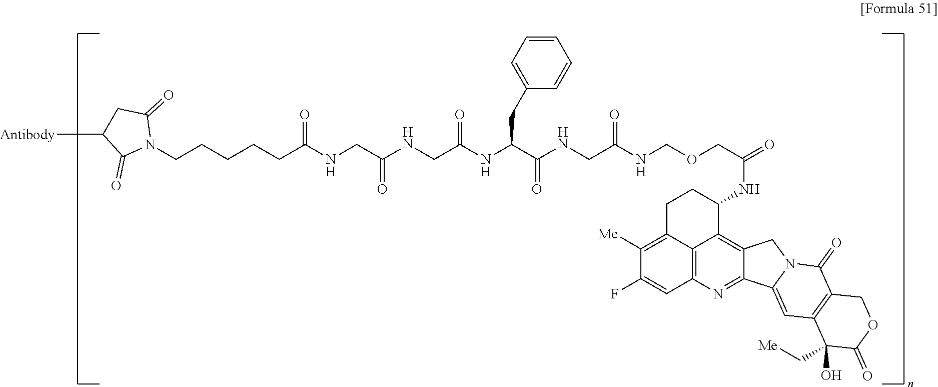

wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8.

[60-1] The method of treatment according to [53-1], wherein the antibody in the antibody-drug conjugate is an anti-HER3 antibody.

[C1] The method according to [60-1], wherein the anti-HER3 antibody is an antibody comprising a heavy chain comprising CDRH1 consisting of an amino acid sequence consisting of amino acid residues 26 to 35 of SEQ ID NO: 3, CDRH2 consisting of an amino acid sequence consisting of amino acid residues 50 to 65 of SEQ ID NO: 3, and CDRH3 consisting of an amino acid sequence consisting of amino acid residues 98 to 106 of SEQ ID NO: 3, and a light chain comprising CDRL1 consisting of an amino acid sequence consisting of amino acid residues 24 to 39 of SEQ ID NO: 4, CDRL2 consisting of an amino acid sequence consisting of amino acid residues 56 to 62 of SEQ ID NO: 4, and CDRL3 consisting of an amino acid sequence consisting of amino acid residues 95 to 103 of SEQ ID NO: 4.

[C2] The method according to [60-1], wherein the anti-HER3 antibody is an antibody comprising a heavy chain comprising a heavy chain variable region consisting of an amino acid sequence consisting of amino acid residues 1 to 117 of SEQ ID NO: 3, and a light chain comprising a light chain variable region consisting of an amino acid sequence consisting of amino acid residues 1 to 113 of SEQ ID NO: 4.

[61-1] The method of treatment according to [60-1], wherein the anti-HER3 antibody is an antibody comprising a heavy chain consisting of an amino acid sequence represented by SEQ ID NO: 3 and a light chain consisting of an amino acid sequence represented by SEQ ID NO: 4.

[62-1] The method of treatment according to [61-1], wherein the anti-HER3 antibody lacks a lysine residue at the carboxyl terminus of the heavy chain.

[63-1] The method of treatment according to any one of [60-1] to [62-1] or [C1] to [C2], wherein the antibody-drug conjugate is represented by the following formula:

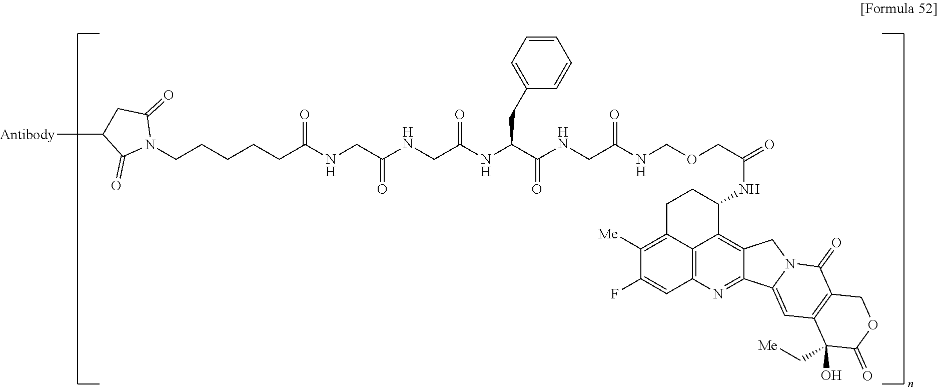

wherein ‘Antibody’ indicates the anti-HER3 antibody conjugated to the drug-linker via a thioether bond, and n indicates an average number of units of the drug-linker conjugated per antibody molecule in the antibody-drug conjugate, wherein n is in the range of from 7 to 8.

[64-1] The method of treatment according to [53-1], wherein the antibody in the antibody-drug conjugate is an anti-TROP2 antibody.

[65-1] The method of treatment according to [64-1], wherein the anti-TROP2 antibody is an antibody comprising a heavy chain comprising CDRH1 consisting of an amino acid sequence consisting of amino acid residues 50 to 54 of SEQ ID NO: 5, CDRH2 consisting of an amino acid sequence consisting of amino acid residues 69 to 85 of SEQ ID NO: 5, and CDRH3 consisting of an amino acid sequence consisting of amino acid residues 118 to 129 of SEQ ID NO: 5, and a light chain comprising CDRL1 consisting of an amino acid sequence consisting of amino acid residues 44 to 54 of SEQ ID NO: 6, CDRL2 consisting of an amino acid sequence consisting of amino acid residues 70 to 76 of SEQ ID NO: 6, and CDRL3 consisting of an amino acid sequence consisting of amino acid residues 109 to 117 of SEQ ID NO: 6.

[66-1] The method of treatment according to [64-1], wherein the anti-TROP2 antibody is an antibody comprising a heavy chain comprising a heavy chain variable region consisting of an amino acid sequence consisting of amino acid residues 20 to 140 of SEQ ID NO: 5, and a light chain comprising a light chain variable region consisting of an amino acid sequence consisting of amino acid residues 21 to 129 of SEQ ID NO: 6.

[67-1] The method of treatment according to [64-1], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

[68-1] The method of treatment according to [67-1], wherein the anti-TROP2 antibody lacks a lysine residue at the carboxyl terminus of the heavy chain.

[69-1] The method of treatment according to any one of [64-1] to [68-1], wherein the antibody-drug conjugate is represented by the following formula:

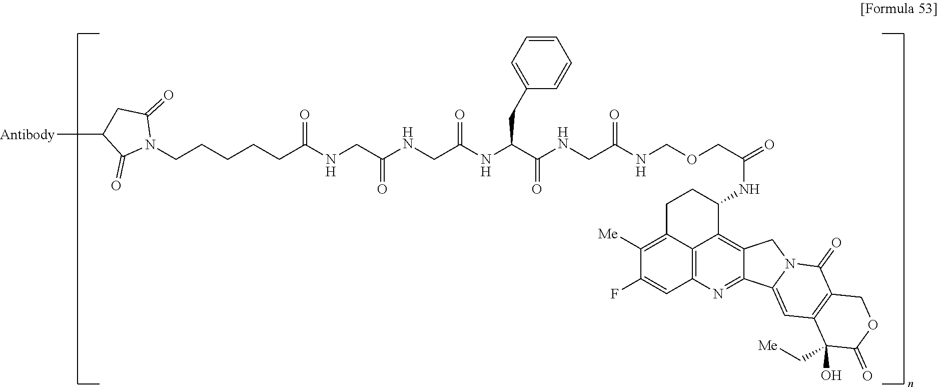

wherein ‘Antibody’ indicates the anti-TROP2 antibody conjugated to the drug-linker via a thioether bond, and n indicates an average number of units of the drug-linker conjugated per antibody molecule in the antibody-drug conjugate, wherein n is in the range of from 3.5 to 4.5.

[70-1] The method of treatment according to [53-1], wherein the antibody in the antibody-drug conjugate is an anti-B7-H3 antibody.

[71-1] The method of treatment according to [70-1], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

[72-1] The method of treatment according to [71-1], wherein the anti-B7-H3 antibody lacks a lysine residue at the carboxyl terminus of the heavy chain.

[73-1] The method of treatment according to any one of [70-1] to [72-1], wherein the antibody-drug conjugate is represented by the following formula:

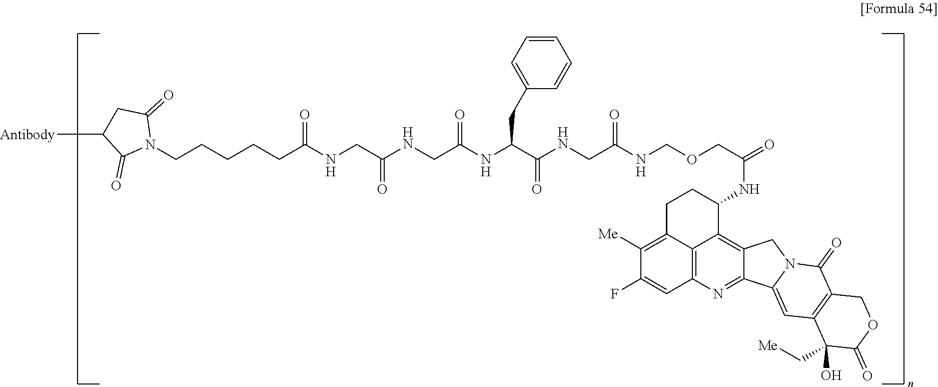

wherein ‘Antibody’ indicates the anti-B7-H3 antibody conjugated to the drug-linker via a thioether bond, and n indicates an average number of units of the drug-linker conjugated per antibody molecule in the antibody-drug conjugate, wherein n is in the range of from 3.5 to 4.5.

[74-1] The method of treatment according to [53-1], wherein the antibody in the antibody-drug conjugate is an anti-GPR20 antibody.

[75-1] The method of treatment according to [74-1], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

[76-1] The method of treatment according to [75-1], wherein the anti-GPR20 antibody lacks a lysine residue at the carboxyl terminus of the heavy chain.

[77-1] The method of treatment according to any one of [74-1] to [76-1], wherein the antibody-drug conjugate is represented by the following formula:

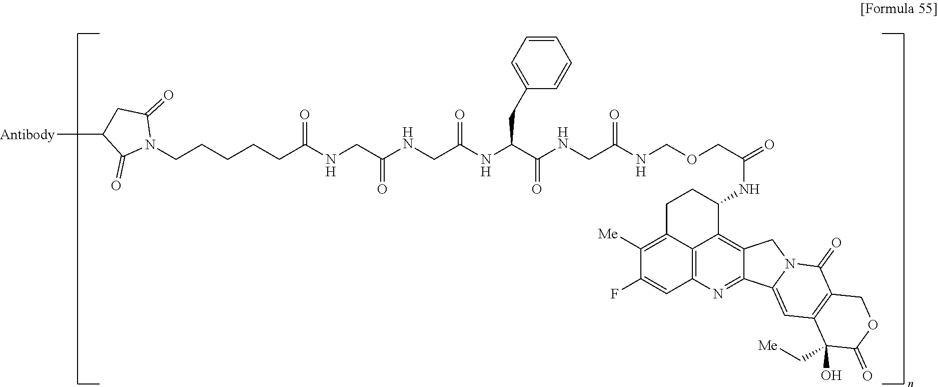

wherein ‘Antibody’ indicates the anti-GPR20 antibody conjugated to the drug-linker via a thioether bond, and n indicates an average number of units of the drug-linker conjugated per antibody molecule in the antibody-drug conjugate, wherein n is in the range of from 7 to 8.

[78-1] The method of treatment according to [53-1], wherein the antibody in the antibody-drug conjugate is an anti-CDH6 antibody.

[79-1] The method of treatment according to [78-1], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

[80-1] The method of treatment according to [79-1], wherein the anti-CDH6 antibody lacks a lysine residue at the carboxyl terminus of the heavy chain.

[81-1] The method of treatment according to any one of [78-1] to [80-1], wherein the antibody-drug conjugate is represented by the following formula:

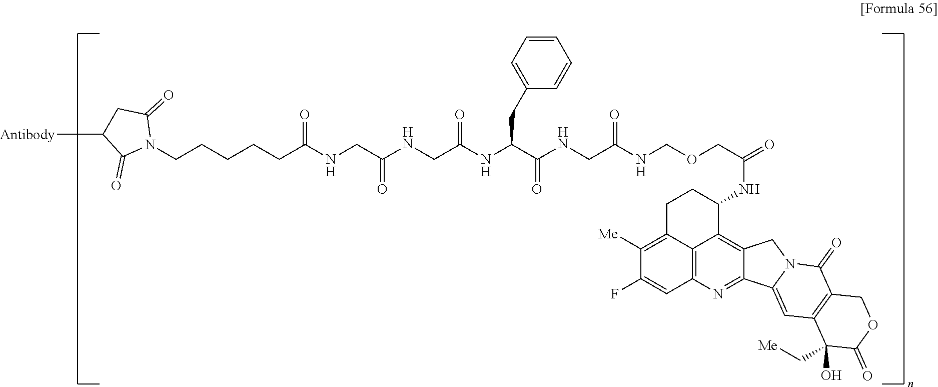

wherein ‘Antibody’ indicates the anti-CDH6 antibody conjugated to the drug-linker via a thioether bond, and n indicates an average number of units of the drug-linker conjugated per antibody molecule in the antibody-drug conjugate, wherein n is in the range of from 7 to 8.

[82-1] The method according to [53-1], wherein the antibody in the antibody-drug conjugate is an anti-MUC1 antibody.

[83-1] The method according to [82-1], wherein the anti-MUC1 antibody is an antibody comprising a heavy chain consisting of an amino acid sequence represented by SEQ ID NO: 13 and a light chain consisting of an amino acid sequence represented by SEQ ID NO: 15.

[84-1] The method according to [83-1], wherein the anti-MUC1 antibody lacks a lysine residue at the carboxyl terminus of the heavy chain.

[85-1] The method according to [82-1], wherein the anti-MUC1 antibody is an antibody comprising a heavy chain consisting of an amino acid sequence represented by SEQ ID NO: 14 and a light chain consisting of an amino acid sequence represented by SEQ ID NO: 15.

[86-1] The method according to [85-1], wherein the anti-MUC1 antibody lacks a lysine residue at the carboxyl terminus of the heavy chain.

[87-1] The method according to any one of [82-1] to [86-1], wherein the antibody-drug conjugate is represented by the following formula:

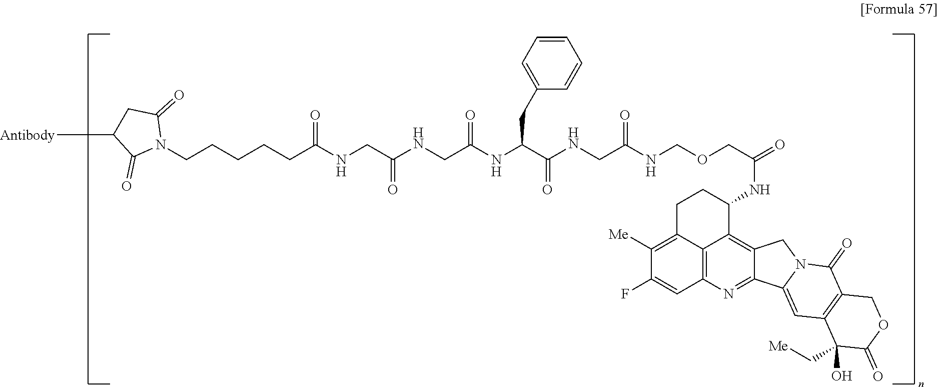

wherein ‘Antibody’ indicates the anti-MUC1 antibody conjugated to the drug-linker via a thioether bond, and n indicates an average number of units of the drug-linker conjugated per antibody molecule in the antibody-drug conjugate, wherein n is in the range of from 7 to 8.

[D1] The method according to [53-1], wherein the antibody in the antibody-drug conjugate is an anti-CD37 antibody.

[D2] The method according to [D1], wherein the anti-CD37 antibody is an antibody selected from the group consisting of
(a) an antibody comprising a heavy chain comprising a heavy chain variable region consisting of an amino acid sequence consisting of amino acid residues 20 to 138 of SEQ ID NO: 16 and a light chain comprising a light chain variable region consisting of an amino acid sequence consisting of amino acid residues 21 to 128 of SEQ ID NO: 19,
(b) an antibody comprising a heavy chain comprising a heavy chain variable region consisting of an amino acid sequence consisting of amino acid residues 20 to 138 of SEQ ID NO: 17 and a light chain comprising a light chain variable region consisting of an amino acid sequence consisting of amino acid residues 21 to 128 of SEQ ID NO: 19, and
(c) an antibody comprising a heavy chain comprising a heavy chain variable region consisting of an amino acid sequence consisting of amino acid residues 20 to 138 of SEQ ID NO: 18 and a light chain comprising a light chain variable region consisting of an amino acid sequence consisting of amino acid residues 21 to 128 of SEQ ID NO: 19.

[D3] The method according to [D1], wherein the anti-CD37 antibody is an antibody selected from the group consisting of
(d) an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19,
(e) an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19, and
(f) an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

[D4] The method according to [D1], wherein the anti-CD37 antibody is an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19.

[D5] The method according to [D4], wherein the anti-CD37 antibody lacks one or two amino acid residues at the carboxyl terminus of the heavy chain.

[D6] The method according to [D4] or [D5], wherein a proline residue at the carboxyl terminus of the heavy chain is amidated.

[D7] The method according to [D1], wherein the anti-CD37 antibody is an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19.

[D8] The method according to [D7], wherein the anti-CD37 antibody lacks one or two amino acid residues at the carboxyl terminus of the heavy chain.

[D9] The method according to [D7] or [D8], wherein a proline residue at the carboxyl terminus of the heavy chain is amidated.

[D10] The method according to [D1], wherein the anti-CD37 antibody is an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

[D11] The method according to [D10], wherein the anti-CD37 antibody lacks one or two amino acid residues at the carboxyl terminus of the heavy chain.

[D12] The method according to [D10] or [D11], wherein a proline residue at the carboxyl terminus of the heavy chain is amidated.

[D13] The method according to any one of [D1] to [D12], wherein the antibody-drug conjugate is represented by the following formula:

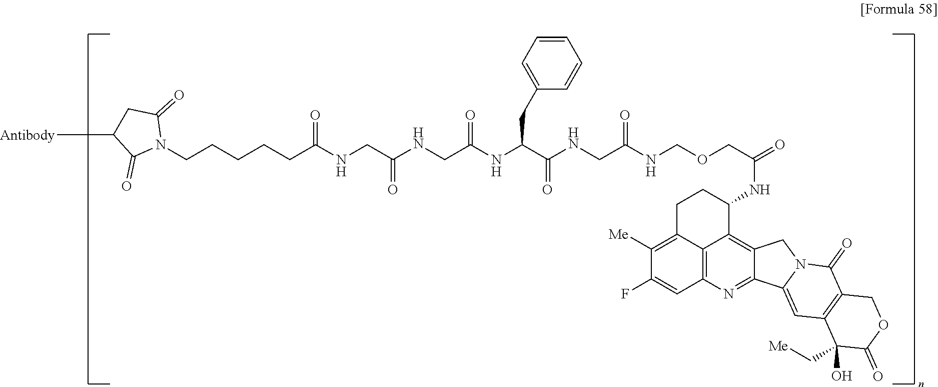

wherein ‘Antibody’ indicates the anti-CD37 antibody conjugated to the drug-linker via a thioether bond, and n indicates an average number of units of the drug-linker conjugated per antibody molecule in the antibody-drug conjugate, wherein n is in the range of from 7 to 8.

[88-1] The method of treatment according to any one of [52-1] to [59-1], wherein the antibody-drug conjugate is trastuzumab deruxtecan (DS-8201a).

[89-1] The method of treatment according to any one of [52-1] to [53-1] and [64-1] to [69-1], wherein the antibody-drug conjugate is datopotamab deruxtecan (DS-1062).

[90-1] The method according to any one of [52-1] to [89-1], [C1] to [C2] and [D1] to [D13], wherein the inhibitor is an EZH2 inhibitor.

[91-1] The method according to [90-1], wherein the inhibitor is tazemetostat or a pharmaceutically acceptable salt thereof.

[92-1] The method according to any one of [52-1] to [89-1], [C1] to [C2] and [D1] to [D13], wherein the inhibitor is an EZH1/2 dual inhibitor.

[93-1] The method according to [92-1], wherein the inhibitor is valemetostat or a pharmaceutically acceptable salt thereof.

[94-1] The method according to [92-1] or [93-1] wherein the inhibitor is valemetostat tosylate.

[95-1] The method according to any one of [52-1] to [94-1], [C1] to [C2] and [D1] to [D13], wherein the antibody-drug conjugate and the inhibitor are separately contained as active components in different formulations, and are administered simultaneously or at different times.

[96-1] The method according to any one of [52-1] to [95-1], [C1] to [C2], [D1] to [D13], and [258-1], wherein the method of treatment is for use in treating at least one selected from the group consisting of breast cancer, gastric cancer, colorectal cancer, lung cancer, esophageal cancer, head-and-neck cancer, gastroesophageal junction adenocarcinoma, biliary tract cancer, Paget's disease, pancreatic cancer, ovarian cancer, bladder cancer, prostate cancer, uterine carcinosarcoma, gastrointestinal stromal tumor, kidney cancer, and sarcoma.

[97-1] The method according to [96-1], wherein the method of treatment is for use in treating breast cancer.

[98-1] The method according to [97-1], wherein the method of treatment is for use in treating triple-negative breast cancer.

[99-1] The method according to [96-1], wherein the method of treatment is for use in treating gastric cancer.

[100-1] The method according to [96-1], wherein the method of treatment is for use in treating ovarian cancer.

[101-1] The method according to [96-1], wherein the method of treatment is for use in treating lung cancer.

[102-1] The method according to [96-1], wherein the method of treatment is for use in treating pancreatic cancer.

[103-1] An antibody-drug conjugate for use in a method of treating a disease, wherein the method comprises administering the antibody-drug conjugate in combination with an inhibitor selected from the group consisting of an EZH1 inhibitor, an EZH2 inhibitor and an EZH1/2 dual inhibitor, wherein the antibody-drug conjugate is an antibody-drug conjugate in which a drug-linker represented by the following formula:

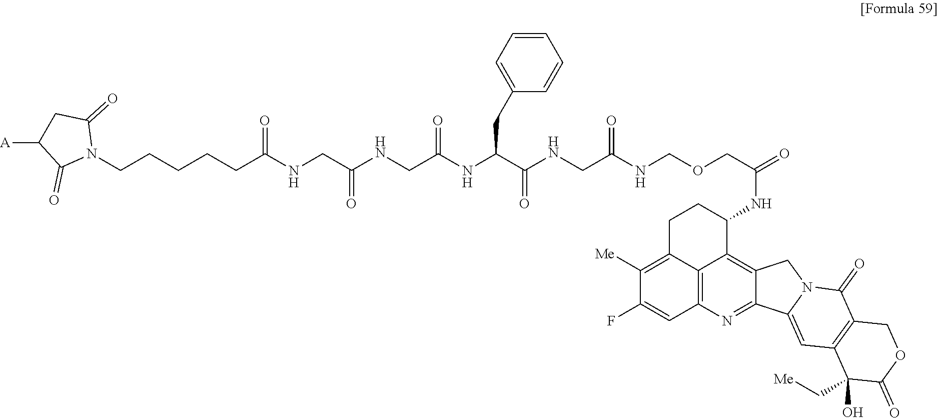

wherein A represents a connecting position to an antibody,
is conjugated to the antibody via a thioether bond in the antibody-drug conjugate.

[104-1] The antibody-drug conjugate for use according to [103-1], wherein the antibody in the antibody-drug conjugate is an anti-HER2 antibody, an anti-HER3 antibody, an anti-TROP2 antibody, an anti-B7-H3 antibody, an anti-GPR20 antibody, an anti-CDH6 antibody, an anti-MUC1 antibody, or an anti-CD37 antibody.

[105-1] The antibody-drug conjugate for use according to [104-1], wherein the antibody in the antibody-drug conjugate is an anti-HER2 antibody.

[106-1] The antibody-drug conjugate for use according to [105-1], wherein the anti-HER2 antibody is an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1, and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2, and CDRL3 consisting of an amino acid sequence consisting of amino acid residues 89 to 97 of SEQ ID NO: 2.

[107-1] The antibody-drug conjugate for use according to [105-1], wherein the anti-HER2 antibody is an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2.

[108-1] The antibody-drug conjugate for use according to [105-1], wherein the anti-HER2 antibody is an antibody comprising a heavy chain consisting of an amino acid sequence represented by SEQ ID NO: 1 and a light chain consisting of an amino acid sequence represented by SEQ ID NO: 2.

[109-1] The antibody-drug conjugate for use according to [105-1], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

[110-1] The antibody-drug conjugate for use according to any one of [105-1] to [109-1], wherein the antibody-drug conjugate is represented by the following formula:

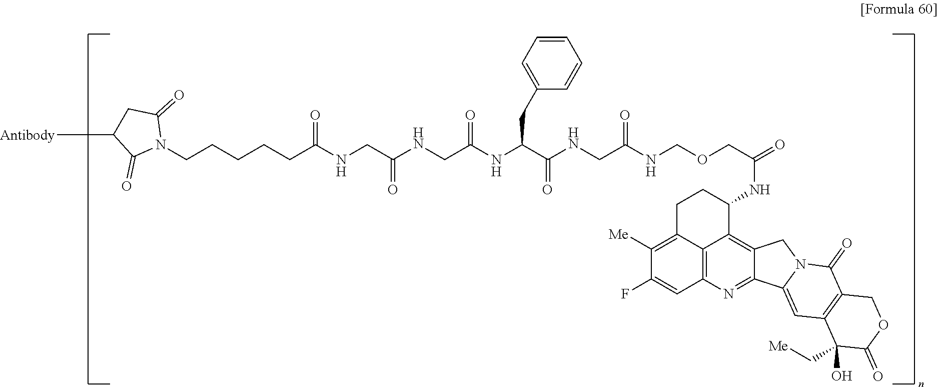

wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8.

[111-1] The antibody-drug conjugate for use according to [104-1], wherein the antibody in the antibody-drug conjugate is an anti-HER3 antibody.

[E1] The antibody-drug conjugate for use according to [111-1], wherein the anti-HER3 antibody is an antibody comprising a heavy chain comprising CDRH1 consisting of an amino acid sequence consisting of amino acid residues 26 to 35 of SEQ ID NO: 3, CDRH2 consisting of an amino acid sequence consisting of amino acid residues 50 to 65 of SEQ ID NO: 3, and CDRH3 consisting of an amino acid sequence consisting of amino acid residues 98 to 106 of SEQ ID NO: 3, and a light chain comprising CDRL1 consisting of an amino acid sequence consisting of amino acid residues 24 to 39 of SEQ ID NO: 4, CDRL2 consisting of an amino acid sequence consisting of amino acid residues 56 to 62 of SEQ ID NO: 4, and CDRL3 consisting of an amino acid sequence consisting of amino acid residues 95 to 103 of SEQ ID NO: 4.

[E2] The antibody-drug conjugate for use according to [111-1], wherein the anti-HER3 antibody is an antibody comprising a heavy chain comprising a heavy chain variable region consisting of an amino acid sequence consisting of amino acid residues 1 to 117 of SEQ ID NO: 3, and a light chain comprising a light chain variable region consisting of an amino acid sequence consisting of amino acid residues 1 to 113 of SEQ ID NO: 4.

[112-1] The antibody-drug conjugate for use according to [111-1], wherein the anti-HER3 antibody is an antibody comprising a heavy chain consisting of an amino acid sequence represented by SEQ ID NO: 3 and a light chain consisting of an amino acid sequence represented by SEQ ID NO: 4.

[113-1] The antibody-drug conjugate for use according to [112-1], wherein the anti-HER3 antibody lacks a lysine residue at the carboxyl terminus of the heavy chain.

[114-1] The antibody-drug conjugate for use according to any one of [111-1] to [113-1] or [E1] to [E2], wherein the antibody-drug conjugate is represented by the following formula:

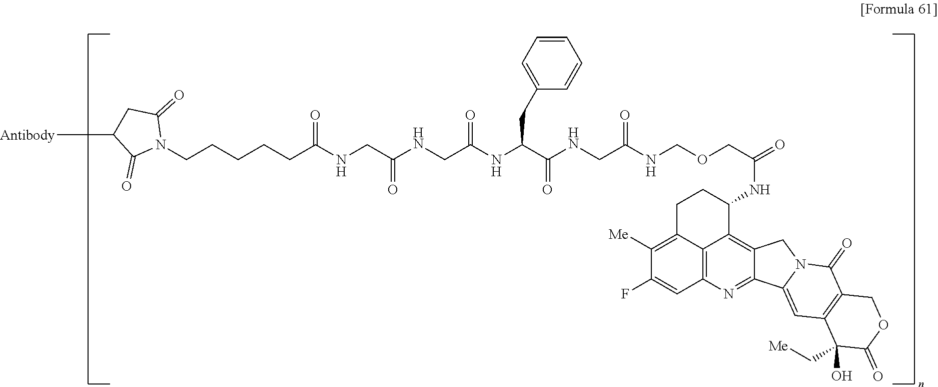

wherein ‘Antibody’ indicates the anti-HER3 antibody conjugated to the drug-linker via a thioether bond, and n indicates an average number of units of the drug-linker conjugated per antibody molecule in the antibody-drug conjugate, wherein n is in the range of from 7 to 8.

[115-1] The antibody-drug conjugate for use according to [104-1], wherein the antibody in the antibody-drug conjugate is an anti-TROP2 antibody.

[116-1] The antibody-drug conjugate for use according to [115-1], wherein the anti-TROP2 antibody is an antibody comprising a heavy chain comprising CDRH1 consisting of an amino acid sequence consisting of amino acid residues 50 to 54 of SEQ ID NO: 5, CDRH2 consisting of an amino acid sequence consisting of amino acid residues 69 to 85 of SEQ ID NO: 5, and CDRH3 consisting of an amino acid sequence consisting of amino acid residues 118 to 129 of SEQ ID NO: 5, and a light chain comprising CDRL1 consisting of an amino acid sequence consisting of amino acid residues 44 to 54 of SEQ ID NO: 6, CDRL2 consisting of an amino acid sequence consisting of amino acid residues 70 to 76 of SEQ ID NO: 6, and CDRL3 consisting of an amino acid sequence consisting of amino acid residues 109 to 117 of SEQ ID NO: 6.

[117-1] The antibody-drug conjugate for use according to [115-1], wherein the anti-TROP2 antibody is an antibody comprising a heavy chain comprising a heavy chain variable region consisting of an amino acid sequence consisting of amino acid residues 20 to 140 of SEQ ID NO: 5, and a light chain comprising a light chain variable region consisting of an amino acid sequence consisting of amino acid residues 21 to 129 of SEQ ID NO: 6.

[118-1] The antibody-drug conjugate for use according to [115-1], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

[119-1] The antibody-drug conjugate for use according to [118-1], wherein the anti-TROP2 antibody lacks a lysine residue at the carboxyl terminus of the heavy chain.

[120-1] The antibody-drug conjugate for use according to any one of [115-1] to [119-1], wherein the antibody-drug conjugate is represented by the following formula:

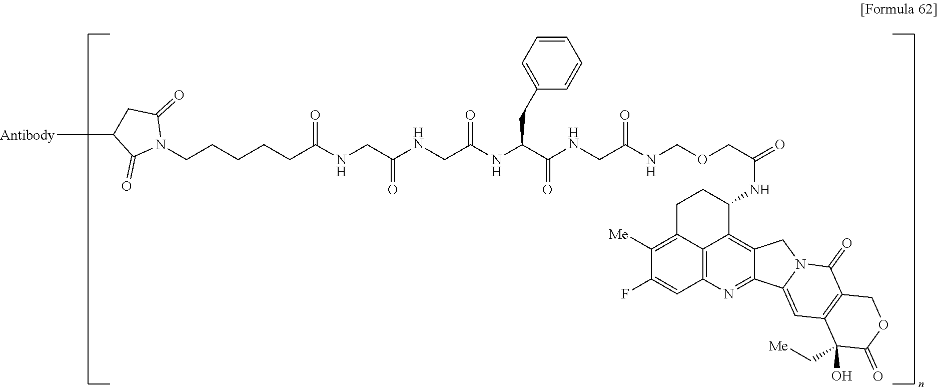

wherein ‘Antibody’ indicates the anti-TROP2 antibody conjugated to the drug-linker via a thioether bond, and n indicates an average number of units of the drug-linker conjugated per antibody molecule in the antibody-drug conjugate, wherein n is in the range of from 3.5 to 4.5.

[121-1] The antibody-drug conjugate for use according to [104-1], wherein the antibody in the antibody-drug conjugate is an anti-B7-H3 antibody.

[122-1] The antibody-drug conjugate for use according to [121-1], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

[123-1] The antibody-drug conjugate for use according to [122-1], wherein the anti-B7-H3 antibody lacks a lysine residue at the carboxyl terminus of the heavy chain.

[124-1] The antibody-drug conjugate for use according to any one of [121-1] to [123-1], wherein the antibody-drug conjugate is represented by the following formula:

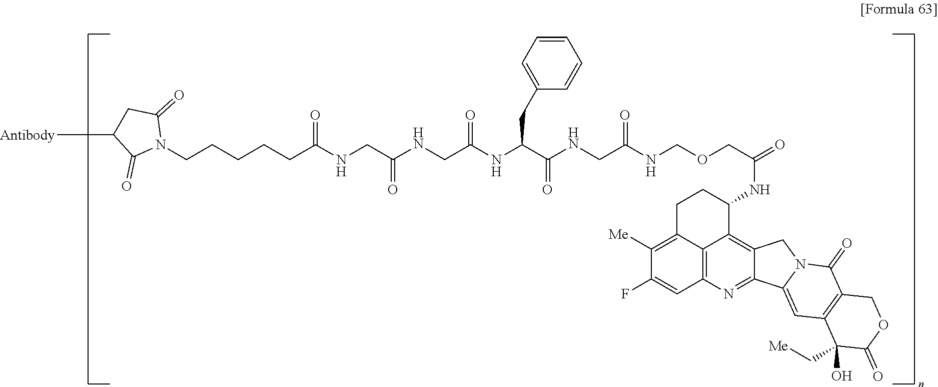

wherein ‘Antibody’ indicates the anti-B7-H3 antibody conjugated to the drug-linker via a thioether bond, and n indicates an average number of units of the drug-linker conjugated per antibody molecule in the antibody-drug conjugate, wherein n is in the range of from 3.5 to 4.5.

[125-1] The antibody-drug conjugate for use according to [104-1], wherein the antibody in the antibody-drug conjugate is an anti-GPR20 antibody.

[126-1] The antibody-drug conjugate for use according to [125-1], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

[127-1] The antibody-drug conjugate for use according to [126-1], wherein the anti-GPR20 antibody lacks a lysine residue at the carboxyl terminus of the heavy chain.

[128-1] The antibody-drug conjugate for use according to any one of [125-1] to [127-1], wherein the antibody-drug conjugate is represented by the following formula:

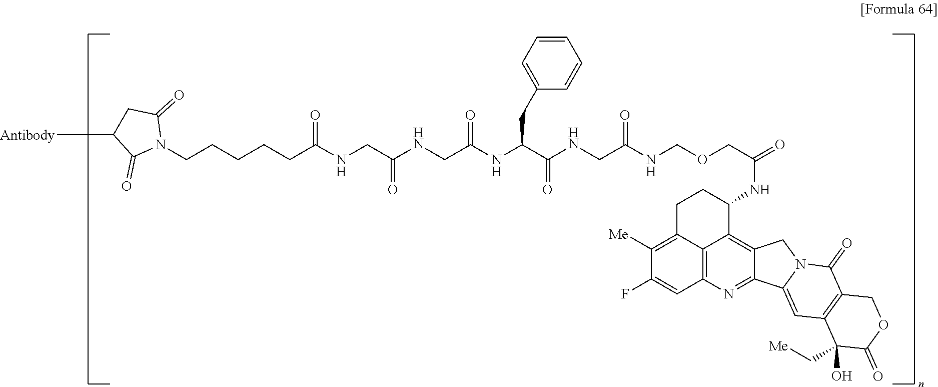

wherein ‘Antibody’ indicates the anti-GPR20 antibody conjugated to the drug-linker via a thioether bond, and n indicates an average number of units of the drug-linker conjugated per antibody molecule in the antibody-drug conjugate, wherein n is in the range of from 7 to 8.

[129-1] The antibody-drug conjugate for use according to [104-1], wherein the antibody in the antibody-drug conjugate is an anti-CDH6 antibody.

[130-1] The antibody-drug conjugate for use according to [129-1], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

[131-1] The antibody-drug conjugate for use according to [130-1], wherein the anti-CDH6 antibody lacks a lysine residue at the carboxyl terminus of the heavy chain.

[132-1] The antibody-drug conjugate for use according to any one of [129-1] to [131-1], wherein the antibody-drug conjugate is represented by the following formula:

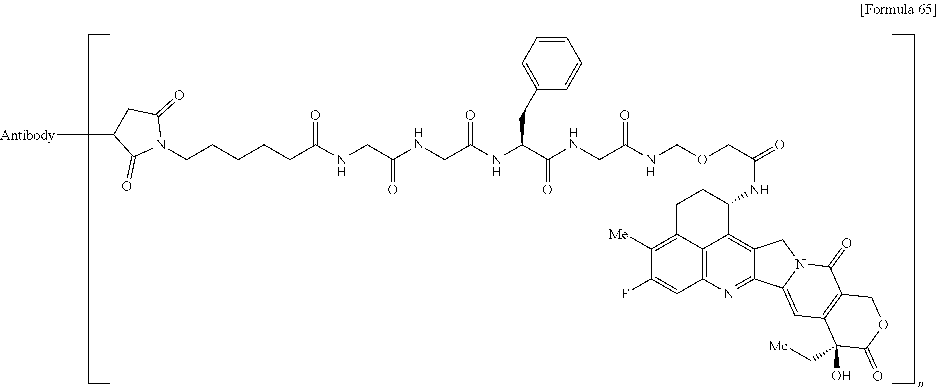

wherein ‘Antibody’ indicates the anti-CDH6 antibody conjugated to the drug-linker via a thioether bond, and n indicates an average number of units of the drug-linker conjugated per antibody molecule in the antibody-drug conjugate, wherein n is in the range of from 7 to 8.

[133-1] The antibody-drug conjugate for use according to [104-1], wherein the antibody in the antibody-drug conjugate is an anti-MUC1 antibody.

[134-1] The antibody-drug conjugate for use according to [133-1], wherein the anti-MUC1 antibody is an antibody comprising a heavy chain consisting of an amino acid sequence represented by SEQ ID NO: 13 and a light chain consisting of an amino acid sequence represented by SEQ ID NO: 15.

[135-1] The antibody-drug conjugate for use according to [134-1], wherein the anti-MUC1 antibody lacks a lysine residue at the carboxyl terminus of the heavy chain.

[136-1] The antibody-drug conjugate for use according to [133-1], wherein the anti-MUC1 antibody is an antibody comprising a heavy chain consisting of an amino acid sequence represented by SEQ ID NO: 14 and a light chain consisting of an amino acid sequence represented by SEQ ID NO: 15.

[137-1] The antibody-drug conjugate for use according to [136-1], wherein the anti-MUC1 antibody lacks a lysine residue at the carboxyl terminus of the heavy chain.

[138-1] The antibody-drug conjugate for use according to any one of [133-1] to [137-1], wherein the antibody-drug conjugate is represented by the following formula:

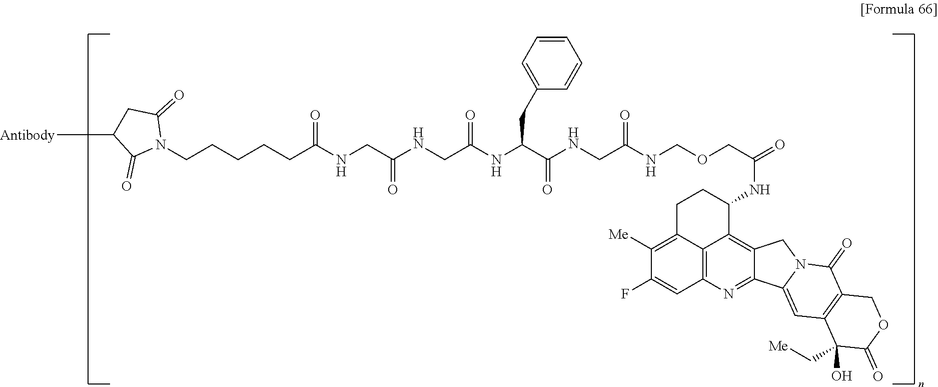

wherein ‘Antibody’ indicates the anti-MUC1 antibody conjugated to the drug-linker via a thioether bond, and n indicates an average number of units of the drug-linker conjugated per antibody molecule in the antibody-drug conjugate, wherein n is in the range of from 7 to 8.

[F1] The antibody-drug conjugate for use according to [104-1], wherein the antibody in the antibody-drug conjugate is an anti-CD37 antibody.

[F2] The antibody-drug conjugate for use according to [F1], wherein the anti-CD37 antibody is an antibody selected from the group consisting of
(a) an antibody comprising a heavy chain comprising a heavy chain variable region consisting of an amino acid sequence consisting of amino acid residues 20 to 138 of SEQ ID NO: 16 and a light chain comprising a light chain variable region consisting of an amino acid sequence consisting of amino acid residues 21 to 128 of SEQ ID NO: 19,
(b) an antibody comprising a heavy chain comprising a heavy chain variable region consisting of an amino acid sequence consisting of amino acid residues 20 to 138 of SEQ ID NO: 17 and a light chain comprising a light chain variable region consisting of an amino acid sequence consisting of amino acid residues 21 to 128 of SEQ ID NO: 19, and
(c) an antibody comprising a heavy chain comprising a heavy chain variable region consisting of an amino acid sequence consisting of amino acid residues 20 to 138 of SEQ ID NO: 18 and a light chain comprising a light chain variable region consisting of an amino acid sequence consisting of amino acid residues 21 to 128 of SEQ ID NO: 19.

[F3] The antibody-drug conjugate for use according to [F1], wherein the anti-CD37 antibody is an antibody selected from the group consisting of
(d) an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19,
(e) an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19, and
(f) an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

[F4] The antibody-drug conjugate for use according to [F1], wherein the anti-CD37 antibody is an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19.

[F5] The antibody-drug conjugate for use according to [F4], wherein the anti-CD37 antibody lacks one or two amino acid residues at the carboxyl terminus of the heavy chain.

[F6] The antibody-drug conjugate for use according to [F4] or [F5], wherein a proline residue at the carboxyl terminus of the heavy chain is amidated.

[F7] The antibody-drug conjugate for use according to [F1], wherein the anti-CD37 antibody is an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19.

[F8] The antibody-drug conjugate for use according to [F7], wherein the anti-CD37 antibody lacks one or two amino acid residues at the carboxyl terminus of the heavy chain.

[F9] The antibody-drug conjugate for use according to [F7] or [F8], wherein a proline residue at the carboxyl terminus of the heavy chain is amidated.

[F10] The antibody-drug conjugate for use according to [F1], wherein the anti-CD37 antibody is an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

[F11] The antibody-drug conjugate for use according to [F10], wherein the anti-CD37 antibody lacks one or two amino acid residues at the carboxyl terminus of the heavy chain.

[F12] The antibody-drug conjugate for use according to [F10] or [F11], wherein a proline residue at the carboxyl terminus of the heavy chain is amidated.

[F13] The antibody-drug conjugate for use according to any one of [F1] to [F12], wherein the antibody-drug conjugate is represented by the following formula:

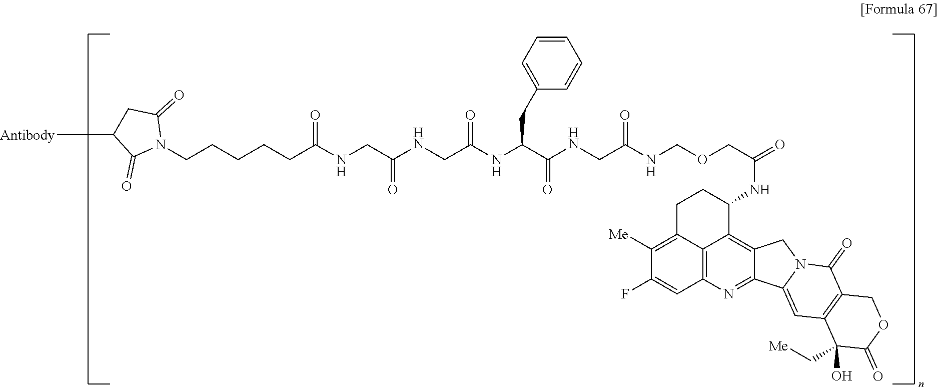

wherein ‘Antibody’ indicates the anti-CD37 antibody conjugated to the drug-linker via a thioether bond, and n indicates an average number of units of the drug-linker conjugated per antibody molecule in the antibody-drug conjugate, wherein n is in the range of from 7 to 8.

[139-1] The antibody-drug conjugate for use according to any one of [103-1] to [110-1], wherein the antibody-drug conjugate is trastuzumab deruxtecan (DS-8201a).

[140-1] The antibody-drug conjugate for use according to any one of [103-1] to [104-1] and [115-1] to [120-1], wherein the antibody-drug conjugate is datopotamab deruxtecan (DS-1062).

[141-1] The antibody-drug conjugate for use according to any one of [103-1] to [140-1], [E1] to [E2] and [F1] to [F13], wherein the inhibitor is an EZH2 inhibitor.

[142-1] The antibody-drug conjugate for use according to [141-1], wherein the inhibitor is tazemetostat or a pharmaceutically acceptable salt thereof.

[143-1] The antibody-drug conjugate for use according to any one of [103-1] to [140-1], [E1] to [E2] and [F1] to [F13], wherein the inhibitor is an EZH1/2 dual inhibitor.

[144-1] The antibody-drug conjugate for use according to [143-1], wherein the inhibitor is valemetostat or a pharmaceutically acceptable salt thereof.

[145-1] The antibody-drug conjugate for use according to [143-1] or [144-1], wherein the inhibitor is valemetostat tosylate.

[146-1] The antibody-drug conjugate for use according to any one of [103-1] to [145-1], [E1] to [E2], and [F1] to [F13], wherein the antibody-drug conjugate and the inhibitor are separately contained as active components in different formulations, and are administered simultaneously or at different times.

[147-1] The antibody-drug conjugate for use according to any one of [103-1] to [146-1], [E1] to [E2], [F1] to [F13], and [259-1], wherein the disease is at least one selected from the group consisting of breast cancer, gastric cancer, colorectal cancer, lung cancer, esophageal cancer, head-and-neck cancer, gastroesophageal junction adenocarcinoma, biliary tract cancer, Paget's disease, pancreatic cancer, ovarian cancer, bladder cancer, prostate cancer, uterine carcinosarcoma, gastrointestinal stromal tumor, kidney cancer, and sarcoma.

[148-1] The antibody-drug conjugate for use according to [147-1], wherein the disease is breast cancer.

[149-1] The antibody-drug conjugate for use according to [148-1], wherein the disease is triple-negative breast cancer.

[150-1] The antibody-drug conjugate for use according to [147-1], wherein the disease is gastric cancer.

[151-1] The antibody-drug conjugate for use according to [147-1], wherein the disease is ovarian cancer.

[152-1] The antibody-drug conjugate for use according to [147-1], wherein the disease is lung cancer.

[153-1] The antibody-drug conjugate for use according to [147-1], wherein the disease is pancreatic cancer.

[154-1] Use of an antibody-drug conjugate or an inhibitor selected from the group consisting of an EZH1 inhibitor, an EZH2 inhibitor and an EZH1/2 dual inhibitor for the manufacture of a medicament for treating a disease by administration of the antibody-drug conjugate and the inhibitor in combination, wherein the antibody-drug conjugate is an antibody-drug conjugate in which a drug-linker represented by the following formula:

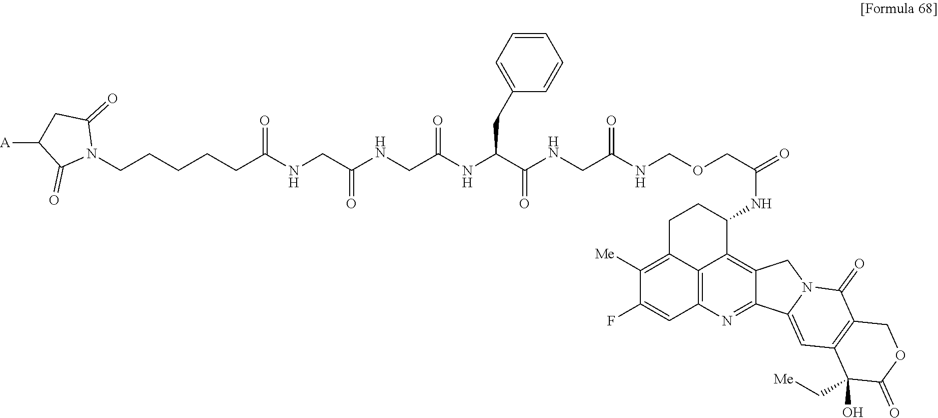

wherein A represents a connecting position to an antibody,
is conjugated to the antibody via a thioether bond in the antibody-drug conjugate.

[155-1] The use according to [154-1], wherein the antibody in the antibody-drug conjugate is an anti-HER2 antibody, an anti-HER3 antibody, an anti-TROP2 antibody, an anti-B7-H3 antibody, an anti-GPR20 antibody, an anti-CDH6 antibody, an anti-MUC1 antibody, or an anti-CD37 antibody.

[156-1] The use according to [155-1], wherein the antibody in the antibody-drug conjugate is an anti-HER2 antibody.

[157-1] The use according to [156-1], wherein the anti-HER2 antibody is an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1, and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2, and CDRL3 consisting of an amino acid sequence consisting of amino acid residues 89 to 97 of SEQ ID NO: 2.

[158-1] The use according to [156-1], wherein the anti-HER2 antibody is an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2.

[159-1] The use according to [156-1], wherein the anti-HER2 antibody is an antibody comprising a heavy chain consisting of an amino acid sequence represented by SEQ ID NO: 1 and a light chain consisting of an amino acid sequence represented by SEQ ID NO: 2.

[160-1] The use according to [156-1], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

[161-1] The use according to any one of [156-1] to [160-1],
wherein the antibody-drug conjugate is represented by the following formula:

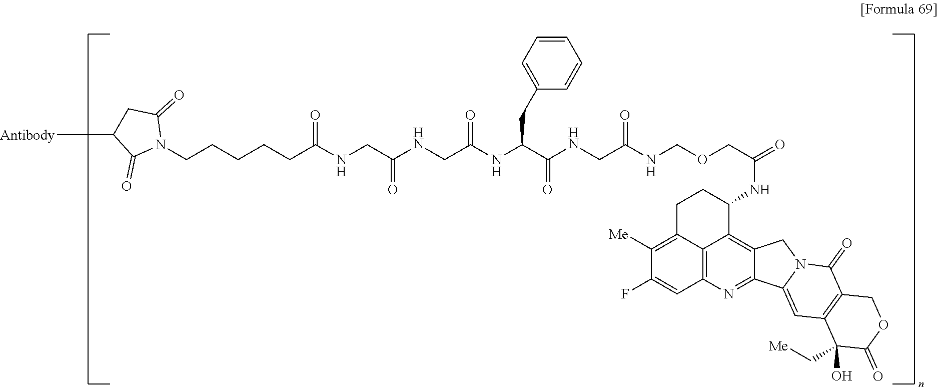

wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8.

[162-1] The use according to [155-1], wherein the antibody in the antibody-drug conjugate is an anti-HER3 antibody.

[G1] The use according to [162-1], wherein the anti-HER3 antibody is an antibody comprising a heavy chain comprising CDRH1 consisting of an amino acid sequence consisting of amino acid residues 26 to 35 of SEQ ID NO: 3, CDRH2 consisting of an amino acid sequence consisting of amino acid residues 50 to 65 of SEQ ID NO: 3, and CDRH3 consisting of an amino acid sequence consisting of amino acid residues 98 to 106 of SEQ ID NO: 3, and a light chain comprising CDRL1 consisting of an amino acid sequence consisting of amino acid residues 24 to 39 of SEQ ID NO: 4, CDRL2 consisting of an amino acid sequence consisting of amino acid residues 56 to 62 of SEQ ID NO: 4, and CDRL3 consisting of an amino acid sequence consisting of amino acid residues 95 to 103 of SEQ ID NO: 4.

[G2] The use according to [162-1], wherein the anti-HER3 antibody is an antibody comprising a heavy chain comprising a heavy chain variable region consisting of an amino acid sequence consisting of amino acid residues 1 to 117 of SEQ ID NO: 3, and a light chain comprising a light chain variable region consisting of an amino acid sequence consisting of amino acid residues 1 to 113 of SEQ ID NO: 4.

[163-1] The use according to [162-1], wherein the anti-HER3 antibody is an antibody comprising a heavy chain consisting of an amino acid sequence represented by SEQ ID NO: 3 and a light chain consisting of an amino acid sequence represented by SEQ ID NO: 4.

[164-1] The use according to [163-1], wherein the anti-HER3 antibody lacks a lysine residue at the carboxyl terminus of the heavy chain.

[165-1] The use according to any one of [162-1] to [164-1] and [G1] to [G2], wherein the antibody-drug conjugate is represented by the following formula:

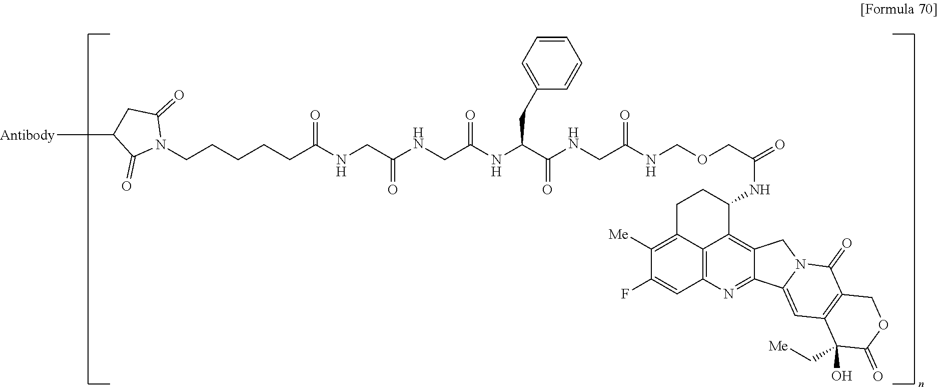

wherein ‘Antibody’ indicates the anti-HER3 antibody conjugated to the drug-linker via a thioether bond, and n indicates an average number of units of the drug-linker conjugated per antibody molecule in the antibody-drug conjugate, wherein n is in the range of from 7 to 8.

[166-1] The use according to [155-1], wherein the antibody in the antibody-drug conjugate is an anti-TROP2 antibody.

[167-1] The use according to [166-1], wherein the anti-TROP2 antibody is an antibody comprising a heavy chain comprising CDRH1 consisting of an amino acid sequence consisting of amino acid residues 50 to 54 of SEQ ID NO: 5, CDRH2 consisting of an amino acid sequence consisting of amino acid residues 69 to 85 of SEQ ID NO: 5, and CDRH3 consisting of an amino acid sequence consisting of amino acid residues 118 to 129 of SEQ ID NO: 5, and a light chain comprising CDRL1 consisting of an amino acid sequence consisting of amino acid residues 44 to 54 of SEQ ID NO: 6, CDRL2 consisting of an amino acid sequence consisting of amino acid residues 70 to 76 of SEQ ID NO: 6, and CDRL3 consisting of an amino acid sequence consisting of amino acid residues 109 to 117 of SEQ ID NO: 6.

[168-1] The use according to [166-1], wherein the anti-TROP2 antibody is an antibody comprising a heavy chain comprising a heavy chain variable region consisting of an amino acid sequence consisting of amino acid residues 20 to 140 of SEQ ID NO: 5, and a light chain comprising a light chain variable region consisting of an amino acid sequence consisting of amino acid residues 21 to 129 of SEQ ID NO: 6.

[169-1] The use according to [166-1], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

[170-1] The use according to [169-1], wherein the anti-TROP2 antibody lacks a lysine residue at the carboxyl terminus of the heavy chain.

[171-1] The use according to any one of [166-1] to [170-1], wherein the antibody-drug conjugate is represented by the following formula:

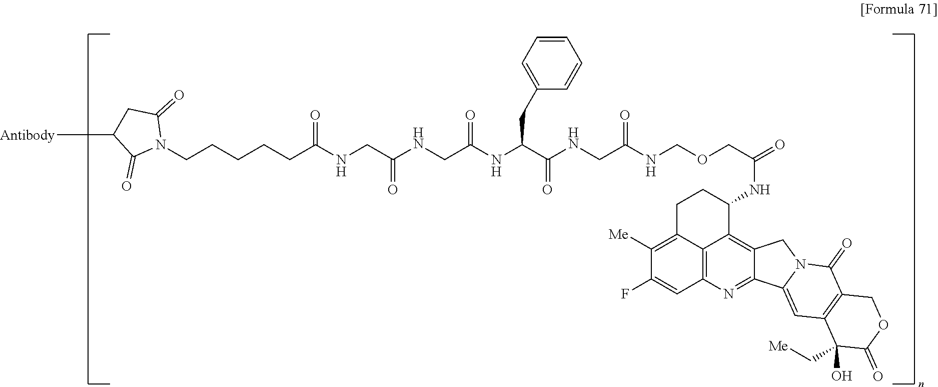

wherein ‘Antibody’ indicates the anti-TROP2 antibody conjugated to the drug-linker via a thioether bond, and n indicates an average number of units of the drug-linker conjugated per antibody molecule in the antibody-drug conjugate, wherein n is in the range of from 3.5 to 4.5.

[172-1] The use according to [155-1], wherein the antibody in the antibody-drug conjugate is an anti-B7-H3 antibody.

[173-1] The use according to [172-1], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

[174-1] The use according to [173-1], wherein the anti-B7-H3 antibody lacks a lysine residue at the carboxyl terminus of the heavy chain.

[175-1] The use according to any one of [172-1] to [174-1], wherein the antibody-drug conjugate is represented by the following formula:

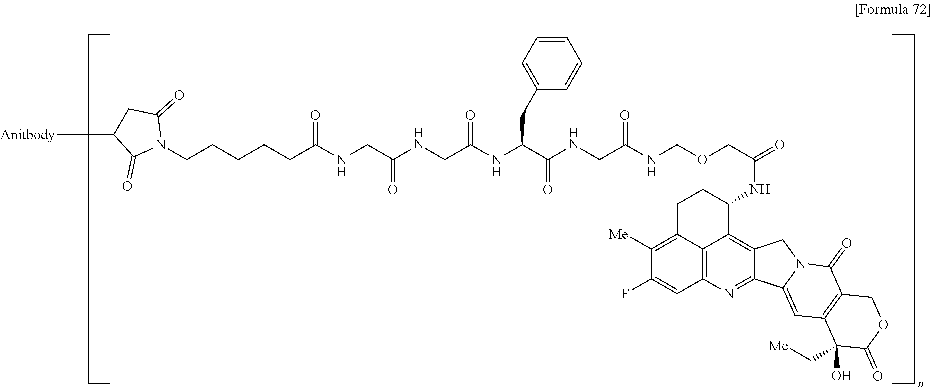

wherein ‘Antibody’ indicates the anti-B7-H3 antibody conjugated to the drug-linker via a thioether bond, and n indicates an average number of units of the drug-linker conjugated per antibody molecule in the antibody-drug conjugate, wherein n is in the range of from 3.5 to 4.5.

[176-1] The use according to [155-1], wherein the antibody in the antibody-drug conjugate is an anti-GPR20 antibody.

[177-1] The use according to [176-1], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

[178-1] The use according to [177-1], wherein the anti-GPR20 antibody lacks a lysine residue at the carboxyl terminus of the heavy chain.

[179-1] The use according to any one of [176-1] to [178-1], wherein the antibody-drug conjugate is represented by the following formula:

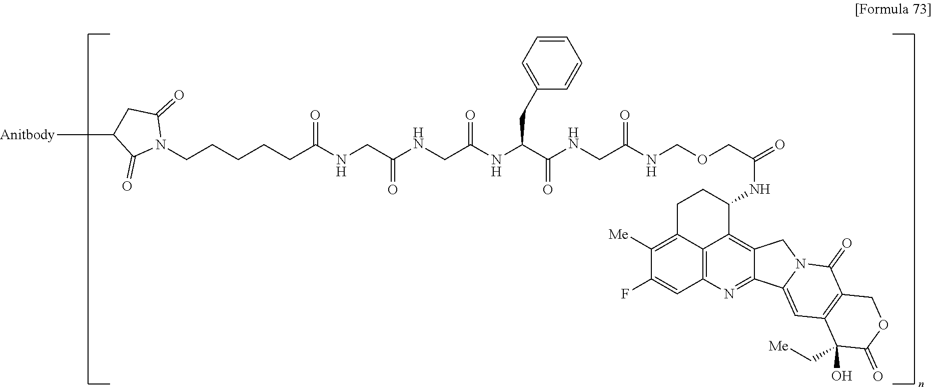

wherein ‘Antibody’ indicates the anti-GPR20 antibody conjugated to the drug-linker via a thioether bond, and n indicates an average number of units of the drug-linker conjugated per antibody molecule in the antibody-drug conjugate, wherein n is in the range of from 7 to 8.

[180-1] The use according to [155-1], wherein the antibody in the antibody-drug conjugate is an anti-CDH6 antibody.

[181-1] The use according to [180-1], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

[182-1] The use according to [181-1], wherein the anti-CDH6 antibody lacks a lysine residue at the carboxyl terminus of the heavy chain.

[183-1] The use according to any one of [180-1] to [182-1], wherein the antibody-drug conjugate is represented by the following formula:

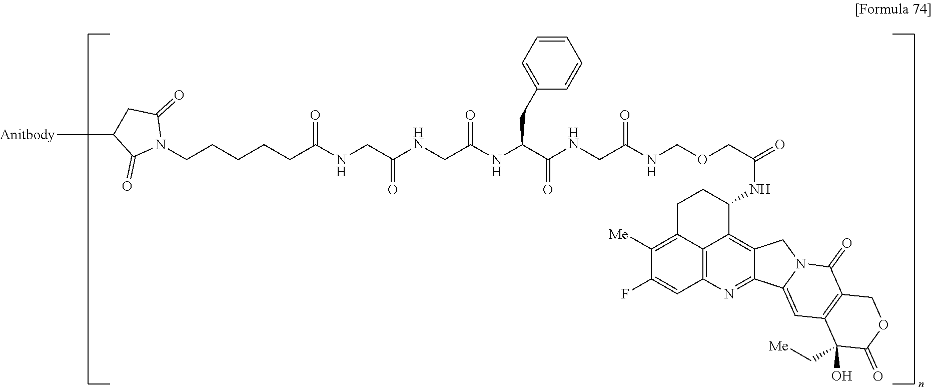

wherein ‘Antibody’ indicates the anti-CDH6 antibody conjugated to the drug-linker via a thioether bond, and n indicates an average number of units of the drug-linker conjugated per antibody molecule in the antibody-drug conjugate, wherein n is in the range of from 7 to 8.

[184-1] The use according to [155-1], wherein the antibody in the antibody-drug conjugate is an anti-MUC1 antibody.

[185-1] The use according to [184-1], wherein the anti-MUC1 antibody is an antibody comprising a heavy chain consisting of an amino acid sequence represented by SEQ ID NO: 13 and a light chain consisting of an amino acid sequence represented by SEQ ID NO: 15.

[186-1] The use according to [185-1], wherein the anti-MUC1 antibody lacks a lysine residue at the carboxyl terminus of the heavy chain.

[187-1] The use according to [184-1], wherein the anti-MUC1 antibody is an antibody comprising a heavy chain consisting of an amino acid sequence represented by SEQ ID NO: 14 and a light chain consisting of an amino acid sequence represented by SEQ ID NO: 15.

[188-1] The use according to [187-1], wherein the anti-MUC1 antibody lacks a lysine residue at the carboxyl terminus of the heavy chain.

[189-1] The use according to any one of [184-1] to [188-1], wherein the antibody-drug conjugate is represented by the following formula:

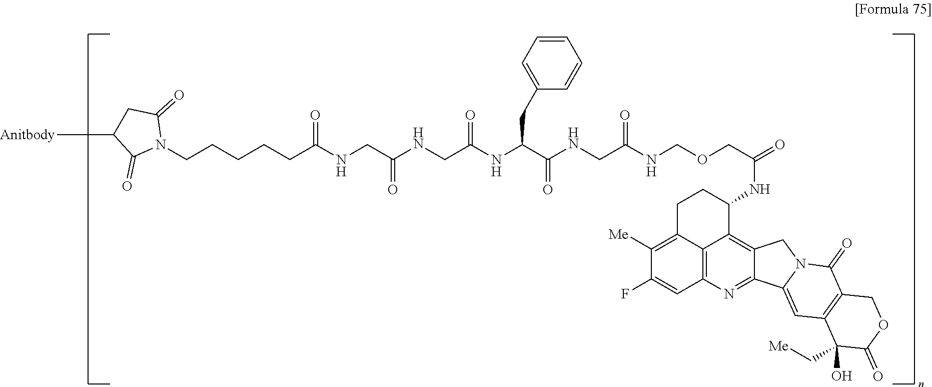

wherein ‘Antibody’ indicates the anti-MUC1 antibody conjugated to the drug-linker via a thioether bond, and n indicates an average number of units of the drug-linker conjugated per antibody molecule in the antibody-drug conjugate, wherein n is in the range of from 7 to 8.

[H1] The use according to [155-1], wherein the antibody in the antibody-drug conjugate is an anti-CD37 antibody.

[H2] The use according to [H1], wherein the anti-CD37 antibody is an antibody selected from the group consisting of
(a) an antibody comprising a heavy chain comprising a heavy chain variable region consisting of an amino acid sequence consisting of amino acid residues 20 to 138 of SEQ ID NO: 16 and a light chain comprising a light chain variable region consisting of an amino acid sequence consisting of amino acid residues 21 to 128 of SEQ ID NO: 19,
(b) an antibody comprising a heavy chain comprising a heavy chain variable region consisting of an amino acid sequence consisting of amino acid residues 20 to 138 of SEQ ID NO: 17 and a light chain comprising a light chain variable region consisting of an amino acid sequence consisting of amino acid residues 21 to 128 of SEQ ID NO: 19, and
(c) an antibody comprising a heavy chain comprising a heavy chain variable region consisting of an amino acid sequence consisting of amino acid residues 20 to 138 of SEQ ID NO: 18 and a light chain comprising a light chain variable region consisting of an amino acid sequence consisting of amino acid residues 21 to 128 of SEQ ID NO: 19.

[H3] The use according to [H1], wherein the anti-CD37 antibody is an antibody selected from the group consisting of
(d) an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19,
(e) an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19, and
(f) an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

[H4] The use according to [H1], wherein the anti-CD37 antibody is an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19.

[H5] The use according to [H4], wherein the anti-CD37 antibody lacks one or two amino acid residues at the carboxyl terminus of the heavy chain.

[H6] The use according to [H4] or [H5], wherein a proline residue at the carboxyl terminus of the heavy chain is amidated.

[H7] The use according to [H1], wherein the anti-CD37 antibody is an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19.

[H8] The use according to [H7], wherein the anti-CD37 antibody lacks one or two amino acid residues at the carboxyl terminus of the heavy chain.

[H9] The use according to [H7] or [H8], wherein a proline residue at the carboxyl terminus of the heavy chain is amidated.

[H10] The use according to [H1], wherein the anti-CD37 antibody is an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

[H11] The use according to [H10], wherein the anti-CD37 antibody lacks one or two amino acid residues at the carboxyl terminus of the heavy chain.

[H12] The use according to [H10] or [H11], wherein a proline residue at the carboxyl terminus of the heavy chain is amidated.

[H13] The use according to any one of [H1] to [H12], wherein the antibody-drug conjugate is represented by the following formula:

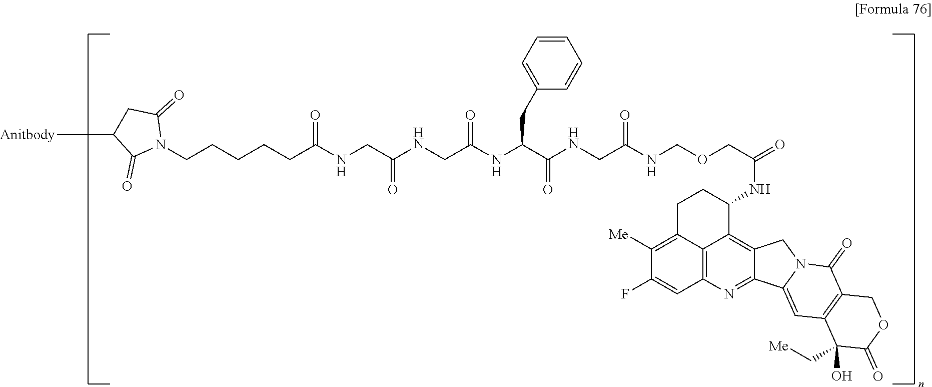

wherein ‘Antibody’ indicates the anti-CD37 antibody conjugated to the drug-linker via a thioether bond, and n indicates an average number of units of the drug-linker conjugated per antibody molecule in the antibody-drug conjugate, wherein n is in the range of from 7 to 8.

[190-1] The use according to any one of [154-1] to [161-1], wherein the antibody-drug conjugate is trastuzumab deruxtecan (DS-8201a).

[191-1] The use according to any one of [154-1] to [155-1] and [166-1] to [171-1], wherein the antibody-drug conjugate is datopotamab deruxtecan (DS-1062).

[192-1] The use according to any one of [154-1] to [191-1], [G1] to [G2] and [H1] to [H13], wherein the inhibitor is an EZH2 inhibitor.

[193-1] The use according to [192-1], wherein the inhibitor is tazemetostat or a pharmaceutically acceptable salt thereof.

[194-1] The use according to any one of [154-1] to [191-1], [G1] to [G2] and [H1] to [H13], wherein the inhibitor is an EZH1/2 dual inhibitor.

[195-1] The use according to [194-1], wherein the inhibitor is valemetostat or a pharmaceutically acceptable salt thereof.

[196-1] The use according to [194-1] or [195-1], wherein the inhibitor is valemetostat tosylate.

[197-1] The use according to any one of [154-1] to [196-1], [G1] to [G2] and [H1] to [H13], wherein the antibody-drug conjugate and the inhibitor are separately contained as active components in different formulations, and are administered simultaneously or at different times.

[198-1] The use according to any one of [154-1] to [197-1], [G1] to [G2], [H1] to [H13], and [260-1], wherein the disease is at least one selected from the group consisting of breast cancer, gastric cancer, colorectal cancer, lung cancer, esophageal cancer, head-and-neck cancer, gastroesophageal junction adenocarcinoma, biliary tract cancer, Paget's disease, pancreatic cancer, ovarian cancer, bladder cancer, prostate cancer, uterine carcinosarcoma, gastrointestinal stromal tumor, kidney cancer, and sarcoma.

[199-1] The use according to [198-1], wherein the disease is breast cancer.

[200-1] The use according to [199-1], wherein the disease is triple-negative breast cancer.

[201-1] The use according to [198-1], wherein the disease is gastric cancer.

[202-1] The use according to [198-1], wherein the disease is ovarian cancer.

[203-1] The use according to [198-1], wherein the disease is lung cancer.

[204-1] The use according to [198-1], wherein the disease is pancreatic cancer.

[205-1] An inhibitor selected from the group consisting of an EZH1 inhibitor, an EZH2 inhibitor and an EZH1/2 dual inhibitor for use in a method of treating a disease, wherein the method comprises administering the inhibitor in combination with an antibody-drug conjugate, wherein the antibody-drug conjugate is an antibody-drug conjugate in which a drug-linker represented by the following formula:

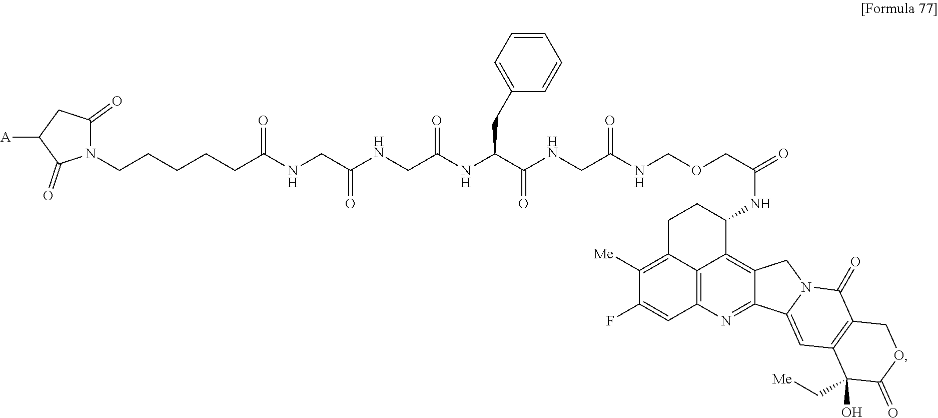

wherein A represents a connecting position to an antibody,
is conjugated to the antibody via a thioether bond in the antibody-drug conjugate.

[206-1] The inhibitor for use according to [205-1], wherein the antibody in the antibody-drug conjugate is an anti-HER2 antibody, an anti-HER3 antibody, an anti-TROP2 antibody, an anti-B7-H3 antibody, an anti-GPR20 antibody, an anti-CDH6 antibody, an anti-MUC1 antibody, or an anti-CD37 antibody.

[207-1] The inhibitor for use according to [206-1], wherein the antibody in the antibody-drug conjugate is an anti-HER2 antibody.

[208-1] The inhibitor for use according to [207-1], wherein the anti-HER2 antibody is an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1, and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2, and CDRL3 consisting of an amino acid sequence consisting of amino acid residues 89 to 97 of SEQ ID NO: 2.

[209-1] The inhibitor for use according to [207-1], wherein the anti-HER2 antibody is an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2.

[210-1] The inhibitor for use according to [207-1], wherein the anti-HER2 antibody is an antibody comprising a heavy chain consisting of an amino acid sequence represented by SEQ ID NO: 1 and a light chain consisting of an amino acid sequence represented by SEQ ID NO: 2.

[211-1] The inhibitor for use according to [207-1], wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

[212-1] The inhibitor for use according to any one of [207-1] to [211-1], wherein the antibody-drug conjugate is represented by the following formula:

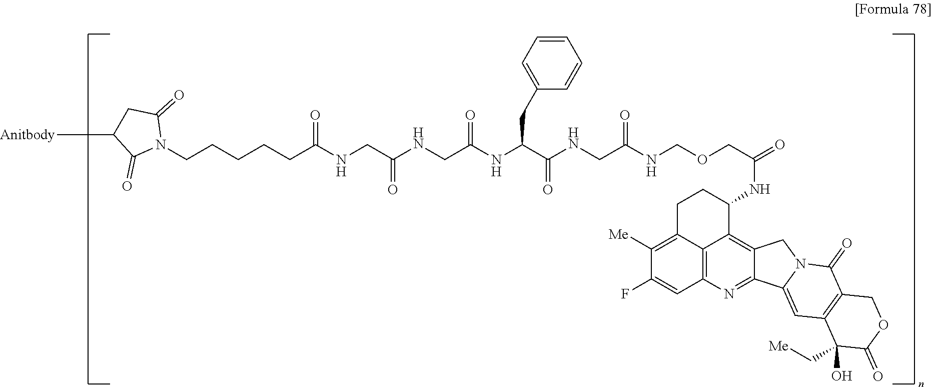

wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8.

[213-1] The inhibitor for use according to [206-1], wherein the antibody in the antibody-drug conjugate is an anti-HER3 antibody.

[I1] The inhibitor for use according to [213-1], wherein the anti-HER3 antibody is an antibody comprising a heavy chain comprising CDRH1 consisting of an amino acid sequence consisting of amino acid residues 26 to 35 of SEQ ID NO: 3, CDRH2 consisting of an amino acid sequence consisting of amino acid residues 50 to 65 of SEQ ID NO: 3, and CDRH3 consisting of an amino acid sequence consisting of amino acid residues 98 to 106 of SEQ ID NO: 3, and a light chain comprising CDRL1 consisting of an amino acid sequence consisting of amino acid residues 24 to 39 of SEQ ID NO: 4, CDRL2 consisting of an amino acid sequence consisting of amino acid residues 56 to 62 of SEQ ID NO: 4, and CDRL3 consisting of an amino acid sequence consisting of amino acid residues 95 to 103 of SEQ ID NO: 4.

[I2] The inhibitor for use according to [213-1], wherein the anti-HER3 antibody is an antibody comprising a heavy chain comprising a heavy chain variable region consisting of an amino acid sequence consisting of amino acid residues 1 to 117 of SEQ ID NO: 3, and a light chain comprising a light chain variable region consisting of an amino acid sequence consisting of amino acid residues 1 to 113 of SEQ ID NO: 4.

[214-1] The inhibitor for use according to [213-1], wherein the anti-HER3 antibody is an antibody comprising a heavy chain consisting of an amino acid sequence represented by SEQ ID NO: 3 and a light chain consisting of an amino acid sequence represented by SEQ ID NO: 4.

[215-1] The inhibitor for use according to [214-1], wherein the anti-HER3 antibody lacks a lysine residue at the carboxyl terminus of the heavy chain.

[216-1] The inhibitor for use according to any one of [213-1] to [215-1] and [I1] to [I2], wherein the antibody-drug conjugate is represented by the following formula:

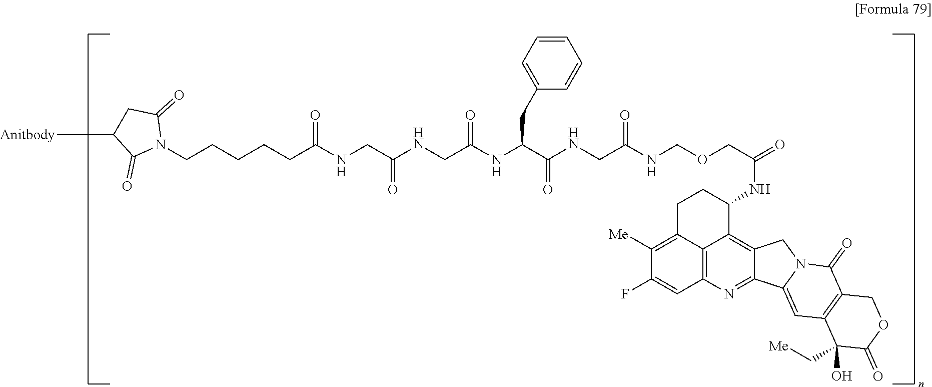

wherein ‘Antibody’ indicates the anti-HER3 antibody conjugated to the drug-linker via a thioether bond, and n indicates an average number of units of the drug-linker conjugated per antibody molecule in the antibody-drug conjugate, wherein n is in the range of from 7 to 8.

[217-1] The inhibitor for use according to [206-1], wherein the antibody in the antibody-drug conjugate is an anti-TROP2 antibody.

[218-1] The inhibitor for use according to [217-1], wherein the anti-TROP2 antibody is an antibody comprising a heavy chain comprising CDRH1 consisting of an amino acid sequence consisting of amino acid residues 50 to 54 of SEQ ID NO: 5, CDRH2 consisting of an amino acid sequence consisting of amino acid residues 69 to 85 of SEQ ID NO: 5, and CDRH3 consisting of an amino acid sequence consisting of amino acid residues 118 to 129 of SEQ ID NO: 5, and a light chain comprising CDRL1 consisting of an amino acid sequence consisting of amino acid residues 44 to 54 of SEQ ID NO: 6, CDRL2 consisting of an amino acid sequence consisting of amino acid residues 70 to 76 of SEQ ID NO: 6, and CDRL3 consisting of an amino acid sequence consisting of amino acid residues 109 to 117 of SEQ ID NO: 6.

[219-1] The inhibitor for use according to [217-1], wherein the anti-TROP2 antibody is an antibody comprising a heavy chain comprising a heavy chain variable region consisting of an amino acid sequence consisting of amino acid residues 20 to 140 of SEQ ID NO: 5, and a light chain comprising a light chain variable region consisting of an amino acid sequence consisting of amino acid residues 21 to 129 of SEQ ID NO: 6.

[220-1] The inhibitor for use according to [217-1], wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

[221-1] The inhibitor for use according to [220-1], wherein the anti-TROP2 antibody lacks a lysine residue at the carboxyl terminus of the heavy chain.

[222-1] The inhibitor for use according to any one of [217-1] to [221-1], wherein the antibody-drug conjugate is represented by the following formula:

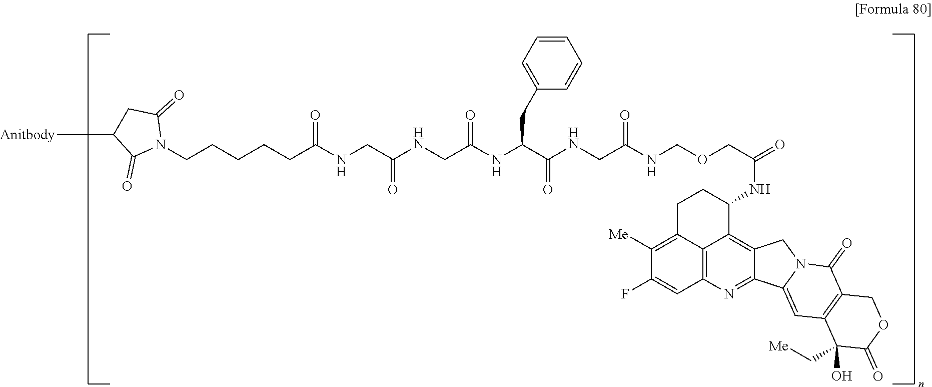

wherein ‘Antibody’ indicates the anti-TROP2 antibody conjugated to the drug-linker via a thioether bond, and n indicates an average number of units of the drug-linker conjugated per antibody molecule in the antibody-drug conjugate, wherein n is in the range of from 3.5 to 4.5.

[223-1] The inhibitor for use according to [206-1], wherein the antibody in the antibody-drug conjugate is an anti-B7-H3 antibody.

[224-1] The inhibitor for use according to [223-1], wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

[225-1] The inhibitor for use according to [224-1], wherein the anti-B7-H3 antibody lacks a lysine residue at the carboxyl terminus of the heavy chain.

[226-1] The inhibitor for use according to any one of [223-1] to [225-1], wherein the antibody-drug conjugate is represented by the following formula:

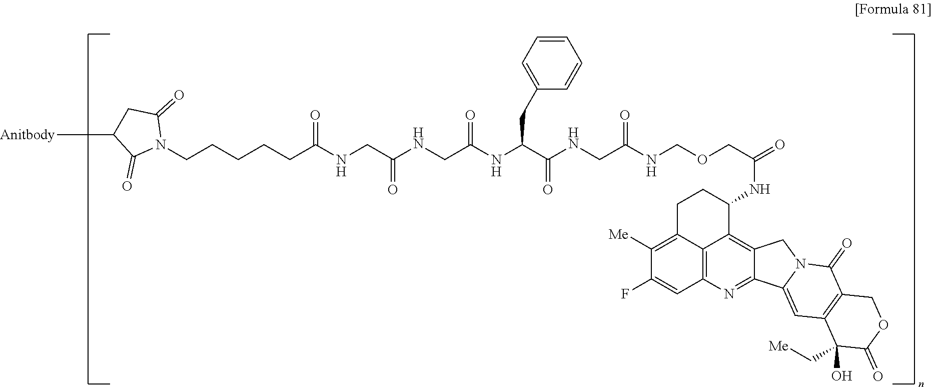

wherein ‘Antibody’ indicates the anti-B7-H3 antibody conjugated to the drug-linker via a thioether bond, and n indicates an average number of units of the drug-linker conjugated per antibody molecule in the antibody-drug conjugate, wherein n is in the range of from 3.5 to 4.5.

[227-1] The inhibitor for use according to [206-1], wherein the antibody in the antibody-drug conjugate is an anti-GPR20 antibody.

[228-1] The inhibitor for use according to [227-1], wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

[229-1] The inhibitor for use according to [228-1], wherein the anti-GPR20 antibody lacks a lysine residue at the carboxyl terminus of the heavy chain.

[230-1] The inhibitor for use according to any one of [227-1] to [229-1], wherein the antibody-drug conjugate is represented by the following formula:

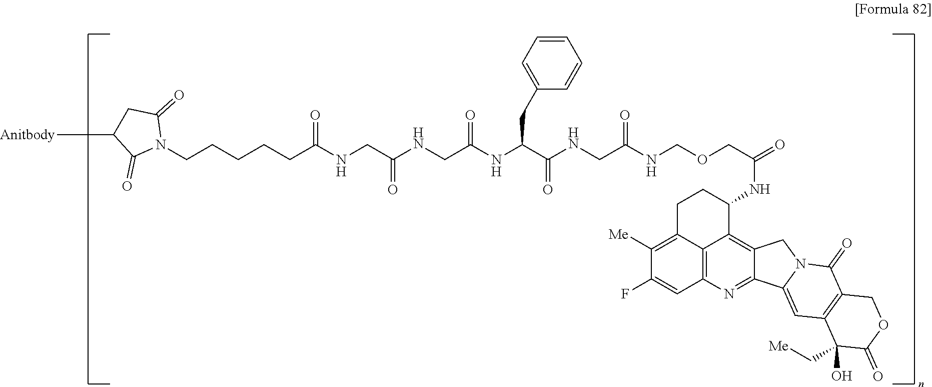

wherein ‘Antibody’ indicates the anti-GPR20 antibody conjugated to the drug-linker via a thioether bond, and n indicates an average number of units of the drug-linker conjugated per antibody molecule in the antibody-drug conjugate, wherein n is in the range of from 7 to 8.

[231-1] The inhibitor for use according to [206-1], wherein the antibody in the antibody-drug conjugate is an anti-CDH6 antibody.

[232-1] The inhibitor for use according to [231-1], wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

[233-1] The inhibitor for use according to [232-1], wherein the anti-CDH6 antibody lacks a lysine residue at the carboxyl terminus of the heavy chain.

[234-1] The inhibitor for use according to any one of [231-1] to [233-1], wherein the anti-CDH6 antibody-drug conjugate is represented by the following formula:

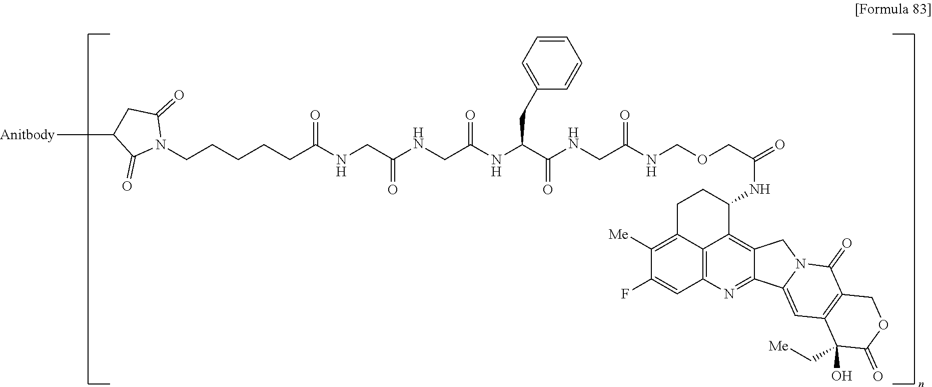

wherein ‘Antibody’ indicates the anti-CDH6 antibody conjugated to the drug-linker via a thioether bond, and n indicates an average number of units of the drug-linker conjugated per antibody molecule in the antibody-drug conjugate, wherein n is in the range of from 7 to 8.

[235-1] The inhibitor for use according to [206-1], wherein the antibody in the antibody-drug conjugate is an anti-MUC1 antibody.

[236-1] The inhibitor for use according to [235-1], wherein the anti-MUC1 antibody is an antibody comprising a heavy chain consisting of an amino acid sequence represented by SEQ ID NO: 13 and a light chain consisting of an amino acid sequence represented by SEQ ID NO: 15.

[237-1] The inhibitor for use according to [236-1], wherein the anti-MUC1 antibody lacks a lysine residue at the carboxyl terminus of the heavy chain.

[238-1] The inhibitor for use according to [235-1], wherein the anti-MUC1 antibody is an antibody comprising a heavy chain consisting of an amino acid sequence represented by SEQ ID NO: 14 and a light chain consisting of an amino acid sequence represented by SEQ ID NO: 15.

[239-1] The inhibitor for use according to [238-1], wherein the anti-MUC1 antibody lacks a lysine residue at the carboxyl terminus of the heavy chain.

[240-1] The inhibitor for use according to any one of [235-1] to [239-1], wherein the antibody-drug conjugate is represented by the following formula:

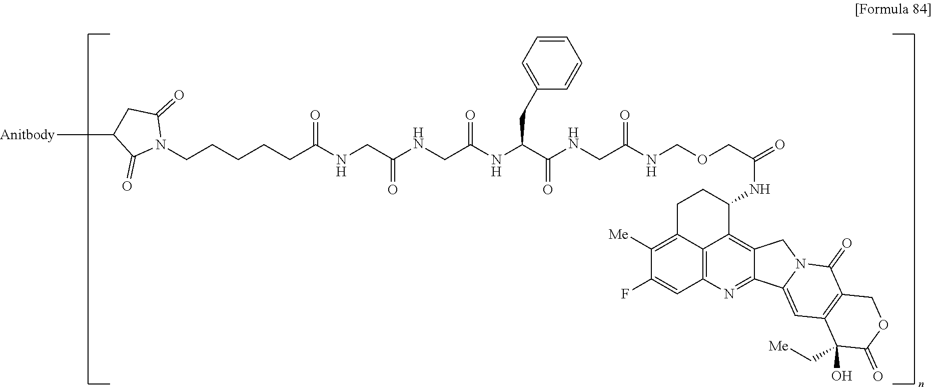

wherein ‘Antibody’ indicates the anti-MUC1 antibody conjugated to the drug-linker via a thioether bond, and n indicates an average number of units of the drug-linker conjugated per antibody molecule in the antibody-drug conjugate, wherein n is in the range of from 7 to 8.

[J1] The inhibitor for use according to [206-1], wherein the antibody in the antibody-drug conjugate is an anti-CD37 antibody.

[J2] The inhibitor for use according to [J1], wherein the anti-CD37 antibody is an antibody selected from the group consisting of
(a) an antibody comprising a heavy chain comprising a heavy chain variable region consisting of an amino acid sequence consisting of amino acid residues 20 to 138 of SEQ ID NO: 16 and a light chain comprising a light chain variable region consisting of an amino acid sequence consisting of amino acid residues 21 to 128 of SEQ ID NO: 19,
(b) an antibody comprising a heavy chain comprising a heavy chain variable region consisting of an amino acid sequence consisting of amino acid residues 20 to 138 of SEQ ID NO: 17 and a light chain comprising a light chain variable region consisting of an amino acid sequence consisting of amino acid residues 21 to 128 of SEQ ID NO: 19, and
(c) an antibody comprising a heavy chain comprising a heavy chain variable region consisting of an amino acid sequence consisting of amino acid residues 20 to 138 of SEQ ID NO: 18 and a light chain comprising a light chain variable region consisting of an amino acid sequence consisting of amino acid residues 21 to 128 of SEQ ID NO: 19.

[J3] The inhibitor for use according to [J1], wherein the anti-CD37 antibody is an antibody selected from the group consisting of
(d) an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19,
(e) an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19, and
(f) an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

[J4] The inhibitor for use according to [J1], wherein the anti-CD37 antibody is an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19.

[J5] The inhibitor for use according to [J4], wherein the anti-CD37 antibody lacks one or two amino acid residues at the carboxyl terminus of the heavy chain.

[J6] The inhibitor for use according to [J4] or [J5], wherein a proline residue at the carboxyl terminus of the heavy chain is amidated.

[J7] The inhibitor for use according to [J1], wherein the anti-CD37 antibody is an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19.

[J8] The inhibitor for use according to [J7], wherein the anti-CD37 antibody lacks one or two amino acid residues at the carboxyl terminus of the heavy chain.

[J9] The inhibitor for use according to [J7] or [J8], wherein a proline residue at the carboxyl terminus of the heavy chain is amidated.

[J10] The inhibitor for use according to [J1], wherein the anti-CD37 antibody is an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

[J11] The inhibitor for use according to [J10], wherein the anti-CD37 antibody lacks one or two amino acid residues at the carboxyl terminus of the heavy chain.

[J12] The inhibitor for use according to [J10] or [J11], wherein a proline residue at the carboxyl terminus of the heavy chain is amidated.

[J13] The inhibitor for use according to any one of [J1] to [J12], wherein the antibody-drug conjugate is represented by the following formula:

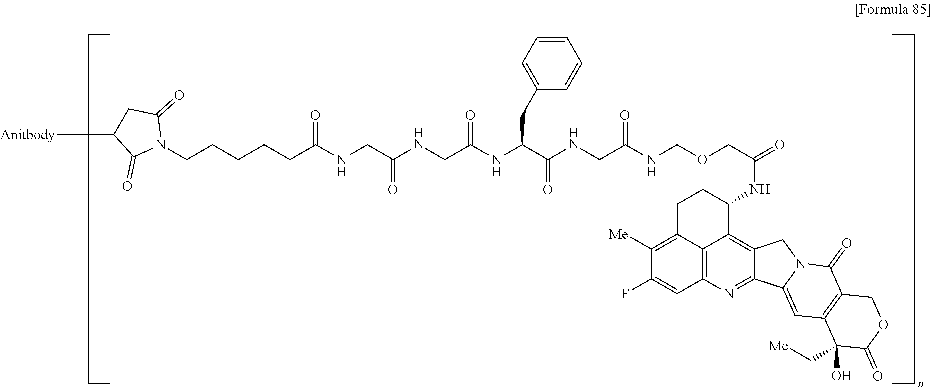

wherein ‘Antibody’ indicates the anti-CD37 antibody conjugated to the drug-linker via a thioether bond, and n indicates an average number of units of the drug-linker conjugated per antibody molecule in the antibody-drug conjugate, wherein n is in the range of from 7 to 8.

[241-1] The inhibitor for use according to any one of [205-1] to [212-1], wherein the antibody-drug conjugate is trastuzumab deruxtecan (DS-8201a).

[242-1] The inhibitor for use according to any one of [205-1] to [206-1] and [217-1] to [222-1], wherein the antibody-drug conjugate is datopotamab deruxtecan (DS-1062).

[243-1] The inhibitor for use according to any one of [205-1] to [242-1], [I1] to [I2] and [J1] to [J13], wherein the inhibitor is an EZH2 inhibitor.

[244-1] The inhibitor for use according to [243-1], wherein the inhibitor is tazemetostat or a pharmaceutically acceptable salt thereof.

[245-1] The inhibitor for use according to any one of [205-1] to [242-1], [I1] to [I2] and [J1] to [J13], wherein the inhibitor is an EZH1/2 dual inhibitor.

[246-1] The inhibitor for use according to [245-1], wherein the inhibitor is valemetostat or a pharmaceutically acceptable salt thereof.

[247-1] The inhibitor for use according to [245-1], or [246-1], wherein the inhibitor is valemetostat tosylate.

[248-1] The inhibitor for use according to any one of [205-1] to [247-1], [I1] to [I2] and [J1] to [J13], wherein the antibody-drug conjugate and the inhibitor are separately contained as active components in different formulations, and are administered simultaneously or at different times.

[249-1] The inhibitor for use according to any one of [205-1] to [248-1], [I1] to [I2], [J1] to [J13], and [261-1], wherein the disease is at least one selected from the group consisting of breast cancer, gastric cancer, colorectal cancer, lung cancer, esophageal cancer, head-and-neck cancer, gastroesophageal junction adenocarcinoma, biliary tract cancer, Paget's disease, pancreatic cancer, ovarian cancer, bladder cancer, prostate cancer, uterine carcinosarcoma, gastrointestinal stromal tumor, kidney cancer, and sarcoma.

[250-1] The inhibitor for use according to [249-1], wherein the disease is breast cancer.

[251-1] The inhibitor for use according to [250-1], wherein the disease is triple-negative breast cancer.

[252-1] The inhibitor for use according to [249-1], wherein the disease is gastric cancer.

[253-1] The inhibitor for use according to [249-1], wherein the disease is ovarian cancer.

[254-1] The inhibitor for use according to [249-1], wherein the disease is lung cancer.

[255-1] The inhibitor for use according to [249-1], wherein the disease is pancreatic cancer.

[256-1] The pharmaceutical product according to any one of [1-1] to [51-1], [A1] to [A2], [B1] to [B13], and [257-1], wherein the pharmaceutical product is a pharmaceutical composition.

[257-1] The pharmaceutical product according to any one of [1-1] to [44-1], [A1] to [A2] and [B1] to [B13], wherein the pharmaceutical product is for use in treating cancer.

[258-1] The method of treatment according to any one of [52-1] to [95-1], [C1] to [C2] and [D1] to [D13], wherein the method of treatment is for treating cancer.

[259-1] The antibody-drug conjugate for use according to any one of [103-1] to [146-1], [E1] to [E2] and [F1] to [F13], wherein the disease is cancer.

[260-1] The use according to any one of [154-1] to [197-1], [G1] to [G2] and [H1] to [H13], wherein the disease is cancer.

[261-1] The inhibitor for use according to any one of [205-1] to [248-1], [I1] to [I2] and [J1] to [J13], wherein the disease is cancer.

### Advantageous Effects of Invention

The present invention provides a pharmaceutical product, wherein a specific antibody-drug conjugate and an inhibitor selected from the group consisting of an EZH1 inhibitor, an EZH2 inhibitor and an EZH1/2 dual inhibitor are administered in combination, and/or a method of treatment, wherein the specific antibody-drug conjugate and the inhibitor selected from the group consisting of an EZH1 inhibitor, an EZH2 inhibitor and an EZH1/2 dual inhibitor are administered in combination to a subject.

### BRIEF DESCRIPTION OF DRAWINGS

FIG. 1 shows the amino acid sequence of the heavy chain of an anti-HER2 antibody (SEQ ID NO: 1).

FIG. 2 shows the amino acid sequence of the light chain of an anti-HER2 antibody (SEQ ID NO: 2).

FIG. 3 shows the amino acid sequence of the heavy chain of an anti-HER3 antibody (SEQ ID NO: 3).

FIG. 4 shows the amino acid sequence of the light chain of an anti-HER3 antibody (SEQ ID NO: 4).

FIG. 5 shows the amino acid sequence of the heavy chain of an anti-TROP2 antibody (SEQ ID NO: 5).

FIG. 6 shows the amino acid sequence of the light chain of an anti-TROP2 antibody (SEQ ID NO: 6).

FIG. 7 shows the amino acid sequence of the heavy chain of an anti-B7-H3 antibody (SEQ ID NO: 7).

FIG. 8 shows the amino acid sequence of the light chain of an anti-B7-H3 antibody (SEQ ID NO: 8).

FIG. 9 shows the amino acid sequence of the heavy chain of an anti-GPR20 antibody (SEQ ID NO: 9).

FIG. 10 shows the amino acid sequence of the light chain of an anti-GPR20 antibody (SEQ ID NO: 10).

FIG. 11 shows the amino acid sequence of the heavy chain of an anti-CDH6 antibody (SEQ ID NO: 11).

FIG. 12 shows the amino acid sequence of the light chain of an anti-CDH6 antibody (SEQ ID NO: 12).

FIG. 13 shows the amino acid sequence of the heavy chain of an anti-MUC1 antibody (PM N54Q) (SEQ ID No: 13).

FIG. 14 shows the amino acid sequence of the heavy chain of an anti-MUC1 antibody (PankoMab) (SEQ ID No: 14).

FIG. 15 shows the amino acid sequence of the light chain of the anti-MUC1 antibodies (PM N54Q and PankoMab) (SEQ ID No: 15).

FIG. 16 is a diagram showing tumor growth suppressing effect in mice with subcutaneously transplanted NCI-N87 cells in single administration groups of HER2-ADC (1) and Compound A respectively, and a combined administration group of HER2-ADC (1) and Compound A.

FIG. 17 is a diagram showing tumor growth suppressing effect in mice with subcutaneously transplanted JIMT-1 cells in single administration groups of HER2-ADC (1) and Compound A respectively, and a combined administration group of HER2-ADC (1) and Compound A.

FIG. 18 is a diagram showing tumor growth suppressing effect in mice with subcutaneously transplanted MDA-MB-453 cells in single administration groups of HER2-ADC (1) and Compound A respectively, and a combined administration group of HER2-ADC (1) and Compound A.

FIG. 19 is a diagram showing tumor growth suppressing effect in mice with subcutaneously transplanted MX-1 cells in single administration groups of HER2-ADC (1) and Compound A respectively, and a combined administration group of HER2-ADC (1) and Compound A.

FIG. 20 shows the amino acid sequence of the heavy chain of an anti-CD37 antibody (hmAb-H541) (SEQ ID No: 16).

FIG. 21 shows the amino acid sequence of the heavy chain of an anti-CD37 antibody (hmAb-H551) (SEQ ID No: 17).

FIG. 22 shows the amino acid sequence of the heavy chain of an anti-CD37 antibody (hmAb-H11a) (SEQ ID No: 18).

FIG. 23 shows the amino acid sequence of the light chain of the anti-CD37 antibody (hmAb-L11) (SEQ ID No: 19).

FIG. 24 is a diagram showing tumor growth suppressing effect in mice with subcutaneously transplanted ZR-75-1 cells in single administration groups of HER2-ADC (1) and Compound A respectively, and a combined administration group of HER2-ADC (1) and Compound A.

FIG. 25 is a diagram showing tumor growth suppressing effect in mice with subcutaneously transplanted Calu-3 cells in single administration groups of HER2-ADC (1) and Compound A respectively, and a combined administration group of HER2-ADC (1) and Compound A.

FIG. 26 is a diagram showing tumor growth suppressing effect in mice with subcutaneously transplanted NCI-H358 cells in single administration groups of TROP2-ADC (1) and Compound A respectively, and a combined administration group of TROP2-ADC (1) and Compound A.

FIG. 27 is a diagram showing tumor growth suppressing effect in mice with subcutaneously transplanted JIMT-1 cells in single administration groups of HER3-ADC (1) and Compound A respectively, and a combined administration group of HER3-ADC (1) and Compound A.

FIG. 28 is a diagram showing tumor growth suppressing effect in mice with subcutaneously transplanted WSU-DLCL2 cells in single administration groups of CD37-ADC (1) and Compound A respectively, and a combined administration group of CD37-ADC (1) and Compound A.

FIG. 29 is a diagram showing tumor growth suppressing effect in mice with subcutaneously transplanted NCI-H660 cells in single administration groups of B7-H3-ADC (1) and Compound A respectively, and a combined administration group of B7-H3-ADC (1) and Compound A.

FIG. 30 shows amino acid sequence of the CDRH1 of heavy chain of anti-HER2 antibody.

FIG. 31 shows amino acid sequence of the CDRH2 of heavy chain of anti-HER2 antibody.

FIG. 32 shows amino acid sequence of the CDRH3 of heavy chain of anti-HER2 antibody.

FIG. 33 shows amino acid sequence of the CDRL1 of light chain of anti-HER2 antibody.

FIG. 34 shows amino acid sequence of the CDRL2 of light chain of anti-HER2 antibody.

FIG. 35 shows amino acid sequence of the CDRL3 of light chain of anti-HER2 antibody.

FIG. 36 shows amino acid sequence of the heavy chain variable region of anti-HER2 antibody.

FIG. 37 shows amino acid sequence of the light chain variable region of anti-HER2 antibody.

FIG. 38 shows amino acid sequence of the CDRH1 of heavy chain of anti-TROP2 antibody.

FIG. 39 shows amino acid sequence of the CDRH2 of heavy chain of anti-TROP2 antibody.

FIG. 40 shows amino acid sequence of the CDRH3 of heavy chain of anti-TROP2 antibody.

FIG. 41 shows amino acid sequence of the CDRL1 of light chain of anti-TROP2 antibody.

FIG. 42 shows amino acid sequence of the CDRL2 of light chain of anti-TROP2 antibody.

FIG. 43 shows amino acid sequence of the CDRL3 of light chain of anti-TROP2 antibody.

FIG. 44 shows amino acid sequence of the heavy chain variable region of anti-TROP2 antibody.

FIG. 45 shows amino acid sequence of the light chain variable region of anti-TROP2 antibody.

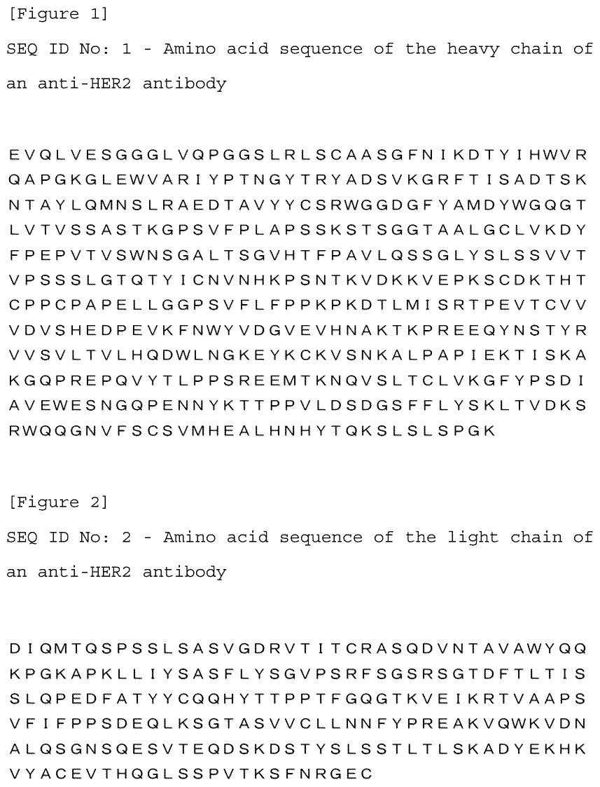

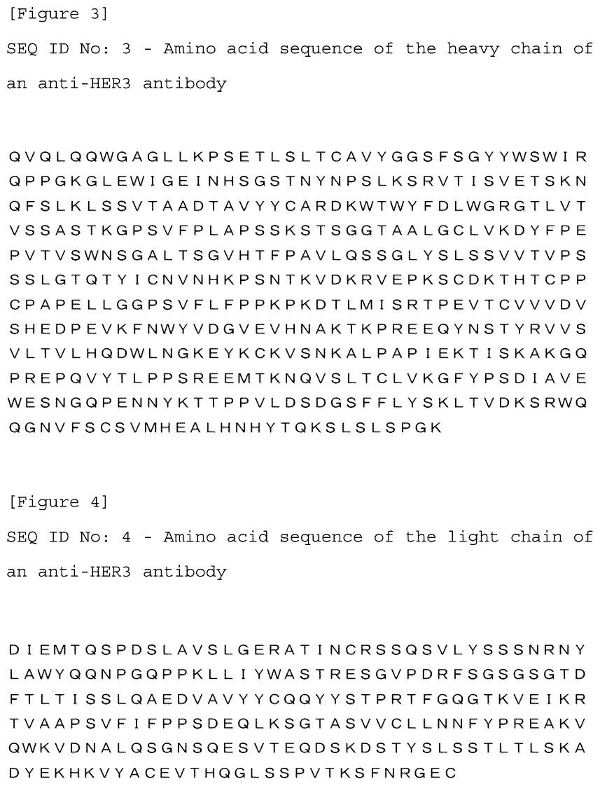

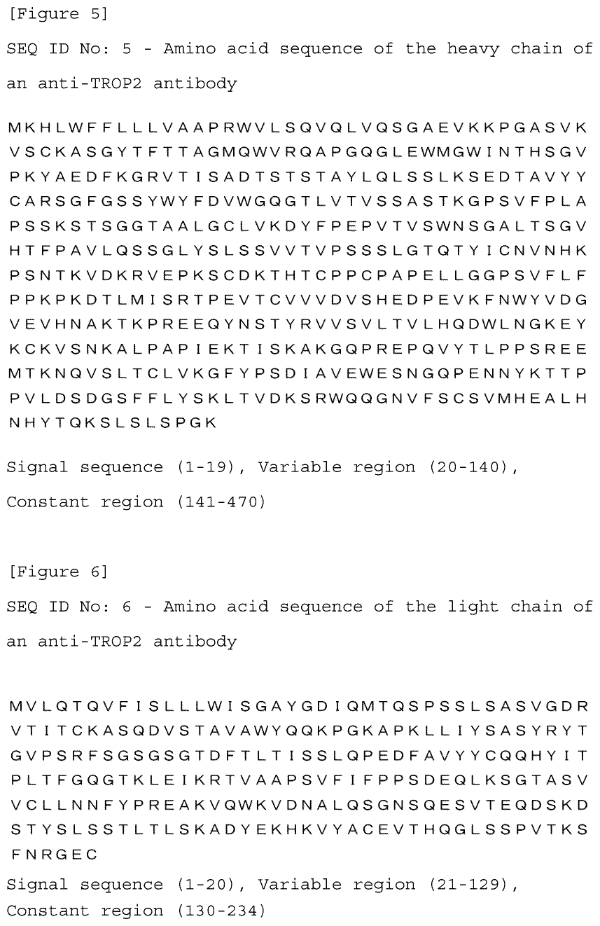

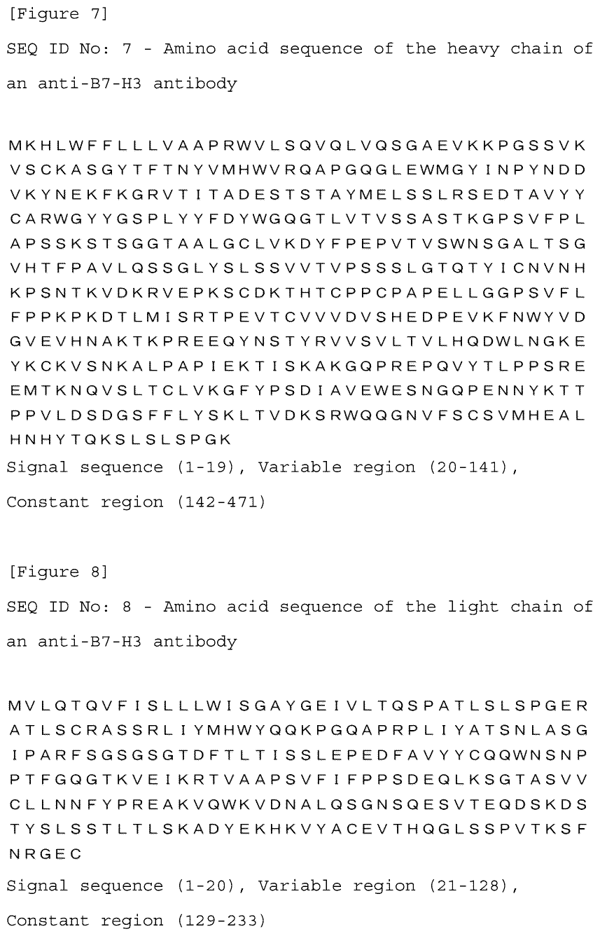

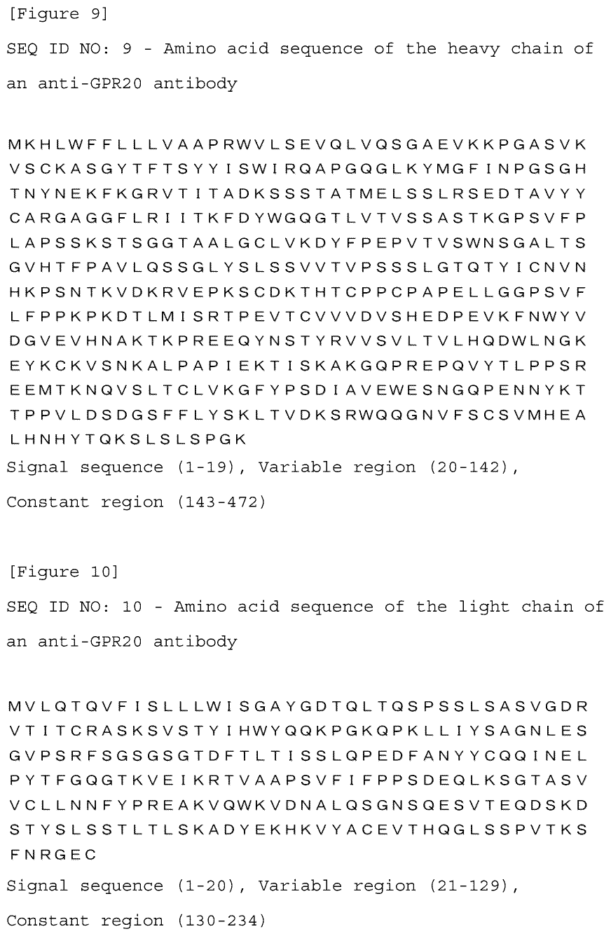

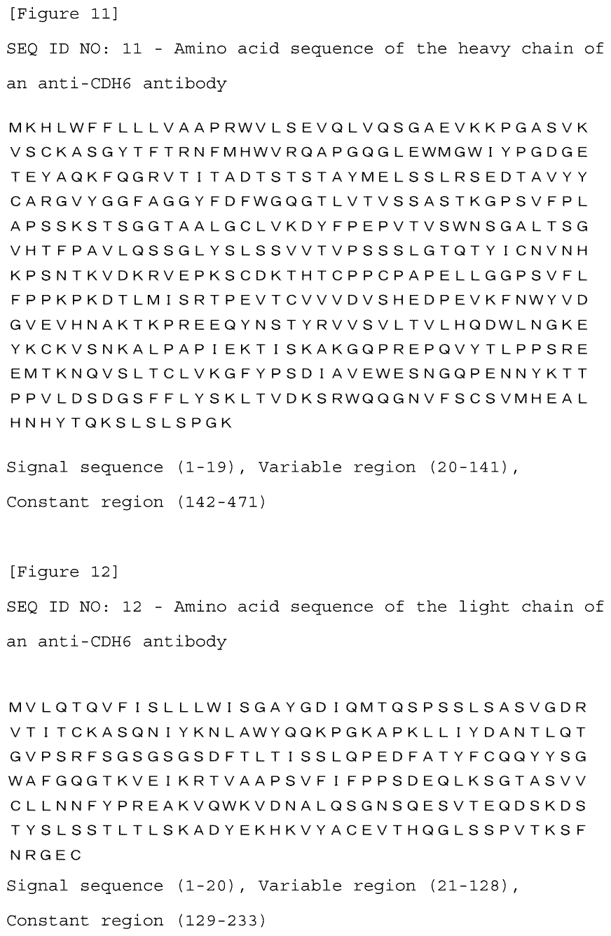

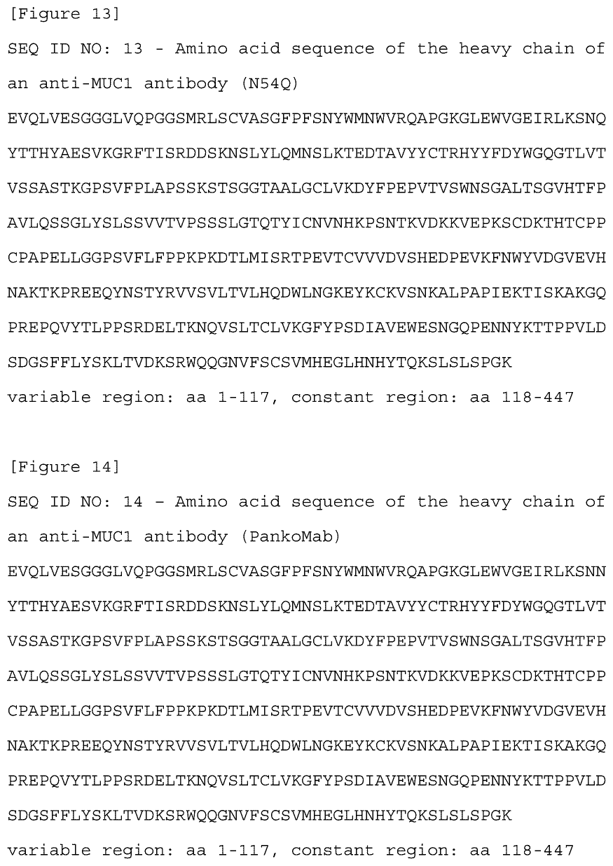

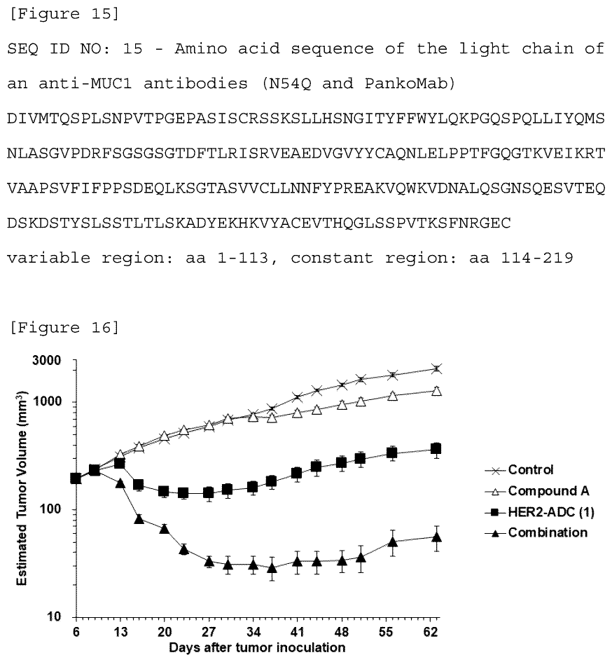

### DESCRIPTION OF EMBODIMENTS

Hereinafter, preferred modes for carrying out the present invention are described. The embodiments described below are given merely for illustrating one example of a typical embodiment of the present invention and are not intended to limit the scope of the present invention.

### 1. Antibody-Drug Conjugate

The antibody-drug conjugate used in the present invention is an antibody-drug conjugate in which a drug-linker represented by the following formula:

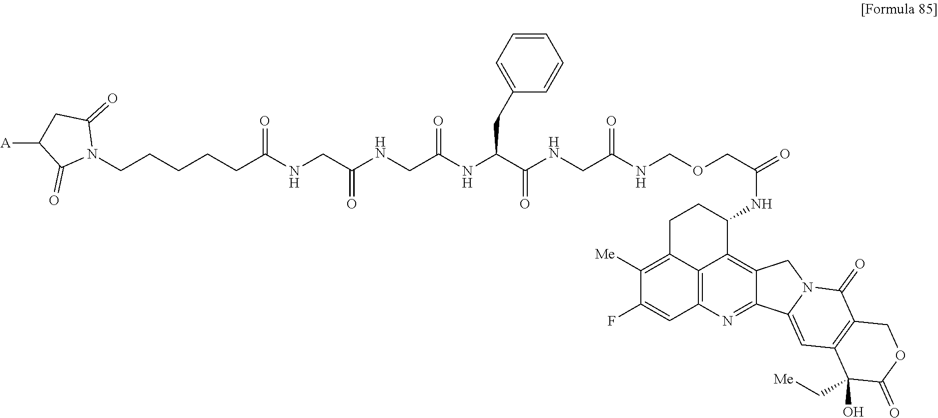

wherein A represents a connecting position to an antibody,
is conjugated to the antibody via a thioether bond.

In the present invention, the partial structure consisting of a linker and a drug in the antibody-drug conjugate is referred to as a “drug-linker”. The drug-linker is connected to a thiol group (in other words, the sulfur atom of a cysteine residue) formed at an interchain disulfide bond site (two sites between heavy chains, and two sites between a heavy chain and a light chain) in the antibody.

The drug-linker of the present invention includes exatecan (IUPAC name: (1S,9S)-1-amino-9-ethyl-5-fluoro-1,2,3,9,12,15-hexahydro-9-hydroxy-4-methyl-10H,13H-benzo[de]pyrano[3′,4′:6,7]indolizino[1,2-b]quinolin-10,13-dione, (also expressed as chemical name: (1S,9S)-1-amino-9-ethyl-5-fluoro-2,3-dihydro-9-hydroxy-4-methyl-1H,12H-benzo[de]pyrano[3′,4′:6,7]indolizino[1,2-b]quinolin-10,13(9H,15H)-dione)), which is a topoisomerase I inhibitor, as a component. Exatecan is a camptothecin derivative having an antitumor effect, represented by the following formula:

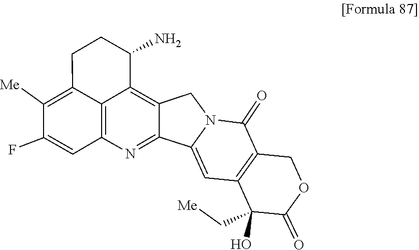 [SMILES: CC[C@@]1(O)C(=O)OCc2c1cc1n(c2=O)Cc2c-1nc1cc(F)c(C)c3c1c2[C@@H](N)CC3]

The antibody-drug conjugate used in the present invention can also be represented by the following formula:

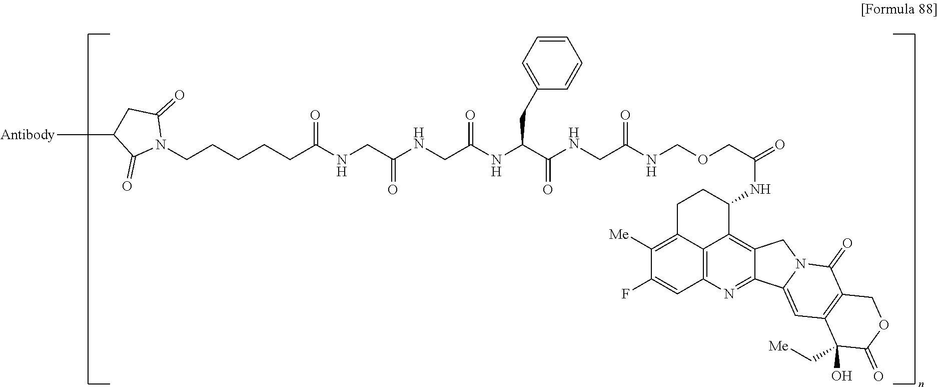

wherein, the drug-linker is conjugated to an antibody via a thioether bond. The meaning of n is the same as that of what is called the average number of conjugated drug molecules (DAR; Drug-to-Antibody Ratio), and indicates the average number of units of the drug-linker conjugated per antibody molecule.

After migrating into cancer cells, the antibody-drug conjugate used in the present invention is cleaved at the linker portion to release the compound represented by the following formula:

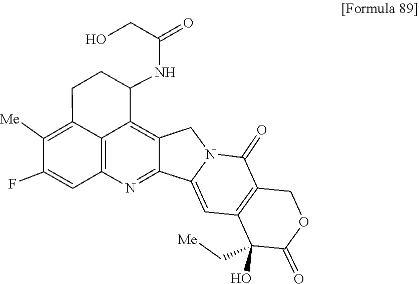 [SMILES: CC[C@@]1(O)C(=O)OCc2c1cc1n(c2=O)Cc2c-1nc1cc(F)c(C)c3c1c2C(NC(=O)CO)CC3]

The aforementioned compound is inferred to be the original source of the antitumor activity of the antibody-drug conjugate used in the present invention, and has been confirmed to have a topoisomerase I inhibitory effect (Ogitani Y. et al., Clinical Cancer Research, 2016, Oct. 15; 22 (20):5097-5108, Epub 2016 Mar. 29.)

The antibody-drug conjugate used in the present invention is also known to have a bystander effect (Ogitani Y. et al., Cancer Science (2016) 107, 1039-1046).

The bystander effect is exerted through a process such that the antibody-drug conjugate used in the present invention is internalized in cancer cells expressing the target, and the aforementioned compound is released and then exerts an antitumor effect also on cancer cells which are present therearound and not expressing the target.

The bystander effect is also exerted as an excellent antitumor effect when the antibody-drug conjugate according to the present invention is used in combination with an inhibitor selected from the group consisting of an EZH1 inhibitor, an EZH2 inhibitor and an EZH1/2 dual inhibitor.

### 2. Antibody in the Antibody-Drug Conjugate

The antibody in the antibody-drug conjugate used in the present invention may be derived from any species and is preferably an antibody derived from a human, a rat, a mouse, or a rabbit. In cases when the antibody is derived from species other than human species, it is preferably chimerized or humanized using a well-known technique. The antibody of the present invention may be a polyclonal antibody or a monoclonal antibody and is preferably a monoclonal antibody.

The antibody in the antibody-drug conjugate used in the present invention is an antibody preferably having the characteristic of being able to target cancer cells, and is preferably an antibody possessing, for example, the property of being able to recognize a cancer cell, the property of being able to bind to a cancer cell, the property of being internalized in a cancer cell, and/or cytocidal activity against cancer cells.

The binding activity of the antibody against cancer cells can be confirmed using flow cytometry. The internalization of the antibody into tumor cells can be confirmed using (1) an assay of visualizing an antibody incorporated in cells under a fluorescence microscope using a secondary antibody (fluorescently labeled) binding to the therapeutic antibody (Cell Death and Differentiation (2008) 15, 751-761), (2) an assay of measuring a fluorescence intensity incorporated in cells using a secondary antibody (fluorescently labeled) binding to the therapeutic antibody (Molecular Biology of the Cell, Vol. 15, 5268-5282, December 2004), or (3) a Mab-ZAP assay using an immunotoxin binding to the therapeutic antibody wherein the toxin is released upon incorporation into cells to inhibit cell growth (Bio Techniques 28: 162-165, January 2000). As the immunotoxin, a recombinant complex protein of a diphtheria toxin catalytic domain and protein G may be used.

The antitumor activity of the antibody can be confirmed in vitro by determining inhibitory activity against cell growth. For example, a cancer cell line overexpressing a target protein for the antibody is cultured, and the antibody is added at varying concentrations into the culture system to determine inhibitory activity against focus formation, colony formation, and spheroid growth. The antitumor activity can be confirmed in vivo, for example, by administering the antibody to a nude mouse with a transplanted cancer cell line highly expressing the target protein, and determining changes in the cancer cells.

Since the compound conjugated in the antibody-drug conjugate exerts an antitumor effect, it is preferred but not essential that the antibody itself should have an antitumor effect. For the purpose of specifically and selectively exerting the cytotoxic activity of the antitumor compound against cancer cells, it is important and also preferred that the antibody should have the property of being internalized to migrate into cancer cells.

The antibody in the antibody-drug conjugate used in the present invention can be obtained by a procedure known in the art. For example, the antibody of the present invention can be obtained using a method usually carried out in the art, which involves immunizing animals with an antigenic polypeptide and collecting and purifying antibodies produced in vivo. The origin of the antigen is not limited to humans, and the animals may be immunized with an antigen derived from a non-human animal such as a mouse, a rat and the like. In this case, the cross-reactivity of antibodies binding to the obtained heterologous antigen with human antigens can be tested to screen for an antibody applicable to a human disease.

Alternatively, antibody-producing cells which produce antibodies against the antigen can be fused with myeloma cells according to a method known in the art (for example, Kohler and Milstein, Nature (1975) 256, p. 495-497; Kennet, R. ed., Monoclonal Antibodies, p. 365-367, Plenum Press, N.Y. (1980)), to establish hybridomas, from which monoclonal antibodies can in turn be obtained.

The antigen can be obtained by genetically engineering host cells to produce a gene encoding the antigenic protein. Specifically, vectors that permit expression of the antigen gene are prepared and transferred to host cells so that the gene is expressed. The antigen thus expressed can be purified. The antibody can also be obtained by a method of immunizing animals with the above-described genetically engineered antigen-expressing cells or a cell line expressing the antigen.

The antibody in the antibody-drug conjugate used in the present invention is preferably a recombinant antibody obtained by artificial modification for the purpose of decreasing heterologous antigenicity to humans such as a chimeric antibody or a humanized antibody, or is preferably an antibody having only the gene sequence of an antibody derived from a human, that is, a human antibody. These antibodies can be produced using a known method.

As the chimeric antibody, an antibody in which antibody variable and constant regions are derived from different species, for example, a chimeric antibody in which a mouse- or rat-derived antibody variable region is connected to a human-derived antibody constant region can be exemplified (Proc. Natl. Acad. Sci. USA, 81, 6851-6855, (1984)).

As the humanized antibody, an antibody obtained by integrating only the complementarity determining region (CDR) of a heterologous antibody into a human-derived antibody (Nature (1986) 321, pp. 522-525), an antibody obtained by grafting a part of the amino acid residues of the framework of a heterologous antibody as well as the CDR sequence of the heterologous antibody to a human antibody by a CDR-grafting method (WO 90/07861), and an antibody humanized using a gene conversion mutagenesis strategy (U.S. Pat. No. 5,821,337) can be exemplified.

As the human antibody, an antibody generated by using a human antibody-producing mouse having a human chromosome fragment including genes of a heavy chain and light chain of a human antibody (see Tomizuka, K. et al., Nature Genetics (1997) 16, p. 133-143; Kuroiwa, Y. et. al., Nucl. Acids Res. (1998) 26, p. 3447-3448; Yoshida, H. et. al., Animal Cell Technology: Basic and Applied Aspects vol. 10, p. 69-73 (Kitagawa, Y., Matsuda, T. and Iijima, S. eds.), Kluwer Academic Publishers, 1999; Tomizuka, K. et. al., Proc. Natl. Acad. Sci. USA (2000) 97, p. 722-727, etc.) can be exemplified. As an alternative, an antibody obtained by phage display, the antibody being selected from a human antibody library (see Wormstone, I. M. et. al, Investigative Ophthalmology & Visual Science. (2002) 43 (7), p. 2301-2308; Carmen, S. et. al., Briefings in Functional Genomics and Proteomics (2002), 1 (2), p. 189-203; Siriwardena, D. et. al., Ophthalmology (2002) 109 (3), p. 427-431, etc.) can be exemplified.

In the antibody in the antibody-drug conjugate used in present invention, modified variants of the antibody are also included. The modified variant refers to a variant obtained by subjecting the antibody according to the present invention to chemical or biological modification. Examples of the chemically modified variant include variants including a linkage of a chemical moiety to an amino acid skeleton, variants including a linkage of a chemical moiety to an N-linked or O-linked carbohydrate chain, etc. Examples of the biologically modified variant include variants obtained by post-translational modification (such as N-linked or O-linked glycosylation, N- or C-terminal processing, deamidation, isomerization of aspartic acid, or oxidation of methionine), and variants in which a methionine residue has been added to the N terminus by being expressed in a prokaryotic host cell. Further, an antibody labeled so as to enable the detection or isolation of the antibody or an antigen according to the present invention, for example, an enzyme-labeled antibody, a fluorescence-labeled antibody, and an affinity-labeled antibody are also included in the meaning of the modified variant. Such a modified variant of the antibody according to the present invention is useful for improving the stability and blood retention of the antibody, reducing the antigenicity thereof, detecting or isolating an antibody or an antigen, and so on.

Further, by regulating the modification of a glycan which is linked to the antibody according to the present invention (glycosylation, defucosylation, etc.), it is possible to enhance antibody-dependent cellular cytotoxic activity. As the technique for regulating the modification of a glycan of antibodies, International Publication No. WO 99/54342, International Publication No. WO 00/61739, International Publication No. WO 02/31140, International Publication No. WO 2007/133855, International Publication No. WO 2013/120066, etc. are known. However, the technique is not limited thereto. In the antibody according to the present invention, antibodies in which the modification of a glycan is regulated are also included.

It is known that a lysine residue at the carboxyl terminus of the heavy chain of an antibody produced in a cultured mammalian cell is deleted (Journal of Chromatography A, 705: 129-134 (1995)), and it is also known that two amino acid residues (glycine and lysine) at the carboxyl terminus of the heavy chain of an antibody produced in a cultured mammalian cell are deleted and a proline residue newly located at the carboxyl terminus is amidated (Analytical Biochemistry, 360: 75-83 (2007)). However, such deletion and modification of the heavy chain sequence do not affect the antigen-binding affinity and the effector function (complement activation, antibody-dependent cellular cytotoxicity, etc.) of the antibody. Therefore, in the antibody according to the present invention, antibodies subjected to such modification and functional fragments of the antibody are also included, and deletion variants in which one or two amino acids have been deleted at the carboxyl terminus of the heavy chain, variants obtained by amidation of the deletion variants (for example, a heavy chain in which the carboxyl terminal proline residue has been amidated), and the like are also included. The type of deletion variant having a deletion at the carboxyl terminus of the heavy chain of the antibody according to the present invention is not limited to the above variants as long as the antigen-binding affinity and the effector function are conserved. The two heavy chains constituting the antibody according to the present invention may be of one type selected from the group consisting of a full-length heavy chain and the above-described deletion variant, or may be of two types in combination selected therefrom. The ratio of the amount of each deletion variant can be affected by the type of cultured mammalian cells which produce the antibody according to the present invention and the culture conditions; however, an antibody in which one amino acid residue at the carboxyl terminus has been deleted in both of the two heavy chains in the antibody according to the present invention can be preferably exemplified.

As isotypes of the antibody according to the present invention, for example, IgG (IgG1, IgG2, IgG3, IgG4) can be exemplified. Preferably, IgG1 or IgG2 can be exemplified.

Examples of antibodies in the antibody-drug conjugate used in the present invention can include, but are not particularly limited to, an anti-HER2 antibody, an anti-HER3 antibody, an anti-TROP2 antibody, an anti-B7-H3 antibody, an anti-GPR20 antibody, an anti-CDH6 antibody, an anti-CD3 antibody, an anti-CD30 antibody, an anti-CD33 antibody, an anti-CD37 antibody, an anti-CD56 antibody, an anti-CD98 antibody, an anti-DR5 antibody, an anti-EGFR antibody, an anti-EPHA2 antibody, an anti-FGFR2 antibody, an anti-FGFR4 antibody, an anti-FOLR1 antibody, an anti-VEGF antibody, an anti-CD20 antibody, an anti-CD22 antibody, an anti-CD70 antibody, an anti-PSMA antibody, an anti-CEA antibody, an anti-Mesothelin antibody, an anti-A33 antibody, an anti-CanAg antibody, an anti-Cripto antibody, an anti-G250 antibody, an anti-MUC1 antibody, an anti-GPNMB antibody, an anti-Integrin antibody, an anti-Tenascin-C antibody, and an anti-SLC44A4 antibody. Moreover, an anti-HER2 antibody, an anti-HER3 antibody, an anti-TROP2 antibody, an anti-B7-H3 antibody, an anti-GPR20 antibody, an anti-CDH6 antibody, an anti-MUC1 antibody, and anti-CD37 antibody, can be preferably exemplified. Further, an anti-HER2 antibody, an anti-HER3 antibody, an anti-TROP2 antibody, an anti-B7-H3 antibody, an anti-GPR20 antibody, an anti-CDH6 antibody, and an anti-MUC1 antibody, can be preferably exemplified.

In the present invention, the term “anti-HER2 antibody” refers to an antibody which binds specifically to HER2 (Human Epidermal Growth Factor Receptor Type 2; ErbB-2), and preferably has an activity of internalization in HER2-expressing cells by binding to HER2.

Examples of the anti-HER2 antibody include trastuzumab (U.S. Pat. No. 5,821,337) and pertuzumab (International Publication No. WO 01/00245). Preferably, trastuzumab can be exemplified.

In the present invention, the term “anti-HER3 antibody” refers to an antibody which binds specifically to HER3 (Human Epidermal Growth Factor Receptor Type 3; ErbB-3), and preferably has an activity of internalization in HER3-expressing cells by binding to HER3.

Examples of the anti-HER3 antibody include patritumab (U3-1287), U1-59 (International Publication No. WO 2007/077028), MM-121 (seribantumab), an anti-ERBB3 antibody described in International Publication No. WO 2008/100624, RG-7116 (lumretuzumab), and LJM-716 (elgemtumab). Preferably, patritumab and U1-59 can be exemplified.

In the present invention, the term “anti-TROP2 antibody” refers to an antibody which binds specifically to TROP2 (TACSTD2: Tumor-associated calcium signal transducer 2; EGP-1), and preferably has an activity of internalization in TROP2-expressing cells by binding to TROP2.

Examples of the anti-TROP2 antibody include hTINA1-H1L1 (International Publication No. WO 2015/098099).

In the present invention, the term “anti-B7-H3 antibody” refers to an antibody which binds specifically to B7-H3 (B cell antigen #7 homolog 3; PD-13; CD276), and preferably has an activity of internalization in B7-H3-expressing cells by binding to B7-H3.

Examples of the anti-B7-H3 antibody include M30-H1-L4 (International Publication No. WO 2014/057687).

In the present invention, the term “anti-GPR20 antibody” refers to an antibody which binds specifically to GPR20 (G Protein-coupled receptor 20), and preferably has an activity of internalization in GPR20-expressing cells by binding to GPR20.

Examples of the anti-GPR20 antibody include h046-H4e/L7 (International Publication No. WO 2018/135501).

In the present invention, the term “anti-CDH6 antibody” refers to an antibody which binds specifically to CDH6 (Cadherin-6), and preferably has an activity of internalization in CDH6-expressing cells by binding to CDH6.

Examples of the anti-CDH6 antibody include H01L02 (International Publication No. WO 2018/212136).

In the present invention, the term “anti-MUC1 antibody” refers to an antibody which binds specifically to MUC1 (Mucin 1), and preferably has an activity of internalization in MUC1-expressing cells by binding to MUC1.

Examples of the anti-MUC1 antibody include PankoMab (Danielczyk, A. et al. (2006) Cancer Immunol. Immunother. 55, 1337-1347) and the PankoMab variant (PM-N54Q) disclosed in the International Publication No. WO 2019/21989.

In the present invention, the term “anti-CD37 antibody” refers to an antibody which binds specifically to CD37, and preferably has an activity of internalization in CD37-expressing cells by binding to CD37.

Examples of the anti-CD37 antibody include, but are not limited to, an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19, an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19, or an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

### 3. Production of the Antibody-Drug Conjugate

A drug-linker intermediate for use in the production of the antibody-drug conjugate according to the present invention is represented by the following formula.

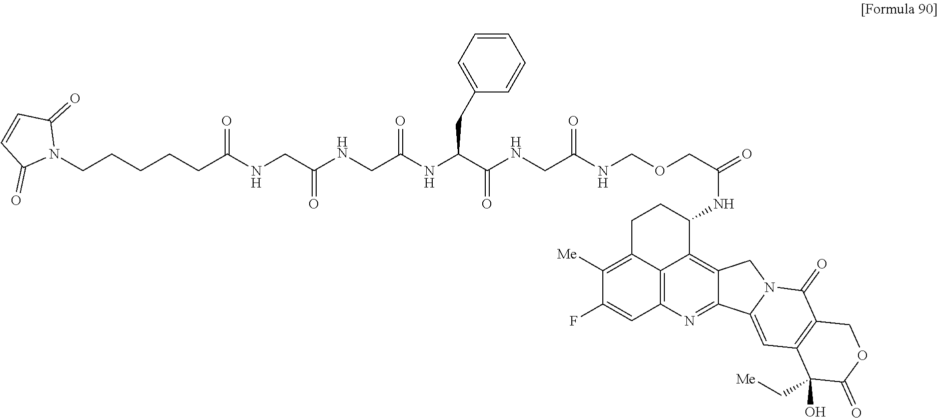 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

The drug-linker intermediate can be expressed as the chemical name N-[6-(2,5-dioxo-2,5-dihydro-1H-pyrrol-1-yl)hexanoyl]glycylglycyl-L-phenylalanyl-N-[(2-{[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]amino}-2-oxoethoxy)methyl]glycinamide, and can be produced with reference to descriptions in International Publication No. WO 2014/057687, International Publication No. WO 2015/098099, International Publication No. WO 2015/115091, International Publication No. WO 2015/155998, International Publication No. WO 2019/044947, and so on.

The antibody-drug conjugate used in the present invention can be produced by reacting the above-described drug-linker intermediate and an antibody having a thiol group (alternatively referred to as a sulfhydryl group).

The antibody having a sulfhydryl group can be obtained by a method well known in the art (Hermanson, G. T, Bioconjugate Techniques, pp. 56-136, pp. 456-493, Academic Press (1996)). For example, by using 0.3 to 3 molar equivalents of a reducing agent such as tris(2-carboxyethyl)phosphine hydrochloride (TCEP) per interchain disulfide within the antibody and reacting with the antibody in a buffer solution containing a chelating agent such as ethylenediamine tetraacetic acid (EDTA), an antibody having a sulfhydryl group with partially or completely reduced interchain disulfides within the antibody can be obtained.

Further, by using 2 to 20 molar equivalents of the drug-linker intermediate per the antibody having a sulfhydryl group, an antibody-drug conjugate in which 2 to 8 drug molecules are conjugated per antibody molecule can be produced.

The average number of conjugated drug molecules per antibody molecule of the antibody-drug conjugate produced can be determined, for example, by a method of calculation based on measurement of UV absorbance for the antibody-drug conjugate and the conjugation precursor thereof at two wavelengths of 280 nm and 370 nm (UV method), or a method of calculation based on quantification through HPLC measurement for fragments obtained by treating the antibody-drug conjugate with a reducing agent (HPLC method).

Conjugation between the antibody and the drug-linker intermediate and calculation of the average number of conjugated drug molecules per antibody molecule of the antibody-drug conjugate can be performed with reference to descriptions in International Publication No. WO 2014/057687, International Publication No. WO 2015/098099, International Publication No. WO 2015/115091, International Publication No. WO 2015/155998, International Publication No. WO 2018/135501, and International Publication No. WO 2018/212136, and so on.

In the present invention, the term “anti-HER2 antibody-drug conjugate” refers to an antibody-drug conjugate such that the antibody in the antibody-drug conjugate according to the invention is an anti-HER2 antibody.

The anti-HER2 antibody is preferably an antibody comprising a heavy chain comprising CDRH1 consisting of an amino acid sequence consisting of amino acid residues 26 to 33 of SEQ ID NO: 1 (SEQ ID NO: 20), CDRH2 consisting of an amino acid sequence consisting of amino acid residues 51 to 58 of SEQ ID NO: 1 (SEQ ID NO: 21), and CDRH3 consisting of an amino acid sequence consisting of amino acid residues 97 to 109 of SEQ ID NO: 1 (SEQ ID NO: 22), and a light chain comprising CDRL1 consisting of an amino acid sequence consisting of amino acid residues 27 to 32 of SEQ ID NO: 2 (SEQ ID NO: 23), CDRL2 consisting of an amino acid sequence consisting of amino acid residues 50 to 52 of SEQ ID NO: 2 (SEQ ID NO: 24), and CDRL3 consisting of an amino acid sequence consisting of amino acid residues 89 to 97 of SEQ ID NO: 2 (SEQ ID NO: 25), more preferably an antibody comprising a heavy chain comprising a heavy chain variable region consisting of an amino acid sequence consisting of amino acid residues 1 to 120 of SEQ ID NO: 1 (SEQ ID NO: 26) and a light chain comprising a light chain variable region consisting of an amino acid sequence consisting of amino acid residues 1 to 107 of SEQ ID NO: 2 (SEQ ID NO: 27), and even more preferably an antibody comprising a heavy chain consisting of an amino acid sequence represented by SEQ ID NO: 1 and a light chain consisting of an amino acid sequence represented by SEQ ID NO: 2, or an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

The average number of units of the drug-linker conjugated per antibody molecule in the anti-HER2 antibody-drug conjugate is preferably 2 to 8, more preferably 3 to 8, even more preferably 7 to 8, even more preferably 7.5 to 8, and even more preferably about 8.

The anti-HER2 antibody-drug conjugate can be produced with reference to descriptions in International Publication No. WO 2015/115091 and so on.

One such antibody-drug conjugate is trastuzumab deruxtecan (DS-8201a), which is composed of a HER2-targeting antibody and a derivative of exatecan. In particular, WO 2015/115091 provides a detailed description of exemplary HER2-targeting antibody-drug conjugates, including trastuzumab deruxtecan (DS-8201a).

In the present invention, the term “anti-HER3 antibody-drug conjugate” refers to an antibody-drug conjugate such that the antibody in the antibody-drug conjugate according to the invention is an anti-HER3 antibody.

The anti-HER3 antibody is preferably an antibody comprising a heavy chain comprising CDRH1 consisting of an amino acid sequence consisting of amino acid residues 26 to 35 of SEQ ID NO: 3, CDRH2 consisting of an amino acid sequence consisting of amino acid residues 50 to 65 of SEQ ID NO: 3, and CDRH3 consisting of an amino acid sequence consisting of amino acid residues 98 to 106 of SEQ ID NO: 3, and a light chain comprising CDRL1 consisting of an amino acid sequence consisting of amino acid residues 24 to 39 of SEQ ID NO: 4, CDRL2 consisting of an amino acid sequence consisting of amino acid residues 56 to 62 of SEQ ID NO: 4, and CDRL3 consisting of an amino acid sequence consisting of amino acid residues 95 to 103 of SEQ ID NO: 4, more preferably an antibody comprising a heavy chain comprising a heavy chain variable region consisting of an amino acid sequence consisting of amino acid residues 1 to 117 of SEQ ID NO: 3, and a light chain comprising a light chain variable region consisting of an amino acid sequence consisting of amino acid residues 1 to 113 of SEQ ID NO: 4, and even more preferably an antibody comprising a heavy chain consisting of an amino acid sequence represented by SEQ ID NO: 3 and a light chain consisting of an amino acid sequence represented by SEQ ID NO: 4, or a variant of the antibody in which a lysine residue at the carboxyl terminus of the heavy chain is deleted.

The average number of units of the drug-linker conjugated per antibody molecule in the anti-HER3 antibody-drug conjugate is preferably 2 to 8, more preferably 3 to 8, even more preferably 7 to 8, even more preferably 7.5 to 8, and even more preferably about 8.

The anti-HER3 antibody-drug conjugate can be produced with reference to descriptions in International Publication No. WO 2015/155998 and so on.

In the present invention, the term “anti-TROP2 antibody-drug conjugate” refers to an antibody-drug conjugate such that the antibody in the antibody-drug conjugate according to the invention is an anti-TROP2 antibody.

The anti-TROP2 antibody is preferably an antibody comprising a heavy chain comprising CDRH1 consisting of an amino acid sequence consisting of amino acid residues 50 to 54 of SEQ ID NO: 5 (SEQ ID NO: 28), CDRH2 consisting of an amino acid sequence consisting of amino acid residues 69 to 85 of SEQ ID NO: 5 (SEQ ID NO: 29), and CDRH3 consisting of an amino acid sequence consisting of amino acid residues 118 to 129 of SEQ ID NO: 5 (SEQ ID NO: 30), and a light chain comprising CDRL1 consisting of an amino acid sequence consisting of amino acid residues 44 to 54 of SEQ ID NO: 6 (SEQ ID NO: 31), CDRL2 consisting of an amino acid sequence consisting of amino acid residues 70 to 76 of SEQ ID NO: 6 (SEQ ID NO: 32), and CDRL3 consisting of an amino acid sequence consisting of amino acid residues 109 to 117 of SEQ ID NO: 6 (SEQ ID NO: 33), more preferably an antibody comprising a heavy chain comprising a heavy chain variable region consisting of an amino acid sequence consisting of amino acid residues 20 to 140 of SEQ ID NO: 5 (SEQ ID NO: 34), and a light chain comprising a light chain variable region consisting of an amino acid sequence consisting of amino acid residues 21 to 129 of SEQ ID NO: 6 (SEQ ID NO: 35), and even more preferably an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6, or a variant of the antibody in which a lysine residue at the carboxyl terminus of the heavy chain is deleted.

The average number of units of the drug-linker conjugated per antibody molecule in the anti-TROP2 antibody-drug conjugate is preferably 2 to 8, more preferably 3 to 5, even more preferably 3.5 to 4.5, and even more preferably about 4.

The anti-TROP2 antibody-drug conjugate can be produced with reference to descriptions in International Publication No. WO 2015/098099 and International Publication No. WO 2017/002776.

One such antibody-drug conjugate is datopotamab deruxtecan, which is composed of a TROP2-targeting antibody and a derivative of exatecan. In particular, WO 2015/098099 and WO 2020/240467 provide a detailed description of exemplary TROP2-targeting antibody-drug conjugates, including datopotamab deruxtecan (DS-1062).

In the present invention, the term “anti-B7-H3 antibody-drug conjugate” refers to an antibody-drug conjugate such that the antibody in the antibody-drug conjugate according to the invention is an anti-B7-H3 antibody.

The anti-B7-H3 antibody is preferably an antibody comprising a heavy chain comprising CDRH1 consisting of an amino acid sequence consisting of amino acid residues 50 to 54 of SEQ ID NO: 7, CDRH2 consisting of an amino acid sequence consisting of amino acid residues 69 to 85 of SEQ ID NO: 7, and CDRH3 consisting of an amino acid sequence consisting of amino acid residues 118 to 130 of SEQ ID NO: 7, and a light chain comprising CDRL1 consisting of an amino acid sequence consisting of amino acid residues 44 to 53 of SEQ ID NO: 8, CDRL2 consisting of an amino acid sequence consisting of amino acid residues 69 to 75 of SEQ ID NO: 8, and CDRL3 consisting of an amino acid sequence consisting of amino acid residues 108 to 116 of SEQ ID NO: 8, more preferably an antibody comprising a heavy chain comprising a heavy chain variable region consisting of an amino acid sequence consisting of amino acid residues 20 to 141 of SEQ ID NO: 7, and a light chain comprising a light chain variable region consisting of an amino acid sequence consisting of amino acid residues 21 to 128 of SEQ ID NO: 8, and even more preferably an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8, or a variant of the antibody in which a lysine residue at the carboxyl terminus of the heavy chain is deleted.

The average number of units of the drug-linker conjugated per antibody molecule in the anti-B7-H3 antibody-drug conjugate is preferably 2 to 8, more preferably 3 to 5, even more preferably 3.5 to 4.5, and even more preferably about 4.

The anti-B7-H3 antibody-drug conjugate used in the present invention can be produced with reference to descriptions in International Publication No. WO 2014/057687 and International Publication No. WO 2017/002776.

In the present invention, the term “anti-GPR20 antibody-drug conjugate” refers to an antibody-drug conjugate such that the antibody in the antibody-drug conjugate is an anti-GPR20 antibody.

The anti-GPR20 antibody is preferably an antibody comprising a heavy chain comprising CDRH1 consisting of an amino acid sequence consisting of amino acid residues 45 to 54 of SEQ ID NO: 9, CDRH2 consisting of an amino acid sequence consisting of amino acid residues 69 to 78 of SEQ ID NO: 9, and CDRH3 consisting of an amino acid sequence consisting of amino acid residues 118 to 131 of SEQ ID NO: 9, and a light chain comprising CDRL1 consisting of an amino acid sequence consisting of amino acid residues 44 to 54 of SEQ ID NO: 10, CDRL2 consisting of an amino acid sequence consisting of amino acid residues 70 to 76 of SEQ ID NO: 10, and CDRL3 consisting of an amino acid sequence consisting of amino acid residues 109 to 117 of SEQ ID NO: 10, more preferably an antibody comprising a heavy chain comprising a heavy chain variable region consisting of an amino acid sequence consisting of amino acid residues 20 to 142 of SEQ ID NO: 9, and a light chain comprising a light chain variable region consisting of an amino acid sequence consisting of amino acid residues 21 to 129 of SEQ ID NO: 10, and even more preferably an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10, or a variant of the antibody in which a lysine residue at the carboxyl terminus of the heavy chain is deleted.

The average number of units of the drug-linker conjugated per antibody molecule in the anti-GPR20 antibody-drug conjugate used in the present invention is preferably 2 to 8, more preferably 3 to 8, even more preferably 7 to 8, even more preferably 7.5 to 8, and even more preferably about 8.

The anti-GPR20 antibody-drug conjugate used in the present invention can be produced with reference to descriptions in International Publication No. WO 2018/135501 and so on.

In the present invention, the term “anti-CDH6 antibody-drug conjugate” refers to an antibody-drug conjugate such that the antibody in the antibody-drug conjugate is an anti-CDH6 antibody.

The anti-CDH6 antibody is preferably an antibody comprising a heavy chain comprising CDRH1 consisting of an amino acid sequence consisting of amino acid residues 45 to 54 of SEQ ID NO: 11, CDRH2 consisting of an amino acid sequence consisting of amino acid residues 69 to 78 of SEQ ID NO: 11, and CDRH3 consisting of an amino acid sequence consisting of amino acid residues 118 to 130 of SEQ ID NO: 11, and a light chain comprising CDRL1 consisting of an amino acid sequence consisting of amino acid residues 44 to 54 of SEQ ID NO: 12, CDRL2 consisting of an amino acid sequence consisting of amino acid residues 70 to 76 of SEQ ID NO: 12, and CDRL3 consisting of an amino acid sequence consisting of amino acid residues 109 to 116 of SEQ ID NO: 12, more preferably an antibody comprising a heavy chain comprising a heavy chain variable region consisting of an amino acid sequence consisting of amino acid residues 20 to 141 of SEQ ID NO: 11 and a light chain comprising a light chain variable region consisting of an amino acid sequence consisting of amino acid residues 21 to 128 of SEQ ID NO: 12, and even more preferably an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12, or a variant of the antibody in which a lysine residue at the carboxyl terminus of the heavy chain is deleted.

The average number of units of the drug-linker conjugated per antibody molecule in the anti-CDH6 antibody-drug conjugate used in the present invention is preferably 2 to 8, more preferably 3 to 8, even more preferably 7 to 8, even more preferably 7.5 to 8, and even more preferably about 8.

The anti-CDH6 antibody-drug conjugate used in the present invention can be produced with reference to descriptions in International Publication No. WO 2018/212136 and so on.

In the present invention, the term “anti-MUC1 antibody-drug conjugate” refers to an antibody-drug conjugate such that the antibody in the antibody-drug conjugate according to the invention is an anti-MUC1 antibody.

The anti-MUC1 antibody is preferably an antibody comprising a heavy chain comprising CDRH1 consisting of an amino acid sequence consisting of amino acid residues 31 to 35 of SEQ ID NO: 13, CDRH2 consisting of an amino acid sequence consisting of amino acid residues 50 to 65 of SEQ ID NO: 13, and CDRH3 consisting of an amino acid sequence consisting of amino acid residues 101 to 106 of SEQ ID NO: 13, and a light chain comprising CDRL1 consisting of an amino acid sequence consisting of amino acid residues 24 to 39 of SEQ ID NO: 15, CDRL2 consisting of an amino acid sequence consisting of amino acid residues 55 to 61 of SEQ ID NO: 15, and CDRL3 consisting of an amino acid sequence consisting of amino acid residues 94 to 102 of SEQ ID NO: 15, more preferably an antibody comprising a heavy chain comprising a heavy chain variable region consisting of an amino acid sequence consisting of amino acid residues 1 to 117 of SEQ ID NO: 13 and a light chain comprising a light chain variable region consisting of an amino acid sequence consisting of amino acid residues 1 to 113 of SEQ ID NO: 15, and even more preferably an antibody comprising a heavy chain consisting of an amino acid sequence consisting of amino acid residues 1 to 447 of SEQ ID NO: 13 and a light chain consisting of an amino acid sequence consisting of amino acid residues 1 to 219 of SEQ ID NO: 15, or a variant of the antibody in which a lysine residue at the carboxyl terminus of the heavy chain is deleted.

Alternatively, the anti-MUC1 antibody is preferably an antibody comprising a heavy chain comprising CDRH1 consisting of an amino acid sequence consisting of amino acid residues 31 to 35 of SEQ ID NO: 14, CDRH2 consisting of an amino acid sequence consisting of amino acid residues 50 to 65 of SEQ ID NO: 14, and CDRH3 consisting of an amino acid sequence consisting of amino acid residues 101 to 106 of SEQ ID NO: 14, and a light chain comprising CDRL1 consisting of an amino acid sequence consisting of amino acid residues 24 to 39 of SEQ ID NO: 15, CDRL2 consisting of an amino acid sequence consisting of amino acid residues 55 to 61 of SEQ ID NO: 15, and CDRL3 consisting of an amino acid sequence consisting of amino acid residues 94 to 102 of SEQ ID NO: 15, more preferably an antibody comprising a heavy chain comprising a heavy chain variable region consisting of an amino acid sequence consisting of amino acid residues 1 to 117 of SEQ ID NO: 14 and a light chain comprising a light chain variable region consisting of an amino acid sequence consisting of amino acid residues 1 to 113 of SEQ ID NO: 15, and even more preferably an antibody comprising a heavy chain consisting of an amino acid sequence consisting of amino acid residues 1 to 447 of SEQ ID NO: 14 and a light chain consisting of an amino acid sequence consisting of amino acid residues 1 to 219 of SEQ ID NO: 15, or a variant of the antibody in which a lysine residue at the carboxyl terminus of the heavy chain is deleted.

The average number of units of the drug-linker conjugated per antibody molecule in the anti-MUC1 antibody-drug conjugate is preferably 2 to 8, more preferably 3 to 8, even more preferably 7 to 8, even more preferably 7.5 to 8, and even more preferably about 8.

The anti-MUC1 antibody-drug conjugate can be produced with reference to descriptions in International Publication No. WO 2019/219891 and so on.

In the present invention, the term “anti-CD37 antibody-drug conjugate” refers to an antibody-drug conjugate such that the antibody in the antibody-drug conjugate according to the invention is an anti-CD37 antibody.

The anti-CD37 antibody is preferably an antibody comprising a heavy chain comprising CDRH1 consisting of an amino acid sequence consisting of amino acid residues 45 to 54 of SEQ ID NO: 16, CDRH2 consisting of an amino acid sequence consisting of amino acid residues 69 to 78 of SEQ ID NO: 16, and CDRH3 consisting of an amino acid sequence consisting of amino acid residues 118 to 127 of SEQ ID NO: 16, and a light chain comprising CDRL1 consisting of an amino acid sequence consisting of amino acid residues 44 to 54 of SEQ ID NO: 19, CDRL2 consisting of an amino acid sequence consisting of amino acid residues 70 to 76 of SEQ ID NO: 19, and CDRL3 consisting of an amino acid sequence consisting of amino acid residues 109 to 117 of SEQ ID NO: 19, more preferably an antibody comprising a heavy chain comprising a heavy chain variable region consisting of an amino acid sequence consisting of amino acid residues 20 to 138 of SEQ ID NO: 16 and a light chain comprising a light chain variable region consisting of an amino acid sequence consisting of amino acid residues 21 to 128 of SEQ ID NO: 19, and even more preferably an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19, or a variant of the antibody in which a lysine residue at the carboxyl terminus of the heavy chain is deleted.

Alternatively, the anti-CD37 antibody is preferably an antibody comprising a heavy chain comprising CDRH1 consisting of an amino acid sequence consisting of amino acid residues 45 to 54 of SEQ ID NO: 17, CDRH2 consisting of an amino acid sequence consisting of amino acid residues 69 to 78 of SEQ ID NO: 17, and CDRH3 consisting of an amino acid sequence consisting of amino acid residues 118 to 127 of SEQ ID NO: 17, and a light chain comprising CDRL1 consisting of an amino acid sequence consisting of amino acid residues 44 to 54 of SEQ ID NO: 19, CDRL2 consisting of an amino acid sequence consisting of amino acid residues 70 to 76 of SEQ ID NO: 19, and CDRL3 consisting of an amino acid sequence consisting of amino acid residues 109 to 117 of SEQ ID NO: 19, more preferably an antibody comprising a heavy chain comprising a heavy chain variable region consisting of an amino acid sequence consisting of amino acid residues 20 to 138 of SEQ ID NO: 17 and a light chain comprising a light chain variable region consisting of an amino acid sequence consisting of amino acid residues 21 to 128 of SEQ ID NO: 19, and even more preferably an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19, or a variant of the antibody in which a lysine residue at the carboxyl terminus of the heavy chain is deleted.

Moreover, the anti-CD37 antibody is preferably an antibody comprising a heavy chain comprising CDRH1 consisting of an amino acid sequence consisting of amino acid residues 45 to 54 of SEQ ID NO: 18, CDRH2 consisting of an amino acid sequence consisting of amino acid residues 69 to 78 of SEQ ID NO: 18, and CDRH3 consisting of an amino acid sequence consisting of amino acid residues 118 to 127 of SEQ ID NO: 18, and a light chain comprising CDRL1 consisting of an amino acid sequence consisting of amino acid residues 44 to 54 of SEQ ID NO: 19, CDRL2 consisting of an amino acid sequence consisting of amino acid residues 70 to 76 of SEQ ID NO: 19, and CDRL3 consisting of an amino acid sequence consisting of amino acid residues 109 to 117 of SEQ ID NO: 19, more preferably an antibody comprising a heavy chain comprising a heavy chain variable region consisting of an amino acid sequence consisting of amino acid residues 20 to 138 of SEQ ID NO: 18 and a light chain comprising a light chain variable region consisting of an amino acid sequence consisting of amino acid residues 21 to 128 of SEQ ID NO: 19, and even more preferably an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19, or a variant of the antibody in which a lysine residue at the carboxyl terminus of the heavy chain is deleted.

The average number of units of the drug-linker conjugated per antibody molecule in the anti-CD37 antibody-drug conjugate is preferably 2 to 8, more preferably 5 to 8, even more preferably 7 to 8, even more preferably 7.5 to 8, and even more preferably about 8.

The anti-CD37 antibody-drug conjugate can be prepared with a similar production method to those described in the patent specifications mentioned in the previous sections for the anti-HER2 antibody, the anti-HER3 antibody, the anti-TROP2 antibody, the anti-B7-H3 antibody, the anti-GPR20 antibody, the anti-CDH6 antibody, and the anti-MUC1 antibody.

The anti-CD37 antibody-drug conjugate can be used for treating any cancers in which CD37 is expressed in the cancers. Examples of cancers include, but are not limited to, B-cell non-Hodgkin lymphoma (NHL) (for example, diffuse large B-cell lymphoma (DLBCL), follicular lymphoma (FL), mantle cell lymphoma (MCL), marginal zone lymphoma (MZL), and Burkitt lymphoma (BL)), chronic lymphocytic leukemia (CLL), T-cell lymphoma (TCL) (for example, peripheral T-cell lymphoma (PTCL), and cutaneous T-cell lymphoma (CTCL)), myelodysplastic syndromes (MDS) and acute myeloid leukemia (AML). Other examples of cancers include, but are not limited to, diffuse large B-cell lymphoma (DLBCL), follicular lymphoma (FL), and chronic lymphocytic leukemia (CLL).

### 4. EZH1 Inhibitor, EZH2 Inhibitor and EZH1/2 Dual Inhibitor

In the present invention, “EZH1 inhibitor” is a drug that has the function of inhibiting EZH1 (Enhancer of zeste homologue 1).

In an embodiment of the present invention, the EZH1 inhibitor has an inhibitory effect against the histone methyltransferase activity of human EZH1, and the IC₅₀ thereof can be 1 μM or less, 500 nM or less, 400 nM or less, 300 nM or less, 200 nM or less, 150 nM or less, 100 nM or less, 90 nM or less, 80 nM or less, 70 nM or less, 60 nM or less, 50 nM or less, 40 nM or less, 30 nM or less, 20 nM or less, 15 nM or less, or 10 nM or less. The IC₅₀ value can be determined based on the method described in, for example, WO2015/141616, Test Example 1; for example, by detecting the inhibitory effect against the activity of EZH1 to transfer S-adenosyl methionine labeled with tritium to a peptide having an EZH1 target sequence (for example, a sequence of the 12th to 40th amino acids of a human histone H3 protein; as for the amino acid sequence of the human histone H3 protein, see, for example, the sequence registered under GenBank registration number: CAB02546.1). The methyltransferase activity of EZH1 can be measured by using a PRC2-EZH1 complex.

Examples of the EZH1 inhibitor that can be used in the present invention include, but are not particularly limited to, (2R)-7-bromo-2-[trans-4-(dimethylamino)cyclohexyl]-N-[(4,6-dimethyl-2-oxo-1,2-dihydropyridin-3-yl)methyl]-2,4-dimethyl-1,3-benzodioxole-5-carboxamide, Valemetostat (IUPAC name: (2R)-7-chloro-2-[trans-4-(dimethylamino)cyclohexyl]-N-[(4,6-dimethyl-2-oxo-1,2-dihydropyridin-3-yl)methyl]-2,4-dimethyl-1,3-benzodioxole-5-carboxamide) and pharmaceutically acceptable salts of these.

In the present invention, “EZH2 inhibitor” is a drug that has the function of inhibiting EZH2.

In an embodiment of the present invention, the EZH2 inhibitor has an inhibitory effect against the histone methyltransferase activity of human EZH2, and the IC₅₀ thereof can be 1 μM or less, 500 nM or less, 400 nM or less, 300 nM or less, 200 nM or less, 150 nM or less, 100 nM or less, 90 nM or less, 80 nM or less, 70 nM or less, 60 nM or less, 50 nM or less, 40 nM or less, 30 nM or less, 20 nM or less, 15 nM or less, or 10 nM or less. The IC₅₀ value can be determined based on the method described in, for example, WO2015/141616, Test Example 2; for example, by detecting the inhibitory effect against the activity of EZH2 to transfer S-adenosyl methionine labeled with tritium to a peptide having an EZH2 target sequence (for example, a sequence of the 12th to 40th amino acids of a human histone H3 protein; as for the amino acid sequence of the human histone H3 protein, see, for example, the sequence registered under GenBank registration number: CAB02546.1). EZH2 methyltransferase activity can be measured by using a PRC2-EZH2 complex.

Examples of the EZH2 inhibitor that can be used in the present invention include, but are not particularly limited to, N-[(1,2-dihydro-4,6-dimethyl-2-oxo-3-pyridinyl)methyl]-3-methyl-1-[(1S)-1-methylpropyl]-6-[6-(1-piperazinyl)-3-pyridinyl]-1H-indole-4-carboxamide, N-((4,6-dimethyl-2-oxo-1,2-dihydropyridin-3-yl)methyl)-5-(ethyl(tetrahydro-2H-piran-4-yl)amino)-4-methyl-4′-(morpholinomethyl)-[1,1′-biphenyl]-3-carboxamide, (2R)-7-bromo-2-[trans-4-(dimethylamino)cyclohexyl]-N-[(4,6-dimethyl-2-oxo-1,2-dihydropyridin-3-yl)methyl]-2,4-dimethyl-1,3-benzodioxole-5-carboxamide, Valemetostat (IUPAC name: (2R)-7-chloro-2-[trans-4-(dimethylamino)cyclohexyl]-N-[(4,6-dimethyl-2-oxo-1,2-dihydropyridin-3-yl)methyl]-2,4-dimethyl-1,3-benzodioxole-5-carboxamide), and pharmaceutically acceptable salts of these.

Examples of the EZH2 inhibitor that can be used in the present invention include (1S,2R,5R)-5-(4-aminoimidazo[4,5-c]pyridin-1-yl)-3-(hydroxymethyl)cyclopent-3-en-1,2-diol and a pharmaceutically acceptable thereof.

Examples of the EZH2 inhibitor that can be used in the present invention further include N-[(6-methyl-2-oxo-4-propyl-1H-pyridin-3-yl)methyl]-1-propan-2-yl-6-[6-(4-propan-2-ylpiperazin-1-yl)pyridin-3-yl]indazole-4-carboxamide and a pharmaceutically acceptable salt thereof.

Examples of the EZH2 inhibitor further include tazemetostat (IUPAC name: N-((4,6-Dimethyl-2-oxo-1,2-dihydropyridin-3-yl)methyl)-5-(ethyl(tetrahydro-2H-piran-4-yl)amino)-4-methyl-4′-(morpholinomethyl)-[1,1′-biphenyl]-3-carboxamide) (E7438 or EPZ-6438) and a pharmaceutically acceptable salt thereof. This compound is disclosed in WO2012/142504, Example 44.

Examples of the EZH2 inhibitor further include Lirametostat (CPI-1205) and a pharmaceutically acceptable salt thereof.

Examples of the EZH2 inhibitor further include N-[(1,2-Dihydro-4,6-dimethyl-2-oxo-3-pyridinyl)methyl]-3-methyl-1-[(1S)-1-methylpropyl]-6-[6-(1-piperazinyl)-3-pyridinyl]-1H-indole-4-carboxamide (GSK126) and a pharmaceutically acceptable salt thereof disclosed in WO2011/140324, Example 270.

Examples of the EZH2 inhibitor that can be used in the present invention further include N-[(1,2-dihydro-6-methyl-2-oxo-4-propyl-3-pyridinyl)methyl]-1-)1-methylethyl)-6-[2-(4-methyl-1-piperazinyl)-4-pyridinyl]-1H-indazole-4-carboxamide and a pharmaceutically acceptable salt thereof.

Examples of the EZH2 inhibitor that can be used in the present invention further include a variety of compounds described in Stazi, G. et al., Expert Opinion on Therapeutic Patents, 27: 7, 797-813, 2017. EZH2 inhibitors are known; for example, EZH2 inhibitors described in the following documents can be used in the present invention: WO2014/100646, WO2015/057859, WO2016/081523, WO2014/144747, WO2015/010078, WO2015/010049, WO2015/200650, WO2015/132765, WO2015/004618, WO2016/066697, WO2014/124418, WO2015/023915, WO2016/130396, WO2015/077193, WO2015/077194, WO2015/193768, WO2016/073956, WO2016/073903, WO2016/102493, WO2016/089804, WO2014/151369.

In the present invention, the EZH2 inhibitor may further have an EZH1 inhibitory effect and may be, for example, an EZH1/2 dual inhibitor. For example, the aforementioned N-[(6-methyl-2-oxo-4-propyl-1H-pyridin-3-yl)methyl]-1-propan-2-yl-6-[6-(4-propan-2-ylpiperazin-1-yl)pyridin-3-yl]indazole-4-carboxamide and a pharmaceutically acceptable salt thereof can be an EZH1/2 dual inhibitor.

In an embodiment of the present invention, an EZH1/2 dual inhibitor has an inhibitory effect against the histone methyltransferase activity of human EZH1, and the IC₅₀ thereof can be 1 μM or less, 500 nM or less, 400 nM or less, 300 nM or less, 200 nM or less, 150 nM or less, 100 nM or less, 90 nM or less, 80 nM or less, 70 nM or less, 60 nM or less, 50 nM or less, 40 nM or less, 30 nM or less, 20 nM or less, 15 nM or less, or 10 nM or less, and, has an inhibitory effect against the histone methyltransferase activity of human EZH2, and the IC₅₀ thereof can be 1 μM or less, 500 nM or less, 400 nM or less, 300 nM or less, 200 nM or less, 150 nM or less, 100 nM or less, 90 nM or less, 80 nM or less, 70 nM or less, 60 nM or less, 50 nM or less, 40 nM or less, 30 nM or less, 20 nM or less, 15 nM or less, or 10 nM or less. The IC₅₀ values for EZH1 and EZH2 each can be determined as mentioned above.

Examples of the EZH1/2 dual inhibitor that can be used in the present invention include, but are not particularly limited to, (2R)-7-bromo-2-[trans-4-(dimethylamino)cyclohexyl]-N-[(4,6-dimethyl-2-oxo-1,2-dihydropyridin-3-yl)methyl]-2,4-dimethyl-1,3-benzodioxole-5-carboxamide, Valemetostat (IUPAC name: (2R)-7-chloro-2-[trans-4-(dimethylamino)cyclohexyl]-N-[(4,6-dimethyl-2-oxo-1,2-dihydropyridin-3-yl)methyl]-2,4-dimethyl-1,3-benzodioxole-5-carboxamide), and pharmaceutically acceptable salts of these.

(2R)-7-bromo-2-[trans-4-(dimethylamino)cyclohexyl]-N-[(4,6-dimethyl-2-oxo-1,2-dihydropyridin-3-yl)methyl]-2,4-dimethyl-1,3-benzodioxole-5-carboxamide is disclosed in WO2015/141616, Example 15, and is a compound having the following structure:

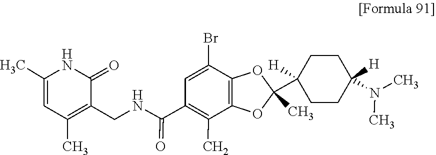

Valemetostat (IUPAC name: (2R)-7-chloro-2-[trans-4-(dimethylamino)cyclohexyl]-N-[(4,6-dimethyl-2-oxo-1,2-dihydropyridin-3-yl)methyl]-2,4-dimethyl-1,3-benzodioxole-5-carboxamide) (DS-3201a) is disclosed in WO2015/141616, Example 35, and is a compound having the following structure:

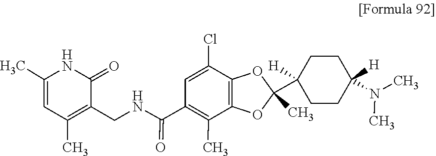

Valemetostat tosylate (IUPAC name: (2R)-7-chloro-2-[trans-4-(dimethylamino)cyclohexyl]-N-[(4,6-dimethyl-2-oxo-1,2-dihydropyridin-3-yl)methyl]-2,4-dimethyl-1,3-benzodioxole-5-carboxamide p-toluene sulfonate) (DS-3201b) (also referred to as “Compound A” in this patent specification) is disclosed in WO2015/141616, Example 80. Compound A can be prepared by referring to the synthetic method disclosed in WO2015/141616.

An inhibitor selected from the group consisting of an EZH1 inhibitor, an EZH2 inhibitor and an EZH1/2 dual inhibitor in the present invention may be present in the form of a pharmaceutically acceptable salt. The pharmaceutically acceptable salt includes either an acid addition salt or a salt with a base, but is preferably an acid addition salt, examples of which can include lower alkanesulfonates such as camsilate (camphorsulfonate), methanesulfonate, trifluoromethanesulfonate, and ethanesulfonate; arylsulfonates such as tosylate (p-toluenesulfonate), and benzenesulfonate; inorganic acid salts such as phosphate, nitrate, perchlorate, and hydrosulfate; hydrohalic acid salts such as hydrochloride, hydrobromide, hydroiodide, and hydrofluoride; organic acid salts such as acetate, malate, fumarate, succinate, citrate, tartrate, oxalate, and maleate; and amino acid salts such as ornithinate, glutamate, and aspartate.

Moreover, the inhibitor selected from the group consisting of an EZH1 inhibitor, an EZH2 inhibitor and an EZH1/2 dual inhibitor and pharmaceutically acceptable salts thereof may also be present in the form of a solvate. For clarification, the solvate may be a solvate of a pharmaceutically acceptable salt of the inhibitor selected from the group consisting of an EZH1 inhibitor, an EZH2 inhibitor and an EZH1/2 dual inhibitor.

### 5. Medicament

Described in the following are a pharmaceutical product and a method of treatment according to the present invention, wherein an antibody-drug conjugate and an inhibitor selected from the group consisting of an EZH1 inhibitor, an EZH2 inhibitor and an EZH1/2 dual inhibitor are administered in combination.

The pharmaceutical product and method of treatment of the present invention may be those in which the antibody-drug conjugate and the inhibitor selected from the group consisting of an EZH1 inhibitor, an EZH2 inhibitor and an EZH1/2 dual inhibitor are separately contained as active components in different formulations and are administered simultaneously or at different times, or may be those in which the antibody-drug conjugate and the inhibitor selected from the group consisting of an EZH1 inhibitor, an EZH2 inhibitor and an EZH1/2 dual inhibitor are contained as active components in a single formulation and administered. The antibody-drug conjugate and the inhibitor selected from the group consisting of an EZH1 inhibitor, an EZH2 inhibitor and an EZH1/2 dual inhibitor may be administered at different intervals when the antibody-drug conjugate and the inhibitor selected from the group consisting of an EZH1 inhibitor, an EZH2 inhibitor and an EZH1/2 dual inhibitor are separately contained as active components in different formulations.

The term “pharmaceutical product” refers to a preparation which is in such form as to permit the biological activity of the active ingredients, either as a composition containing all the active ingredients (for simultaneous administration), or as a combination of separate compositions (a combined preparation) each containing at least one but not all of the active ingredients (for administration sequentially or simultaneously), and which contains no additional components which are unacceptably toxic to a subject to which the product would be administered. Such product can be sterile.

The pharmaceutical product and method of treatment of the present invention can be used for treating cancer, and can be preferably used for treating at least one disease selected from the group consisting of breast cancer (including triple-negative breast cancer and luminal breast cancer), gastric cancer (also called gastric adenocarcinoma), colorectal cancer (also called colon and rectal cancer, and including colon cancer and rectal cancer), lung cancer (including small cell lung cancer and non-small cell lung cancer), esophageal cancer, head-and-neck cancer (including salivary gland cancer and pharyngeal cancer), gastroesophageal junction adenocarcinoma, biliary tract cancer (including bile duct cancer), Paget's disease, pancreatic cancer, ovarian cancer, uterine carcinosarcoma, urothelial cancer, prostate cancer, bladder cancer, gastrointestinal stromal tumor, uterine cervix cancer, squamous cell carcinoma, peritoneal cancer, liver cancer, hepatocellular cancer, endometrial cancer, kidney cancer, vulval cancer, thyroid cancer, penis cancer, leukemia, malignant lymphoma, non-Hodgkin lymphoma (for example, diffuse large B-cell lymphoma (DLBCL), follicular lymphoma (FL), mantle cell lymphoma (MCL), marginal zone lymphoma (MZL), Burkitt lymphoma (BL)), chronic lymphocytic leukemia, T-cell lymphoma (TCL) (for example, peripheral T-cell lymphomas (PTCL), cutaneous T cell lymphoma (CTCL)), acute myeloblastic leukemia (AML), plasmacytoma, myeloma, glioblastoma multiforme, sarcoma, osteosarcoma, and melanoma; and can more preferably be used for treating at least one cancer selected from the group consisting of breast cancer, gastric cancer, colorectal cancer, lung cancer, esophageal cancer, head-and-neck cancer, gastroesophageal junction adenocarcinoma, biliary tract cancer, Paget's disease, pancreatic cancer, ovarian cancer, bladder cancer, prostate cancer, uterine carcinosarcoma, gastrointestinal stromal tumor, kidney cancer, and sarcoma; and can more preferably be used for treating at least one cancer selected from the group consisting of non-Hodgkin lymphoma (for example, diffuse large B-cell lymphoma (DLBCL), follicular lymphoma (FL), mantle cell lymphoma (MCL), marginal zone lymphoma (MZL), Burkitt lymphoma (BL)) and chronic lymphocytic leukemia; and can more preferably be used for treating at least one cancer selected from the group consisting of T-cell lymphoma (TCL) (for example, peripheral T-cell lymphomas (PTCL), cutaneous T cell lymphoma (CTCL)) and acute myeloblastic leukemia (AML).

In one embodiment, the pharmaceutical product and method of treatment of the present invention can be used for treating breast cancer. In another embodiment, the pharmaceutical product and method of treatment of the present invention can be used for treating triple-negative breast cancer. In another embodiment, the pharmaceutical product and method of treatment of the present invention can be used for treating HER2 low-expressing breast cancer.

In one embodiment, the pharmaceutical product and method of treatment of the present invention can be used for treating lung cancer. In another embodiment, the pharmaceutical product and method of treatment of the present invention can be used for treating non-small cell lung cancer.

In one embodiment, the pharmaceutical product and method of treatment of the present invention can be used for treating non-Hodgkin lymphoma. In another embodiment, the pharmaceutical product and method of treatment of the present invention can be used for treating diffuse large B-cell lymphoma (DLBCL).

In one embodiment, the pharmaceutical product and method of treatment of the present invention can be used for treating prostate cancer.

In one embodiment, the pharmaceutical product and method of treatment of the present invention can be used for treating ovarian cancer.

Among the antibody-drug conjugates used in the present invention, the kind of antibody preferably used in the antibody-drug conjugate can be determined by examining the type of cancer and tumor markers. For example, in the case that HER2 expression is found in the cancer, an anti-HER2 antibody-drug conjugate can be preferably used; in the case that HER3 expression is found in the cancer, an anti-HER3 antibody-drug conjugate can be preferably used; in the case that TROP2 expression is found in the cancer, an anti-TROP2 antibody-drug conjugate can be preferably used; in the case that B7-H3 expression is found in the cancer, an anti-B7-H3 antibody-drug conjugate can be preferably used; in the case that GPR20 expression is found in the cancer, an anti-GPR20 antibody-drug conjugate can be preferably used; in the case that CDH6 expression is found in the cancer, an anti-CDH6 antibody-drug conjugate can be preferably used; in the case that MUC1 expression is found in the cancer, an anti-MUC1 antibody-drug conjugate can be preferably used; and in the case that CD37 expression is found in the cancer, an anti-CD37 antibody-drug conjugate can be preferably used.

The presence or absence of HER2, HER3, TROP2, B7-H3, GPR20, CDH6, and MUC1, and other tumor markers, can be checked by, for example, collecting tumor tissue from a cancer patient, and subjecting the formalin fixed paraffin embedded specimen (FFPE) to an examination at a gene product (protein) level, such as an immunohistochemistry (IHC) method, a flow cytometry, a western blot method, or an examination at a gene transcription level, such as an in situ hybridization method (ISH), a quantitative PCR method (q-PCR), or a microarray analysis; alternatively, it can also be checked by collecting cell-free blood circulating tumor DNA (ctDNA) from a cancer patient and subjecting to an examination which uses a method such as next-generation sequencing (NGS). These methods can be applied to the presence or absence of CD37.

In the case that the antibody-drug conjugate used in the present invention is an anti-HER2 antibody-drug conjugate, the pharmaceutical product and method of treatment of the present invention can be preferably used not only for HER2-overexpressing cancer but also for HER2 low-expressing cancer and HER2-mutated cancer.

In the present invention, the term “HER2-overexpressing cancer” is not particularly limited as long as it is recognized as HER2-overexpressing cancer by those skilled in the art. Preferred examples of the HER2-overexpressing cancer can include cancer given a score of 3+ for the expression of HER2 in an immunohistochemical method, and cancer given a score of 2+ for the expression of HER2 in an immunohistochemical method and determined as positive for the expression of HER2 in an in situ hybridization method. The in situ hybridization method of the present invention includes a fluorescence in situ hybridization method (FISH) and a dual color in situ hybridization method (DISH).

In the present invention, the term “HER2 low-expressing cancer” is not particularly limited as long as it is recognized as HER2 low-expressing cancer by those skilled in the art. Preferred examples of the HER2 low-expressing cancer can include cancer given a score of 2+ for the expression of HER2 in an immunohistochemical method and determined as negative for the expression of HER2 in an in situ hybridization method, and cancer given a score of 1+ for the expression of HER2 in an immunohistochemical method. Another example of the HER2 low-expressing cancer can include, but is not particularly limited to, cancer given a score of >0 and <1+ for the expression of HER2 in an immunohistochemical method.

The method for scoring the degree of HER2 expression by the immunohistochemical method, or the method for determining positivity or negativity to HER2 expression by the in situ hybridization method is not particularly limited as long as it is recognized by those skilled in the art. Examples of the method can include a method described in the 4th edition of the guidelines for HER2 testing, breast cancer (developed by the Japanese Pathology Board for Optimal Use of HER2 for Breast Cancer).

The HER2 low-expressing cancer for which the pharmaceutical product and method of treatment of the present invention can be used is preferably HER2 low-expressing breast cancer, HER2 low-expressing gastric cancer, HER2 low-expressing colorectal cancer, or HER2 low-expressing non-small cell lung cancer, and is more preferably HER2 low-expressing breast cancer.

The pharmaceutical product and method of treatment of the present invention can be preferably used for mammals, and can be more preferably used for humans.

The pharmaceutical product and method of treatment of the present invention can be preferably used for a maintenance therapy. The pharmaceutical product and method of treatment of the present invention may be used for the purpose of preventing the recurrence of a tumor after initial chemotherapy.

In the present invention, the term “maintenance therapy” is used to mean treatment that is given to help prevent cancer from coming back after it has disappeared following an initial therapy. The term is defined on the basis of “maintenance therapy” in the following reference.

NCI Dictionaries, “maintenance therapy”, NCI Dictionary of Cancer Terms [online]. National Cancer Institute [retrieved on 2023 Apr. 10]. Retrieved from <cancer.gov/publications/dictionaries/cancer-terms/def/maintenance-therapy>.

The antitumor effect of the pharmaceutical product and method of treatment of the present invention can be confirmed by, for example, generating a model in which cancer cells are transplanted to a test animal, and measuring reduction in tumor volume, life-prolonging effects due to applying the pharmaceutical product and method of treatment of the present invention. Furthermore, comparison with the antitumor effect of single administration of each of the antibody-drug conjugate and the inhibitor selected from the group consisting of an EZH1 inhibitor, an EZH2 inhibitor and an EZH1/2 dual inhibitor used in the present invention can provide confirmation of the combined effect of the antibody-drug conjugate and the inhibitor selected from the group consisting of an EZH1 inhibitor, an EZH2 inhibitor and an EZH1/2 dual inhibitor used in the present invention.

In addition, the antitumor effect of the pharmaceutical product and method of treatment of the present invention can be confirmed, in a clinical study, with the Response Evaluation Criteria in Solid Tumors (RECIST) evaluation method, WHO's evaluation method, Macdonald's evaluation method, measurement of body weight, and other methods; and can be determined by indicators such as Complete response (CR), Partial response (PR), Progressive disease (PD), Objective response rate (ORR), Duration of response (DoR), Progression-free survival (PFS), and Overall survival (OS).

The foregoing methods can provide confirmation of superiority in terms of the antitumor effect of the pharmaceutical product and method of treatment of the present invention compared to existing pharmaceutical products and methods of treatment for cancer therapy.

The pharmaceutical product and method of treatment of the present invention can retard growth of cancer cells, suppress their proliferation, and further can kill cancer cells. These effects can allow cancer patients to be free from symptoms caused by cancer or can achieve an improvement in the QOL of cancer patients and attain a therapeutic effect by sustaining the lives of the cancer patients. Even if the pharmaceutical product and method of treatment do not accomplish the killing of cancer cells, they can achieve higher QOL of cancer patients while achieving longer-term survival, by inhibiting or controlling the growth of cancer cells. Furthermore, the pharmaceutical product and method of treatment of the present invention can show a sustained antitumor effect.

The pharmaceutical product and method of treatment of the present invention are not associated with severe body weight loss.

The pharmaceutical product of the present invention can be expected to exert a therapeutic effect by application as systemic therapy to patients, and additionally, by local application to cancer tissues.

The pharmaceutical product of the present invention includes a pharmaceutical composition containing at least one pharmaceutically suitable ingredient. The pharmaceutically suitable ingredient can be suitably selected and applied from formulation additives or the like that are generally used in the art, in accordance with the dosage, administration concentration or the like of the antibody-drug conjugate used in the present invention and the inhibitor selected from the group consisting of an EZH1 inhibitor, an EZH2 inhibitor and an EZH1/2 dual inhibitor. For example, the antibody-drug conjugate used in the present invention can be administered as a pharmaceutical product containing a buffer such as a histidine buffer, an excipient such as sucrose or trehalose, and a surfactant such as Polysorbate 80 or 20. The pharmaceutical product containing the antibody-drug conjugate used in the present invention can be preferably used as an injection, can be more preferably used as an aqueous injection or a lyophilized injection, and can be even more preferably used as a lyophilized injection.

In the case that the pharmaceutical product containing the antibody-drug conjugate used in the present invention is an aqueous injection, it can be preferably diluted with a suitable diluent and then given as an intravenous infusion. For the diluent, a dextrose solution, physiological saline, and the like, can be exemplified, and a dextrose solution can be preferably exemplified, and a 5% dextrose solution can be more preferably exemplified.

In the case that the pharmaceutical product containing the antibody-drug conjugate used in the present invention is a lyophilized injection, it can be preferably dissolved in water for injection, subsequently a required amount can be diluted with a suitable diluent and then given as an intravenous infusion. For the diluent, a dextrose solution, physiological saline, and the like, can be exemplified, and a dextrose solution can be preferably exemplified, and a 5% dextrose solution can be more preferably exemplified.

Examples of the administration route which may be used to administer the pharmaceutical product of the present invention include intravenous, intradermal, subcutaneous, intramuscular, and intraperitoneal routes; and preferably include an intravenous route.

The antibody-drug conjugate used in the present invention can be administered to a human once at intervals of 1 to 180 days, and can be preferably administered once a week, once every 2 weeks, once every 3 weeks, or once every 4 weeks, and can be even more preferably administered once every 3 weeks. Also, the antibody-drug conjugate used in the present invention can be administered at a dose of about 0.001 to 100 mg/kg, and can be preferably administered at a dose of 0.8 to 12.4 mg/kg. In the case that the antibody-drug conjugate used in the present invention is an anti-HER2 antibody-drug conjugate, it can be preferably administered once every 3 weeks at a dose of 0.8 mg/kg, 1.6 mg/kg, 3.2 mg/kg, 5.4 mg/kg, 6.4 mg/kg, 7.4 mg/kg, or 8 mg/kg. In the case that the antibody-drug conjugate used in the present invention is an anti-HER3 antibody-drug conjugate, it can be preferably administered once every 3 weeks at a dose of 1.6 mg/kg, 3.2 mg/kg, 4.8 mg/kg, 5.6 mg/kg, 6.4 mg/kg, 8.0 mg/kg, 9.6 mg/kg, or 12.8 mg/kg. In the case that the antibody-drug conjugate used in the present invention is an anti-TROP2 antibody-drug conjugate, it can be preferably administered once every 3 weeks at a dose of 0.27 mg/kg, 0.5 mg/kg, 1.0 mg/kg, 2.0 mg/kg, 4.0 mg/kg, 6.0 mg/kg, or 8.0 mg/kg.

The dosage amount of an inhibitor selected from the group consisting of an EZH1 inhibitor, an EZH2 inhibitor and an EZH1/2 dual inhibitor according to the present invention is not particularly limited as long as it is an effective amount for treating a target disease and appropriately selected depending on the age, body weight, symptom, health condition and disease progression of the patient. The frequency of administration is not particularly limited and can be appropriately selected depending on the purpose. For example, the dosage amount per day is administered once a day or divided into a plurality of doses and administered separately. When the medical agent of the present invention is administered to a human, the dosage amount of an active ingredient ranges from about 0.01 mg/kg (body weight) to about 500 mg/kg (body weight), and preferably, about 0.1 mg/kg (body weight) to about 100 mg/kg (body weight). For administration to a human, the dosage amount per day is preferably administered once a day or divided into 2 to 4 portions, which are administered separately at appropriate intervals.

The pharmaceutical product and method of treatment of the present invention may further contain a cancer therapeutic agent other than the antibody-drug conjugate and the inhibitor selected from the group consisting of an EZH1 inhibitor, an EZH2 inhibitor and an EZH1/2 dual inhibitor according to the present invention. The pharmaceutical product and method of treatment of the present invention can also be administered in combination with another cancer therapeutic agent, thereby enhancing the antitumor effect. Other cancer therapeutic agents to be used for such purpose may be administered to a subject simultaneously, separately, or sequentially with the pharmaceutical composition of the present invention, or may be administered with varying each dosage interval. Such cancer therapeutic agents are not limited as long as they are agents having antitumor activity, and can be exemplified by at least one selected from the group consisting of irinotecan (CPT-11), cisplatin, carboplatin, oxaliplatin, fluorouracil (5-FU), gemcitabine, capecitabine, paclitaxel, docetaxel, doxorubicin, epirubicin, cyclophosphamide, mitomycin C, tegafur-gimeracil-oteracil combination, cetuximab, panitumumab, bevacizumab, ramucirumab, regorafenib, trifluridine-tipiracil combination, gefitinib, erlotinib, afatinib, methotrexate, pemetrexed, tamoxifen, toremifene, fulvestrant, leuprorelin, goserelin, letrozole, anastrozole, progesterone formulation, trastuzumab, pertuzumab, and lapatinib.

The pharmaceutical product and method of treatment of the present invention can also be used in combination with radiotherapy. For example, a cancer patient may receive radiotherapy before and/or after or simultaneously with receiving therapy with the pharmaceutical product of the present invention.

The pharmaceutical product and method of treatment of the present invention can also be used as an adjuvant chemotherapy in combination with a surgical procedure. The pharmaceutical product of the present invention may be administered for the purpose of diminishing the size of a tumor before a surgical procedure (referred to as pre-operative adjuvant chemotherapy or neoadjuvant therapy), or may be administered after a surgical procedure for the purpose of preventing the recurrence of a tumor (referred to as post-operative adjuvant chemotherapy or adjuvant therapy).

### EXAMPLES

The present invention is specifically described in view of the examples shown below. However, the present invention is not limited to these. Further, it is by no means to be interpreted in a limited way.

### Example 1: Production of the Anti-HER2 Antibody-Drug Conjugate (1)

In accordance with a production method described in International Publication No. WO 2015/115091 with use of an anti-HER2 antibody (an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2), an anti-HER2 antibody-drug conjugate in which a drug-linker represented by the following formula:

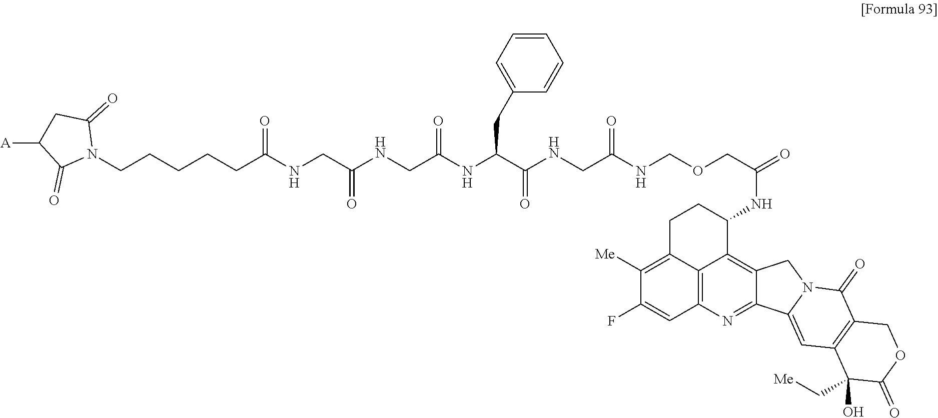

wherein A represents a connecting position to an antibody,
is conjugated to the anti-HER2 antibody via a thioether bond (hereinafter referred to as the “HER2-ADC (1)” (trastuzumab deruxtecan/DS-8201a)) was produced. The DAR of the HER2-ADC (1) is 7.7 or 7.8.

### Example 2: Production of the Anti-TROP2 Antibody-Drug Conjugate (1)

In accordance with a production method described in International Publication No. WO 2015/098099 and International Publication No. 2017/002776 with use of an anti-TROP2 antibody (an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6), an anti-TROP2 antibody-drug conjugate in which a drug-linker represented by the following formula:

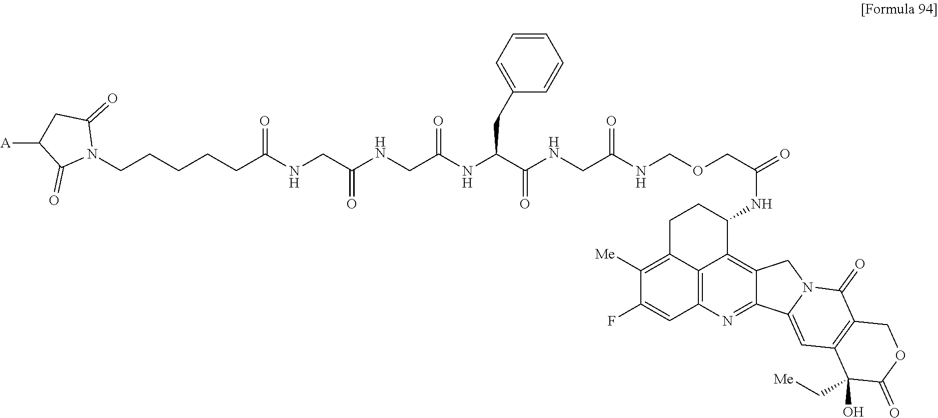

wherein A represents the connecting position to an antibody,
is conjugated to the anti-TROP2 antibody via a thioether bond (hereinafter referred to as the “TROP2-ADC (1)”) was produced. The DAR of TROP2-ADC (1) is 3.5 to 4.5.

### Example 3: Production of the Anti-HER3 Antibody-Drug Conjugate (1)

In accordance with a production method described in International Publication No. WO 2015/155998 with use of an anti-HER3 antibody (an antibody comprising a heavy chain consisting of an amino acid sequence represented by SEQ ID NO: 3 and a light chain consisting of an amino acid sequence represented by SEQ ID NO: 4), an anti-HER3 antibody-drug conjugate in which a drug-linker represented by the following formula:

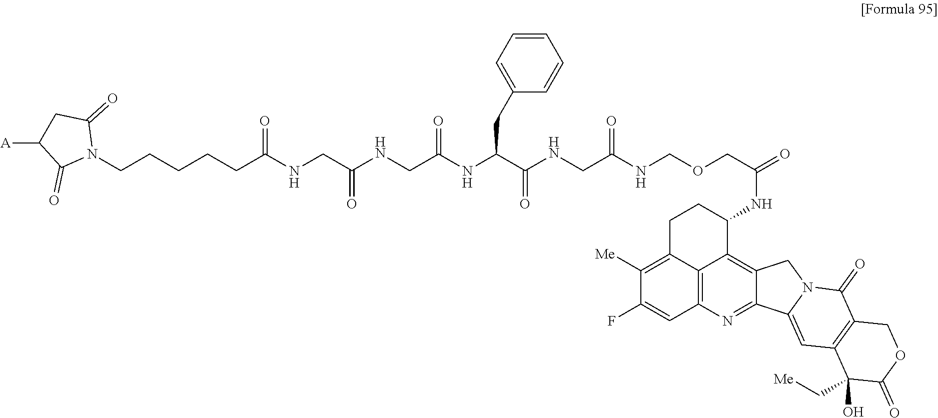

wherein A represents a connecting position to an antibody,
is conjugated to the anti-HER3 antibody via a thioether bond (hereinafter referred to as the “HER3-ADC (1)”) was produced. The DAR of the HER3-ADC (1) is 7.6.

### Example 4: Production of the Anti-CDH6 Antibody-Drug Conjugate (1)

In accordance with a production method described in International Publication No. WO 2018/212136 with use of an anti-CDH6 antibody (an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12), an anti-CDH6 antibody-drug conjugate in which a drug-linker represented by the following formula:

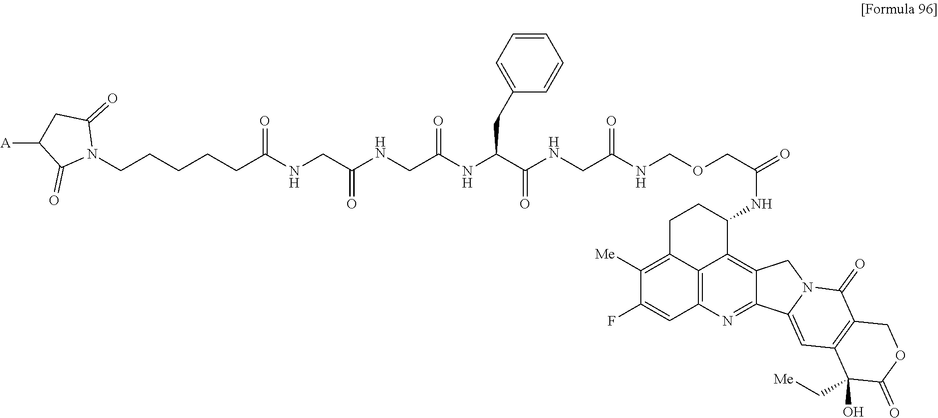

wherein A represents a connecting position to an antibody,
is conjugated to the anti-CDH6 antibody via a thioether bond (hereinafter referred to as the “CDH6-ADC (1)”) was produced. The DAR of CDH6-ADC (1) is 7.8.

### Example 5: Antitumor Study (1)

Measurement and calculation formula: In all studies, the major axis and minor axis of tumors were measured once a week or twice a week with an electronic digital caliper, and the estimated tumor volume (mm³) was calculated. The calculation formula is as shown below.

$$Estimated tumor volume ( {mm}^{3} ) = 1 / 2 \times Major axis ( mm ) \times {[ Minor axis ( mm ) ]}^{2}$$

Tumor growth inhibition (TGI) was calculated in accordance with the following calculation formula.

$$Tumor growth inhibition ( \% ) = 100 \times ( 1 - T / C )$$

T: Average estimated tumor volume of mice of test substance administration group
C: Average estimated tumor volume of mice of control group

The HER2-ADC (1) was diluted with ABS buffer (10 mM acetate buffer [pH 5.5], 5% sorbitol), and intravenously administered in a fluid volume of 10 mL/kg to the tail vein. The Compound A was orally administered by a feed mix containing 0.3% of Compound A.

These methods are common to Examples 5 to 8, and Examples 11 to 16. In Example 12 to Example 16, TROP2-ADC (1), HER3-ADC (1), CD37-ADC (1), or B7-H3-ADC (1) was used in the evaluation. Moreover, the Compound A was orally administered by a feed mix containing 0.25% of Compound A in Example 12 to Example 16.

Human gastric cancer cell line NCI-N87, which was purchased from ATCC (American Type Culture Collection), was suspended into DPBS (Dulbecco's phosphate-buffered saline) and subcutaneously transplanted at 1×10⁷ cells into the right side of each female SCID (Severe Combined Immunodeficient) mouse (CLEA Japan, Inc.) (Day 0). Six days later, the mice were divided into groups based on estimated tumor volumes. The Compound A was orally administered by a feed mix containing 0.3% of Compound A at discretion for 28 consecutive days from Day 6 to Day 34. The HER2-ADC (1) was intravenously administered to the tail vein at a dose of 2 mg/kg on Day 10. None of the single and combined administration groups exhibited any particular notable finding such as severe body weight loss. The results of a combination of HER2-ADC (1) and Compound A are shown in FIG. 16. On Day 63, single administration of Compound A showed TGI of 38%, and single administration of HER2-ADC (1) showed TGI of 82%. On the other hand, combined administration of Compound A and HER2-ADC (1) showed TGI of 97%. Compared to HER2-ADC (1) alone and Compound A alone, a combination of HER2-ADC (1) and Compound A exerted a sustained excellent antitumor effect.

### Example 6: Antitumor Study (2)

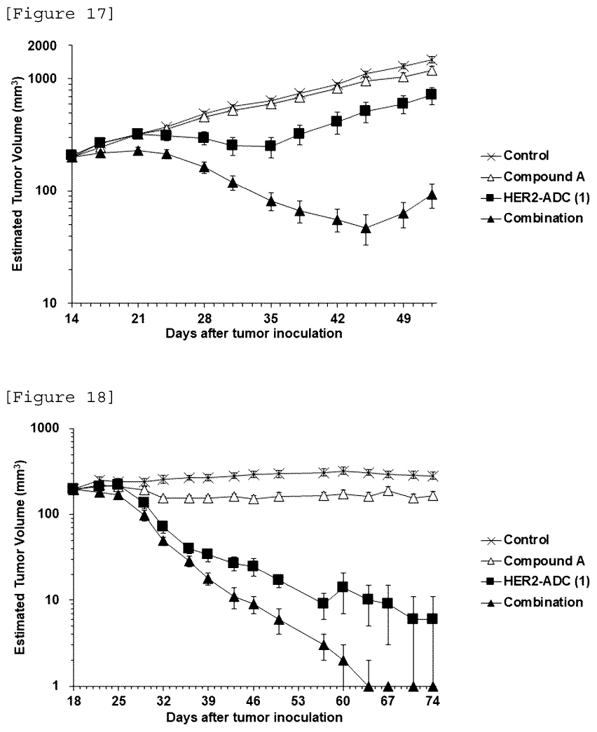

Human breast cancer cell line JIMT-1, which was purchased from DSMZ (Deutsche Sammlung von Mikroorganismen und Zellkulturen GmbH), was suspended into DPBS and subcutaneously transplanted at 5×10⁶ cells into the right side of each female SCID mouse (CLEA Japan, Inc.) (Day 0). Fourteen days later, the mice were divided into groups based on estimated tumor volumes. The Compound A was orally administered by a feed mix containing 0.3% of Compound A at discretion for 28 consecutive days from Day 14 to Day 42. The HER2-ADC (1) was intravenously administered to the tail vein at a dose of 10 mg/kg on Day 18. None of the single and combined administration groups exhibited any particular notable finding such as severe body weight loss. The results of a combination of HER2-ADC (1) and Compound A are shown in FIG. 17. On Day 52, single administration of Compound A showed TGI of 19%, and single administration of HER2-ADC (1) showed TGI of 52%. On the other hand, combined administration of Compound A and HER2-ADC (1) showed TGI of 94%. Compared to HER2-ADC (1) alone and Compound A alone, a combination of HER2-ADC (1) and Compound A exerted a sustained excellent antitumor effect.

### Example 7: Antitumor Study (3)

Human breast cancer cell line MDA-MB-453, which was purchased from ATCC, was suspended into 50% Matrigel matrix and subcutaneously transplanted at 1×10⁷ cells into the right side of each female SCID mouse (CLEA Japan, Inc.) (Day 0). Eighteen days later, the mice were divided into groups based on estimated tumor volumes. The Compound A was orally administered by a feed mix containing 0.3% of Compound A at discretion for 28 consecutive days from Day 18 to Day 46. The HER2-ADC (1) was intravenously administered to the tail vein at a dose of 1 mg/kg on Day 22. None of the single and combined administration groups exhibited any particular notable finding such as severe body weight loss. The results of a combination of HER2-ADC (1) and Compound A are shown in FIG. 18. On Day 74, single administration of Compound A showed TGI of 41%, and single administration of HER2-ADC (1) showed TGI of 98%. On the other hand, combined administration of Compound A and HER2-ADC (1) showed TGI of 100%. Compared to HER2-ADC (1) alone and Compound A alone, a combination of HER2-ADC (1) and Compound A exerted a sustained excellent antitumor effect.

### Example 8: Antitumor Study (4)

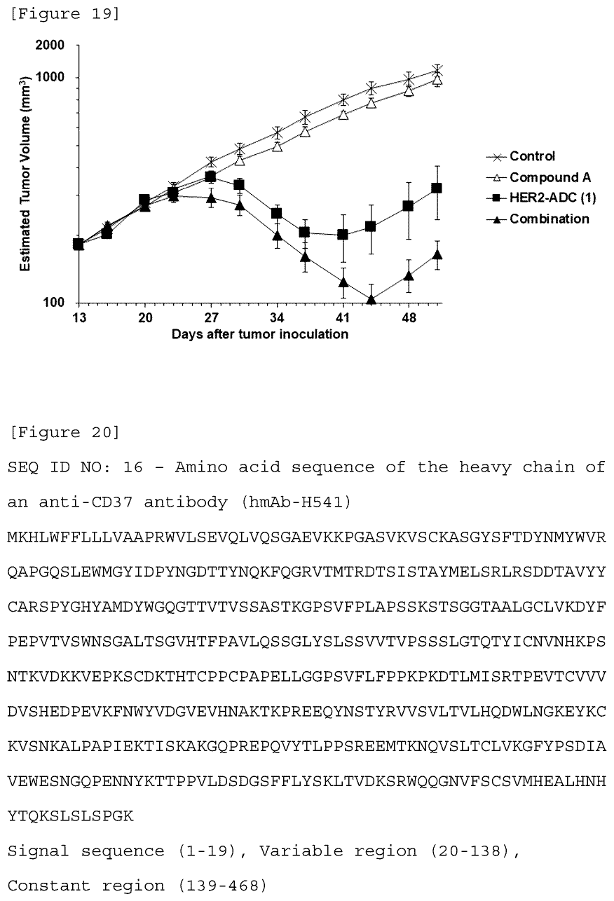

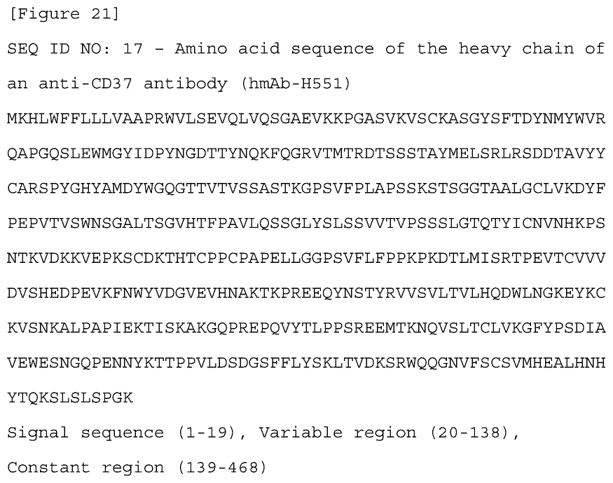

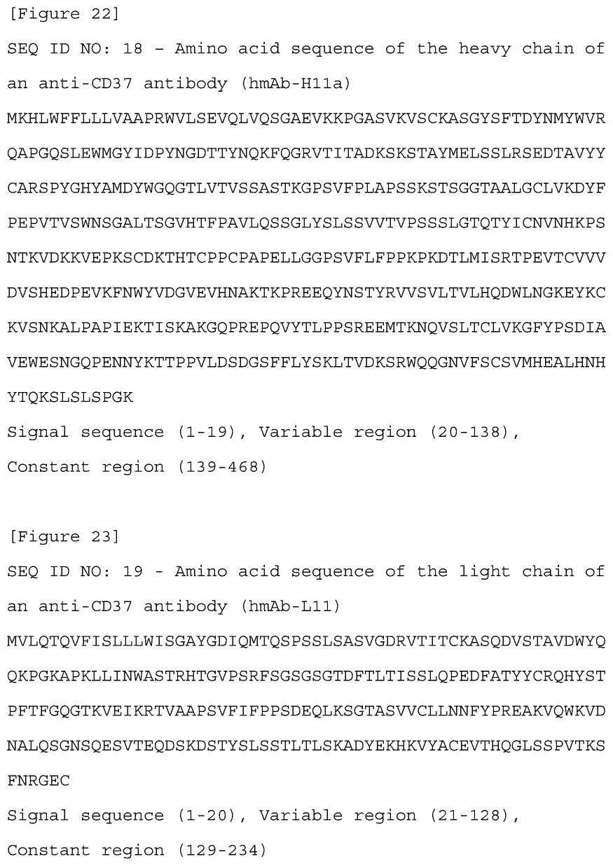

Human breast cancer cell line MX-1, which was purchased from CLS (Cell Lines Service), was suspended into 50% Matrigel matrix and subcutaneously transplanted at 5×10⁶ cells into the right side of each female SCID mouse (CLEA Japan, Inc.) (Day 0). Thirteen days later, the mice were divided into groups based on estimated tumor volumes. The Compound A was orally administered by a feed mix containing 0.3% of Compound A at discretion for 28 consecutive days from Day 13 to Day 41. The HER2-ADC (1) was intravenously administered to the tail vein at a dose of 3 mg/kg on Day 17. None of the single and combined administration groups exhibited any particular notable finding such as severe body weight loss. The results of a combination of HER2-ADC (1) and Compound A are shown in FIG. 19. On Day 51, single administration of Compound A showed TGI of 9%, and single administration of HER2-ADC (1) showed TGI of 70%. On the other hand, combined administration of Compound A and HER2-ADC (1) showed TGI of 85%. Compared to HER2-ADC (1) alone and Compound A alone, a combination of HER2-ADC (1) and Compound A exerted a sustained excellent antitumor effect.

### Example 9: Production of the Anti-CD37 Antibody-Drug Conjugate (1)

In accordance with a similar production method to those described in Examples 1-4 with use of an anti-CD37 antibody (an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19), an anti-CD37 antibody-drug conjugate in which a drug-linker represented by the following formula:

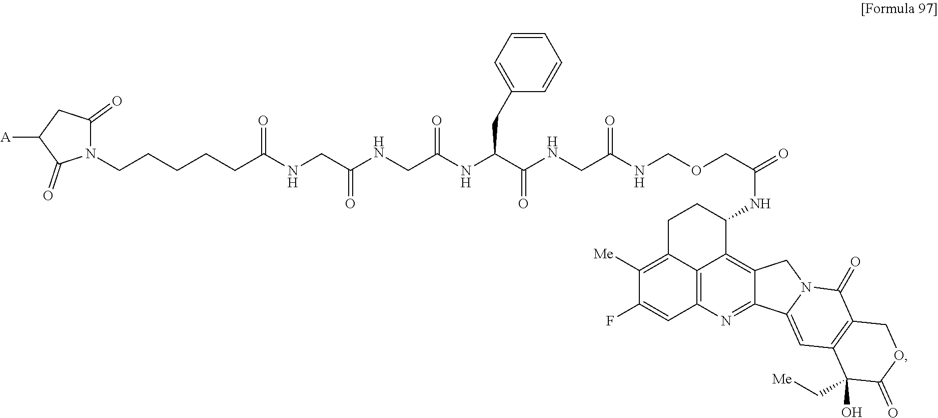

wherein A represents a connecting position to an antibody,
is conjugated to the anti-CD37 antibody via a thioether bond (hereinafter referred to as the “CD37-ADC (1)”) was produced. The DAR of the CD37-ADC (1) is 7.8.

### Example 10: Production of the Anti-B7-H3 Antibody-Drug Conjugate (1)

In accordance with a production method described in International Publication No. WO 2014/057687 with use of an anti-B7-H3 antibody (an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8), an anti-B7-H3 antibody-drug conjugate in which a drug-linker represented by the following formula:

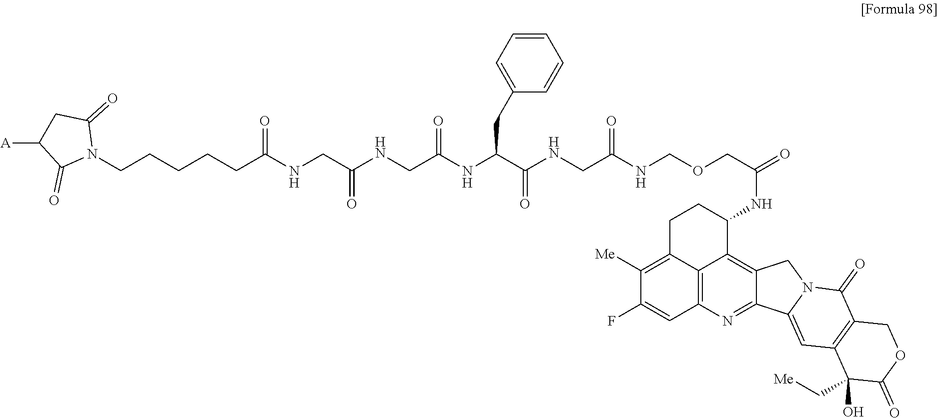

wherein A represents a connecting position to an antibody,
is conjugated to the anti-B7-H3 antibody via a thioether bond (hereinafter referred to as the “B7-H3-ADC (1)”) was produced. The DAR of the B7-H3-ADC (1) is 4.0.

### Example 11: Antitumor Study (5)

Human breast cancer cell line ZR-75-1, which was purchased from ATCC (American Type Culture Collection), was suspended into Matrigel matrix and subcutaneously transplanted at 1×10⁷ cells into the right side of each female NSG mouse (The Jackson Laboratory Japan, Inc.) (Day 0), each of which was treated weekly with 0.02 mg of estradiol cypionate administered subcutaneously from Day 0. Fourteen days later, the mice were divided into groups based on estimated tumor volumes. The Compound A was orally administered by a feed mix containing 0.3% of Compound A at discretion for 28 consecutive days from Day 14 to Day 42. The HER2-ADC (1) was intravenously administered to the tail vein at a dose of 5 mg/kg on Day 14. None of the single and combined administration groups exhibited any particular notable finding such as severe body weight loss. The results of a combination of HER2-ADC (1) and Compound A are shown in FIG. 24. On Day 49, single administration of Compound A showed TGI of 53%, and single administration of HER2-ADC (1) showed TGI of 61%. On the other hand, combined administration of Compound A and HER2-ADC (1) showed TGI of 76%. Compared to HER2-ADC (1) alone and Compound A alone, a combination of HER2-ADC (1) and Compound A exerted a sustained excellent antitumor effect.

### Example 12: Antitumor Study (6)

Human non-small lung cancer cell line Calu-3, which was purchased from ATCC (American Type Culture Collection), was suspended into Matrigel matrix and subcutaneously transplanted at 3×10⁶ cells into the right side of each female SCID mouse (The Jackson Laboratory Japan, Inc.) (Day 0). Fourteen days later, the mice were divided into groups based on estimated tumor volumes. The Compound A was orally administered by a feed mix containing 0.3% of Compound A at discretion for 28 consecutive days from Day 14 to Day 42. The HER2-ADC (1) was intravenously administered to the tail vein at a dose of 1 mg/kg on Day 14. None of the single and combined administration groups exhibited any particular notable finding such as severe body weight loss. The results of a combination of HER2-ADC (1) and Compound A are shown in FIG. 25. On Day 35, single administration of Compound A showed TGI of 20%, and single administration of HER2-ADC (1) showed TGI of 76%. On the other hand, combined administration of Compound A and HER2-ADC (1) showed TGI of 95%. Compared to HER2-ADC (1) alone and Compound A alone, a combination of HER2-ADC (1) and Compound A exerted a sustained excellent antitumor effect.

### Example 13: Antitumor Study (7)

Human non-small lung cancer cell line NCI-H358, which was purchased from ATCC (American Type Culture Collection), was suspended into 50% Matrigel matrix and subcutaneously transplanted at 3×10⁶ cells into the right side of each female SCID mouse (CLEA Japan, Inc.) (Day 0). Twenty-four days later, the mice were divided into groups based on estimated tumor volumes. The Compound A was orally administered by a feed mix containing 0.25% of Compound A at discretion for 42 consecutive days from Day 24 to Day 66. The TROP2-ADC (1) was intravenously administered to the tail vein at a dose of 10 mg/kg on Day 24 and Day 45. None of the single and combined administration groups exhibited any particular notable finding such as severe body weight loss. The results of a combination of TROP2-ADC (1) and Compound A are shown in FIG. 26. On Day 115, single administration of Compound A showed TGI of 20%, and single administration of TROP2-ADC (1) showed TGI of 78%. On the other hand, combined administration of Compound A and TROP2-ADC (1) showed TGI of 93%. Compared to TROP2-ADC (1) alone and Compound A alone, a combination of TROP2-ADC (1) and Compound A exerted a sustained excellent antitumor effect.

### Example 14: Antitumor Study (8)

Human breast cancer cell line JIMT-1, which was purchased from DSMZ (Deutsche Sammlung von Mikroorganismen und Zellkulturen GmbH), was suspended into saline and subcutaneously transplanted at 5×10⁶ cells into the right side of each female SCID mouse (CLEA Japan, Inc.) (Day 0). Eleven days later, the mice were divided into groups based on estimated tumor volumes. The Compound A was orally administered by a feed mix containing 0.25% of Compound A at discretion for 28 consecutive days from Day 11 to Day 39. The HER3-ADC (1) was intravenously administered to the tail vein at a dose of 10 mg/kg on Day 11. None of the single and combined administration groups exhibited any particular notable finding such as severe body weight loss. The results of a combination of HER3-ADC (1) and Compound A are shown in FIG. 27. On Day 46, single administration of Compound A showed TGI of 41%, and single administration of HER3-ADC (1) showed TGI of 66%. On the other hand, combined administration of Compound A and HER3-ADC (1) showed TGI of 77%. Compared to HER3-ADC (1) alone and Compound A alone, a combination of HER3-ADC (1) and Compound A exerted a sustained excellent antitumor effect.

### Example 15: Antitumor Study (9)

Human diffuse large B-cell lymphoma cell line WSU-DLCL2, which was purchased from DSMZ (Deutsche Sammlung von Mikroorganismen und Zellkulturen GmbH), was suspended into DPBS and subcutaneously transplanted at 1×10⁷ cells into the right side of each female SCID mouse (CLEA Japan, Inc.) (Day 0). Sixteen days later, the mice were divided into groups based on estimated tumor volumes. The Compound A was orally administered by a feed mix containing 0.25% of Compound A at discretion for 28 consecutive days from Day 16 to Day 44. The CD37-ADC (1) was intravenously administered to the tail vein at a dose of 1 mg/kg on Day 16. None of the single and combined administration groups exhibited any particular notable finding such as severe body weight loss. The results of a combination of CD37-ADC (1) and Compound A are shown in FIG. 28. On Day 44, single administration of Compound A showed TGI of 31%, and single administration of CD37-ADC (1) showed TGI of 43%. On the other hand, combined administration of Compound A and CD37-ADC (1) showed TGI of 78%. Compared to CD37-ADC (1) alone and Compound A alone, a combination of CD37-ADC (1) and Compound A exerted a sustained excellent antitumor effect.

### Example 16: Antitumor Study (10)

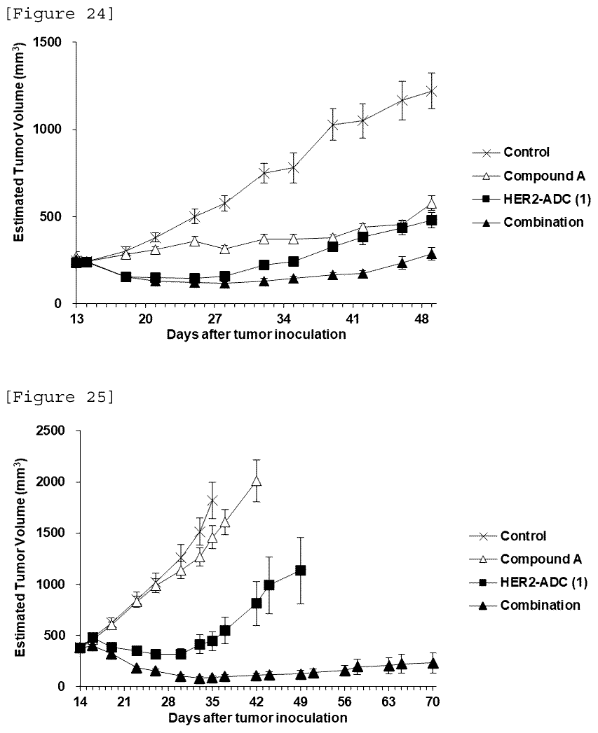

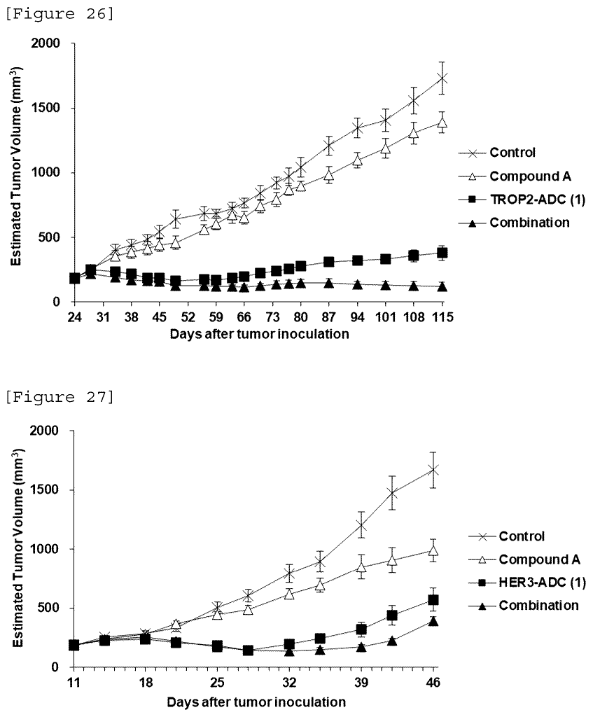

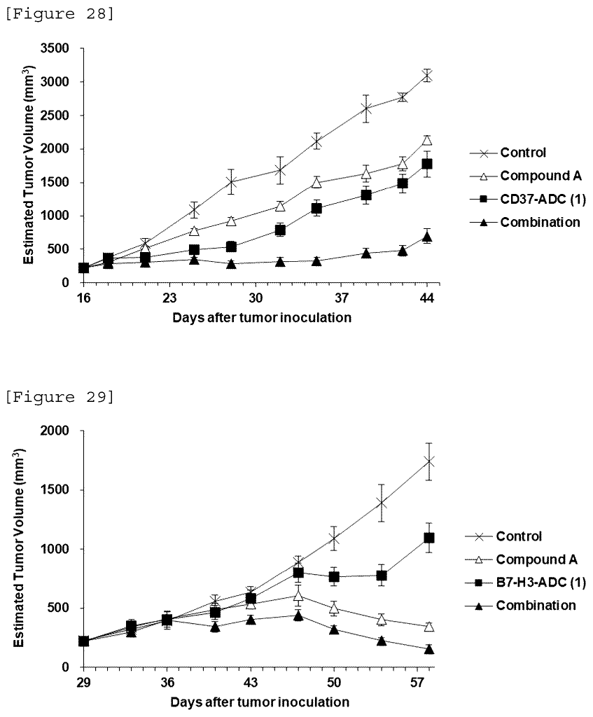

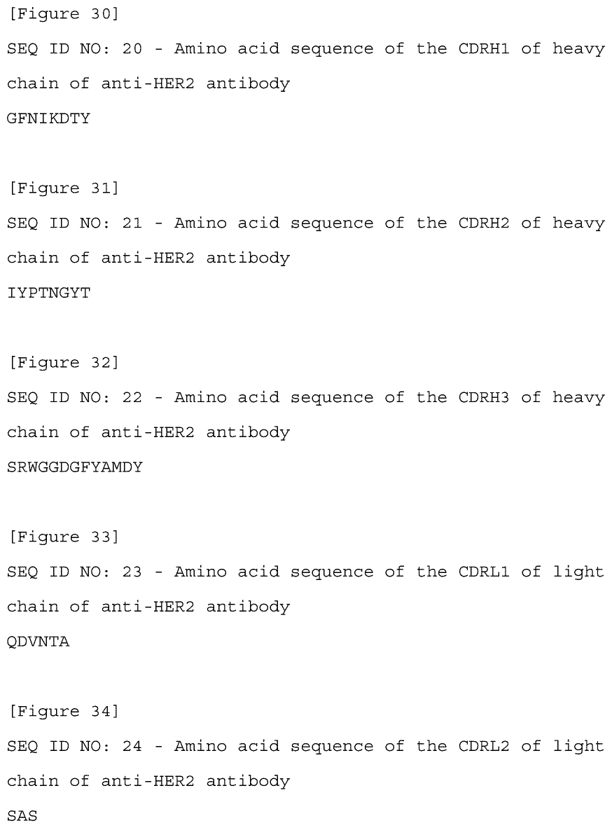

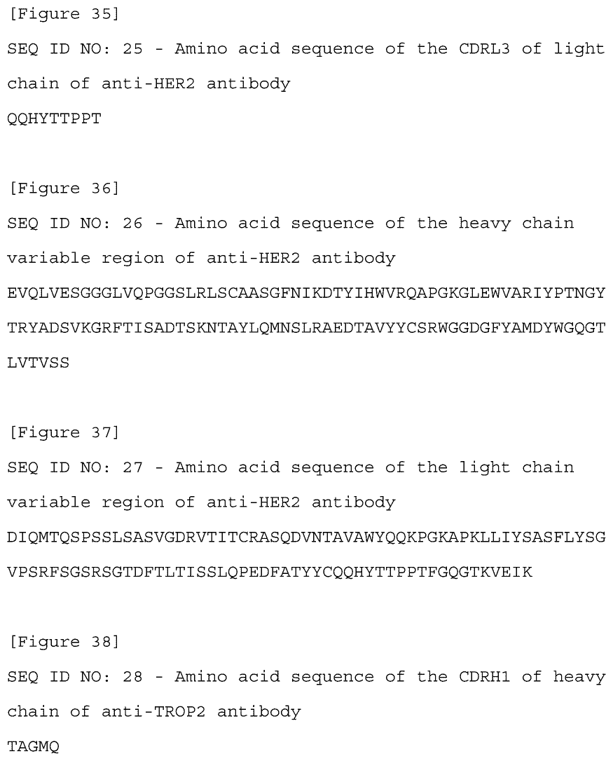

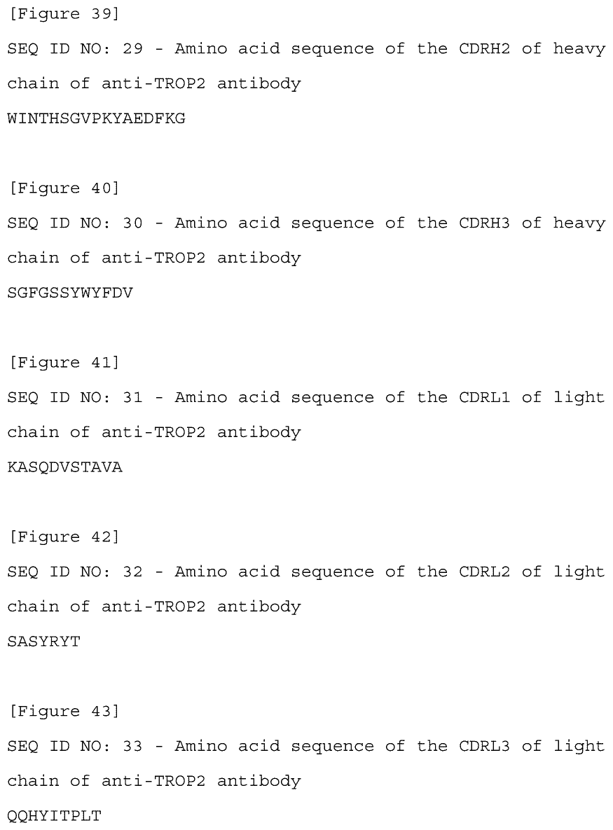

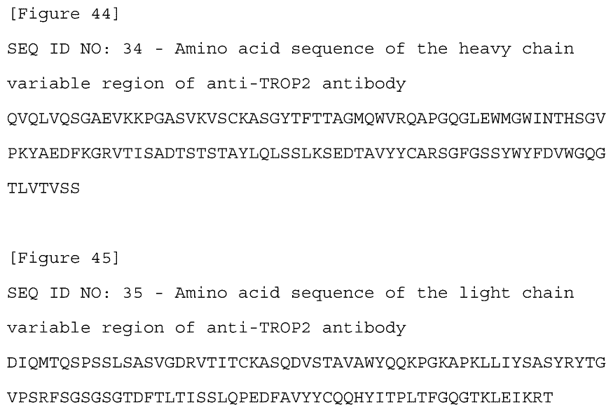

Human neuroendocrine prostate cancer cell line NCI-H660, which was purchased from ATCC (American Type Culture Collection), was suspended into 50% Matrigel matrix and subcutaneously transplanted at 2×10⁶ cells into the right side of each castrated male SCID mouse (CLEA Japan, Inc.) (Day 0). Twenty-nine days later, the mice were divided into groups based on estimated tumor volumes. The Compound A was orally administered by a feed mix containing 0.25% of Compound A at discretion for 29 consecutive days from Day 29 to Day 58. The B7-H3-ADC (1) was intravenously administered to the tail vein at a dose of 0.3 mg/kg on Day 29 and Day 43. None of the single and combined administration groups exhibited any particular notable finding such as severe body weight loss. The results of a combination of B7-H3-ADC (1) and Compound A are shown in FIG. 29. On Day 58, single administration of Compound A showed TGI of 80%, and single administration of B7-H3-ADC (1) showed TGI of 37%. On the other hand, combined administration of Compound A and B7-H3-ADC (1) showed TGI of 91%. Compared to B7-H3-ADC (1) alone and Compound A alone, a combination of B7-H3-ADC (1) and Compound A exerted a sustained excellent antitumor effect.

### Free Text of Sequence Listing

SEQ ID NO: 1—Amino acid sequence of the heavy chain of an anti-HER2 antibody
SEQ ID NO: 2—Amino acid sequence of the light chain of an anti-HER2 antibody
SEQ ID NO: 3—Amino acid sequence of the heavy chain of an anti-HER3 antibody
SEQ ID NO: 4—Amino acid sequence of the light chain of an anti-HER3 antibody
SEQ ID NO: 5—Amino acid sequence of the heavy chain of an anti-TROP2 antibody
SEQ ID NO: 6—Amino acid sequence of the light chain of an anti-TROP2 antibody
SEQ ID NO: 7—Amino acid sequence of the heavy chain of an anti-B7-H3 antibody
SEQ ID NO: 8—Amino acid sequence of the light chain of an anti-B7-H3 antibody
SEQ ID NO: 9—Amino acid sequence of the heavy chain of an anti-GPR20 antibody
SEQ ID NO: 10—Amino acid sequence of the light chain of an anti-GPR20 antibody
SEQ ID NO: 11—Amino acid sequence of the heavy chain of an anti-CDH6 antibody
SEQ ID NO: 12—Amino acid sequence of the light chain of an anti-CDH6 antibody
SEQ ID NO: 13—Amino acid sequence of the heavy chain of an anti-MUC1 antibody (N54Q)
SEQ ID NO: 14—Amino acid sequence of the heavy chain of an anti-MUC1 antibody (PankoMab)
SEQ ID NO: 15—Amino acid sequence of the light chain of an anti-MUC1 antibody (N54Q and PankoMab)
SEQ ID NO: 16—Amino acid sequence of the heavy chain of an anti-CD37 antibody (hmAb-H541)
SEQ ID NO: 17—Amino acid sequence of the heavy chain of an anti-CD37 antibody (hmAb-H551)
SEQ ID NO: 18—Amino acid sequence of the heavy chain of an anti-CD37 antibody (hmAb-H11a)
SEQ ID NO: 19—Amino acid sequence of the light chain of the anti-CD37 antibodies (hmAb-L11)
SEQ ID NO: 20—Amino acid sequence of the CDRH1 of heavy chain of anti-HER2 antibody
SEQ ID NO: 21—Amino acid sequence of the CDRH2 of heavy chain of anti-HER2 antibody
SEQ ID NO: 22—Amino acid sequence of the CDRH3 of heavy chain of anti-HER2 antibody
SEQ ID NO: 23—Amino acid sequence of the CDRL1 of light chain of anti-HER2 antibody
SEQ ID NO: 24—Amino acid sequence of the CDRL2 of light chain of anti-HER2 antibody
SEQ ID NO: 25—Amino acid sequence of the CDRL3 of light chain of anti-HER2 antibody
SEQ ID NO: 26—Amino acid sequence of the heavy chain variable region of anti-HER2 antibody
SEQ ID NO: 27—Amino acid sequence of the light chain variable region of anti-HER2 antibody
SEQ ID NO: 28—Amino acid sequence of the CDRH1 of heavy chain of anti-TROP2 antibody
SEQ ID NO: 29—Amino acid sequence of the CDRH2 of heavy chain of anti-TROP2 antibody
SEQ ID NO: 30—Amino acid sequence of the CDRH3 of heavy chain of anti-TROP2 antibody
SEQ ID NO: 31—Amino acid sequence of the CDRL1 of light chain of anti-TROP2 antibody
SEQ ID NO: 32—Amino acid sequence of the CDRL2 of light chain of anti-TROP2 antibody
SEQ ID NO: 33—Amino acid sequence of the CDRL3 of light chain of anti-TROP2 antibody
SEQ ID NO: 34—Amino acid sequence of the heavy chain variable region of anti-TROP2 antibody
SEQ ID NO: 35—Amino acid sequence of the light chain variable region of anti-TROP2 antibody

1. A pharmaceutical product comprising an antibody-drug conjugate and an inhibitor selected from the group consisting of an EZH1 inhibitor, an EZH2 inhibitor and an EZH1/2 dual inhibitor for administration in combination, wherein
the antibody-drug conjugate is an antibody-drug conjugate in which a drug-linker represented by the following formula: 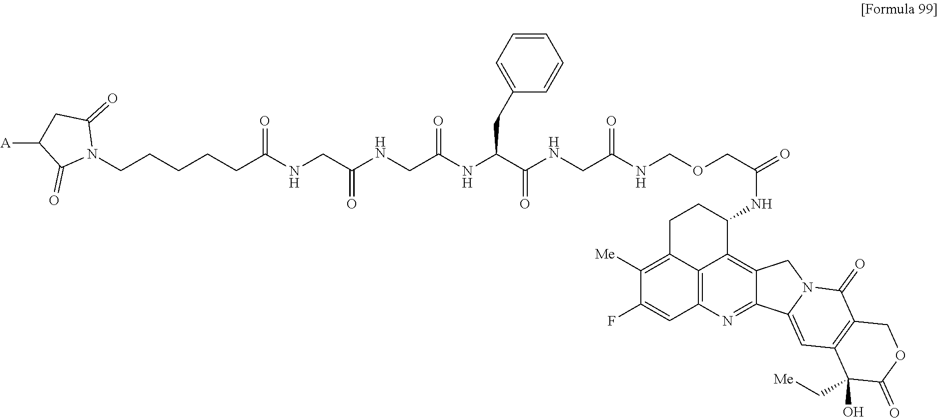
wherein A represents a connecting position to an antibody,
is conjugated to the antibody via a thioether bond.

2. The pharmaceutical product according to claim 1, wherein the antibody in the antibody-drug conjugate is an anti-HER2 antibody, an anti-HER3 antibody, an anti-TROP2 antibody, an anti-B7-H3 antibody, an anti-GPR20 antibody, an anti-CDH6 antibody, an anti-MUC1 antibody, or an anti-CD37 antibody.

3. The pharmaceutical product according to claim 2, wherein the antibody in the antibody-drug conjugate is an anti-HER2 antibody.

4. The pharmaceutical product according to claim 3, wherein the anti-HER2 antibody is an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1, and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2, and CDRL3 consisting of an amino acid sequence consisting of amino acid residues 89 to 97 of SEQ ID NO: 2.

5. The pharmaceutical product according to claim 3, wherein the anti-HER2 antibody is an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2.

6. The pharmaceutical product according to claim 3, wherein the anti-HER2 antibody is an antibody comprising a heavy chain consisting of an amino acid sequence represented by SEQ ID NO: 1 and a light chain consisting of an amino acid sequence represented by SEQ ID NO: 2.

7. The pharmaceutical product according to claim 3, wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

8. The pharmaceutical product according to any one of claims 3 to 7, wherein the antibody-drug conjugate is represented by the following formula: 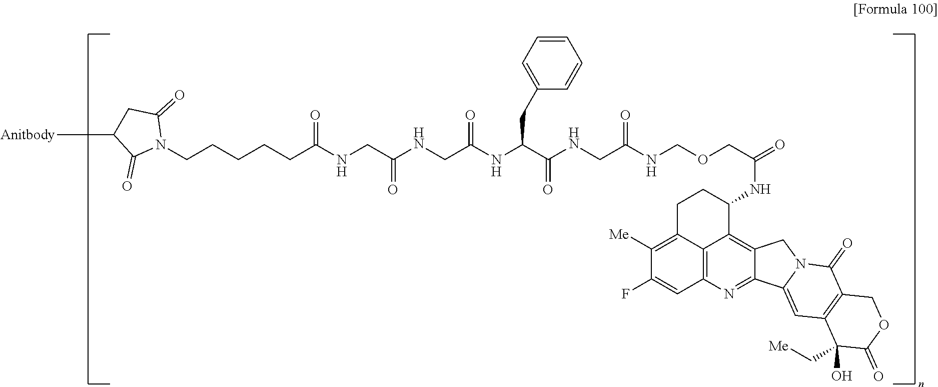
wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8.

9. The pharmaceutical product according to claim 2, wherein the antibody in the antibody-drug conjugate is an anti-HER3 antibody.

10. The pharmaceutical product according to claim 9, wherein the anti-HER3 antibody is an antibody comprising a heavy chain comprising CDRH1 consisting of an amino acid sequence consisting of amino acid residues 26 to 35 of SEQ ID NO: 3, CDRH2 consisting of an amino acid sequence consisting of amino acid residues 50 to 65 of SEQ ID NO: 3, and CDRH3 consisting of an amino acid sequence consisting of amino acid residues 98 to 106 of SEQ ID NO: 3, and a light chain comprising CDRL1 consisting of an amino acid sequence consisting of amino acid residues 24 to 39 of SEQ ID NO: 4, CDRL2 consisting of an amino acid sequence consisting of amino acid residues 56 to 62 of SEQ ID NO: 4, and CDRL3 consisting of an amino acid sequence consisting of amino acid residues 95 to 103 of SEQ ID NO: 4.

11. The pharmaceutical product according to claim 9, wherein the anti-HER3 antibody is an antibody comprising a heavy chain comprising a heavy chain variable region consisting of an amino acid sequence consisting of amino acid residues 1 to 117 of SEQ ID NO: 3, and a light chain comprising a light chain variable region consisting of an amino acid sequence consisting of amino acid residues 1 to 113 of SEQ ID NO: 4.

12. The pharmaceutical product according to claim 9, wherein the anti-HER3 antibody is an antibody comprising a heavy chain consisting of an amino acid sequence represented by SEQ ID NO: 3 and a light chain consisting of an amino acid sequence represented by SEQ ID NO: 4.

13. The pharmaceutical product according to claim 12, wherein the anti-HER3 antibody lacks a lysine residue at the carboxyl terminus of the heavy chain.

14. The pharmaceutical product according to any one of claims 9 to 13, wherein the antibody-drug conjugate is represented by the following formula: 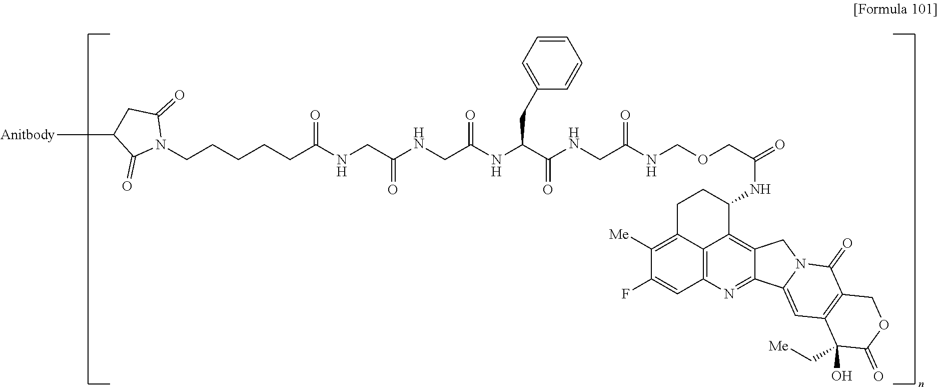
wherein ‘Antibody’ indicates the anti-HER3 antibody conjugated to the drug-linker via a thioether bond, and n indicates an average number of units of the drug-linker conjugated per antibody molecule in the antibody-drug conjugate, wherein n is in the range of from 7 to 8.

15. The pharmaceutical product according to claim 2, wherein the antibody in the antibody-drug conjugate is an anti-TROP2 antibody.

16. The pharmaceutical product according to claim 15, wherein the anti-TROP2 antibody is an antibody comprising a heavy chain comprising CDRH1 consisting of an amino acid sequence consisting of amino acid residues 50 to 54 of SEQ ID NO: 5, CDRH2 consisting of an amino acid sequence consisting of amino acid residues 69 to 85 of SEQ ID NO: 5, and CDRH3 consisting of an amino acid sequence consisting of amino acid residues 118 to 129 of SEQ ID NO: 5, and a light chain comprising CDRL1 consisting of an amino acid sequence consisting of amino acid residues 44 to 54 of SEQ ID NO: 6, CDRL2 consisting of an amino acid sequence consisting of amino acid residues 70 to 76 of SEQ ID NO: 6, and CDRL3 consisting of an amino acid sequence consisting of amino acid residues 109 to 117 of SEQ ID NO: 6.

17. The pharmaceutical product according to claim 15, wherein the anti-TROP2 antibody is an antibody comprising a heavy chain comprising a heavy chain variable region consisting of an amino acid sequence consisting of amino acid residues 20 to 140 of SEQ ID NO: 5, and a light chain comprising a light chain variable region consisting of an amino acid sequence consisting of amino acid residues 21 to 129 of SEQ ID NO: 6.

18. The pharmaceutical product according to claim 15, wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

19. The pharmaceutical product according to claim 18, wherein the anti-TROP2 antibody lacks a lysine residue at the carboxyl terminus of the heavy chain.

20. The pharmaceutical product according to any one of claims 15 to 19, wherein the antibody-drug conjugate is represented by the following formula: 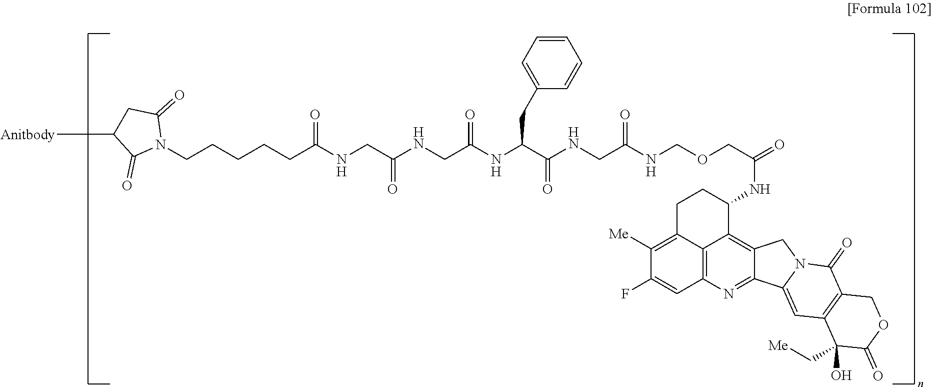
wherein ‘Antibody’ indicates the anti-TROP2 antibody conjugated to the drug-linker via a thioether bond, and n indicates an average number of units of the drug-linker conjugated per antibody molecule in the antibody-drug conjugate, wherein n is in the range of from 3.5 to 4.5.

21. The pharmaceutical product according to claim 2, wherein the antibody in the antibody-drug conjugate is an anti-B7-H3 antibody.

22. The pharmaceutical product according to claim 21, wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

23. The pharmaceutical product according to claim 22, wherein the anti-B7-H3 antibody lacks a lysine residue at the carboxyl terminus of the heavy chain.

24. The pharmaceutical product according to any one of claims 21 to 23, wherein the antibody-drug conjugate is represented by the following formula: 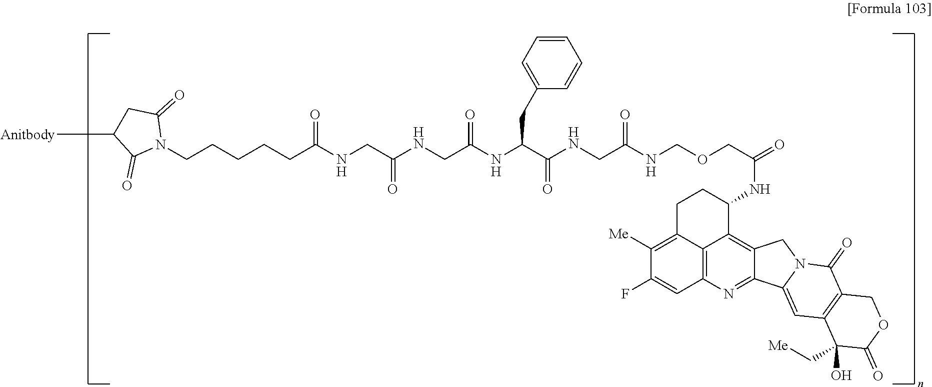
wherein ‘Antibody’ indicates the anti-B7-H3 antibody conjugated to the drug-linker via a thioether bond, and n indicates an average number of units of the drug-linker conjugated per antibody molecule in the antibody-drug conjugate, wherein n is in the range of from 3.5 to 4.5.

25. The pharmaceutical product according to claim 2, wherein the antibody in the antibody-drug conjugate is an anti-GPR20 antibody.

26. The pharmaceutical product according to claim 25, wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

27. The pharmaceutical product according to claim 26, wherein the anti-GPR20 antibody lacks a lysine residue at the carboxyl terminus of the heavy chain.

28. The pharmaceutical product according to any one of claims 25 to 27, wherein the antibody-drug conjugate is represented by the following formula: 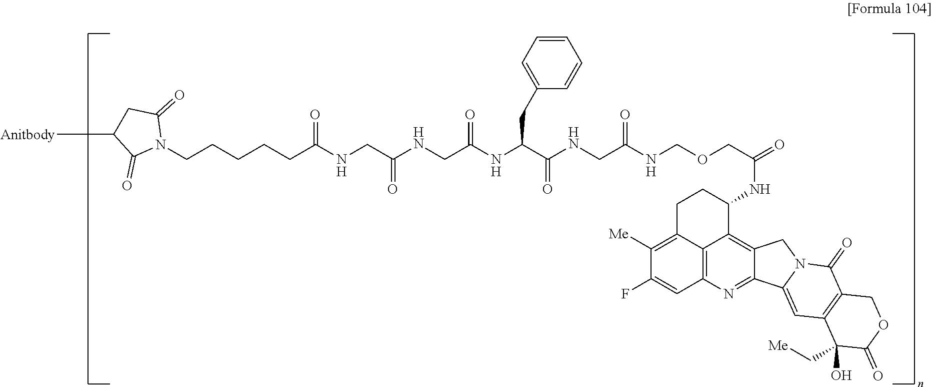
wherein ‘Antibody’ indicates the anti-GPR20 antibody conjugated to the drug-linker via a thioether bond, and n indicates an average number of units of the drug-linker conjugated per antibody molecule in the antibody-drug conjugate, wherein n is in the range of from 7 to 8.

29. The pharmaceutical product according to claim 2, wherein the antibody in the antibody-drug conjugate is an anti-CDH6 antibody.

30. The pharmaceutical product according to claim 29, wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

31. The pharmaceutical product according to claim 30, wherein the anti-CDH6 antibody lacks a lysine residue at the carboxyl terminus of the heavy chain.

32. The pharmaceutical product according to any one of claims 29 to 31, wherein the antibody-drug conjugate is represented by the following formula: 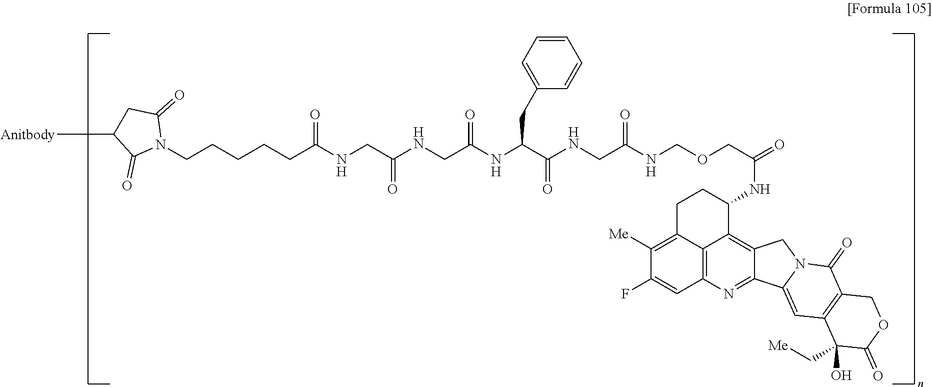
wherein ‘Antibody’ indicates the anti-CDH6 antibody conjugated to the drug-linker via a thioether bond, and n indicates an average number of units of the drug-linker conjugated per antibody molecule in the antibody-drug conjugate, wherein n is in the range of from 7 to 8.

33. The pharmaceutical product according to claim 2, wherein the antibody in the antibody-drug conjugate is an anti-MUC1 antibody.

34. The pharmaceutical product according to claim 33, wherein the anti-MUC1 antibody is an antibody comprising a heavy chain consisting of an amino acid sequence represented by SEQ ID NO: 13 and a light chain consisting of an amino acid sequence represented by SEQ ID NO: 15.

35. The pharmaceutical product according to claim 34, wherein the anti-MUC1 antibody lacks a lysine residue at the carboxyl terminus of the heavy chain.

36. The pharmaceutical product according to claim 33, wherein the anti-MUC1 antibody is an antibody comprising a heavy chain consisting of an amino acid sequence represented by SEQ ID NO: 14 and a light chain consisting of an amino acid sequence represented by SEQ ID NO: 15.

37. The pharmaceutical product according to claim 36, wherein the anti-MUC1 antibody lacks a lysine residue at the carboxyl terminus of the heavy chain.

38. The pharmaceutical product according to any one of claims 33 to 37, wherein the antibody-drug conjugate is represented by the following formula: 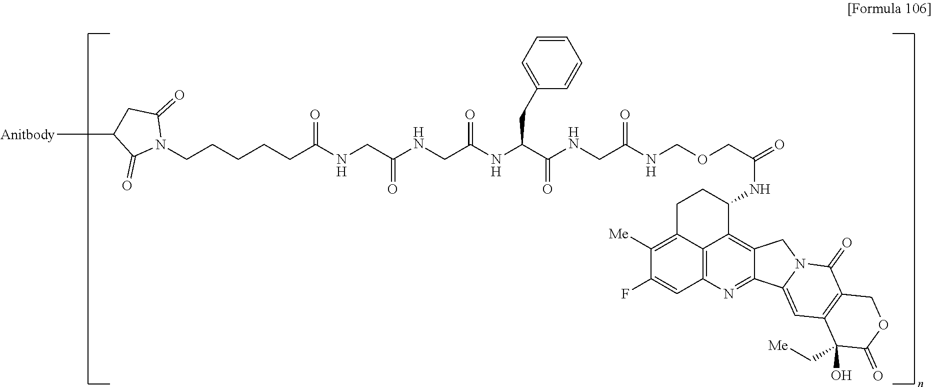
wherein ‘Antibody’ indicates the anti-MUC1 antibody conjugated to the drug-linker via a thioether bond, and n indicates an average number of units of the drug-linker conjugated per antibody molecule in the antibody-drug conjugate, wherein n is in the range of from 7 to 8.

39. The pharmaceutical product according to claim 2, wherein the antibody in the antibody-drug conjugate is an anti-CD37 antibody.

40. The pharmaceutical product according to claim 39, wherein the anti-CD37 antibody is an antibody selected from the group consisting of
(a) an antibody comprising a heavy chain comprising a heavy chain variable region consisting of an amino acid sequence consisting of amino acid residues 20 to 138 of SEQ ID NO: 16 and a light chain comprising a light chain variable region consisting of an amino acid sequence consisting of amino acid residues 21 to 128 of SEQ ID NO: 19,
(b) an antibody comprising a heavy chain comprising a heavy chain variable region consisting of an amino acid sequence consisting of amino acid residues 20 to 138 of SEQ ID NO: 17 and a light chain comprising a light chain variable region consisting of an amino acid sequence consisting of amino acid residues 21 to 128 of SEQ ID NO: 19, and
(c) an antibody comprising a heavy chain comprising a heavy chain variable region consisting of an amino acid sequence consisting of amino acid residues 20 to 138 of SEQ ID NO: 18 and a light chain comprising a light chain variable region consisting of an amino acid sequence consisting of amino acid residues 21 to 128 of SEQ ID NO: 19.

41. The pharmaceutical product according to claim 39, wherein the anti-CD37 antibody is an antibody selected from the group consisting of
(d) an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19,
(e) an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19, and
(f) an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

42. The pharmaceutical product according to claim 39, wherein the anti-CD37 antibody is an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19.

43. The pharmaceutical product according to claim 42, wherein the anti-CD37 antibody lacks one or two amino acid residues at the carboxyl terminus of the heavy chain.

44. The pharmaceutical product according to claim 42 or claim 43, wherein a proline residue at the carboxyl terminus of the heavy chain is amidated.

45. The pharmaceutical product according to claim 39, wherein the anti-CD37 antibody is an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19.

46. The pharmaceutical product according to claim 45, wherein the anti-CD37 antibody lacks one or two amino acid residues at the carboxyl terminus of the heavy chain.

47. The pharmaceutical product according to claim 45 or claim 46, wherein a proline residue at the carboxyl terminus of the heavy chain is amidated.

48. The pharmaceutical product according to claim 39, wherein the anti-CD37 antibody is an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

49. The pharmaceutical product according to claim 48, wherein the anti-CD37 antibody lacks one or two amino acid residues at the carboxyl terminus of the heavy chain.

50. The pharmaceutical product according to claim 48 or claim 49, wherein a proline residue at the carboxyl terminus of the heavy chain is amidated.

51. The pharmaceutical product according to any one of claims 39 to 50, wherein the antibody-drug conjugate is represented by the following formula: 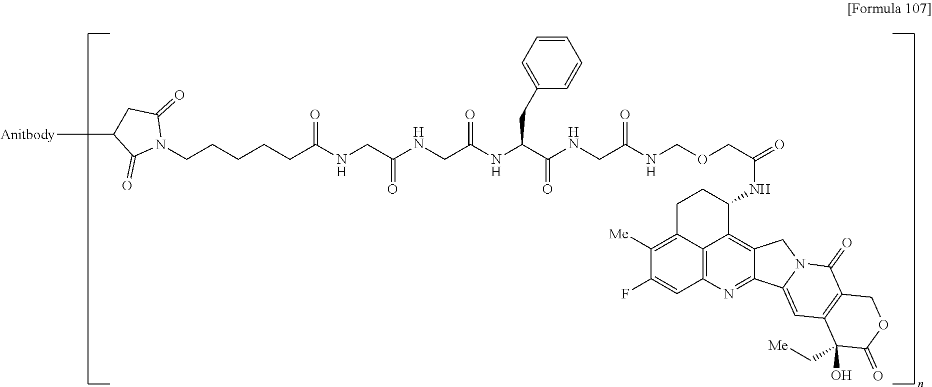
wherein ‘Antibody’ indicates the anti-CD37 antibody conjugated to the drug-linker via a thioether bond, and n indicates an average number of units of the drug-linker conjugated per antibody molecule in the antibody-drug conjugate, wherein n is in the range of from 7 to 8.

52. The pharmaceutical product according to any one of claims 1 to 8, wherein the antibody-drug conjugate is trastuzumab deruxtecan (DS-8201a).

53. The pharmaceutical product according to any one of claims 1, 2 and 15 to 20, wherein the antibody-drug conjugate is datopotamab deruxtecan (DS-1062).

54. The pharmaceutical product according to any one of claims 1 to 53, wherein the inhibitor is an EZH2 inhibitor.

55. The pharmaceutical product according to claim 54, wherein the inhibitor is tazemetostat or a pharmaceutically acceptable salt thereof.

56. The pharmaceutical product according to any one of claims 1 to 53, wherein the inhibitor is an EZH1/2 dual inhibitor.

57. The pharmaceutical product according to claim 56, wherein the inhibitor is valemetostat or a pharmaceutically acceptable salt thereof.

58. The pharmaceutical product according to claim 56 or claim 57, wherein the inhibitor is valemetostat tosylate.

59. The pharmaceutical product according to any one of claims 1 to 58, wherein the antibody-drug conjugate and the inhibitor are separately contained as active components in different formulations, and are administered simultaneously or at different times.

60. The pharmaceutical product according to any one of claims 1 to 59, wherein the pharmaceutical product is for use in treating at least one selected from the group consisting of breast cancer, gastric cancer, colorectal cancer, lung cancer, esophageal cancer, head-and-neck cancer, gastroesophageal junction adenocarcinoma, biliary tract cancer, Paget's disease, pancreatic cancer, ovarian cancer, bladder cancer, prostate cancer, uterine carcinosarcoma, gastrointestinal stromal tumor, kidney cancer, and sarcoma.

61. The pharmaceutical product according to claim 60, wherein the pharmaceutical product is for use in treating breast cancer.

62. The pharmaceutical product according to claim 61, wherein the pharmaceutical product is for use in treating triple-negative breast cancer.

63. The pharmaceutical product according to claim 60, wherein the pharmaceutical product is for use in treating gastric cancer.

64. The pharmaceutical product according to claim 60, wherein the pharmaceutical product is for use in treating ovarian cancer.

65. The pharmaceutical product according to claim 60, wherein the pharmaceutical product is for use in treating lung cancer.

66. The pharmaceutical product according to claim 60, wherein the pharmaceutical product is for use in treating pancreatic cancer.

67. An antibody-drug conjugate for use in a method of treating a disease, wherein the method comprises administering the antibody-drug conjugate in combination with an inhibitor selected from the group consisting of an EZH1 inhibitor, an EZH2 inhibitor and an EZH1/2 dual inhibitor, wherein the antibody-drug conjugate is an antibody-drug conjugate in which a drug-linker represented by the following formula: 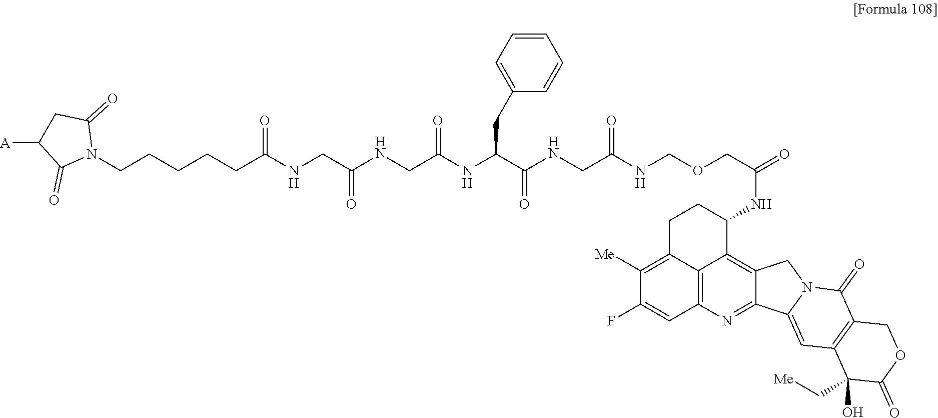
wherein A represents a connecting position to an antibody,
is conjugated to the antibody via a thioether bond in the antibody-drug conjugate.

68. The antibody-drug conjugate for use according to claim 67, wherein the antibody in the antibody-drug conjugate is an anti-HER2 antibody, an anti-HER3 antibody, an anti-TROP2 antibody, an anti-B7-H3 antibody, an anti-GPR20 antibody, an anti-CDH6 antibody, an anti-MUC1 antibody, or an anti-CD37 antibody.

69. The antibody-drug conjugate for use according to claim 68, wherein the antibody in the antibody-drug conjugate is an anti-HER2 antibody.

70. The antibody-drug conjugate for use according to claim 69, wherein the anti-HER2 antibody is an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1, and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2, and CDRL3 consisting of an amino acid sequence consisting of amino acid residues 89 to 97 of SEQ ID NO: 2.

71. The antibody-drug conjugate for use according to claim 69, wherein the anti-HER2 antibody is an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2.

72. The antibody-drug conjugate for use according to claim 69, wherein the anti-HER2 antibody is an antibody comprising a heavy chain consisting of an amino acid sequence represented by SEQ ID NO: 1 and a light chain consisting of an amino acid sequence represented by SEQ ID NO: 2.

73. The antibody-drug conjugate for use according to claim 69, wherein the anti-HER2 antibody is an antibody comprising a heavy chain consisting of an amino acid sequence consisting of amino acid residues 1 to 449 of SEQ ID NO: 1 and a light chain consisting of an amino acid sequence consisting of amino acid residues 1 to 214 of SEQ ID NO: 2.

74. The antibody-drug conjugate for use according to any one of claims 69 to 73,
wherein the antibody-drug conjugate is represented by the following formula: 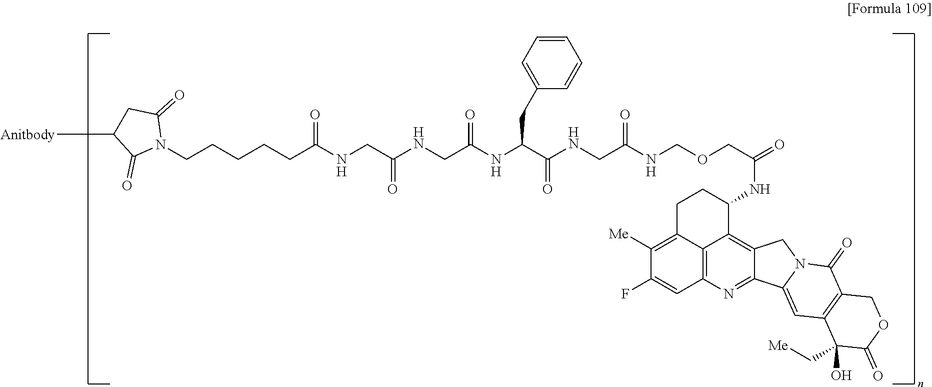
wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8.

75. The antibody-drug conjugate for use according to claim 68, wherein the antibody in the antibody-drug conjugate is an anti-HER3 antibody.

76. The antibody-drug conjugate for use according to claim 75, wherein the anti-HER3 antibody is an antibody comprising a heavy chain comprising CDRH1 consisting of an amino acid sequence consisting of amino acid residues 26 to 35 of SEQ ID NO: 3, CDRH2 consisting of an amino acid sequence consisting of amino acid residues 50 to 65 of SEQ ID NO: 3, and CDRH3 consisting of an amino acid sequence consisting of amino acid residues 98 to 106 of SEQ ID NO: 3, and a light chain comprising CDRL1 consisting of an amino acid sequence consisting of amino acid residues 24 to 39 of SEQ ID NO: 4, CDRL2 consisting of an amino acid sequence consisting of amino acid residues 56 to 62 of SEQ ID NO: 4, and CDRL3 consisting of an amino acid sequence consisting of amino acid residues 95 to 103 of SEQ ID NO: 4.

77. The antibody-drug conjugate for use according to claim 75, wherein the anti-HER3 antibody is an antibody comprising a heavy chain comprising a heavy chain variable region consisting of an amino acid sequence consisting of amino acid residues 1 to 117 of SEQ ID NO: 3, and a light chain comprising a light chain variable region consisting of an amino acid sequence consisting of amino acid residues 1 to 113 of SEQ ID NO: 4.

78. The antibody-drug conjugate for use according to claim 75, wherein the anti-HER3 antibody is an antibody comprising a heavy chain consisting of an amino acid sequence represented by SEQ ID NO: 3 and a light chain consisting of an amino acid sequence represented by SEQ ID NO: 4.

79. The antibody-drug conjugate for use according to claim 78, wherein the anti-HER3 antibody lacks a lysine residue at the carboxyl terminus of the heavy chain.

80. The antibody-drug conjugate for use according to any one of claims 75 to 79, wherein the antibody-drug conjugate is represented by the following formula: 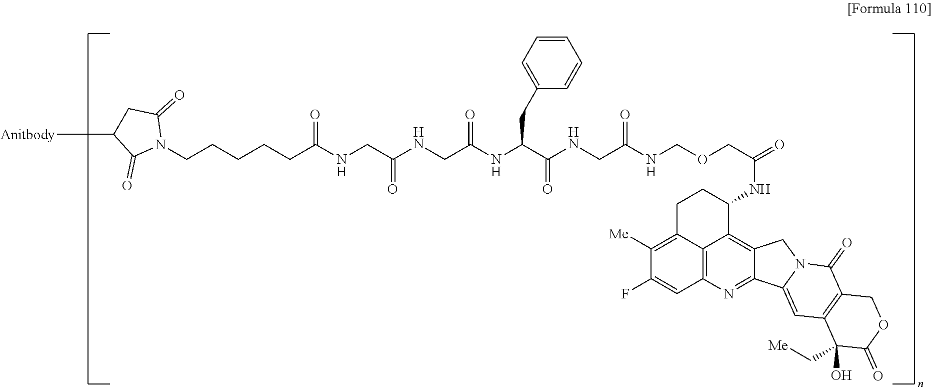
wherein ‘Antibody’ indicates the anti-HER3 antibody conjugated to the drug-linker via a thioether bond, and n indicates an average number of units of the drug-linker conjugated per antibody molecule in the antibody-drug conjugate, wherein n is in the range of from 7 to 8.

81. The antibody-drug conjugate for use according to claim 68, wherein the antibody in the antibody-drug conjugate is an anti-TROP2 antibody.

82. The antibody-drug conjugate for use according to claim 81, wherein the anti-TROP2 antibody is an antibody comprising a heavy chain comprising CDRH1 consisting of an amino acid sequence consisting of amino acid residues 50 to 54 of SEQ ID NO: 5, CDRH2 consisting of an amino acid sequence consisting of amino acid residues 69 to 85 of SEQ ID NO: 5, and CDRH3 consisting of an amino acid sequence consisting of amino acid residues 118 to 129 of SEQ ID NO: 5, and a light chain comprising CDRL1 consisting of an amino acid sequence consisting of amino acid residues 44 to 54 of SEQ ID NO: 6, CDRL2 consisting of an amino acid sequence consisting of amino acid residues 70 to 76 of SEQ ID NO: 6, and CDRL3 consisting of an amino acid sequence consisting of amino acid residues 109 to 117 of SEQ ID NO: 6.

83. The antibody-drug conjugate for use according to claim 81, wherein the anti-TROP2 antibody is an antibody comprising a heavy chain comprising a heavy chain variable region consisting of an amino acid sequence consisting of amino acid residues 20 to 140 of SEQ ID NO: 5, and a light chain comprising a light chain variable region consisting of an amino acid sequence consisting of amino acid residues 21 to 129 of SEQ ID NO: 6.

84. The antibody-drug conjugate for use according to claim 81, wherein the anti-TROP2 antibody is an antibody comprising a heavy chain consisting of an amino acid sequence consisting of amino acid residues 20 to 470 of SEQ ID NO: 5 and a light chain consisting of an amino acid sequence consisting of amino acid residues 21 to 234 of SEQ ID NO: 6.

85. The antibody-drug conjugate for use according to claim 84, wherein the anti-TROP2 antibody lacks a lysine residue at the carboxyl terminus of the heavy chain.

86. The antibody-drug conjugate for use according to any one of claims 81 to 85, wherein the antibody-drug conjugate is represented by the following formula: 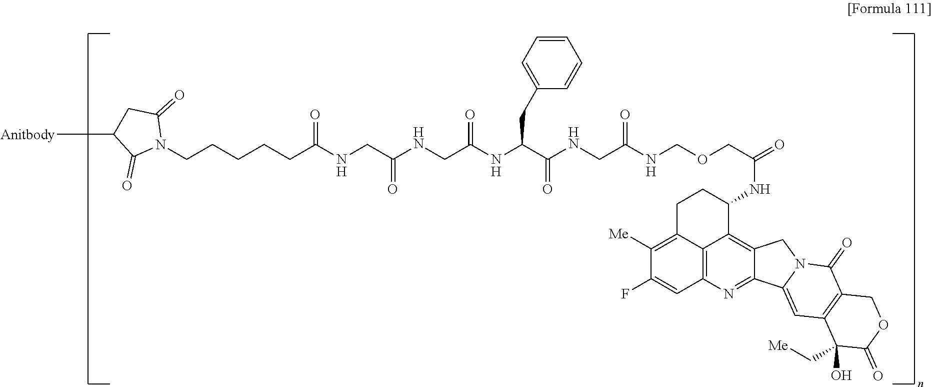
wherein ‘Antibody’ indicates the anti-TROP2 antibody conjugated to the drug-linker via a thioether bond, and n indicates an average number of units of the drug-linker conjugated per antibody molecule in the antibody-drug conjugate, wherein n is in the range of from 3.5 to 4.5.

87. The antibody-drug conjugate for use according to claim 68, wherein the antibody in the antibody-drug conjugate is an anti-B7-H3 antibody.

88. The antibody-drug conjugate for use according to claim 87, wherein the anti-B7-H3 antibody is an antibody comprising a heavy chain consisting of an amino acid sequence consisting of amino acid residues 20 to 471 of SEQ ID NO: 7 and a light chain consisting of an amino acid sequence consisting of amino acid residues 21 to 233 of SEQ ID NO: 8.

89. The antibody-drug conjugate for use according to claim 88, wherein the anti-B7-H3 antibody lacks a lysine residue at the carboxyl terminus of the heavy chain.

90. The antibody-drug conjugate for use according to any one of claims 87 to 89, wherein the antibody-drug conjugate is represented by the following formula: 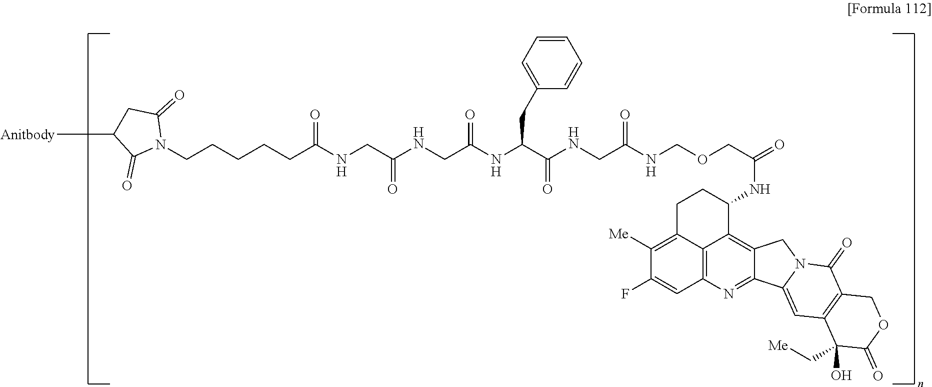
wherein ‘Antibody’ indicates the anti-B7-H3 antibody conjugated to the drug-linker via a thioether bond, and n indicates an average number of units of the drug-linker conjugated per antibody molecule in the antibody-drug conjugate, wherein n is in the range of from 3.5 to 4.5.

91. The antibody-drug conjugate for use according to claim 68, wherein the antibody in the antibody-drug conjugate is an anti-GPR20 antibody.

92. The antibody-drug conjugate for use according to claim 91, wherein the anti-GPR20 antibody is an antibody comprising a heavy chain consisting of an amino acid sequence consisting of amino acid residues 20 to 472 of SEQ ID NO: 9 and a light chain consisting of an amino acid sequence consisting of amino acid residues 21 to 234 of SEQ ID NO: 10.

93. The antibody-drug conjugate for use according to claim 92, wherein the anti-GPR20 antibody lacks a lysine residue at the carboxyl terminus of the heavy chain.

94. The antibody-drug conjugate for use according to any one of claims 91 to 93, wherein the antibody-drug conjugate is represented by the following formula: 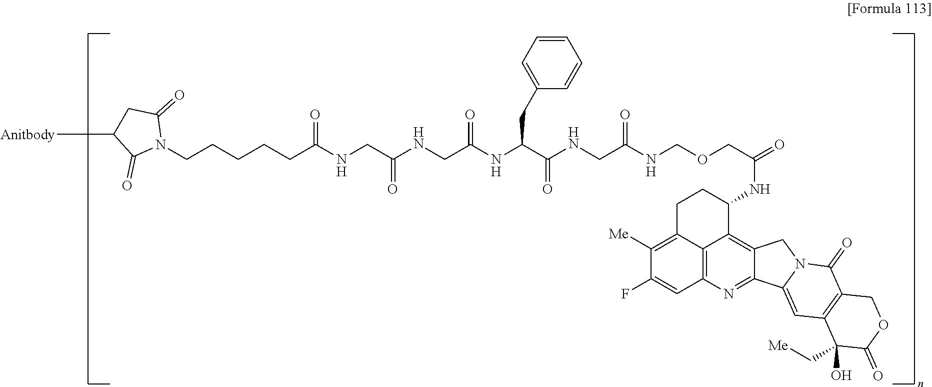
wherein ‘Antibody’ indicates the anti-GPR20 antibody conjugated to the drug-linker via a thioether bond, and n indicates an average number of units of the drug-linker conjugated per antibody molecule in the antibody-drug conjugate, wherein n is in the range of from 7 to 8.

95. The antibody-drug conjugate for use according to claim 68, wherein the antibody in the antibody-drug conjugate is an anti-CDH6 antibody.

96. The antibody-drug conjugate for use according to claim 95, wherein the anti-CDH6 antibody is an antibody comprising a heavy chain consisting of an amino acid sequence consisting of amino acid residues 20 to 471 of SEQ ID NO: 11 and a light chain consisting of an amino acid sequence consisting of amino acid residues 21 to 233 of SEQ ID NO: 12.

97. The antibody-drug conjugate for use according to claim 96, wherein the anti-CDH6 antibody lacks a lysine residue at the carboxyl terminus of the heavy chain.

98. The antibody-drug conjugate for use according to any one of claims 95 to 97, wherein the antibody-drug conjugate is represented by the following formula: 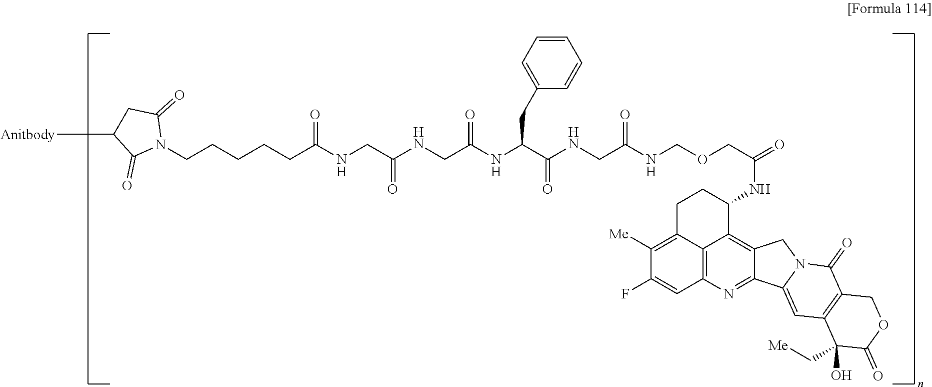
wherein ‘Antibody’ indicates the anti-CDH6 antibody conjugated to the drug-linker via a thioether bond, and n indicates an average number of units of the drug-linker conjugated per antibody molecule in the antibody-drug conjugate, wherein n is in the range of from 7 to 8.

99. The antibody-drug conjugate for use according to claim 68, wherein the antibody in the antibody-drug conjugate is an anti-MUC1 antibody.

100. The antibody-drug conjugate for use according to claim 99, wherein the anti-MUC1 antibody is an antibody comprising a heavy chain consisting of an amino acid sequence represented by SEQ ID NO: 13 and a light chain consisting of an amino acid sequence represented by SEQ ID NO: 15.

101. The antibody-drug conjugate for use according to claim 100, wherein the anti-MUC1 antibody lacks a lysine residue at the carboxyl terminus of the heavy chain.

102. The antibody-drug conjugate for use according to claim 99, wherein the anti-MUC1 antibody is an antibody comprising a heavy chain consisting of an amino acid sequence represented by SEQ ID NO: 14 and a light chain consisting of an amino acid sequence represented by SEQ ID NO: 15.

103. The antibody-drug conjugate for use according to claim 102, wherein the anti-MUC1 antibody lacks a lysine residue at the carboxyl terminus of the heavy chain.

104. The antibody-drug conjugate for use according to any one of claims 99 to 103, wherein the antibody-drug conjugate is represented by the following formula: 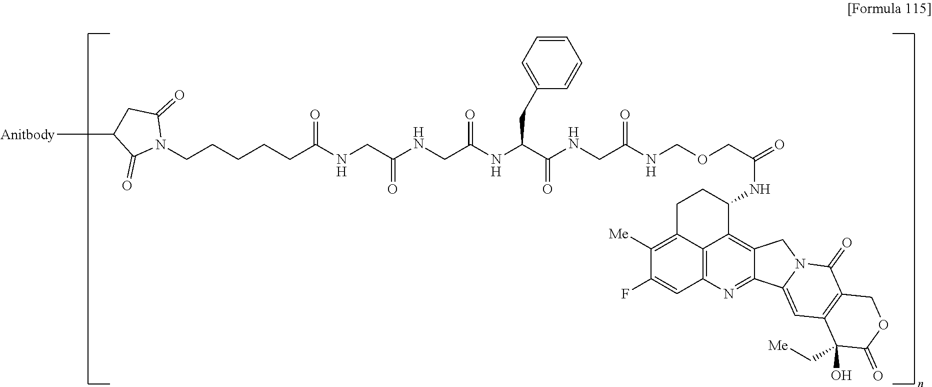
wherein ‘Antibody’ indicates the anti-MUC1 antibody conjugated to the drug-linker via a thioether bond, and n indicates an average number of units of the drug-linker conjugated per antibody molecule in the antibody-drug conjugate, wherein n is in the range of from 7 to 8.

105. The antibody-drug conjugate for use according to claim 68, wherein the antibody in the antibody-drug conjugate is an anti-CD37 antibody.

106. The antibody-drug conjugate for use according to claim 105, wherein the anti-CD37 antibody is an antibody selected from the group consisting of
(a) an antibody comprising a heavy chain comprising a heavy chain variable region consisting of an amino acid sequence consisting of amino acid residues 20 to 138 of SEQ ID NO: 16 and a light chain comprising a light chain variable region consisting of an amino acid sequence consisting of amino acid residues 21 to 128 of SEQ ID NO: 19,
(b) an antibody comprising a heavy chain comprising a heavy chain variable region consisting of an amino acid sequence consisting of amino acid residues 20 to 138 of SEQ ID NO: 17 and a light chain comprising a light chain variable region consisting of an amino acid sequence consisting of amino acid residues 21 to 128 of SEQ ID NO: 19, and
(c) an antibody comprising a heavy chain comprising a heavy chain variable region consisting of an amino acid sequence consisting of amino acid residues 20 to 138 of SEQ ID NO: 18 and a light chain comprising a light chain variable region consisting of an amino acid sequence consisting of amino acid residues 21 to 128 of SEQ ID NO: 19.

107. The antibody-drug conjugate for use according to claim 105, wherein the anti-CD37 antibody is an antibody selected from the group consisting of
(d) an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19,
(e) an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19, and
(f) an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

108. The antibody-drug conjugate for use according to claim 105, wherein the anti-CD37 antibody is an antibody comprising a heavy chain consisting of an amino acid sequence consisting of amino acid residues 20 to 468 of SEQ ID NO: 16 and a light chain consisting of an amino acid sequence consisting of amino acid residues 21 to 234 of SEQ ID NO: 19.

109. The antibody-drug conjugate for use according to claim 108, wherein the anti-CD37 antibody lacks one or two amino acid residues at the carboxyl terminus of the heavy chain.

110. The antibody-drug conjugate for use according to claim 108 or claim 109, wherein a proline residue at the carboxyl terminus of the heavy chain is amidated.

111. The antibody-drug conjugate for use according to claim 105, wherein the anti-CD37 antibody is an antibody comprising a heavy chain consisting of an amino acid sequence consisting of amino acid residues 20 to 468 of SEQ ID NO: 17 and a light chain consisting of an amino acid sequence consisting of amino acid residues 21 to 234 of SEQ ID NO: 19.

112. The antibody-drug conjugate for use according to claim 111, wherein the anti-CD37 antibody lacks one or two amino acid residues at the carboxyl terminus of the heavy chain.

113. The antibody-drug conjugate for use according to claim 111 or claim 112, wherein a proline residue at the carboxyl terminus of the heavy chain is amidated.

114. The antibody-drug conjugate for use according to claim 105, wherein the anti-CD37 antibody is an antibody comprising a heavy chain consisting of an amino acid sequence consisting of amino acid residues 20 to 468 of SEQ ID NO: 18 and a light chain consisting of an amino acid sequence consisting of amino acid residues 21 to 234 of SEQ ID NO: 19.

115. The antibody-drug conjugate for use according to claim 114, wherein the anti-CD37 antibody lacks one or two amino acid residues at the carboxyl terminus of the heavy chain.

116. The antibody-drug conjugate for use according to claim 114 or claim 115, wherein a proline residue at the carboxyl terminus of the heavy chain is amidated.

117. The antibody-drug conjugate for use according to any one of claims 105 to 116, wherein the antibody-drug conjugate is represented by the following formula: 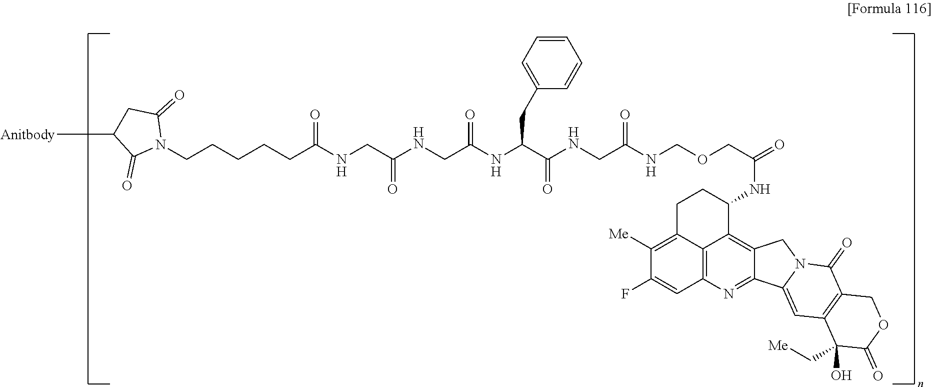
wherein ‘Antibody’ indicates the anti-CD37 antibody conjugated to the drug-linker via a thioether bond, and n indicates an average number of units of the drug-linker conjugated per antibody molecule in the antibody-drug conjugate, wherein n is in the range of from 7 to 8.

118. The antibody-drug conjugate for use according to any one of claims 67 to 74, wherein the antibody-drug conjugate is trastuzumab deruxtecan (DS-8201a).

119. The antibody-drug conjugate for use according to any one of claims 67 to 68 and 81 to 86, wherein the antibody-drug conjugate is datopotamab deruxtecan (DS-1062).

120. The antibody-drug conjugate for use according to any one of claims 67 to 119, wherein the inhibitor is an EZH2 inhibitor.

121. The antibody-drug conjugate for use according to claim 120, wherein the inhibitor is tazemetostat or a pharmaceutically acceptable salt thereof.

122. The antibody-drug conjugate for use according to any one of claims 67 to 119, wherein the inhibitor is an EZH1/2 dual inhibitor.

123. The antibody-drug conjugate for use according to claim 122, wherein the inhibitor is valemetostat or a pharmaceutically acceptable salt thereof.

124. The antibody-drug conjugate for use according to claim 122 or claim 123, wherein the inhibitor is valemetostat tosylate.

125. The antibody-drug conjugate for use according to any one of claims 67 to 124, wherein the antibody-drug conjugate and the inhibitor are separately contained as active components in different formulations, and are administered simultaneously or at different times.

126. The antibody-drug conjugate for use according to any one of claims 67 to 125, wherein the disease is at least one selected from the group consisting of breast cancer, gastric cancer, colorectal cancer, lung cancer, esophageal cancer, head-and-neck cancer, gastroesophageal junction adenocarcinoma, biliary tract cancer, Paget's disease, pancreatic cancer, ovarian cancer, bladder cancer, prostate cancer, uterine carcinosarcoma, gastrointestinal stromal tumor, kidney cancer, and sarcoma.

127. The antibody-drug conjugate for use according to claim 126, wherein the disease is breast cancer.

128. The antibody-drug conjugate for use according to claim 127, wherein the disease is triple-negative breast cancer.

129. The antibody-drug conjugate for use according to claim 126, wherein the disease is gastric cancer.

130. The antibody-drug conjugate for use according to claim 126, wherein the disease is ovarian cancer.

131. The antibody-drug conjugate for use according to claim 126, wherein the disease is lung cancer.

132. The antibody-drug conjugate for use according to claim 126, wherein the disease is pancreatic cancer.
